(19) United States
(12) Patent Application Publication
(10) Pub. No.: US 2026/0022141 A1
(43) Pub. Date: Jan. 22, 2026

(54) SIV ENVELOPE TRIMER

(71) Applicant: The Scripps Research Institute, La Jolla, CA (US)

(72) Inventors: Raiees ANDRABI, La Jolla, CA (US); Dennis BURTON, La Jolla, CA (US)

(21) Appl. No.: 19/277,534

(22) Filed: Jul. 23, 2025

Related U.S. Application Data
(63) Continuation of application No. 17/231,415, filed on Apr. 15, 2021, now Pat. No. 12,398,177.
Application No. 17/231,415 is a continuation-in-part of application No. PCT/US2019/056867, filed on Oct. 18, 2019.
(60) Provisional application No. 62/747,650, filed on Oct. 18, 2018.

(51) Int. Cl. C07K 5/08; A61K 9/127; A61K 9/51; A61K 39/00; A61K 39/21; A61K 39/39; A61K 47/08; A61K 47/10; A61P 31/18; C12N 7/00; C12N 15/86; G01N 33/569

(52) U.S. Cl. CPC C07K 5/08; A61K 9/127; A61K 9/51; A61K 39/21; A61K 39/39; A61K 47/08; A61K 47/10; A61P 31/18; C12N 7/00; C12N 15/86; G01N 33/56988; A61K 2039/545; A61K 2039/55505; A61K 2039/55555; A61K 2039/55566; C12N 2740/15022; C12N 2740/15034; C12N 2750/14143

(57) ABSTRACT

The present application relates to epitope-targeted SIV and HIV vaccines. The invention provides novel envelope glycoproteins which may be utilized as HIV-1 vaccine immunogens, antigens for crystallization, and for identification of broadly neutralizing antibodies. The invention encompasses preparation and purification of immunogenic compositions which are formulated into vaccines of the present invention.

### RELATED APPLICATIONS AND INCORPORATION BY REFERENCE

This application is a continuation of U.S. application Ser. No. 17/231,415 filed Apr. 15, 2021 (now allowed) which is a continuation-in-part of international patent application Serial No. PCT/US2019/056867 filed Oct. 18, 2019, which published as PCT Publication No. WO 2020/081895 on Apr. 23, 2020, and which claims priority to U.S. provisional application Ser. No. 62/747,650, filed on Oct. 18, 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under grant number AI100663 awarded by the National Institutes of Health. The government has certain rights in the invention.

### SEQUENCE STATEMENT

The instant application contains a Sequence Listing which has been submitted electronically and is hereby incorporated by reference in its entirety. Said XML copy, was created on Jul. 17, 2025 amended on Oct. 16, 2025, is named ReplacementY7969_04051SL.xml and is 427,095 bytes in size.

### FIELD OF THE INVENTION

The present invention relates to epitope-targeted SIV and HIV vaccines. The invention relates to epitopes that elicit antibodies that are broadly neutralizing against HIV. The invention provides epitopes that efficiently stimulate germline antibody production. The invention provides novel envelope glycoproteins which may be utilized as HIV-1 vaccine immunogens, antigens for crystallization, and for identification of broadly neutralizing antibodies. The invention encompasses preparation and purification of immunogenic compositions which are formulated into vaccines of the present invention.

### BACKGROUND OF THE INVENTION

AIDS, or Acquired Immunodeficiency Syndrome, is caused by human immunodeficiency virus (HIV) and is characterized by several clinical features including wasting syndromes, central nervous system degeneration and profound immunosuppression that results in opportunistic infections and malignancies. HIV is a member of the lentivirus family of animal retroviruses, which include the visna virus of sheep and the bovine, feline, and simian immunodeficiency viruses (SIV). Two closely related types of HIV, designated HIV-I and HIV-2, have been identified thus far, of which HIV-I is by far the most common cause of AIDS. However, HIV-2, which differs in genomic structure and antigenicity, causes a similar clinical syndrome.

An infectious HIV particle consists of two identical strands of RNA, each approximately 9.2 kb long, packaged within a core of viral proteins. This core structure is surrounded by a phospholipid bilayer envelope derived from the host cell membrane that also includes virally-encoded membrane proteins (Abbas et al., Cellular and Molecular Immunology, 4th edition, W.B. Saunders Company, 2000, p. 454). The HIV genome has the characteristic 5′-LTR-Gag-Pol-Env-LTR-3′ organization of the retrovirus family. Long terminal repeats (LTRs) at each end of the viral genome serve as binding sites for transcriptional regulatory proteins from the host and regulate viral integration into the host genome, viral gene expression, and viral replication.

The HIV genome encodes several structural proteins. The gag gene encodes structural proteins of the nucleocapsid core and matrix. The pol gene encodes reverse transcriptase (RT), integrase (IN), and viral protease (PR) enzymes required for viral replication. The tat gene encodes a protein that is required for elongation of viral transcripts. The rev gene encodes a protein that promotes the nuclear export of incompletely spliced or unspliced viral RNAs. The vif gene product enhances the infectivity of viral particles. The vpr gene product promotes the nuclear import of viral DNA and regulates G2 cell cycle arrest. The vpu and nef genes encode proteins that down regulate host cell CD4 expression and enhance release of virus from infected cells. The env gene encodes the viral envelope glycoprotein that is translated as a 160-kilodalton (kDa) precursor (gp160) and cleaved by a cellular protease to yield the external 120-kDa envelope glycoprotein (gp120) and the transmembrane 41-kDa envelope glycoprotein (gp41), which are required for the infection of cells (Abbas et al., Cellular and Molecular Immunology, 4th edition, W.B. Saunders Company, 2000, pp. 454-456). gp140 is a modified form of the Env glycoprotein, which contains the external 120-kDa envelope glycoprotein portion and the extracellular part of the gp41 portion of Env and has characteristics of both gp120 and gp41. The nef gene is conserved among primate lentiviruses and is one of the first viral genes that is transcribed following infection. In vitro, several functions have been described, including down-regulation of CD4 and MHC class I surface expression, altered T-cell signaling and activation, and enhanced viral infectivity.

HIV infection initiates with gp120 on the viral particle binding to the CD4 and chemokine receptor molecules (e.g., CXCR4, CCR5) on the cell membrane of target cells such as CD4+ T-cells, macrophages and dendritic cells. The bound virus fuses with the target cell and reverse transcribes the RNA genome. The resulting viral DNA integrates into the cellular genome, where it directs the production of new viral RNA, and thereby viral proteins and new virions. These virions bud from the infected cell membrane and establish productive infections in other cells. This process also kills the originally infected cell. HIV can also kill cells indirectly because the CD4 receptor on uninfected T-cells has a strong affinity for gp120 expressed on the surface of infected cells. In this case, the uninfected cells bind, via the CD4 receptor-gp120 interaction, to infected cells and fuse to form a syncytium, which cannot survive. Destruction of CD4+ T-lymphocytes, which are critical to immune defense, is a major cause of the progressive immune dysfunction that is the hallmark of AIDS disease progression. The loss of CD4+ T cells seriously impairs the body's ability to fight most invaders, but it has a particularly severe impact on the defenses against viruses, fungi, parasites and certain bacteria, including mycobacteria.

Research on the Env glycoprotein has shown that the virus has many effective protective mechanisms with few vulnerabilities (Wyatt & Sodroski, Science. 1998 Jun. 1 9; 280(5371):1884-8). For fusion with its target cells, HIV-I uses a trimeric Env complex containing gp120 and gp41 subunits (Burton et al., Nat Immunol. 2004 March; 5(3):233-6). The fusion potential of the Env complex is triggered by engagement of the CD4 receptor and a coreceptor, usually CCR5 or CXCR4. Neutralizing antibodies seem to work either by binding to the mature trimer on the virion surface and preventing initial receptor engagement events, or by binding after virion attachment and inhibiting the fusion process (Parren & Burton, Adv Immunol. 200 I; 77:195-262). In the latter case, neutralizing antibodies may bind to epitopes whose exposure is enhanced or triggered by receptor binding. However, given the potential antiviral effects of neutralizing antibodies, it is not unexpected that HIV-I has evolved multiple mechanisms to protect it from antibody binding (Johnson & Desrosiers, Annu Rev Med. 2002; 53:499-518).

Most experimental HIV-I vaccines tested in human and/or non-human primate suggests that a successful vaccine incorporate immunogens that elicit broad neutralizing antibodies (bnAbs) and robust cell-mediated immunity. HIV-I envelope glycoprotein (Env) is the main viral protein involved in the entry of the virus and is also the primary target for neutralizing antibodies, but due to immune evasion strategies and extreme sequence variability of Envs, generation of bnAbs has been a daunting task (Phogat S, Wyatt R. Curr Pharm Des. 2007; 13:213-27, Phogat S, et al. J Intern Med. 2007 262:26-43, Karlsson Hedestam G B, et al Nat Rev Microbial. 2008 6:143-55).

### SUMMARY OF THE INVENTION

HIV and chimpanzee SIV Envs, the target of potentially protective neutralizing antibodies, display about 60% sequence conservation at the amino acid level, and HIV V2-apex bnAbs have been shown to neutralize certain chimpanzee SIV isolates, including the SIVcpzPtt isolate MT145, suggesting cross-species conservation of this epitope. The invention provides engineered molecules derived from SIV isolates that bind to broadly neutralizing HIV antibodies. In certain embodiments, the engineered molecules mimic structures and epitopes such as but not limited to V2 or V3.

In certain examples herein, the engineered molecules include SIVcpzPtt-based trimers including from the MT145 isolate and mutants of the MT145 isolate comprising amino acid substitutions, such as Asn at position 171 by Lys, which display native trimer-like properties, and selectively binds V2 apex bnAbs. In certain embodiments, the engineered molecules are designed to bind to germline or germline-reverted antibodies. For example, molecules comprising the V2 apex epitope of MT145K are effective in activating V2-apex-specific B cell precursors.

The Applicants determined the structure of the MT145K trimer by cryo-EM at a global resolution of 4.1 Å. The overall architecture displays similarity to HIV Env trimers (Julien et al., 2013a; Lyumkis et al., 2013; Ozorowski et al., 2017; Pancera et al., 2014). In addition, the glycan shield composition of MT145K closely resembled that of HIV Env glycans but is sufficiently different in detailed positioning of the glycans to exclude binding of all HIV bnAbs except for those directed to the V2 apex. MT145K trimer immunization in a V2 apex unmutated common ancestor (UCA)-expressing knock-in mouse model reveals induction of a predominantly V2-apex-site neutralizing Ab response that is reproducible and cross-neutralizes a related set of HIV isolates.

The engineered molecules are useful as immungens in HIV vaccination strategies. In V2-apex bnAb unmutated common ancestor (UCA) H chain only knock-in mice, MT145K trimer-derived molecules reproducibly elicited Abs able to neutralize the autologous virus and several V2-apex Ab sensitive viruses. The specificities of the nAbs are dependent on the glycan at N160 and a lysine on strand C of the V2. Boosting with a cocktail of HIV Env trimers successfully recalled the V2 apex specific nAb responses and generated some enhanced heterologous neutralization.

In an aspect, the invention provides an engineered or non-naturally occurring molecule from Simian Immunodeficiency Virus (SIV) that binds to a broadly neutralizing antibody (bnAb) that binds to HIV. In an embodiment of the invention, the bnAb binds to the V2 apex region of HIV. In an embodiment, the bnAb is a germline or germline reverted HIV bnAb. In an embodiment, the bnAb comprises one, two, three, four, five, or six CDRs of a bnAB described herein, including without limitation sequences in Table 2, or comprises one or more complementarity determining regions (CDRs) of a heavy chain variable domain, and/or one or more CDRs of a light chain variable domain.

In certain embodiments, the engineered or non-naturally occurring molecule binds to a HIV bnAB which comprises an amino acid sequence at least 50% identical, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to PG9 or to CH01 antibody.

In an embodiment of the invention, the molecule comprises a complex of gp120 and gp41. In certain embodiments, the molecules comprise all or part of a subunit, such as the Env protein of SIV. Env subunits and epitopes therefrom, particularly immunogenic epitopes are useful in the invention. Thus, in certain embodiments, the molecules comprise amino acids from SIV grafted into another protein or peptide, for example but without limitation HIV, HIV env, or other molecule capable of presenting an SIV V2 apex region and/or V2 apex epitope of the molecules herein. In an embodiment, the molecule comprises a gp140. In an embodiment, the molecule comprises a complex of gp120 and gp41. In an embodiment, the molecule comprises gp120 and gp140 that are covalently linked. In an embodiment, the molecule comprises gp120 and gp140 fusion. In an embodiment, the molecule comprises an SIV trimer. In an embodiment, the molecule comprises a chimeric trimer, such as but not limited to a chimeric trimer combining an SIV sequence within the V2 apex region that binds to a bnAb and HIV sequences elsewhere. In an embodiment, the molecule is a stabilized trimer, such as without limitation, a SOSIP, NFL, or UFO trimer.

In an embodiment, the SIV, V2 apex region, or V2 apex epitope comprises or is from SIVcpzPtt, SIVcpzPts, or SIVgor. In an embodiment, the V2 apex region, or V2 apex epitope comprises or is from the SIVcpzPit isolate MT145 or other V2-apex sequence set forth herein. In an embodiment, the V2 apex region, or V2 apex epitope comprises one or more amino acid substitutions that improve binding to a HIV broadly neutralizing antibody (bnAb) or to a germline precursor of the HIV bnAb. In an embodiment, the V2 apex epitope is carried on an env protein or fragments altered by deletion, insertion, or substitution of amino acids to disrupt an immunogenic or immunodominant epitope other than the epitope that binds to a bnAb. I an embodiment, the V5 loop is altered. In an embodiment, one or more amino acids is substituted with a basic amino acid, e.g., arginine or lysine. In an embodiment, the amino acid corresponding to position 171 of the MT145 isolate is or is substituted with lysine.

As described herein, in certain embodiments of the invention, useful V2 apex sequences comprise two glycans, thus two glycosylated aspargine residues, and basic amino acid residues, preferably three or four basic amino acid residues, in a four-amino acid sequence, for example, without limitation, Xxx-Lys-Lys-Lys (SEQ ID NO:1), Lys-Xxx-Lys-Lys (SEQ ID NO: 2), Lys-Lys-Xxx-Lys (SEQ ID NO:3), Lys-Lys-Lys-Xxx (SEQ ID NO:4), or Lys-Lys-Lys-Lys (SEQ ID NO:5). In certain embodiments, the molecule comprises Asn-Xxxₐ-Asn-Xxx_{b}-XXX_{c}-Xxx_{d}, wherein Xxxₐ comprises two or three amino acids, Xxx_{b} comprises five to seven amino acids, and Xxx_{c} comprises four amino acids, each of the four amino acid selected from Lys or Arg (SEQ ID NO: 6).

In certain embodiments, molecules of the invention comprise the amino acid sequence N-C-X₁-F-N-X₂-X₃-X₄-X₅-X₆-X₇-X₈-X₉-X₁₀-X₁₁-X₁₂-X₁₃-X₁₄-X₁₅-X₁₆: wherein X₁ comprises S, T, N, or F; X₂ comprises I, V, T, Q, or M; X₃ comprises S or T; X₄ comprises S or T; X₅ comprises S, E, or G; X₆ comprises I, L, V or F; X₇ comprises K or R; X₈ comprises G or D; X₉ comprises K, R, E, or Q; X₁₀ comprises K, R, E, or Q; X₁₁ comprises K, R, E, or Q; X₁₂ comprises K, E, or Q; X₁₃ comprises E, T, V, I, or M; X₁₄ comprises Y or K; X₁₅ comprises A or S; and X₁₆ comprises F, I, or L (SEQ ID NO:7). In certain embodiments, at least three of X₉, X₁₀, X₁₁, and X₁₂ comprise basic amino acid residues (SEQ ID NO:8). In certain embodiments, all of X₉, X₁₀, X₁₁, and X₁₂ comprise basic amino acid residues (SEQ ID NO:9). In certain embodiments, X₉, X₁₀, X₁₁, and X₁₂ each comprise K (SEQ ID NO:10). In one such embodiment, the amino acid sequence comprises NCSFNVTTELRDKKRQVYSL (SEQ ID NO:11). In another embodiment, the amino acid sequence comprises NCSFNVTTELRDKKRKVYSL (SEQ ID NO:12). In another embodiment, the amino acid sequence comprises NCSFNVTTELRDKKKKEYSF (SEQ ID NO: 13). In another embodiment, the amino acid sequence comprises NCSFNATTELRDKKKKEYAL (SEQ ID NO:14).

In certain embodiments, molecules of the invention comprise Asn-Cys-Ser-Phe-Asn-Val-Thr-Thr-Glu-Leu-Arg-Asp-Lys-Lys-Arg-Gln-Val-Tyr-Ser-Leu-Phe-Phe-Tyr (SEQ ID NO: 15), or Asn-Cys-Ser-Phe-Asn-Val-Thr-Thr-Glu-Leu-Arg-Asp-Lys-Lys-Arg-Gln-Val-Tyr-Ser-Leu-Phe-Tyr (SEQ ID NO:16), or Asn-Cys-Ser-Phe-Asn-Val-Thr-Thr-Glu-Leu-Arg-Asp-Lys-Lys-Arg-Lys-Val-Tyr-Ser-Leu-Phe-Phe-Tyr (SEQ ID NO:17), or Asn-Cys-Ser-Phe-Asn-Val-Thr-Thr-Glu-Leu-Arg-Asp-Lys-Lys-Arg-Lys-Val-Tyr-Ser-Leu-Phe-Tyr (SEQ ID NO: 18), or Asn-Cys-Ser-Phe-Asn-Val-Thr-Thr-Glu-Leu-Arg-Asp-Lys-Lys-Lys-Lys-Glu-Tyr-Ser-Phe-Phe-Phe-Tyr (SEQ ID NO:19); or Asn-Cys-Ser-Phe-Asn-Val-Thr-Thr-Glu-Leu-Arg-Asp-Lys-Lys-Lys-Lys-Glu-Tyr-Ser-Phe-Phe-Tyr (SEQ ID NO:20); or Asn-Cys-Ser-Phe-Asn-Ala-Thr-Thr-Glu-Leu-Arg-Asp-Lys-Lys-Lys-Lys-Glu-Tyr-Ala-Leu-Phe-Phe-Tyr (SEQ ID NO:21), or Asn-Cys-Ser-Phe-Asn-Ala-Thr-Thr-Glu-Leu-Arg-Asp-Lys-Lys-Lys-Lys-Glu-Tyr-Ala-Leu-Phe-Tyr (SEQ ID NO: 22).

In an aspect, the invention provides a nucleic acid encoding the molecule from Simian Immunodeficiency Virus (SIV) that binds to a broadly neutralizing antibody (bnAb) that binds to HIV. In an embodiment, the nucleic acid comprises RNA. In an embodiment, the nucleic acid comprises DNA. In an embodiment, the nucleic acid is operably linked to a regulatory element operable in a cell. In certain such embodiments, the regulatory element is operable in a eukaryotic cell. In certain such embodiments, the regulatory element is operable in a prokaryotic cell. The invention further provides vectors, such as but not limited to viral vectors, comprising the nucleic acids and regulatory elements. In an embodiment of the invention, the viral vector is AAV. The invention further provides cell which comprise the nucleic acids, including cells that express the molecules of the invention.

In an aspect, the invention provides a method of eliciting an immune response in a mammal comprising administering an immunogenic composition comprising a molecule of the invention. In an embodiment, a method of stimulating an HIV bnAb is provided. In certain embodiments, the bnAb is a germline antibody or a germline reverted antibody. In an embodiment of the invention, the mammal is a human. In an embodiment, the mammal is a non-human primate, such as but not limited to a chimpanzee or gorilla. In an embodiment, the mammal is one capable of expressing one or more elements of a human immune system, for example a transgenic animal such as but not limited to a mouse. In one such embodiment, the mammal comprises and is capable of expressing a human germline or germline reverted antibody or germline or germline reverted heavy chain variable domain or light chain variable domain. In another embodiment, the mammal comprises and is capable of expressing a repertoire of human germline genes.

In certain embodiments, a molecule of the invention is administered one time. In other embodiments, a molecule of the invention is administered multiple times, such as twice, three times, or more. In certain embodiments, more than one molecule is administered, such as two, three, four, or more molecules. When immunogenic compositions are administered at more than one time point, the molecules administered at each time can be the same or different. For example, two or more that are different molecules can be administered concurrently or sequentially. Alternatively, the same molecule can be administered on two or more occasions. Without being bound by theory, the molecules are administered in a course of immunization designed to favor production of bnAbs. bnAbs can be favored when a selected epitope that elicits bnAbs is present in multiple immunizations while other epitopes of the immunogen differ from one immunization to another.

In certain embodiments, one molecule of the invention is administered at a first time point to initiate an immune response, such as, for example, to stimulate germline antibody-expressing B cells, and again at a later time to boost an immune response. In certain embodiments, one molecule of the invention is administered at a first time point and another different molecule of the invention is administered at a later time to selectively boost the antibody response against an epitope common to both molecules that binds to a HIV bnAb.

In certain embodiments, a molecule of the invention is administered at a first time point to initiate an immune response, such as, for example, to stimulate germline antibody-expressing B cells, and an HIV molecule, antigen, or vaccine is administered at a later time point to boost the response against an HIV epitope that binds to a HIV bnAb.

In certain embodiments, the germline antibodies that are stimulated are bnAbs. In certain embodiments, the germline antibodies are precursors of bnAbs. In certain such embodiments, the germline antibodies undergo somatic hypermutation and selection during the course of an immune response to higher affinity and/or specificity bnAbs.

In certain embodiments, a molecule of the invention is administered to boost or reactivate memory B cells.

The invention provides immunogenic compostions comprising the molecules of the invention. In certain embodiments, a molecule of the invention is comprised in a composition or administered with an adjuvant. In certain embodiments, the adjuvant comprises a lecithin. In certain embodiments, the lecithin is (a) combined with an acrylic polymer, (b) in a coated oil droplet in an oil-in-water emulsion, or (c) in an acrylic polymer in an oil-in-water emulsion. In certain embodiments the adjuvant is ISCOMATRIX or Adjuplex. In certain embodiments the adjuvant comprises alum.

In certain embodiments, the immunogenic composition comprises a liposome or a nanoparticle. In certain embodiments, the molecule of the invention is fixed. In an embodiment, the fixing agent can be glutaraldehyde. In an embodiment, the trimer or immunogen comprising the trimer is quenched with glycine.

In an aspect, the invention provides a method of engineering a molecule or immunogen capable of eliciting a broadly neutralizing antibody (bnAb) directed to HIV. The method comprises identifying a viral structure such as a viral epitope of SIV or other immunodeficiency virus that is conserved in HIV, selecting or designing bnAb that binds to the epitope, and designing a molecule or immunogen that comprises the conserved epitope, optionally modified to bind to a germline or germline derived antibody precursor of the bnAb. In certain embodiments, the structure, region or epitope is that of V2 of SIV.

In an aspect of the invention, there is provided a method of identifying or selecting a broadly neutralizing component of binding pair wherein such component binds to HIV and is broadly neutralizing. The method comprises contacting a molecule of the invention with a candidate binding pair component and selecting or identifying the component as broadly neutralizing if it binds to a molecule of the invention. In certain embodiments, the broadly neutralizing component comprises a bnAb. In certain embodiments, the broadly neutralizing component comprises a germline or germline reverted bnAb. In certain embodiments, the broadly neutralizing component is a germline or germline reverted antibody that is a precursor of a bnAb.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1D. Design of a chimpanzee Env-stabilized trimer and binding to V2 apex bnAbiGL Abs. A. Structural arrangement of the V2 apex bnAb core epitope region on BG505.664 soluble Env trimer (modified from (Garces et al., 2015) (PDB: 5CEZ)). The ribbon representation of V1V2 loop strands that form the trimer apex show a cluster of positively charged lysine-rich peptide regions (HXB2-R166-K171: R or K residues shown as blue spheres) and the two glycans N156 and N160 (depicted in green spheres with lines). The side chains of the positively charged residues intersperse with the side chains of residues from adjacent protomers to form a continuous positively charged surface at the tip of the trimer to provide a minimal V2 apex bnAb epitope. B. Amino-acid sequence alignment of strand B and C V2 of HXB2 (SEQ ID NO:23), HIV CRF250 (SEQ ID NO:20), CAP256.SU (SEQ ID NO:22), chimpanzee SIV MT145 WT (SEQ ID NO:16) and its V2-modified variant (Q171K), MT145K (SEQ ID NO:28). Glutamine (Q) at position 171 (shown in red) was substituted with lysine (K) in MT145 Env to gain binding to V2 apex bnAb inferred germline (iGL) Abs. C. ELISA binding of mature V2 apex bnAbs, PG9, CAP256.09 and CH01 and their iGL versions to WT MT145 (red) and MT145K SOSIP trimers. D. Octet binding curves (association: 120s (180-300) and dissociation: 240s (300-540)) of CAP256 UCA and CH01 iGL Abs and their respective mature Ab versions (CAP256.09 and CH01) to MT145K trimer, its glycan knock-out (N160K) variant, K-rich core epitope substituted variants and the corresponding monomeric gp120. The Abs were immobilized on human IgG Fc capture biosensors and 1 uM trimer or gp120 proteins used as analytes. The binding response is shown as nanometer (nm).

FIG. 2A-2E. Cryo-EM structure of the MT145K trimer. A. Schematic showing MT145K SOSIP soluble trimer design from its full-length gp160 Env sequence. The gp120 constant (C1-C5) and variable (V1-V5) regions and the gp41 regions (fusion peptide (FP), heptad repeat (HR1 and HR2), membrane proximal external region (MPER), transmembrane (TM) and cytoplasmic tail (CT)) are indicated. The N-linked glycan positions for each NXT/S residue are labeled according to the HIV HXB2 numbering scheme. The SOSIP trimer stabilizing modifications include: (i) disulfide bond: A501C-T605C, (ii) R6 cleavage site, (iii) 1559P, and (iv) 664-residue truncation in gp41 MPER. The substitution to incorporate a K-residue at position 171 (Q171K) to gain binding for V2 apex iGL Abs is indicated in blue. B-C. Side and top views of the unliganded MT145K trimer model based on the cryo-EM density map at ˜4.1 Å resolution. Ribbon representations of the MT145K trimer spike, in which the subunits gp120 (cornflower blue) and gp41 (orange) are depicted on one protomer. The gp120 variable loops (V1-V5) positioned to the trimer periphery are depicted in different colors (V1: khaki, V2: red, V3: magenta, V4: yellow and V5: chartreuse). The fusion peptide region of gp41 is shown in cyan. Glycan sugar residues modeled based on density are represented in forest green stick form. D. Superimposition of variable loops (V1-V5) and fusion peptide region for MT145K and unliganded HIV clade A BG505 (PDB: 4ZMJ) SOSIP trimers. The dotted lines indicate regions in the V-loops or FP for which the observed electron density was absent or unclear. E. Structural comparison of gp41 regions of MT145K (orange) and BG505 (grey) trimers. The gp41 structural elements overall show a similar arrangement except for the fusion peptide region (colored cyan on MT145K and pink on BG505), that is exposed on the BG505 trimer but remains hidden in a pocket inside the MT145K trimer.

FIG. 3A-3C. Site-specific glycoform composition of MT145K trimer. A. Site-specific glycoform quantification of the MT145K SOSIP soluble trimer. MT145K trimers from transiently transfected HEK293T cell expressed supernatants were affinity purified by the quaternary trimer-specific antibody, PGT145. The purified MT145K trimers were treated separately with three proteases: trypsin, chymotrypsin and elastase and the digests were enriched for glycopeptides and analysed by LC-ESI MS. The individual glycan composition of the N-linked glycan sites (n=26) is represented by bar graphs that indicate the relative abundance of each glycoform species and are derived from the mean of two analytical replicates. The pie charts summarize the proportion of glycoforms for each site and this information is color coded; oligomannose-type (green) and complex/hybrid glycans (pink). B. HILIC-UPLC profiles of the total N-linked glycans released from MT145K trimers. The proportions of oligomannose plus hybrid glycan contents and complex-type glycans are represented in green and pink colors, respectively. C. Modeled glycan shields for MT145K and BG505 SOSIP trimers. Man₉GlcNAc₂ oligomannose-type glycans were docked and rigid-body-fitted at each of the corresponding Env glycan positions using the MT145K structure (determined in this study (PDB: submit)) and the unliganded BG505 SOSIP.664 trimer structure ((Kwon et al., 2015) PDB: 4ZMJ). Top and side views of the trimers are shown and the individual glycan sites are labelled and color-coded based on the content of oligomannose; green (100-80%), orange (79-20%) and pink (19-0%).

FIG. 4A-4B. Antigenic profile of the MT145K trimer. A. HIV-1 Env-specific mAbs were used to characterize the antigenicity of the MT145K Env trimer. MAbs targeting neutralizing and non-neutralizing epitope specificities, including V2-apex, N332-V3, linear V3, CD4bs, CD4i and gp120-41 interface were tested with MT145K and BG505 Env-encoding pseudoviruses in a TZM-b1 cell-based reporter assay. The reciprocal IC₅₀ neutralization titers for each virus are indicated as dot plots; plots for individual epitope specificities are depicted separately. The neutralization sensitivity comparison of BG505 and MT145K viruses against the mAb panel shows a selectively potent neutralization of MT145K by V2 apex bnAbs but no other bnAbs, except a single gp120-gp41 interface bnAb, 35022. BG505 virus was neutralized by bnAbs targeting diverse Env sites. B. The above mAb panel was further tested with PGT145 Ab-purified MT145K trimer and GNL-purified MT145K gp120 monomer by ELISA. The binding, represented as EC₅₀ binding titers, shows selective binding of MT145K by V2 apex bnAbs. Two of the gp120-gp41 interface bnAbs and a CD4i mAb also showed significant binding to MT145K trimer. Four of the non-neutralizing mAbs specific to a linear V3 epitope exhibited binding to MT145K gp120, but not to the trimer.

FIG. 5A-5F. A close-up view of regions on the MT145K trimer that correspond to those recognized by HIV bnAbs on HIV trimers. A. V2 apex bnAb binding region: cryo-EM model of PGT145 bnAb (HC: transparent sandy brown; LC: transparent orchid) in complex with BG505 SOSIP trimer depicting VIV2 loops in ribbon representation ((Lee et al., 2017) PDB: 5V8L). The strand C K-rich region (¹⁶⁶RDKKQK¹⁷¹ (SEQ ID NO:355); red spheres) and the glycan N160 (forest green sticks) that form the epitope for PGT145 bnAb are indicated. The elements in the core epitope interact with the CDRL1 loop and the long CDRH3 loop that penetrates through glycans to reach the positively charged surface underneath. Both glycan N160 and the positively charged protein residues are conserved between BG505 HIV-1 and MT145K SIV Env trimers. B. V3-glycan bnAb binding region: cryo-EM model of PGT128 bnAb (HC: transparent sandy brown; LC: transparent orchid) in complex with the BG505 SOSIP trimer ((Lee et al., 2015) PDB: 5ACO). The V3 loop protein backbone residues (³²⁴GDIR³²⁷ (SEQ ID NO:356); depicted in purple spheres) and the glycans N301 and N332 form the bnAb epitope and are shown to interact with the antibody CDR loops. The MT145K trimer has a glycan at N334 rather than N332 and the glycan points away from the expected location of the PGT128 Ab paratope. In addition, MT145K Env has glycans at two positions N412, (positioned differently on HIV Env) and N442 (absent on HIV Envs) and particularly the latter will clash with PGT128 CDRH2 and prevent it from interacting with the protein part of the epitope. C. The gp120-gp41 interface bnAb binding region: cryo-EM model of PGT151 bnAb bound to a membrane-extracted clade B JRFL Env trimer. The structure depicts PGT151 bnAb CDRs interacting with gp120 and the gp41 interface regions ((Lee et al., 2016); PDB: 5FUU). PGT151 CDRH3 interacts with the epitope formed by the protein backbone (in both gp120 and gp41) including the fusion peptide (depicted in pink) and the gp120 (N88, N448) and gp41 (N611 and N637) glycans (not shown). PGT151 Ab CDR loops interact with the FP region on the BG505 trimer. The MT145K trimer FP region (cyan) remains hidden inside the trimer. D. Cryo-EM model of 2-domain human sCD4 with B41 SOSIP trimer ((Ozorowski et al., 2017); PDB: 5VN3). The structure shows how the Phe43 residue on sCD4 stacks into the Env cavity lining Trp427. This Trp427 cavity is conserved between HIV-1 and MT145K Envs to accommodate CD4 binding. E-F. CD4bs bnAb binding region: crystal structure of VRC01 bnAb in complex with 93TH057 gp120 ((Zhou et al., 2010) PDB: 3NGB). The structure depicts VRC01 CDRH3, CDRL3 and CDRL1 loops interacting with the protein residues in loop D (HXB2: 278-282) and the glycan at N276. The MT145K trimer lacks the N276 glycan and bears glycan N236 (unique to SIV Env) in place of N234 that would clash with the VRC01 CDRL1 loop. Additionally, the MT145K Env trimer has a longer gp120-V5 loop due to a 6-amino acid insertion at HIV HXB2-456 residue that would shift the loop such that it clashes with the CDRH2 the VRC01 Ab.

FIG. 6A-6D. Immunogenicity of MT145-WT compared to MT145K trimers in CH01 UCA HC-only knock-in mice. A. Schematic showing immunization schedule of CH01 UCA “HC-only” KI mice with MT145-WT and engineered MT145K trimers. The CH01 UCA “HC-only” KI mice were immunized twice with 25 μg of the soluble trimer with GLA-SA as adjuvant. Time points for immunization and bleeds are indicated. B. ELISA binding of the MT145-WT and MT145K group trimer-immunized CH01 UCA “HC-only” KI mice serum samples (pre-bleed (Pre), two-weeks post prime (Bleed #1) and two-weeks post boost-1 (Bleed #2)) with soluble MT145K SOSIP and its glycan knock-out variant (MT145K N160K) trimers. C. Neutralization titrations of the MT145-WT and MT145K group trimer immunized CH01 UCA “HC-only” KI mice sera (pre-bleed (Pre), post prime (Bleed #1) and post boost-1 (Bleed #2)) with MT145K virus and a CH01-sensitive virus (Q23_17). 3-fold diluted sera were tested against the viruses in a TZM-b1 reporter cell assay. D. ID₅₀ neutralization titers of the MT145-WT and MT145K group trimer-immunized CH01 UCA “HC-only” KI mice sera (pre- and post-immunization bleed time points). Neutralization was assessed against the priming immunogen-matched autologous viruses in each group (MT145-WT group: MT145-WT virus, and MT145K group: MT145K virus), the N160 glycan knock-out variant of MT145K virus (MT145K N160A) and a CH01-sensitive virus, Q23 17. The numerical values shown in the table represent the ID₅₀ neutralization titers of the immune serum samples and were calculated by non-linear regression method from the percent neutralizations of serum titrations with virus.

FIG. 7A-7C. Immunizations combining chimpanzee SIV MT145K trimer with HIV trimers in CH01 UCA HC-only knock-in mice. A. Schematic showing immunization schedule of CH01 UCA “HC-only” KI mice with a combination of chimpanzee SIV MT145K trimer and HIV Env-derived trimers. A group of 5 animals was immunized with two doses of MT145K trimer (prime: week-0 and boost-1: week-4) followed by further boosting (boost-2 at week-8) with an HIV Env derived 3-trimer cocktail (C108, WITO and ZM197-ZM233V1V2). The VIV2 loops on trimer cartoons are depicted in red to highlight the region is shared across HIV and SIV Env trimers. The CH01 UCA “HC-only” KI mice were immunized with 25 μg of the soluble MT145K trimer or HIV trimer cocktail (25 μg total protein) with GLA-SE as adjuvant. Time points for the immunization and the bleeds are indicated. B. Neutralization of the pre- and post-immune sera were carried out against a panel of viruses in the TZM-b1 reporter cell assay. The numerical values in the table represent ID₅₀ neutralization titers of the pre-bleed (Pre) and post-prime (Bleed #1), post boost-1 (Bleed #2) and post boost-2 (Bleed #3) serum samples. Neutralization was assessed against the priming immunogen-matched autologous virus, MT145K, its N160 glycan knock-out variant, boosting immunogen-matched, CH01 sensitive, and global panel HIV Env-encoding viruses. The numerical values shown in the table represent the ID₅₀ serum neutralization titers and were calculated by non-linear regression method from the percent neutralizations of serum titrations with virus. The IC₅₀ neutralization titers for the CH01 mature Ab against the virus panel are shown. C. The ID₅₀ virus neutralization titers of the Bleed #4 serum samples collected post boost-2 immunization with an HIV three-trimer cocktail. The neutralization of the post-immune sera was assessed against the priming immunogen-matched autologous virus, MT145K, the boosting immunogen-matched, CH01 sensitive viruses, and global panel HIV Env-encoding viruses. Each filled circle in the plot represents virus ID₅₀ neutralization values for the individual animals; an asterisk (*) indicates that 50% neutralization was not reached at a 1:100 serum dilution.

FIG. 8A-8B. Design and purification of SIVcpzPtt envelope derived SOSIP.664 trimers. A. Amino-acid sequence alignment of 4 SIVcpzPtt envelope sequences (GAB1 (SEQ ID NO: 25), MB897 (SEQ ID NO:26), EK505 (SEQ ID NO:27) and MT145 (SEQ ID NO:28)) with reference HIV-1 Env sequence, HXB2 (SEQ ID NO:24), showing SOSIP.664 trimer stabilizing modifications. The soluble SOSIP.664 trimer modifications include: (i) incorporation of a disulfide bond between residue 501 of gp120 (A501C) and residue 605 of gp41 (T605C), (ii) replacing naturally occurring gp120 and gp41 cleavage site with R6-cleavage site (shown in blue), (iii) 1559P substitution in the gp41, (iv) and a truncation in the gp41 subunit at residue 664. B. Size Exclusion Chromatography (SEC) profiles of Gallanthus nivalis lectin (GNL) purified trimers on Superdex 200 Increase 10/300 GL column. The SEC profiles show the aggregate/trimer-dimer, trimer and monomer peaks of the GNL-purified proteins. The MT145 SOSIP.664 trimer showed substantial protein fractions that assemble as a trimer.

FIG. 9A-9D. MT145K SOSIP.664 forms native-like trimers that are thermostable. Size exclusion chromatography (SEC) of PGT145 antibody purified MT145K SOSIP.664 trimer reveals trimer eluting as a single peak with no aggregation. B. SDS-PAGE of MT145K trimer protein under non-reducing (NR) and reducing (R) conditions (reducing agent DTT was added). The MT145K trimer is efficiently cleaved into gp120 and gp41 subunits. C. Negative Stain Electron-Microscopy (NS-EM) of MT145K trimer: 2D class averages show that trimers adopt well-ordered, native-like conformations. D. Thermostablity of the MT145K trimer. Thermal denaturation of the MT145K trimer by Differential Scanning calorimetry (DSC) reveals a major trimer population melting at a Tm of 65.4° C.

FIG. 10A-10B. Design of inferred germline (iGL) versions of V2 apex bnAbs, PG9 and CH01. The heavy chain (VH), light chain (VL) and the diversity (D) and the joining (J) regions in the mature antibodies were reverted to their corresponding inferred germline genes. The amino-acid reversions from mature VH (panel A) and VL (panel B) chains to germline residues are indicated by asterisks. The VH_iGL and the VL_iGL chains were paired and expressed as soluble antibodies. The GenBank accession numbers and SEQ ID NOS for the heavy and light chain inferred germline versions of PG9 and CH01 broadly neutralizing antibodies are PG9_VDJH_iGL: Accession MK825341 (SEQ ID NO:32); PG9VJL_iGL: Accession MK825342 (SEQ ID NO:41); CH01VDJH_iGL: Accession MK825343 (SEQ ID NO:49); and CH01VIL_iGL: Accession MK825344 (SEQ ID NO:58). Depicted PG9-related sequences are identified in the Sequence Listing as follows: IGHV3-33-05: SEQ ID NO:29; PG9_HC: SEQ ID NO:30; PG9VH_iGL: SEQ ID NO: 31; IGHJ6-03: SEQ ID NO:36; IGHD3-3-01: SEQ ID NO:37; IGLV2-14-01: SEQ ID NO: 38; PG9_LC: SEQ ID NO:39; PG9VL_iGL: SEQ ID NO:40; IGLJ3-02: SEQ ID NO:45. Depicted CH01-related sequences are identified in the Sequence Listing as follows: IGHV3-20-01: SEQ ID NO:46; CH01_HC: SEQ ID NO:47; CHO1VH_iGL: SEQ ID NO:48; IGHJ2-01: SEQ ID NO: 53; IGHD3-10-01: SEQ ID NO:54; IGKV3-20-01: SEQ ID NO:55; CH01_LC: SEQ ID NO: 56; CH01VL_iGL: SEQ ID NO:57; IGKJ1-01: SEQ ID NO:62.

FIG. 11A-11D. Structural analysis of MT145K by cryo-electron microscopy. A. Cryo-EM data processing flow diagram resulting in a density map at ˜4.1 Å global resolution. B. FSC between two independently refined data half sets. C. Local resolution in the MT145K density map. The peptide part of MT145K is resolved primarily in the 3.0-4.0 Å range, whereas the glycan shield overall is resolved to significantly lower resolutions. D. Angular distribution of the angularly refined data class giving rise to the final reconstruction.

FIG. 12. Comparison of Env trimer architectures of class I fusion proteins. Prefusion SIV Env (MT145K; current study) follows an overall organization similar to other known prefusion class I fusion proteins. Fusion subunits (shown in orange) are most commonly organized membrane-proximally (RSV F deviating somewhat from this trend). Fusion subunits are largely shielded and capped by receptor-recognizing subunits (shown in cornflower blue) in the meta-stable prefusion state. SIV Env is most similar to HIV-1 Env; however, MT145K is less compact than the displayed HIV-1 Env trimer (BG505 isolate). The more open organization of MT145K may be the result of its fusion peptide being inserted into a pocket at the gp120/gp41 interface as opposed to the fusion peptide found on the outside of the HIV-1 Env trimer (MT145K: current study, HIV-1: PDB 4ZMJ (Kwon et al., 2015); Influenza A HINI: PDB 1RD8 (Stevens et al., 2004); Ebola virus GP: PDB 5KEL (Pallesen et al., 2016); MERS-COV S: PDB 5W9J (Pallesen et al., 2017); RSV F: PDB 4JHW (Mclellan et al., 2013).

FIG. 13A-13B. Negative ion TOF MS of released glycans from Chimpanzee SIV, MT145K gp120 and gp41. A. Driftscope extracted singly charged ions of released glycans from MT145K-gp120. B. Driftscope extracted singly charged ions of released glycans from MT145K-gp41.

FIG. 14A-14B. Phylogeny of lentiviruses and the glycan shield density. A. Phylogenetic relationships among the envelope sequences derived from various lentiviruses; tree constructed by maximum likelihood method. These lentiviruses infect host species that include equine (EIAV), feline (FIV), bovine (BIV), caprine/ovine (CAEV, OMVVS and MVV), simian (SIV) and humans (HIV). B. The number of amino acids and the number of Potential N-linked Glycan Sites (PNGS: predicted by N-GlycoSite tool at Los Alamos HIV database) in the full-length envelopes are listed for each lentivirus. The glycan shield density for each virus is represented as % Env residues that encode PNGS motifs and was calculated by (number of PNGS/Env length×100). The glycan shield density shows a gradual increase from EIAV through CAEV, and plateaus in some of the SIV species including SIVcpzPtt, the one that has been shown to have crossed into humans.

FIG. 15. Amino-acid sequence alignment of SIV envelopes with HXB2 HIV-1 Env as a reference depicting SIV Env residues equivalent to HIV bnAb epitopes. The amino-acid alignment of SIVcpzPtt envelope sequences compared to the HXB2 reference sequence. The protein residues (highlighted in grey) and the N-glycosylation sites (depicted in red) for each bnAb epitope region (V2-apex, V3-N332, CD4bs and gp120-gp41 interface) are shown. Eleven regions of 23 aligned Env isolates are depicted, indicated by amino acid numbers: i) 156-175, ii) 320-353, iii) 409-447, iv) 256-291, v) 228-245, vi) 448-467, vii) 84-93, viii) 448-455, ix) 507 534, x) 605-614, and xi) 621-646. The eleven regions of each of the 23 isolates are represented in the Sequence Listing as follows: 1) B.FR.1983.HXB2-LAI-IIIB-BRU.K03455: SEQ ID NOS: 63-73; 2) CPZ.CM.2005.SIVcpzMT145.DQ373066: SEQ ID NOS: 74-84; 3) CPZ.CM.2005.SIVcpzMB897.EF535994: SEQ ID NOS: 85-95; 4) CPZ.CM.2005.SIVcpzLB7.DQ373064: SEQ ID NOS: 96-106; 5) CPZ.CM.2001.SIVcpzCAM13.AY169968: SEQ ID NOS: 107-117; 6) CPZ.GA.1988.GAB1.X₅₂₁₅₄: SEQ ID NOS: 118-128; 7) CPZ.CM.2005.SIVcpzMB66.DQ373063: SEQ ID NOS: 129-139; 8) CPZ.GA.2006.SIVcpzGab4.GQ217539: SEQ ID NOS: 140-150; 9) CPZ.CM.2004.Ptt_04Cam155.FR686510: SEQ ID NOS: 151-161; 10) CPZ.CM.1998.CAM3.AF115393: SEQ ID NOS: 162-172; 11) CPZ.TZ.2001.TAN2_69.DQ374657: SEQ ID NOS: 173-183; 12) CPZ.TZ.2002.TAN3_1.DQ374658: SEQ ID NOS: 184-194; 13) CPZ.TZ.2000.TAN1.AF447763: SEQ ID NOS: 195-205; 14) CPZ.TZ.2006.TAN5.JN091691: SEQ ID NOS: 206-216; 15) CPZ.TZ.2009.UG38.JN091690: SEQ ID NOS: 217-227; 16) CPZ.TZ.2006.SIVcpzTAN13.JQ768416: SEQ ID NOS: 228-238; 17) CPZ.CD.2006.BF1167.JQ866001: SEQ ID NOS: 239-249; 18) CPZ.CD.1990.ANT.U42720: SEQ ID NOS: 250-260; 19) CPZ.GA.1988.SIVcpzGAB2.AF382828: SEQ ID NOS: 261-271; 20) CPZ.CM.2005.SIVcpzEK505.DQ373065: SEQ ID NOS: 272-282; 21) CPZ.CM.1998.CAM5.AJ271369: SEQ ID NOS: 283-293; 22) CPZ.US.1985.US_Marilyn.AF103818: SEQ ID NOS: 294-304; 23) CPZ.CM.2005.SIVcpzDP943.EF535993: SEQ ID NOS: 305-315.

FIG. 16. Antigenic profile of various MT145K Env forms with HIV Env-specific mAbs. Neutralizing and non-neutralizing HIV Env-specific mAbs targeting various epitope specificities, including V2-apex, V3-N332, linear V3, CD4bs, CD4i and gp120-gp41 interface were tested with MT145K and BG505 Env-encoding pseudoviruses in a neutralization assay and against the MT145K SOSIP trimer and MT145K gp120 monomer by ELISA. The reciprocal IC₅₀ neutralization titers against the MT145K and BG505 viruses and the 50% ELISA binding titers (EC₅₀ binding with trimer and gp120 proteins) for each mAb are shown.

FIG. 17A-17E. Molecular details of the regions on the MT145K trimer that correspond to bnAb epitopes on HIV trimers. A. Crystal structure of PG9 (PDB: 3U2S (McLellan et al., 2011)) bound to ZM109 VIV2 apex and docked onto BG505. The long CDRH3 of PG9 interacts with the K-rich region of the V2 apex and the glycans at N160. Both glycan/peptide epitope elements are conserved between MT145K and HIV-1 BG505 Env and, hence, PG9 binds strongly to both. B-C. Crystal structures of V3-N332 bnAbs PGT121 (5T3Z (Gristick et al., 2016)) and PGT135 (PDB: 4JM2 (Kong et al., 2013)) interacting with their epitopes at the base of the V3 loop. The N332 glycan required by these bnAbs is absent on MT145K Env and the N334 glycan is projecting away from the epitopes and is not a functional substitute. In addition, the glycans N412 and N442, the latter being unique to the SIV Env, would clash with the CDRs of these bnAbs, thus preventing interaction. D-E. Cryo-EM reconstruction of CD4bs bnAb 3BNC117, bound to BG505 trimer (PDB: 5V8M (Lee et al., 2017)). The lack of 3BNC117 binding with MT145K SIV Env trimer correlates with significant sequence variation in loop D and a clash of bnAb CDR loops with the N236 glycan that is unique to SIV. Moreover, a longer MT145 V5 loop would introduce a clash with 3BNC117 CDRH2.

FIG. 18A-18C. Cross-reactivity of CRF250 trimer-induced “on” and “off” target Ab responses with chimpanzee SIV MT145K trimer. A. CD45.2-encoded CH01 UCA B cells were transferred into CD45.1 wild-type (WT) mice at 5 dilutions (1:1, 1:10⁴, 1:10⁵, 1:10⁶ and 1:10⁷). Mice were immunized with CRF250 trimer at week-0 (prime) and boosted at week-4 (boost). A dose of 25 μg of the trimer protein/mouse/immunization was administered with adjuvant GLA-SE. B. Heatmap showing ELISA binding of the CRF250 trimer prime and boosted serum Ab responses of CH01 UCA B cell diluted mice with CRF250 (HIV subtype AG) and MT145K (chimpanzee SIV) Env trimers and their N160K variants. Chimpanzee SIV MT145K trimer shares cross-reactivity only at the V2-apex bnAb epitope with HIV trimers. EC50 binding titers for each serum/protein combination are shown; red color indicates stronger binding; green indicates weaker binding. CRF250 trimer-induced post prime (2 wk) Ab responses exhibit cross-reactive binding with MT145K trimer almost exclusively through the V2-apex bnAb site; no binding with MT145K N160K trimer variant. CRF250 trimer-induced post boost (6 wk) Ab responses, in addition cross-reacting at the V2-apex bnAb site, show minimal cross-reactive binding of the CRF250 trimer Env backbone off-target (N160K-dependent) Ab responses with MT145K trimer backbone (2/16 animals). C. Scatter dot plots with bars showing the frequencies of CH01 specific class-switched IgG B cells from CRF250 trimer immunization post-prime and post-boost splenic samples as determined by next generation sequencing (NGS). The CH01 IgG frequencies are derived from Ig sequencing data of the total splenic CH01 specific B cells (IgM+IgG). Each dot represents an individual mouse and the bar height in each dilution groups indicates median CH01 IgG frequencies.

FIG. 19. Amino-acid sequence of the Chimpanzee Simian Immunodeficiency virus (SIVcpz) envelope trimer construct, MT145K dV5 SOSIP.664 (SEQ ID NO:316), showing SOSIP.664 trimer stabilizing modifications. The soluble SOSIP.664 trimer modifications include: (i) replacement of N-terminus envelope gp120 signal peptide sequence with tissue plasminogen activator (TPA) signal sequence for higher protein expression (shown in yellow) (ii) incorporation of a disulfide bond between residue 501 of gp120 (A501C) and residue 605 of gp41 (T605C) (shown in turquoise), (iii) replacing naturally occurring gp120 and gp41 cleavage site with R6-cleavage site (shown in red), (iv) 1559P substitution in the gp41 (shown in grey), (v) and a truncation in the gp41 subunit at residue 664 by inserting a stop codon (indicated by an asterisk (*)). Underlined residues (KD) indicate V5 deletion position.

FIG. 20A-20D. Immunogenicity of the MT145KdV5 SOSIP trimer version versus MT145K SOSIP trimer in CH01 HC-only KI mouse model. A. Negative Stain Electron-Microscopy (NS-EM) of MT145KdV5 trimer: 2D class averages show trimers adopting 100% native-like conformations. B. BioLayer Interferometry (BLI) binding responses of the HIV Env specific mAbs and V2-apex bnAb IGLs PG9, CH01, CAP256 UCA to the soluble MT145KdV5 SOSIP trimer. C. Schematic showing immunization schedule of CH01 UCA HC-only KI mice with MT145KdV5 and MT145K trimers. The mice were immunized twice with 25 μg of the soluble trimer with glucopyranosyl lipid adjuvant stable emulsion (GLA-SE) as adjuvant. Time points for immunization and bleeds are indicated. D. Neutralizing antibody responses induced by MT145KdV5 and MT145K SOSIP trimer immunogens in CH01 UCA HC-only KI mice. Neutralizing activities of serum samples (pre-bleed; 2 weeks post prime, Bleed #1; and 2 weeks post boost-1, Bleed #2) were tested with MT145K and CH01 Ab sensitive viruses (Q23_17, CRF250 and C108) in TZM-b1 reporter cell assay. Each dot in the plots represents virus ID₅₀ neutralization titers that were calculated by a non-linear regression method from the percent neutralizations of serum titrations with the corresponding viruses. The ID₅₀ neutralization titers between MT145KdV5 (red) and MT145K (blue) trimer immunized animal groups were compared by two-tailed unpaired Mann-Whitney test and the P-value for each comparison is indicated. P-values of less than 0.05 are treated as significant. Overall MT145KdV5 SOSIP trimer induced superior nAb responses as compared to the parent MT145K SOSIP trimer.

FIG. 21A-21C: Immunogenicity of MT145KdV5 SOSIP trimer immunogen in non-human primate model. A. Schematic showing immunization schedule of non-human primates (NHPs) vaccinated with MT145KdV5 SOSIP trimer. A group of 6 animals was immunized twice with MT145KdV5 trimer (prime, week 0; boost-1, week 8). The NHPs were immunized with 100 μg of the soluble MT145KdV5 trimer with Alum/ISCO-MPLA adjuvants. Time points for the immunizations and the bleeds are indicated. B. ELISA binding of polyclonal serum antibodies to MT145KdV5 SOSIP trimer. EC50 binding titers of pre-bleed (PB), post prime (1 wk, 2 wk, 4 wk and 8 wk) and post boost (9 wk and 10 wk) serum samples with MT145KdV5 SOSIP trimer. C. Neutralization curves of 10 wk immune serum IgG samples against the autologous MT145KdV5 virus. Purified IgGs from 10 wk immune serum samples were tested against the MT145KdV5 virus in TZM-b1 reporter cell-based assay. IC₅₀ virus neutralization titers for each serum IgG sample are shown. V2-apex bnAb, PGT145 was control for the neutralization assay.

### DETAILED DESCRIPTION OF THE INVENTION

The invention provides molecules which comprise regions and epitopes based on Env of various SIV isolates and advantageously bind to and are capable of stimulating production of bnAbs of HIV and display V2 apex epitopes associated with that. Virus isolates may display epitopes that are conserved across species boundaries. For example, SIV displays epitopes observed to bind to SIV and HIV antibodies, and it is theorized that such epitopes are both important for virus function and provide a means for crossing of species boundaries. Nevertheless, such epitopes may not be highly immunogenic, thus immune responses may be dominated by antibodies that bind to other more variable viral epitopes. Such variable epitopes may not be present on a broad range of viral isolates such that vaccines presenting those epitopes will not be protective against a broad range of viral isolates. Moreover, epitopes that might stimulate bnAbs may be inaccessible to antibody binding, one consequence being limited stimulation of germline B cells making antibodies capable of recognizing such epitopes. Accordingly, the invention provides engineered virus components and vaccines that display epitopes that bind to and are capable of efficiently stimulating antibodies that broadly neutralize HIV. Moreover, the invention compositions and methods of making and using immunogenic molecules that efficiently stimulate germline B cells and antibody production by those cells.

One of the Env sites that has shown great promise for vaccine targeting is the V2 apex bnAb epitope (Andrabi et al., 2015; Gorman et al., 2016; Voss et al., 2017). This bnAb epitope sits at the 3-fold axis of the trimer and is primarily formed by a patch rich in positively charged lysine residues and protected by two glycans at HXB2 HIV-1 reference positions N160 and N156/N173 that are part of the Env glycan shield. The bnAb precursors targeting this site possess a long anionic heavy-chain complementarity-determining region 3 (CDRH3) that penetrates the glycan shield to reach the protein epitope surface underneath. BnAb prototypes within this class interact with the V2 apex bnAb protein-glycan core epitope through common germline-encoded motifs and are, thus, targetable by unique trimers that bind with their germline Ab versions. Hence, the germline-priming immunogens to this site could be based directly on native-like trimer configurations. Other features of this site include: a) V2 apex bnAbs are elicited frequently in humans that make bnAbs, b) they emerge early in infection, and c) they possess relatively low levels of somatic mutation compared to most other HIV Env bnAbs.

Among the major HIV Env bnAb specificities the V2 apex site is a bnAb site that consistently exhibits cross-group neutralizing activity with virus Envs derived from HIV-1 group M, N, O and P, and in addition, V2-apex bnAbs display cross-neutralizing activity with the Simian Immunodeficiency Virus (SIV) isolates that infect chimpanzees (SIVcpzPtt [Pan troglodytes troglodytes], SIVcpzPts [Pan troglodytes schweinfurthii]) and gorillas (SIVgor). The sought to design molecules useful as immunogens, based on features of SIVcpzPtt Env, that could guide an immunofocused response to the HIV V2 apex bnAb epitope. The applicants elected a stable trimer as a useful format for the design of SIVcpzPtt-based molecules and immunogens. The SIV based immunogens of the invention are capable of enriching V2 apex-specific B cells, and owing to V2 apex species cross-conservation, help guide a V2-focused neutralizing Ab response when coupled with HIV trimers in a sequential prime/boost immunization strategy. In such an immunization scheme, overall Env backbone sequence diversity in combination with conservation of the V2 apex bnAb epitope in sequentially administered immunogens also reduces germinal center competition for V2 apex-specific B cells, favors a B cell recall response to the V2 apex bnAb epitope, and reduces off-target Env-specific responses.

The invention pertains to the identification, design, synthesis and isolation of molecules from SIV proteins, peptides, fragments, and mutants thereof that bind to HIV directed antibodies. The invention also pertains to molecules from SIV proteins, peptides, fragments, and mutants thereof used as immunogens to stimulate HIV directed antibodies, especially bnAbs that bind to HIV as well as nucleic acids encoding the same. The immunogens may comprise proteins, peptides, fragments, such as the various trimers disclosed herein The present invention also relates to homologues, derivatives and variants of the sequences of the mutant trimers and nucleic acids encoding the same, wherein it is preferred that the homologue, derivative or variant have at least 50%, at least 60%, at least 70%, at least 75%, at least 80%, at least 85%, at least 90%, at least 93%, at least 95%, at least 97%, at least 98% or at least 99% homology or identity with the sequence of the mutant trimers and nucleic acids encoding the same. It is noted that within this specification, homology to sequences of the mutant proteins and nucleic acids encoding the same refers to the homology of the homologue, derivative or variant to the binding site of the mutant proteins and nucleic acids encoding the same.

The invention still further relates to nucleic acid sequences expressing the proteins, peptides, fragments, trimers, and mutants disclosed herein, or homologues, variants or derivatives thereof and the epitopes presented thereon. One of skill in the art will know, recognize and understand techniques used to create such. Additionally, one of skill in the art will be able to incorporate such a nucleic acid sequence into an appropriate vector, allowing for production of the amino acid sequence of mutant proteins and nucleic acids encoding the same or a homologue, variant or derivative thereof.

The proteins, peptides, fragments, trimers, and mutants disclosed herein, or homologues, variants or derivatives thereof and the epitopes presented thereon may also be useful in screening for ligands that bind to said epitopes. Such ligands preferably block the epitopes and thus prevent infection.

Where used herein and unless specifically indicated otherwise, the following terms are intended to have the following meanings in addition to any broader (or narrower) meanings the terms might enjoy in the art:

The term “isolated” or “non-naturally occurring” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art may readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

In an advantageous embodiment, a tag may be utilized for purification or biotinylation. The tag for purification may be a his tag. In another embodiment, the tag for biotinylation may be an avi-tag. Other tags are contemplated for purification, however, purification may be accomplished without a tag. In another embodiment, antibody (such as, not limited to, a broadly neutralizing antibody) affinity columns are contemplated. In another embodiment, lectin columns are contemplated.

Native-like soluble trimers can be made by several methods that all involve stabilizing associations between envelope protein subunits. See, e.g., P. Dosenovic et al, “Immunization for HIV-1 broadly neutralizing antibodies in human Ig knockin mice,” Cell, 161:1-11, 2015; J. G. Jardine et al, “Priming a broadly neutralizing antibody response to HIV-1 using a germline targeting immunogen,” Science, doi: 10.1126/science.aac5894, 2015 and R. W. Sanders et al, “HIV-1 neutralalizing antibodies induced by native-like envelope trimers,” Science, doi: 10.1 126/science.aac4223, 2015.

The proteins and compounds of the invention need not be isolated in the sense defined above, however.

Compounds which have a chemical structure selected using the invention, wherein said compounds are neutralizing antibody binders, form a further aspect of the invention; and, such compounds may be used in methods of medical treatments, such as for diagnosis, preventing or treating HIV or for eliciting antibodies for diagnosis of HIV, including use in vaccines. Further, such compounds may be used in the preparation of medicaments for such treatments or prevention, or compositions for diagnostic purposes. The compounds may be employed alone or in combination with other treatments, vaccines or preventatives; and, the compounds may be used in the preparation of combination medicaments for such treatments or prevention, or in kits containing the compound and the other treatment or preventative.

The invention relates to immunogenic molecules that bind to and stimulate antibody production, particularly molecules that efficiently stimulate germline antibodies that are broadly neutralizing and/or precursors to antibodies that are broadly neutralizing against epitopes common to a variety of HIV isolates. The common epitopes may be evolutionarily selected and not especially immunogenic. Thus it can be beneficial to present antigenic molecules to the immune system that efficiently stimulate antibodies that bind to the common epitope while avoiding antibodies directed at epitopes that are not broadly neutralizing. Accordingly, the invention provides molecules engineered from SIV env regions designed to bind to and stimulate germline antibodies that are broadly neutralizing HIV antibodies or are precursors of broadly neutralizing HIV antibodies.

As described herein, in certain embodiments of the invention, useful V2 apex sequences comprise two glycans, thus two glycosylated aspargine residues, and basic amino acid residues, preferably three or four basic amino acid residues, in a four-amino acid sequence, for example, without limitation, Xxx-Lys-Lys-Lys (SEQ ID NO:1), Lys-Xxx-Lys-Lys (SEQ ID NO: 2), Lys-Lys-Xxx-Lys (SEQ ID NO:3), Lys-Lys-Lys-XXX (SEQ ID NO:4), or Lys-Lys-Lys-Lys (SEQ ID NO:5). In certain embodiments, the molecule comprises Asn-Xxxₐ-Asn-Xxx_{b}-XXX_{c}-Xxxₐ, wherein Xxxₐ comprises two or three amino acids, Xxx_{b} comprises five to seven amino acids, and Xxxx comprises four amino acids, each of the four amino acid selected from Lys or Arg.

In certain embodiments, molecules of the invention comprise the amino acid sequence N-C-X₁-F-N-X₂-X₃-X₄-X₅-X₆-X₇-X₈-X₉-X₁₀-X₁₁-X₁₂-X₁₃-X₁₄-X₁₅-X₁₆ (SEQ ID NO:7): wherein X₁ comprises S, T, N, or F; X₂ comprises I, V, T, Q, or M; X₃ comprises S or T; X₄ comprises S or T; X₅ comprises S, E, or G; X₆ comprises I, L, V or F; X₇ comprises K or R; X₈ comprises G or D; X₉ comprises K, R, E, or Q; X₁₀ comprises K, R, E, or Q; X₁₁ comprises K, R, E, or Q; X₁₂ comprises K, E, or Q; X₁₃ comprises E, T, V, I, or M; X₁₄ comprises Y or K; X is comprises A or S; and X₁₆ comprises F, I, or L. In certain embodiments, at least three of X₉, X₁₀, X₁₁, and X₁₂ comprise basic amino acid residues. (SEQ ID NO: 8) In certain embodiments, all of X₉, X₁₀, X₁₁, and X₁₂ comprise basic amino acid residues. (SEQ ID NO: 9) In certain embodiments, X₉, X₁₀, X₁₁, and X₁₂ each comprise K. (SEQ ID NO: 10) In one such embodiment, the amino acid sequence comprises NCSFNVTTELRDKKRQVYSL (SEQ ID NO:11). In another embodiment, the amino acid sequence comprises NCSFNVTTELRDKKRKVYSL (SEQ ID NO:12). In another embodiment, the amino acid sequence comprises NCSFNVTTELRDKKKKEYSF (SEQ ID NO: 13). In another embodiment, the amino acid sequence comprises NCSFNATTELRDKKKKEYAL (SEQ ID NO:14).

In certain embodiments, molecules of the invention comprise Asn-Cys-Ser-Phe-Asn-Val-Thr-Thr-Glu-Leu-Arg-Asp-Lys-Lys-Arg-Gln-Val-Tyr-Ser-Leu-Phe-Phe-Tyr (SEQ ID NO: 15), or Asn-Cys-Ser-Phe-Asn-Val-Thr-Thr-Glu-Leu-Arg-Asp-Lys-Lys-Arg-Gln-Val-Tyr-Ser-Leu-Phe-Tyr (SEQ ID NO: 16), or Asn-Cys-Ser-Phe-Asn-Val-Thr-Thr-Glu-Leu-Arg-Asp-Lys-Lys-Arg-Lys-Val-Tyr-Ser-Leu-Phe-Phe-Tyr (SEQ ID NO:17), or Asn-Cys-Ser-Phe-Asn-Val-Thr-Thr-Glu-Leu-Arg-Asp-Lys-Lys-Arg-Lys-Val-Tyr-Ser-Leu-Phe-Tyr (SEQ ID NO:18), or Asn-Cys-Ser-Phe-Asn-Val-Thr-Thr-Glu-Leu-Arg-Asp-Lys-Lys-Lys-Lys-Glu-Tyr-Ser-Phe-Phe-Phe-Tyr (SEQ ID NO:19); or Asn-Cys-Ser-Phe-Asn-Val-Thr-Thr-Glu-Leu-Arg-Asp-Lys-Lys-Lys-Lys-Glu-Tyr-Ser-Phe-Phe-Tyr (SEQ ID NO:20); or Asn-Cys-Ser-Phe-Asn-Ala-Thr-Thr-Glu-Leu-Arg-Asp-Lys-Lys-Lys-Lys-Glu-Tyr-Ala-Leu-Phe-Phe-Tyr (SEQ ID NO:21), or Asn-Cys-Ser-Phe-Asn-Ala-Thr-Thr-Glu-Leu-Arg-Asp-Lys-Lys-Lys-Lys-Glu-Tyr-Ala-Leu-Phe-Tyr (SEQ ID NO: 22).

The bnAb stimulating engineered V2 apex region sequences of the invention, such as but not limited to MT145K, may be incorporated in engineered env proteins and protein fragments. Exemplary, non-limiting examples of env proteins with GenBank sequence accession numbers are set forth in the table See below. See also public databases such as hiv.lanl.gov/components/sequence/HIV/search/search.html. Along with the V2 apex region sequences, the env proteins and fragments can comprise sequences set forth in the table below (e.g., env sequences of naturally occurring SIV variants and escape mutants isolated from infected individuals) or be similar in sequence to such isolated variants. Thus, alternative embodiments of env proteins and fragments have at least 50%, at least 55%, at least 60%, at least 65%, at least 70%, at least 75%, at least 80%, at least 85%, at least 90%, at least 95%, at least 96%, at least 97%, at least 98%, at least 99% identity to the SIV env proteins in Table 1. (See hiv.lanl.gov/components/sequence/HIV/search/search.html)

TABLE 1
Exemplary SIV ENV proteins

| Patient ID | Accession | SEQ ID NO: | Name | Subtype | Country |
|---|---|---|---|---|---|
| Marylin(4288) | AF103818 | 317 | US_Marilyn | CPZ | United States |
| CAM3(4290) | AF115393 | 318 | CAM3 | CPZ | Cameroon |
| GAB2(3694) | AF382828 | 319 | SIVcpzGAB2 | CPZ | Gabon |
| Ch-06(27172) | AF447763 | 320 | TAN1 | CPZ | Tanzania |
| CAM5(4289) | AJ271369 | 321 | CAM5 | CPZ | Cameroon |
| CAM13(6922) | AY169968 | 322 | SIVcpzCAM13 | CPZ | Cameroon |
| MB66(23595) | DQ373063 | 323 | SIVcpzMB66 | CPZ | Cameroon |
| LB7(41638) | DQ373064 | 324 | SIVcpzLB7 | CPZ | Cameroon |
| EK505(50579) | DQ373065 | 325 | SIVcpzEK505 | CPZ | Cameroon |
| MT145(23809) | DQ373066 | 326 | SIVcpzMT145 | CPZ | Cameroon |
| Ch-64(3056) | DQ374657 | 327 | TAN2_69 | CPZ | Tanzania |
| CH-45(3137) | DQ374658 | 328 | TAN3_1 | CPZ | Tanzania |
| Ch-06(27172) | EF394356 | 329 | TAN1 | CPZ | Tanzania |
| Ch-64(3056) | EF394357 | 330 | TAN2 | CPZ | Tanzania |
| CH-45(3137) | EF394358 | 331 | TAN3 | CPZ | Tanzania |
| DP943(23593) | EF535993 | 332 | SIVcpzDP943 | CPZ | Cameroon |
| MB897(23594) | EF535994 | 333 | SIVcpzMB897 | CPZ | Cameroon |
| ID10(30898) | FJ424863 | 334 | SIVgorCP2135con | GOR | Cameroon |
| ID9(30899) | FJ424864 | 335 | SIVgorCP2139_1con | GOR | Cameroon |
| ID9(30899) | FJ424865 | 336 | SIVgorCP2139_2con | GOR | Cameroon |
| ID9(30899) | FJ424866 | 337 | SIVgor2139_287 | GOR | Cameroon |
| ID1(13725) | FJ424871 | 338 | SIVgorCP684con | GOR | Cameroon |
| Cam155(38651) | FR686510 | 339 | Ptt_04Cam155 | CPZ | Cameroon |
| Cam155(38651) | FR686511 | 340 | Ptt_09Cam155 | CPZ | Cameroon |
| Gab4(47562) | GQ217539 | 341 | SIVcpzGab4 | CPZ | Gabon |
| Ch-58(44459) | JN091690 | 342 | UG38 | CPZ | Tanzania |
| Ch-071(32642) | JN091691 | 343 | TAN5 | CPZ | Tanzania |
| EK505(50579) | JN835460 | 344 | SIVcpzEK505.c2 | CPZ | Cameroon |
| MB897(23594) | JN835461 | 345 | SIVcpzMB897.c2 | CPZ | Cameroon |
| MT145(23809) | JN835462 | 346 | SIVcpzMT145.c2 | CPZ | Cameroon |
| Ch-080(32641) | JQ768416 | 347 | SIVcpzTAN13 | CPZ | Tanzania |
| BF1167(32614) | JQ866001 | 348 | BF1167 | CPZ | Congo |
| LB715(41737) | JX178450 | 349 | LB715 | CPZ | Cameroon |
| BPID1(78681) | KP004989 | 350 | SIVgor-BPID1 | GOR | Cameroon |
| BPID15(78682) | KP004990 | 351 | SIVgor-BPID15 | GOR | Cameroon |
| BQID2(78683) | KP004991 | 352 | SIVgor-BQID2 | GOR | Cameroon |
| ANT(65) | U42720 | 353 | ANT | CPZ | Congo |
| GAB1(66) | X52154 | 354 | GAB1 | CPZ | Gabon |

Molecules of the invention may be further engineered to remove or reduce the immunogenicity of dominant epitopes that might otherwise interfere with a desired immune response. Such dominant env regions have been characterized and include, without limitation, epitopes located in the V1 (a.a. 111-130) and V3 (a.a. 311-330) domains and near the amino terminal part (a.a. 601-619) of the transmembrane gp36 of SIVmac251. Many immunodominant regions are similar among SIVs and HIVs (see e.g., Benichou, S. et al., 1993, Virology 194:870). As demonstrated herein, deletion of a portion of the immunodominant V5-loop from an engineered MT145K molecule results in improved induction of cross-neutralizing antibody responses compared to the parent SIV molecule.

As disclosed and exemplified herein, germline reverted antibodies can be designed by comparing the heavy and light chains of an isolated bnAb that binds to an epitope of interest to nucleotide sequences and/or the amino acid sequences potentially encoded by germline V gene segments. For example, one can obtain a germline reverted antibody starting from the amino acid sequence of an antibody directed to an antigen or epitope of choice and identifying germline encoded antibody segments (i.e., heavy chain V, D, and J gene segments and light chain V and J gene segments) close in sequence to the starting antibody. Germline variable genes are cataloged, for example at VBASE2 (Retter I, Althaus H H, Münch R, Müller W: VBASE2, an integrative V gene database. Nucleic Acids Res. 2005 Jan. 1; 33 (Database issue): D671-4). Starting from a known or preselected antibody and moving to such a database can be advantageous as the paired heavy and light chains will be compatible and likely to be well represented in the repertoire of expressed germline genes.

The invention provides germline reverted heavy and light chain variable domains, including heavy and light chain CDR regions that bind to molecules of the invention. The following are exemplary and bind to certain engineered V2 regions described herein.

TABLE 2
Germline reverted heavy and light chains and Kabat CDRs

| | SEQ | |
|---|---|---|
| | ID | |
| | NO: | |
| PG9_VDJH_iGL | 32 | QVQLVESGGGVVQPGRSLRLSCAASGFTFSSYGMHWVRQAPGKGLEWVAV |
| GenBank | | ISYDGSNKYYADSVKGRFTISRDNSKNTLYLQMNSLRAEDTAVYYCAREA |
| Accession: | | GGPDYRNGYNYYDFWSGYYYYYYMDVWGKGTTVTVSS |
| MK825341 | | |
| CDR-H1 | 33 | SYGMH |
| CDR-H2 | 34 | VISYDGSNKYYADSVKG |
| CDR-H3 | 35 | EAGGPDYRNGYNYYDFWSGYYYYYYMDV |
| PG9VJL_iGL | 41 | QSALTQPASVSGSPGQSITISCTGTSSDVGGYNYVSWYQQHPGKAPKLMI |
| GenBank | | YEVSNRPSGVSNRFSGSKSGNTASLTISGLQAEDEADYYCSSYTSSSTWV |
| Accession: | | FGGGTKLTVL |
| MK825342 | | |
| CDR-L1 | 42 | TGTSSDVGGYNYVS |
| CDR-L2 | 43 | EVSNRPS |
| CDR-L3 | 44 | SSYTSSSTWV |
| CH01VDJH_iGL | 49 | EVQLVESGGGVVRPGGSLRLSCAASGFTFDDYGMSWVRQAPGKGLEWVSG |
| GenBank | | INWNGGSTGYADSVKGRFTISRDNAKNSLYLQMNSLRAEDTALYHCARGT |
| Accession: | | DYTIDDAGIHYYGSGSYWYFDLWGRGTLVTVSS |
| MK825343 | | |
| CDR-H1 | 50 | DYGMS |
| CDR-H2 | 51 | GINWNGGSTGYADSVKG |
| CDR-H3 | 52 | GTDYTIDDAGIHYYGSGSYWYFDL |
| CH01VJL_iGL | 58 | EIVLTQSPGTLSLSPGERATLSCRASQSVSSSYLAWYQQKPGQAPRLLIY |
| GenBank | | GASSRATGIPDRFSGSGSGTDFTLTISRLEPEDFAVYYCQQYGSSPWTFG |
| Accession: | | QGTKVEIK |
| MK825344 | | |
| CDR-L1 | 59 | RASQSVSSSYLA |
| CDR-L2 | 60 | GASSRAT |
| CDR-L3 | 61 | QQYGSSPWT |

Germline reverted antibodies such as the above provide for antibody design or selection. Using the CDRs of the germline reverted antibodies, bnAbs can be designed, or evolved by mutation and selection, that bind to the V2 apex region of HIV and SIV.

Broadly neutralizing antibodies of the invention include those set forth above and antibodies which comprise one, two, three, four, five, or all six of the CDRs set forth in Table 2, or comprise a heavy chain comprising one, two or three VH CDRs, or comprise a light chain comprising one, two or three VL CDRs. In certain embodiments, in bnAbs that bind to the V2 apex of HIV and/or SIV the CDRs, there can be conservative substitutions, as set forth elsewhere herein. The invention further includes bnAbs in which the heavy chains and/or light chains have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identity to germline reverted antibodies set forth. In certain such embodiments, variation in the CDRs is conservative.

Germline genes that bind to an epitope of interest can also be identified by screening libraries of human heavy and light chain genes, for example by phage display. Another method is by immunization and preparation of monoclonal antibodies from a mouse that expresses human antibody genes. Preferably hybridomas are prepared before there is substantial somatic mutation and selection. As mentioned, depending on the method employed, it is advantageous to select germline antibody gene segments that are well represented in the repertoire of expressed human antibodies, also taking into account compatibility of various heavy chain-light chain pairings. In certain embodiments, it is advantageous to select V-D, D-J, V-D-J (heavy chain) or V-J (light chain) combinations that are well represented.

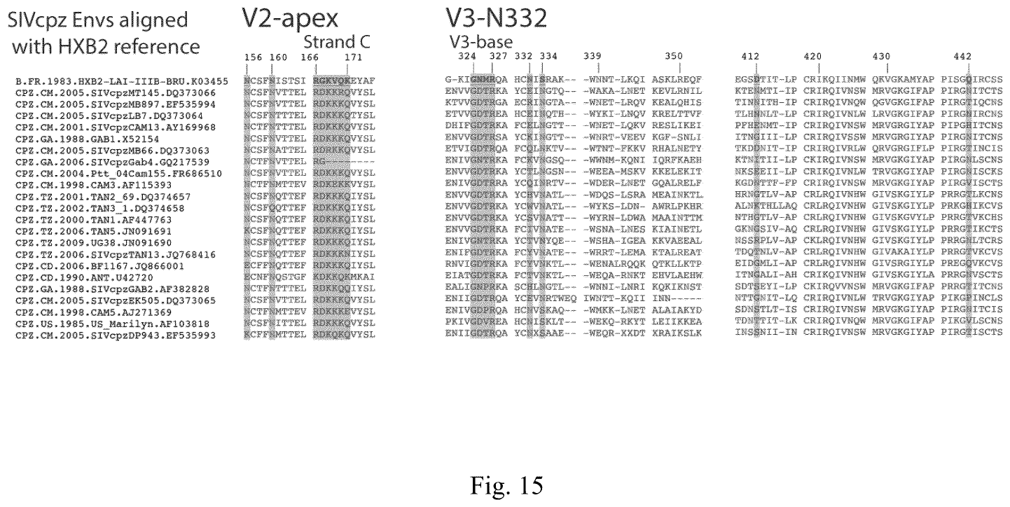

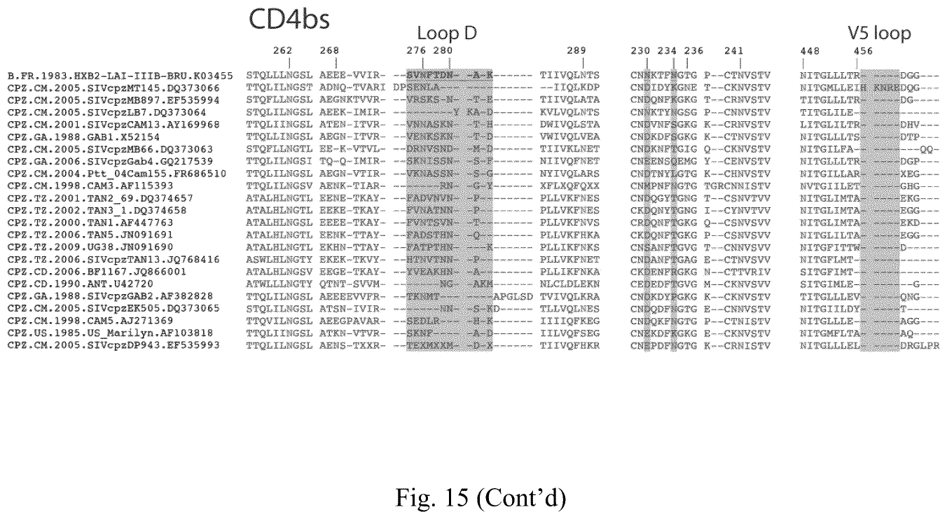

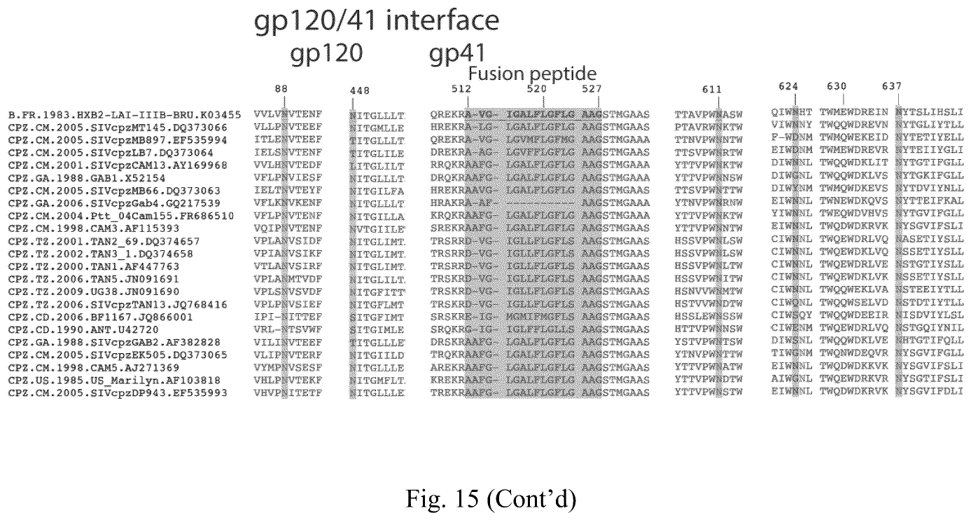

Amino acid sequences of the V2 apex regions of many SIV isolates have been determined and can be used or compared with HIV isolates to identify useful conserved epitopes that would be bound by broadly neutralizing antibodies. FIG. 15 herein, for example provides a comparison of SIVcpz Envs with an HBX2 HIV reference. An analysis may also include modeling or determination of 3D structure to identify interactions with bnAbs. Similar analysis can be done for other HIV reference isolates and SIV counterparts based on conserved structures, regions, and epitopes of the V2 apex, as well other conserved structures, regions, and epitopes that comprise bnAb binding sites to find suitable bases for development of SIV-based molecules and immunogens that bind to and stimulate HIV antibodies.

The HIV envelope protein (Env) is the target of broadly neutralizing antibodies (bnAbs) in natural infection. Env is a membrane protein composed of a trimer of gpl20 and gp41 subunits that contains a high degree of sequence diversity and a surface that is shielded by N-linked glycans. The bnAbs that target Env often have unusual features such as a long complementarity-determining region (CDR) H3, high levels of somatic hypermutation (SHM), and insertions and deletions (INDELS). Furthermore, most of the bnAbs recognize complex epitopes that are typically non-linear and have both protein and glycan components.

HIV type I (HIV-I) envelope is a noncovalent trimer of gp120-gp41 heterodimers, and its lability has hindered structural studies. SOSIP gp140 is a soluble, proteolytically mature form of the HIV-I envelope wherein gp120-gp41 interactions are stabilized via a disulfide bond and gp41 contains an additional trimer-stabilizing point mutation. The isolation of a substantially pure preparation of SOSIP gp140 trimers derived from KNH1144, a subtype A isolate was described in Iyer S P et al., AIDS Res Hum Retroviruses. 2007 June; 23(6):817-28. Following initial purification, the only significant contaminant was higher-order gp140 aggregates; however, 0.05% Tween 20 quantitatively converted these aggregates into trimers. The surfactant effect was rapid, dose dependent, and similarly effective for a subtype B SOSIP gp140.

Surfactant-treated SOSIP gp140 retained favorable antigenicity and formed compact trimers 12-13 nm in size as determined by electron microscopy. Iyer S P et al., AIDS Res Hum Retroviruses. 2007 June; 23(6):817-28 provides a description of homogeneous, cleaved HIV-I envelope trimers. These proteins may be useful as vaccine immunogens and for studying structure-function relationships within the HIV-I envelope glycoproteins.

Soluble, stabilized, proteolytically cleaved, trimeric proteins may be generated by engineering an intermolecular disulphide bond between gp120 and gp41 (SOS), combined with a single residue change, 1559P, within gp41 (SOSIP). SOSIP gp140 proteins based on the subtype A HIV-I strain KNHI 144 form particularly homogenous trimers compared to a prototypic strain (JR-FL, subtype B). Described in U.S. Pat. No. 7,939,083 are the determinants of this enhanced stability which are located in the N-terminal region of KNHI 1144 gp41 and that, when substituted into heterologous Env sequences (e.g., JR-FL and Ba-L) they have a similarly beneficial effect on trimer stability. These stabilized trimers retain the epitopes for several neutralizing antibodies and related agents (CD4-IgG2, b12, 2G12, 2F5 and 4E10) and the CD4-IgG2 molecule, so that the overall antigenic structure of the gp140 protein has not been adversely impaired by the trimer-stabilizing substitutions.

The HIV-I envelope glycoprotein (Env) is a trimer of heterodimers composed of two non-covalently associated subunits; the receptor-binding gp120, and the fusion machinery-containing gp41. Each subunit is derived from a gp160 precursor glycoprotein following cleavage by cellular furins (Wyatt R & Sodroski J (1998) Science 280(5371): 1884-1888). HIV-I gp120 binds the CD4 molecule on the surface of human target T cells to initiate the viral entry process, and following co-receptor engagement, fusion is mediated by gp41 (Dalgleish A G, et al. (1984) Nature 312(5996):763-767; McDougal J S, et al. (1986) J Immunol 137(9):2937-2944; mKarlsson Hedestam G B, et al. (2008) Nat Rev Microbial 6 (2): 143-155). The surface-exposed HIV-I Env trimer is the sole target for antibodies capable of neutralizing the virus (Burton D R, et al. (2004) Nat Immunol 5(3):233-236). Recently, a myriad of Env-directed broadly neutralizing antibodies (bnAbs) were isolated from numerous HIV-I-infected individuals, demonstrating that the human B cell response can effectively inhibit this variable pathogen (Wu X, et al. (2010) Science 329(5993):856-861; Walker L M, et al. (2009) Science 326(5950):285-289; Walker L M, et al. (2011) Nature 477(7365):466-470; Huang J, et al. (2012) Nature 491(7424):406-412; Scharf L, et al. (2014) Antibody 8ANC195 reveals a site of broad vulnerability on the HIV-I envelope spike. Cell reports 7(3):785-795; Klein F, et al. (2012) J Exp Med 209(8):1469-1479). Infection of macaques by a chimeric model virus, SHIV, can be prevented by prior passive immunization of all bnAbs so far tested, confirming the capacity of neutralizing antibodies to prevent HIV infection (Mascola J R, et al. (1999) J Virol 73(5):4009-4018; Hessell A J, et al. (2009) PLOS Pathog 5(5):el000433; Moldt B, et al. (2012) Proc Natl Acad Sci USA 109(46):18921-18925; Barouch D H, et al. (2013) Therapeutic efficacy of potent neutralizing HIV-I-specific monoclonal antibodies in SHIV-infected rhesus monkeys. Nature 503(7475):224-228).

Along with virus-specific T cells, an efficacious HIV-I vaccine therefore would likely need to generate bnAbs targeting Env. Although the premise is simple, in actuality, it is a tremendous challenge without precedent in the history of vaccinology. The difficulty to vaccinate against HIV arises from the extensive variability of Env present on the large number of HIV-I isolates simultaneously circulating in the human population as well as other mechanisms of immune evasion selected for by strong pressure from the human immune system.

Generally, vaccine-generated antibodies using either or both gp120 or gp41 sequences do not recognize native Env on the surface of cells or virus, do not neutralize primary isolates in vitro, and do not prevent infection in laboratory animals (Burton D R, et al. (2011) Proc Natl Acad Sci USA 108(27):11181-11186; Sundling C, et al. (2012) Science translational medicine 4(142):142ra196; Tran K, et al. (2014) Vaccine-elicited primate antibodies use a distinct approach to the HIV-I primary receptor binding site informing vaccine redesign. Proc Natl Acad Sci USA 111(7):E738-747). Non-neutralizing antibodies directed to the major variable region two (V2) of gp120 are associated with modest efficacy in a single human clinical trial (Haynes B F, et al. (2012) N Engl J Med 366(14):1275-1286; Zolla-Pazner S, et al. (2014) Vaccine-induced IgG antibodies to VIV2 regions of multiple HIV-I subtypes correlate with decreased risk of HIV-I infection. PLOS One 9(2):e87572), while, in general, Env-elicited antibodies fail to demonstrate protection in previous human clinical trials (Jones N G, et al. (2009) Vaccine 27(7):1136-1140; Rerks-Ngarm S, et al. (2009) N Engl J Med 361(23):2209-2220; Yates N L, et al. (2014) Vaccine-induced Env V1-V2 IgG3 correlates with lower HIV-I infection risk and declines soon after vaccination. Science translational medicine 6(228):228ra239).

Many Env-based trimeric candidate immunogens are engineered to eliminate cleavage between gp120 and gp41 (so called uncleaved gp140 trimers), usually generating imperfect mimetics of the functional spike based on antigenic profiling or EM analysis (Tran K, et al. (2014) Proc Natl Acad Sci US All 1(7):E738-747; Ringe R P, et al. (2013) Proc Natl Acad Sci USA 110(45):18256-18261). As a group, the defined, or presumed to be, disordered trimers (in adjuvant) generate high self-binding antibody titers. However, these vaccine-elicited antibodies do not efficiently neutralize most HIV-I primary isolates, that is, strains representative of those circulating in the human population (Sundling C, et al. (2012) Science translational medicine 4(142):142ral96; Chakrabarti B K, et al. (2013) J Virol 87(24):13239-13251; Kovacs J M, et al. (2012) Proc Natl Acad Sci USA 109(30):12111-12116; Nkolola J P, et al. (2014) Comparison of multiple adjuvants on the stability and immunogenicity of a clade C HIV-I gp140 trimer. Vaccine 32(18):2109-2116). Antibodies elicited by these immunogens target epitopes exposed only on the free gp120 and trimeric post-fusion forms of gp41 or disordered gp140s and thus are ineffective at accessing their epitopes buried within the ordered, quaternary structure achieved in the native Env spike. Applicants recently described the limitations of two CD4bs-directed non-bnAbs, (GE148 and GE136) generated following immunization of uncleaved gp140 trimers (YU2 gp140-foldon) in non-human primates (NHP). Non-bnAbs, represented by GE136 and 148, can only neutralize the sensitive so-called “tier I viruses” that are not representative of the more neutralization resistant tier 2-like primary isolates circulating in the human population. Using crystallography, EM reconstructions, paratope scanning and molecular modeling, Applicants determined that these vaccine-elicited antibodies fail to reach the CD4bs due to steric barriers imposed by quaternary packing of the native Env on neutralization resistant primary isolates, a property that Applicants use to Applicants' advantage in the negative-selection strategy presented here (Tran K, et al. (2014) Proc Natl Acad Sci USA 11 1(7):E738-747).

The cumulative historical data have led to the hypothesis that a more faithful mimic of the HIV-I spike that better recapitulates the native, pre-fusion form of Env, selectively displaying neutralizing determinants while occluding non-neutralizing determinants, may better elicit antibodies capable of accessing the native spike. A soluble Env mimetic, containing a disulfide linkage between gp120 and gp41 (SOS), first described in the 2000s, and further developed over the next decade, displays many of these properties, culminating in the determination of the high resolution structures of the well-ordered BG505 SOSIP trimers by crystallography and EM (Lyumkis D, et al. (2013) Science 342(6165):1484-1490; Julien J P, et al. (2013) Science 342(6165):1477-1483; Sanders R W, et al. (2013) PLOS Pathog 9(9):el003618; Depetris R S, et al. (2012) J Biol Chem 287(29):24239-24254). A sub-nanometer EM reconstruction of KNHI 144 SOSIP is also available but does not provide atomic level details (Bartesaghi A, Merk A, Borgnia M J, Milne J L, & Subramaniam S (2013) Nat Struct Mol Biol 20(12):1352-1357). The BG505 SOSIP and KNH1144 SOSIP trimers are derived from the Env sequences of the subtype A BG505 and KNHI 144 strains. These soluble trimers possess an engineered disulfide linkage between the gp120 and gp41 (at residues 501C and 605C, respectively) and an additional mutation in the heptad repeat I (HRI) of gp41 (1559P) that facilitates trimerization (Binley J M, et al. (2000) J Virol 74 (2): 627-643; Sanders R W, et al. (2002) J Virol 76(17):8875-8889). A truncation of the membrane proximal external region (MPER) at residue 664 enhances expression while decreasing aggregation is incorporated into the so-called BG505 SOSIP.664 trimers (Sanders R W, et al. (2013) PLOS Pathog 9(9):el003618; Sanders R W, et al. (2002) J Virol 76(17):8875-8889). Although SOSIP molecules based on other HIV-I primary strains were attempted over the past decade, the BG505- and KNHI 144-derived SOSIP trimers are the two limited examples of SOSIPs that yield homogeneous trimers suitable for high resolution biophysical and structural analysis. The structural explanation for the difficulty to readily transfer the SOSIP design to other HIV-I strain-derived sequences is not yet fully understood and would be valuable information to broaden the trimer design horizon.

Since the initial soluble native-like BG505 SOPIP.664 Env trimer was confirmed to adopt a near-native conformation by high-resolution structural analysis, multiple efforts to produce stable, soluble Env mimetics derived from multiple HIV-1 strains were pursued [Javiers, sanders]. Multiple solutions to this objective include the improved cleavage-independent NFL trimers, UFOs and modified SOSIPs. Both the SOSIP and NFL well-ordered trimers are efficiently recognized by broadly neutralizing antibodies (bnAbs) which arise sporadically during the course of natural infection. In some cases, including the important advances described here, have been used to isolate such bnAbs. One approach to elicit tier 2 neutralizing Abs has been to immunize the existing well-ordered trimers using prime: boosting in selected animal models. For BG505 and 16055 native-like trimers this approach does elicit tier 2 neutralizing antibodies, but of limited cross-reactive breadth (REFS).

Most cross-conserved sites on the HIV Env spike are occluded by evolved, incorporated self-N-glycans, limiting naïve B cell recognition of the underlying polypeptide surface. The exceptions are the protein surfaces of the primary receptor CD4 binding site (CD4bs) and the furin cleavage site (proximal to the gp120:41 interface). Infrequently, during the course of the natural HIV infection process, bnAbs are elicited to these aforementioned sites of vulnerability. In addition, other bnAbs directed to the V2 apex, the 332N-glycan supersite and to the fusion peptide or the high-mannose patch are elicited during the course of chronic HIV infection (REFS). However, prior to the present invention, rarely, if ever, have such bnAbs been elicited by vaccination of Env formulated with adjuvant.

After decades of development, advances in soluble HIV-1 Env mimics design permits the generation of a diverse array of native-like trimers (Ward and Wilson, 2017. The HIV-1 envelope glycoprotein structure: nailing down a moving target. Immunol Rev 275:21-32; Karlssonet al., 2017. Evolution of B cell analysis and Env trimer redesign. Immunol Rev 275:183-202). The successful development of the soluble SOSIP trimers provided proof-of-principle (Sanders et al, 2013. A next-generation cleaved, soluble HIV-1 Env trimer, BG505 SOSIP.664 gp140, expresses multiple epitopes for broadly neutralizing but not non-neutralizing antibodies. PLOS Pathog 9: e1003618) forming a prefusion native-like conformation (Lyumkis et al., 2013. Cryo-EM structure of a fully glycosylated soluble cleaved HIV-1 envelope trimer. Science 342:1484-1490; Julien et al, 2013. Crystal structure of a soluble cleaved HIV-1 envelope trimer. Science 342:1477-1483; Garces et al., 2015. Affinity Maturation of a Potent Family of HIV Antibodies Is Primarily Focused on Accommodating or Avoiding Glycans. Immunity 43:1053-1063; Pancera et al., 2014. Structure and immune recognition of trimeric pre-fusion HIV-1 Env. Nature 514:455-461). The SOSIP gp140 trimer is proteolytically cleaved by cellular furins to gp120 and gp41 subunits and covalently linked by an engineered intra-protomer disulfide bond A501C-T605C (SOS). These trimers also require mutation (1559P) in the gp41 heptad repeat 1 (HR1) to maintain well-ordered oligomers, as well as expression of exogenous furin for full conformational integrity (Sanders et al., 2013. A next-generation cleaved, soluble HIV-1 Env trimer, BG505 SOSIP.664 gp140, expresses multiple epitopes for broadly neutralizing but not non-neutralizing antibodies. PLOS Pathog 9: e1003618; Guenaga et al., 2015. Well-Ordered Trimeric HIV-1 Subtype B and C Soluble Spike Mimetics Generated by Negative Selection Display Native-like Properties. PLOS Pathog 11: e1004570; Julien et al., 2015. Design and structure of two HIV-1 clade C SOSIP.664 trimers that increase the arsenal of native-like Env immunogens. Proc Natl Acad Sci USA 112:11947-11952; de Taeye et al. 2015. Immunogenicity of Stabilized HIV-1 Envelope Trimers with Reduced Exposure of Non-neutralizing Epitopes. Cell 163:1702-1715; Pugach et al. 2015. A native-like SOSIP.664 trimer based on an HIV-1 subtype B env gene. J Virol 89:3380-3395; Ringe et al. 2013. Cleavage strongly influences whether soluble HIV-1 envelope glycoprotein trimers adopt a native-like conformation. Proc Natl Acad Sci USA 110:18256-18261; Ringe et al. 2015. Influences on the Design and Purification of Soluble, Recombinant Native-Like HIV-1 Envelope Glycoprotein Trimers. J Virol 89:12189-12210; Ringe et al. 2017. Reducing V3 Antigenicity and Immunogenicity on Soluble, Native-Like HIV-1 Env SOSIP Trimers. J Virol 91; Ahmedet al. 2017. Stabilization of a soluble, native-like trimeric form of an efficiently cleaved Indian HIV-1 clade C envelope glycoprotein. J Biol Chem 292:8236-8243; Sanders et al. 2002. Stabilization of the soluble, cleaved, trimeric form of the envelope glycoprotein complex of human immunodeficiency virus type 1. J Virol 76:8875-8889; Binley et al. 2000. A recombinant human immunodeficiency virus type 1 envelope glycoprotein complex stabilized by an intermolecular disulfide bond between the gp120 and gp41 subunits is an antigenic mimic of the trimeric virion-associated structure. J Virol 74:627-643). In the past years, Applicants developed an improved native-like trimer design, generating well-ordered soluble Env mimics that are fully cleavage-independent, termed native flexibly linked (NFL) trimers. This design uses a flexible linker (two copies of Gly4-Ser, “G4S” (SEQ ID NO:357)) to replace the natural cleavage site and sequence (Sharma et al. 2015. Cleavage-independent HIV-1 Env trimers engineered as soluble native spike mimetics for vaccine design. Cell Rep 11:539-550). The flexible linker between the natural C-terminus of gp120 and N-terminus of gp41, allows the un-cleaved trimers to achieve a native-like conformation without the need of furin for precursor processing. However, the original NFL trimer design contains the 1559P mutation (Sharma et al. 2015. Cleavage-independent HIV-1 Env trimers engineered as soluble native spike mimetics for vaccine design. Cell Rep 11:539-550) that was initially identified in the SOSIP context to disfavor the post fusion state (Sanders et al. 2013. A next-generation cleaved, soluble HIV-1 Env trimer, BG505 SOSIP.664 gp140, expresses multiple epitopes for broadly neutralizing but not non-neutralizing antibodies. PLOS Pathog 9: e1003618). Both the original SOSIP and NFL designs do not form a high percentage of well-ordered trimers in all Env contexts. In the original NFL design, it is relatively inefficient in generating high yields of trimers derived from clade C strains, such as 16055 (Guenaga et al. 2015. Structure-Guided Redesign Increases the Propensity of HIV Env To Generate Highly Stable Soluble Trimers. J Virol 90:2806-2817). To improve trimer design, Applicants incorporated residues from BG505 (called trimer-derived (TD) residues) into 16055 NFLs, substantially improving the propensity to form native-like trimers (Guenaga et al. 2015. Structure-Guided Redesign Increases the Propensity of HIV Env To Generate Highly Stable Soluble Trimers. J Virol 90:2806-2817) and the elicitation of tier 2 clade C neutralizing antibodies (Martinez-Murillo et al., GB. 2017. Particulate Array of Well-Ordered HIV Clade C Env Trimers Elicits Neutralizing Antibodies that Display a Unique V2 Cap Approach. Immunity 46:804-817 e807; Dubrovskaya et al. 2017. Targeted N-glycan deletion at the receptor-binding site retains HIV Env NFL trimer integrity and accelerates the elicited antibody response. PLOS Pathog 13: e1006614). Further improvements on the TD design by targeted glycine substitutions at helix-to-coil transitions that disfavor the post-fusion state of Env (TD CC+, namely “TD+”), significantly improve trimer homogeneity, yield, stability and antigenicity, resulting in the first high-resolution clade C Env structure (Guenaga et al. 2017. Glycine Substitution at Helix-to-Coil Transitions Facilitates the Structural Determination of a Stabilized Subtype C HIV Envelope Glycoprotein. Immunity 46:792-803 e793).

Applicants believe that the glycine changes may lower the activation potential of the gp41 (and Env) to change conformation, and therefore results in better behaved trimers in a lower energy well from the “activation state” to spring to the next conformation. In a simple model, gp41 is essentially spring-loaded and constrained by gp120 until receptor binding. These mutations may contribute to reducing the springiness.

Assays for screening for neutralizing antibodies are known in the art. A neutralization assay approach has been described previously (Binley J M, et al., (2004). Comprehensive Cross-Clade Neutralization Analysis of a Panel of Anti-Human Immunodeficiency Virus Type 1 Monoclonal Antibodies. J Viral. 78:13232-13252). Pseudotyped viruses may be generated by co-transfecting cells with at least two plasmids encoding the soluble Env cDNA of the present invention and the rest of the HIV genome separately. In the HIV genome encoding vector, the Env gene may be replaced by the firefly luciferase gene. Transfectant supematants containing pseudotyped virus may be co-incubated overnight with B cell supematants derived from activation of an infected donor's primary peripheral blood mononuclear cells (PBMCs). Cells stably transfected with and expressing CD4 plus the CCR5 and CXCR4 coreceptors may be added to the mixture and incubated for 3 days at 37° C. Infected cells may be quantified by luminometry.

In another embodiment of the present invention, the soluble envelope glycoproteins of the present invention may be crystallized in combination with any neutralizing antibodies, including those identified by the above methods, to determine the exact molecular surface where the soluble envelope glycoprotein binds with the neutralizing antibody to design HIV-I immunogens.

It is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of as used herein comprise the terms “consisting of, “consists” and “consists of, as well as the terms “consisting essentially of, “consists essentially” and “consists essentially of. It is noted that in this disclosure and particularly in the claims and/or paragraphs, terms such as “comprises”, “comprised”, “comprising” and the like can have the meaning attributed to it in U. S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention. It may be advantageous in the practice of the invention to be in compliance with Art. 53(c) EPC and Rule 28(b) and (c) EPC. Nothing herein is intended as a promise.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +1-5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one” or “X or more”, where X is a number and understand to mean X or increases one by one of X, such as one or more or at least one member(s) or “X or more” of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

All references cited in the present specification are hereby incorporated by reference in their entirety. In particular, the teachings of all references herein specifically referred to are incorporated by reference. Andrabi et al., 2019 is incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In this description of the invention, reference is made to the accompanying drawings that form a part hereof, and in which are shown by way of illustration only of specific embodiments in which the invention may be practiced. It is to be understood that other embodiments may be utilized and structural or logical changes may be made without departing from the scope of the present invention. The description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Unless specifically stated or obvious from context, as used herein, the term “or” is understood to be inclusive. Unless specifically stated or obvious from context, as used herein, the terms “a,” “an,” and “the” are understood to be singular or plural.

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
Fab, the fragment which contains a monovalent antigen-binding fragment of an antibody molecule can be produced by digestion of whole antibody with the enzyme papain to yield an intact light chain and a portion of one heavy chain;
Fab′, the fragment of an antibody molecule can be obtained by treating whole antibody with pepsin, followed by reduction, to yield an intact light chain and a portion of the heavy chain; two Fab′ fragments are obtained per antibody molecule;
F(ab′)₂, the fragment of the antibody that can be obtained by treating whole antibody with the enzyme pepsin without subsequent reduction; F(ab′)₂ is a dimer of two Fab′ fragments held together by two disulfide bonds;
scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

A “neutralizing antibody” may inhibit the entry of HIV-I virus F with a neutralization index >1.5 or >2.0. Broad and potent neutralizing antibodies may neutralize greater than about 50% of HIV-I viruses (from diverse clades and different strains within a clade) in a neutralization assay. The inhibitory concentration of the monoclonal antibody may be less than about 25 mg/ml to neutralize about 50% of the input virus in the neutralization assay.

It should be understood that the proteins, including the antibodies and/or antigens of the invention may differ from the exact sequences illustrated and described herein. Thus, the invention contemplates deletions, additions and substitutions to the sequences shown, so long as the sequences function in accordance with the methods of the invention. In this regard, particularly preferred substitutions may generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, praline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

As used herein the term “transgene” may be used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the Gag, Pol, Env, Nef, RT, and Int sequences of the invention may be altered in these ways.

As regards codon optimization, the nucleic acid molecules of the invention have a nucleotide sequence that encodes the antigens of the invention and can be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens can be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. Any suitable method of codon optimization may be used. Such methods, and the selection of such methods, are well known to those of skill in the art. In addition, there are several companies that will optimize codons of sequences, such as Genart. Thus, the nucleotide sequences of the invention can readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87:2264-2268, modified as in Karlin & Altschul, Proc. Natl. Acad. Sci. USA 1993; 90:5873-5877.

Another example of a mathematical algorithm used for comparison of sequences is the algorithm of Myers & Miller, CABIOS 1988; 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85:2444-2448. Advantageous for use according to the present invention is the WU-BLAST (Washington University BLAST) version 2.0 software. WU-BLAST version 2.0 executable programs for several UNIX platforms can be downloaded from the Washington University BLAST website. This program is based on WU-BLAST version 1.4, which in turn is based on the public domain NCBI-BLAST version 1.4 (Altschul & Gish, 1996, Local alignment statistics, Doolittle ed., Methods in Enzymology 266:460-480; Altschul et al., Journal of Molecular Biology 1990; 215:403-410; Gish & States, 1993; Nature Genetics 3:266-272; Karlin & Altschul, 1993; Proc. Natl. Acad. Sci. USA 90:5873-5877; all of which are incorporated by reference herein).

The various recombinant nucleotide sequences and antibodies and/or antigens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the antibodies and/or antigens of the present invention may be used in accordance with the present invention. In certain embodiments, the antigens and/or antibodies of the present invention may be used in vitro (such as using cell-free expression systems) and/or in cultured cells grown in vitro in order to produce the encoded HIV-antigens and/or antibodies which may then be used for various applications such as in the production of proteinaceous vaccines. For such applications, any vector that allows expression of the antigens and/or antibodies in vitro and/or in cultured cells may be used.

For applications where it is desired that the antibodies and/or antigens be expressed in vivo, for example when the transgenes of the invention are used in DNA or DNA-containing vaccines, any vector that allows for the expression of the antibodies and/or antigens of the present invention and is safe for use in vivo may be used. In preferred embodiments the vectors used are safe for use in humans, mammals and/or laboratory animals.

For the antibodies and/or antigens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, intrans, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the transgenes of the invention. For example, suitable promoters and/or enhancers can be selected from the Eukaryotic Promoter Database (EPDB).

The present invention relates to a recombinant vector expressing a foreign epitope. Advantageously, the epitope is an SIV or HIV epitope or combination thereof. In an advantageous embodiment, the SIV or HIV epitope is a soluble envelope glycoprotein, however, the present invention may encompass additional HIV antigens, epitopes or immunogens. Advantageously, the additional epitope is an HIV antigen, HIV epitope or an HIV immunogen, such as, but not limited to, the HIV antigens, HIV epitopes or HIV immunogens of U.S. Pat. Nos. 7,341,731; 7,335,364; 7,329,807; 7,323,553; 7,320,859; 7,311,920; 7,306,798; 7,285,646; 7,285,289; 7,285,271; 7,282,364; 7,273,695; 7,270,997; 7,262,270; 7,244,819; 7,244,575; 7,232,567; 7,232,566; 7,223,844; 7,223,739; 7,223,534; 7,223,368; 7,220,554; 7,214,530; 7,211,659; 7,211,432; 7,205,159; 7,198,934; 7,195,768; 7,192,555; 7,189,826; 7,189,522; 7,186,507; 7,179,645; 7,175,843; 7,172,761; 7,169,550; 7,157,083; 7,153,509; 7,147,862; 7,141,550; 7,129,219; 7,122,188; 7,118,859; 7,118,855; 7,118,751; 7,118,742; 7,105,655; 7,101,552; 7,097,971; 7,097,842; 7,094,405; 7,091,049; 7,090,648; 7,087,377; 7,083,787; 7,070,787; 7,070,781; 7,060,273; 7,056,521; 7,056,519; 7,049,136; 7,048,929; 7,033,593; 7,030,094; 7,022,326; 7,009,037; 7,008,622; 7,001,759; 6,997,863; 6,995,008; 6,979,535; 6,974,574; 6,972,126; 6,969,609; 6,964,769; 6,964,762; 6,958,158; 6,956,059; 6,953,689; 6,951,648; 6,946,075; 6,927,031; 6,919,319; 6,919,318; 6,919,077; 6,913,752; 6,911,315; 6,908,617; 6,908,612; 6,902,743; 6,900,010; 6,893,869; 6,884,785; 6,884,435; 6,875,435; 6,867,005; 6,861,234; 6,855,539; 6,841,381 6,841,345; 6,838,477; 6,821,955; 6,818,392; 6,818,222; 6,815,217; 6,815,201; 6,812,026; 6,812,025; 6,812,024; 6,808,923; 6,806,055; 6,803,231; 6,800,613; 6,800,288; 6,797,811; 6,780,967; 6,780,598; 6,773,920; 6,764,682; 6,761,893; 6,753,015; 6,750,005; 6,737,239; 6,737,067; 6,730,304; 6,720,310; 6,716,823; 6,713,301; 6,713,070; 6,706,859; 6,699,722; 6,699,656; 6,696,291; 6,692,745; 6,670,181; 6,670,115; 6,664,406; 6,657,055; 6,657,050; 6,656,471; 6,653,066; 6,649,409; 6,649,372; 6,645,732; 6,641,816; 6,635,469; 6,613,530; 6,605,427; 6,602,709; 6,602,705; 6,600,023; 6,596,477; 6,596,172; 6,593,103; 6,593,079; 6,579,673; 6,576,758; 6,573,245; 6,573,040; 6,569,418; 6,569,340; 6,562,800; 6,558,961; 6,551,828; 6,551,824; 6,548,275; 6,544,780; 6,544,752; 6,544,728; 6,534,482; 6,534,312; 6,534,064; 6,531,572; 6,531,313; 6,525,179; 6,525,028; 6,524,582; 6,521,449; 6,518,030; 6,518,015; 6,514,691; 6,514,503; 6,511,845; 6,511,812; 6,511,801; 6,509,313; 6,506,384; 6,503,882; 6,495,676; 6,495,526; 6,495,347; 6,492,123; 6,489,131; 6,489,129; 6,482,614; 6,479,286; 6,479,284; 6,465,634; 6,461,615; 6,458,560; 6,458,527; 6,458,370; 6,451,601; 6,451,592; 6,451,323; 6,436,407; 6,432,633; 6,428,970; 6,428,952; 6,428,790; 6,420,139; 6,416,997; 6,410,318; 6,410,028; 6,410,014; 6,407,221; 6,406,710; 6,403,092; 6,399,295; 6,392,013; 6,391,657; 6,384,198; 6,380,170; 6,376,170; 6,372,426; 6,365,187; 6,358,739; 6,355,248; 6,355,247; 6,348,450; 6,342,372; 6,342,228; 6,338,952; 6,337,179; 6,335,183; 6,335,017; 6,331,404; 6,329,202; 6,329,173; 6,328,976; 6,322,964; 6,319,666; 6,319,665; 6,319,500; 6,319,494; 6,316,205; 6,316,003; 6,309,633; 6,306,625; 6,296,807; 6,294,322; 6,291,239; 6,291,157; 6,287,568; 6,284,456; 6,284,194; 6,274,337; 6,270,956; 6,270,769; 6,268,484; 6,265,562; 6,265,149; 6,262,029; 6,261,762; 6,261,571; 6,261,569; 6,258,599; 6,258,358; 6,248,332; 6,245,331; 6,242,461; 6,241,986; 6,235,526; 6,235,466; 6,232,120; 6,228,361; 6,221,579; 6,214,862; 6,214,804; 6,210,963; 6,210,873; 6,207,185; 6,203,974; 6,197,755; 6,197,531; 6,197,496; 6,194,142; 6,190,871; 6,190,666; 6,168,923; 6,156,302; 6,153,408; 6,153,393; 6,153,392; 6,153,378; 6,153,377; 6,146,635; 6,146,614; 6,143,876 6,140,059; 6,140,043; 6,139,746; 6,132,992; 6,124,306; 6,124,132; 6,121,006; 6,120,990; 6,114,507; 6,114,143; 6,110,466; 6,107,020; 6,103,521; 6,100,234; 6,099,848; 6,099,847; 6,096,291; 6,093,405; 6,090,392; 6,087,476; 6,083,903; 6,080,846; 6,080,725; 6,074,650; 6,074,646; 6,070,126; 6,063,905; 6,063,564; 6,060,256; 6,060,064; 6,048,530; 6,045,788; 6,043,347; 6,043,248; 6,042,831; 6,037,165; 6,033,672; 6,030,772; 6,030,770; 6,030,618; 6,025,141; 6,025,125; 6,020,468; 6,019,979; 6,017,543; 6,017,537; 6,015,694; 6,015,661; 6,013,484; 6,013,432; 6,007,838; 6,004,811; 6,004,807; 6,004,763; 5,998,132; 5,993,819; 5,989,806; 5,985,926; 5,985,641; 5,985,545; 5,981,537; 5,981,505; 5,981,170; 5,976,551; 5,972,339; 5,965,371; 5,962,428; 5,962,318; 5,961,979; 5,961,970; 5,958,765; 5,958,422; 5,955,647; 5,955,342; 5,951,986; 5,951,975; 5,942,237; 5,939,277; 5,939,074; 5,935,580; 5,928,930; 5,928,913; 5,928,644; 5,928,642; 5,925,513; 5,922,550; 5,922,325; 5,919,458; 5,916,806; 5,916,563; 5,914,395; 5,914,109; 5,912,338; 5,912,176; 5,912,170; 5,906,936; 5,895,650; 5,891,623; 5,888,726; 5,885,580 5,885,578; 5,879,685; 5,876,731; 5,876,716; 5,874,226; 5,872,012; 5,871,747; 5,869,058; 5,866,694; 5,866,341; 5,866,320; 5,866,319; 5,866,137; 5,861,290; 5,858,740; 5,858,647; 5,858,646; 5,858,369; 5,858,368; 5,858,366; 5,856,185; 5,854,400; 5,853,736; 5,853,725; 5,853,724; 5,852,186; 5,851,829; 5,851,529; 5,849,475; 5,849,288; 5,843,728; 5,843,723; 5,843,640; 5,843,635; 5,840,480; 5,837,510; 5,837,250; 5,837,242; 5,834,599; 5,834,441; 5,834,429; 5,834,256; 5,830,876; 5,830,641; 5,830,475; 5,830,458; 5,830,457; 5,827,749; 5,827,723; 5,824,497; 5,824,304; 5,821,047; 5,817,767; 5,817,754; 5,817,637; 5,817,470; 5,817,318; 5,814,482; 5,807,707; 5,804,604; 5,804,371; 5,800,822; 5,795,955; 5,795,743; 5,795,572; 5,789,388; 5,780,279; 5,780,038; 5,776,703; 5,773,260; 5,770,572; 5,766,844; 5,766,842; 5,766,625; 5,763,574; 5,763,190; 5,762,965; 5,759,769; 5,756,666; 5,753,258; 5,750,373; 5,747,641; 5,747,526; 5,747,028; 5,736,320; 5,736,146; 5,733,760; 5,731,189; 5,728,385; 5,721,095; 5,716,826; 5,716,637; 5,716,613; 5,714,374; 5,709,879; 5,709,860; 5,709,843; 5,705,331; 5,703,057; 5,702,707; 5,698,178; 5,688,914; 5,686,078; 5,681,831; 5,679,784; 5,674,984; 5,672,472; 5,667,964; 5,667,783; 5,665,536; 5,665,355; 5,660,990; 5,658,745; 5,658,569; 5,643,756; 5,641,624; 5,639,854; 5,639,598; 5,637,677; 5,637,455; 5,633,234; 5,629,153; 5,627,025; 5,622,705; 5,614,413; 5,610,035; 5,607,831; 5,606,026; 5,601,819; 5,597,688; 5,593,972; 5,591,829; 5,591,823; 5,589,466; 5,587,285; 5,585,254; 5,585,250; 5,580,773; 5,580,739; 5,580,563; 5,573,916; 5,571,667; 5,569,468; 5,558,865; 5,556,745; 5,550,052; 5,543,328; 5,541,100; 5,541,057; 5,534,406; 5,529,765; 5,523,232; 5,516,895; 5,514,541; 5,510,264; 5,500,161; 5,480,967; 5,480,966; 5,470,701; 5,468,606; 5,462,852; 5,459,127; 5,449,601; 5,447,838; 5,447,837; 5,439,809; 5,439,792; 5,418,136; 5,399,501; 5,397,695; 5,391,479; 5,384,240; 5,374,519; 5,374,518; 5,374,516; 5,364,933; 5,359,046; 5,356,772; 5,354,654; 5,344,755; 5,335,673; 5,332,567; 5,320,940; 5,317,009; 5,312,902; 5,304,466; 5,296,347; 5,286,852; 5,268,265; 5,264,356; 5,264,342; 5,260,308; 5,256,767; 5,256,561; 5,252,556; 5,230,998; 5,230,887; 5,227,159; 5,225,347; 5,221,610 5,217,861; 5,208,321; 5,206,136; 5,198,346; 5,185,147; 5,178,865; 5,173,400; 5,173,399; 5,166,050; 5,156,951; 5,135,864; 5,122,446; 5,120,662; 5,103,836; 5,100,777; 5,100,662; 5,093,230; 5,077,284; 5,070,010; 5,068,174; 5,066,782; 5,055,391; 5,043,262; 5,039,604; 5,039,522; 5,030,718; 5,030,555; 5,030,449; 5,019,387; 5,013,556; 5,008,183; 5,004,697; 4,997,772; 4,983,529; 4,983,387; 4,965,069; 4,945,082; 4,921,787; 4,918,166; 4,900,548; 4,888,290; 4,886,742; 4,885,235; 4,870,003; 4,869,903; 4,861,707; 4,853,326; 4,839,288; 4,833,072 and 4,795,739.

In another embodiment, HIV, or immunogenic fragments thereof, may be utilized as the HIV epitope. For example, the HIV nucleotides of U.S. Pat. Nos. 7,393,949, 7,374,877, 7,306,901, 7,303,754, 7,173,014, 7,122,180, 7,078,516, 7,022,814, 6,974,866, 6,958,211, 6,949,337, 6,946,254, 6,896,900, 6,887,977, 6,870,045, 6,803,187, 6,794,129, 6,773,915, 6,768,004, 6,706,268, 6,696,291, 6,692,955, 6,656,706, 6,649,409, 6,627,442, 6,610,476, 6,602,705, 6,582,920, 6,557,296, 6,531,587, 6,531,137, 6,500,623, 6,448,078, 6,429,306, 6,420,545, 6,410,013, 6,407,077, 6,395,891, 6,355,789, 6,335,158, 6,323,185, 6,316,183, 6,303,293, 6,300,056, 6,277,561, 6,270,975, 6,261,564, 6,225,045, 6,222,024, 6,194,391, 6,194,142, 6,162,631, 6,114,167, 6,114,109, 6,090,392, 6,060,587, 6,057,102, 6,054,565, 6,043,081, 6,037,165, 6,034,233, 6,033,902, 6,030,769, 6,020,123, 6,015,661, 6,010,895, 6,001,555, 5,985,661, 5,980,900, 5,972,596, 5,939,538, 5,912,338, 5,869,339, 5,866,701, 5,866,694, 5,866,320, 5,866,137, 5,864,027, 5,861,242, 5,858,785, 5,858,651, 5,849,475, 5,843,638, 5,840,480, 5,821,046, 5,801,056, 5,786,177, 5,786,145, 5,773,247, 5,770,703, 5,756,674, 5,741,706, 5,705,612, 5,693,752, 5,688,637, 5,688,511, 5,684,147, 5,665,577, 5,585,263, 5,578,715, 5,571,712, 5,567,603, 5,554,528, 5,545,726, 5,527,895, 5,527,894, 5,223,423, 5,204,259, 5,144,019, 5,051,496 and 4,942,122 are useful for the present invention.

Any epitope recognized by an HIV antibody may be used in the present invention. For example, the anti-HIV antibodies of U.S. Pat. Nos. 6,984,721, 6,972,126, 6,949,337, 6,946,465, 6,919,077, 6,916,475, 6,911,315, 6,905,680, 6,900,010, 6,825,217, 6,824,975, 6,818,392, 6,815,201, 6,812,026, 6,812,024, 6,797,811, 6,768,004, 6,703,019, 6,689,118, 6,657,050, 6,608,179, 6,600,023, 6,596,497, 6,589,748, 6,569,143, 6,548,275, 6,525,179, 6,524,582, 6,506,384, 6,498,006, 6,489,131, 6,465,173, 6,461,612, 6,458,933, 6,432,633, 6,410,318, 6,406,701, 6,395,275, 6,391,657, 6,391,635, 6,384,198, 6,376,170, 6,372,217, 6,344,545, 6,337,181, 6,329,202, 6,319,665, 6,319,500, 6,316,003, 6,312,931, 6,309,880, 6,296,807, 6,291,239, 6,261,558, 6,248,514, 6,245,331, 6,242,197, 6,241,986, 6,228,361, 6,221,580, 6,190,871, 6,177,253, 6,146,635, 6,146,627, 6,146,614, 6,143,876, 6,132,992, 6,124,132, RE36,866, 6,114,143, 6,103,238, 6,060,254, 6,039,684, 6,030,772, 6,020,468, 6,013,484, 6,008,044, 5,998,132, 5,994,515, 5,993,812, 5,985,545, 5,981,278, 5,958,765, 5,939,277, 5,928,930, 5,922,325, 5,919,457, 5,916,806, 5,914,109, 5,911,989, 5,906,936, 5,889,158, 5,876,716, 5,874,226, 5,872,012, 5,871,732, 5,866,694, 5,854,400, 5,849,583, 5,849,288, 5,840,480, 5,840,305, 5,834,599, 5,831,034, 5,827,723, 5,821,047, 5,817,767, 5,817,458, 5,804,440, 5,795,572, 5,783,670, 5,776,703, 5,773,225, 5,766,944, 5,753,503, 5,750,373, 5,747,641, 5,736,341, 5,731,189, 5,707,814, 5,702,707, 5,698,178, 5,695,927, 5,665,536, 5,658,745, 5,652,138, 5,645,836, 5,635,345, 5,618,922, 5,610,035, 5,607,847, 5,604,092, 5,601,819, 5,597,896, 5,597,688, 5,591,829, 5,558,865, 5,514,541, 5,510,264, 5,478,753, 5,374,518, 5,374,516, 5,344,755, 5,332,567, 5,300,433, 5,296,347, 5,286,852, 5,264,221, 5,260,308, 5,256,561, 5,254,457, 5,230,998, 5,227,159, 5,223,408, 5,217,895, 5,180,660, 5,173,399, 5,169,752, 5,166,050, 5,156,951, 5,140,105, 5,135,864, 5,120,640, 5,108,904, 5,104,790, 5,049,389, 5,030,718, 5,030,555, 5,004,697, 4,983,529, 4,888,290, 4,886,742 and 4,853,326, are also useful for the present invention.

The vectors used in accordance with the present invention should typically be chosen such that they contain a suitable gene regulatory region, such as a promoter or enhancer, such that the antigens and/or antibodies of the invention can be expressed.

For example, when the aim is to express the antibodies and/or antigens of the invention in vitro, or in cultured cells, or in any prokaryotic or eukaryotic system for the purpose of producing the protein(s) encoded by that antibody and/or antigen, then any suitable vector can be used depending on the application. For example, plasmids, viral vectors, bacterial vectors, protozoal vectors, insect vectors, baculovirus expression vectors, yeast vectors, mammalian cell vectors, and the like, can be used. Suitable vectors can be selected by the skilled artisan taking into consideration the characteristics of the vector and the requirements for expressing the antibodies and/or antigens under the identified circumstances.

When the aim is to express the antibodies and/or antigens of the invention in vivo in a subject, for example in order to generate an immune response against an HIV-I antigen and/or protective immunity against HIV-I, expression vectors that are suitable for expression on that subject, and that are safe for use in vivo, should be chosen. For example, in some embodiments it may be desired to express the antibodies and/or antigens of the invention in a laboratory animal, such as for pre-clinical testing of the HIV-I immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. Any vectors that are suitable for such uses can be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

The nucleotide sequences and vectors of the invention can be delivered to cells, for example if aim is to express and the HIV-I antigens in cells in order to produce and isolate the expressed proteins, such as from cells grown in culture. For expressing the antibodies and/or antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies and/or antigens can be carried out in any suitable type of host cells, such as bacterial cells, yeast, insect cells, and mammalian cells. The antibodies and/or antigens of the invention can also be expressed using including in vitro transcription/translation systems. All of such methods are well known by those skilled in the art, and one of skill in the art would readily be able to select a suitable method depending on the nature of the nucleotide sequences, vectors, and cell types used.

In preferred embodiments, the nucleotide sequences, antibodies and/or antigens of the invention are administered in vivo, for example where the aim is to produce an immunogenic response in a subject. A “subject” in the context of the present invention may be any animal. For example, in some embodiments it may be desired to express the transgenes of the invention in a laboratory animal, such as for pre-clinical testing of the HIV-I immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. In preferred embodiments the subject is a human, for example a human that is infected with, or is at risk of infection with, HIV-1.

For such in vivo applications the nucleotide sequences, antibodies and/or antigens of the invention are preferably administered as a component of an immunogenic composition comprising the nucleotide sequences and/or antigens of the invention in admixture with a pharmaceutically acceptable carrier. The immunogenic compositions of the invention are useful to stimulate an immune response against HIV-1 and may be used as one or more components of a prophylactic or therapeutic vaccine against HIV-1 for the prevention, amelioration or treatment of AIDS. The nucleic acids and vectors of the invention are particularly useful for providing genetic vaccines, i.e. vaccines for delivering the nucleic acids encoding the antibodies and/or antigens of the invention to a subject, such as a human, such that the antibodies and/or antigens are then expressed in the subject to elicit an immune response.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri (caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, CA). (PEG).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AIK (SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71 (3): 538-44; Ahmad-Nejad, P. et al (2002) Eur. J. Immunol. 32 (7): 1958-68; poly IC or poly AU acids, polyarginine with or without CpG (also known in the art as IC3 1; see Schellack, C. et al (2003) Proceedings of the 34th Annual Meeting of the German Society of Immunology; Lingnau, K. et al (2002) Vaccine 20 (29-30): 3498-508), Juva Vax™ (U.S. Pat. No. 6,693,086), certain natural substances (e.g., wax D fromMycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169 (7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, AK. et al (2004) 22 (13-14): 1791-8), and the CCR5 inhibitor CMPD167 (see Veazey, R S. et al (2003) J. Exp. Med. 198:1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A M. et al (2001) J. Immunol. 167 (6): 3398-405), polyphosphazenes (Allcock, H. R. (1998) App. Organometallic Chem. 12 (10-11): 659-666; Payne, L. G. et al (1995) Pharm. Biotechnol. 6:473-93), cytokines such as, but not limited to, IL-2, IL-4, GM-CSF, IL-12, IL-15 IGF-1, IFN-α, IFN-, and IFN-γ (Boyer et al., (2002) J. Liposome Res. 121:137-142; WOOI/095919), immunoregulatory proteins such as CD40L (ADX40; see, for example, WO03/063899), and the CD1a ligand of natural killer cells (also known as CRONY or a-galactosyl ceramide; see Green, T. D. et al, (2003) J. Virol. 77 (3): 2046-2055), immunostimulatory fusion proteins such as IL-2 fused to the Fe fragment of immunoglobulins (Barouch et al., Science 290:486-492, 2000) and co-stimulatory molecules B7. 1 and B7.2 (Boyer), all of which can be administered either as proteins or in the form of DNA, on the same expression vectors as those encoding the antigens of the invention or on separate ex In an advantageous embodiment, the adjuvants may be lecithin combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)).pression vectors.

The immunogenic compositions can be designed to introduce the nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12:1529-1533; Xiang et al., (1994) Virology 199:132-140; Webster et al., (1994) Vaccine 12:1495-1498; Davis et al., (1994) Vaccine 12:1503-1509; and Davis et al., (1993) Hum. Mol. Gen. 2:1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of inducing an immune response against HIV in a subject by administering an immunogenic composition of the invention, preferably comprising an adenovirus vector containing DNA encoding one or more of the epitopes of the invention, one or more times to a subject wherein the epitopes are expressed at a level sufficient to induce a specific immune response in the subject. Such immunizations can be repeated multiple times at time intervals of at least 2, 4 or 6 weeks (or more) in accordance with a desired immunization regime.

The immunogenic compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other HIV immunogens can be administered at the same time or at different times as part of an overall immunization regime, e.g., as part of a prime-boost regimen or other immunization protocol. In an advantageous embodiment, the other HIV immunogen is env, preferably the HIV env trimer.

Many other HIV immunogens are known in the art, one such preferred immunogen is HIVA (described in WO 01/47955), which can be administered as a protein, on a plasmid (e.g., pTHr.HIVA) or in a viral vector (e.g., MVA.RIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an epitope of the present invention, a nucleic acid encoding an epitope of the invention or an expression vector, preferably a VSV vector, encoding an epitope of the invention, and wherein the immunogens are administered in an amount or expressed at a level sufficient to induce an HIV-specific immune response in the subject. The HIV-specific immune response can include an HIV-specific T-cell immune response or an HIV-specific B-cell immune response. Such immunizations can be done at intervals, preferably of at least 2-6 or more weeks.

The present invention will be further illustrated in the following Examples which are given for illustration purposes only and are not intended to limit the invention in any way.

Although the present invention and its advantages have been described in detail, it should be understood that various changes, substitutions and alterations can be made herein without departing from the spirit and scope of the invention as defined in the appended claims.

### EXAMPLES

### Example 1: Experimental Procedures

### SIV Envelope Trimer Design, its Expression and Purification.

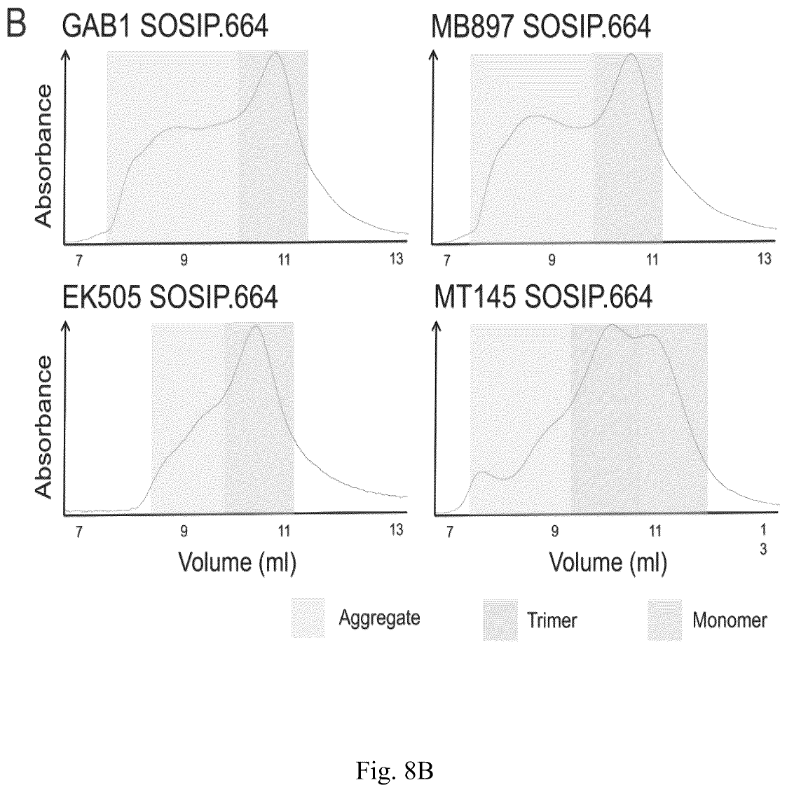

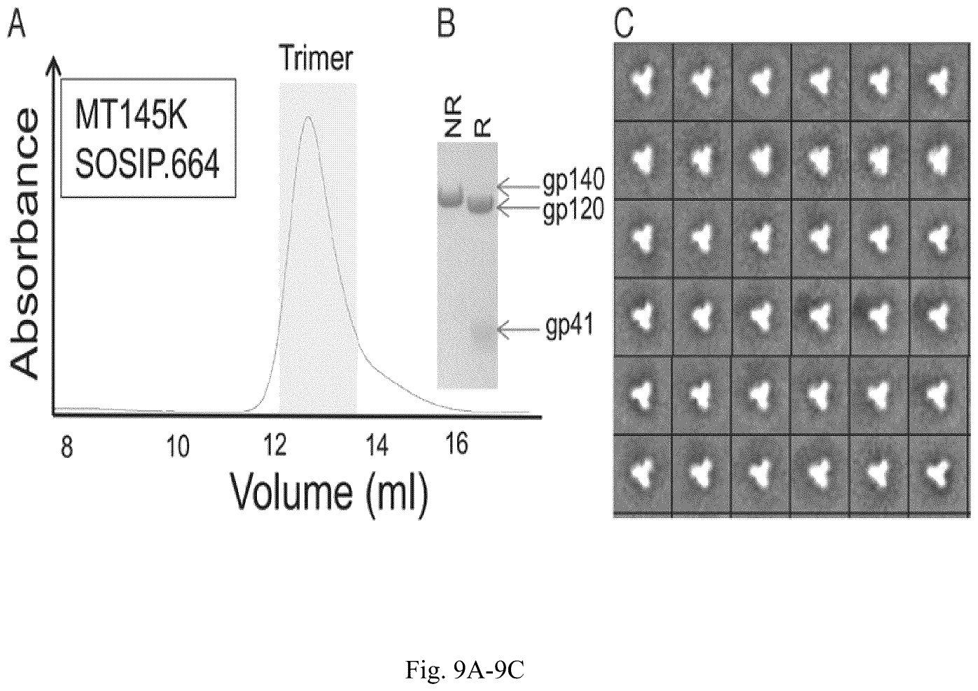

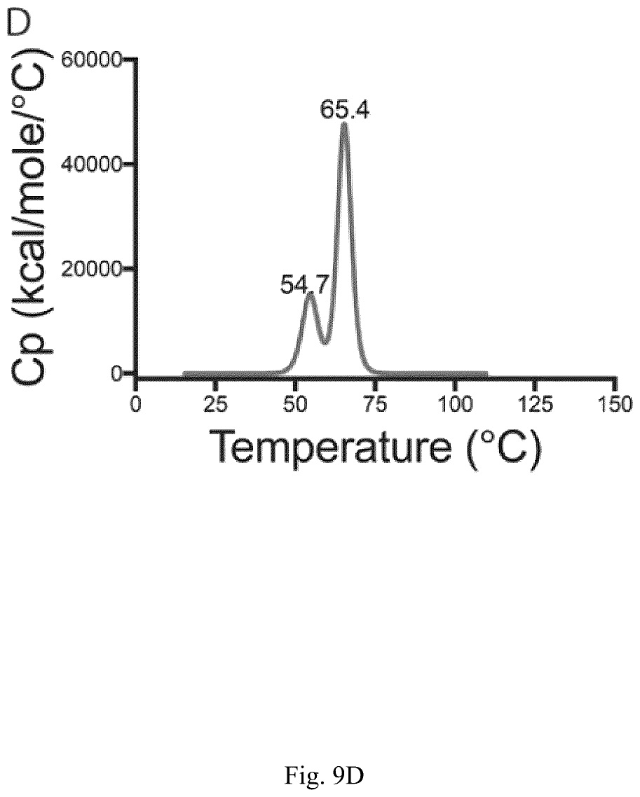

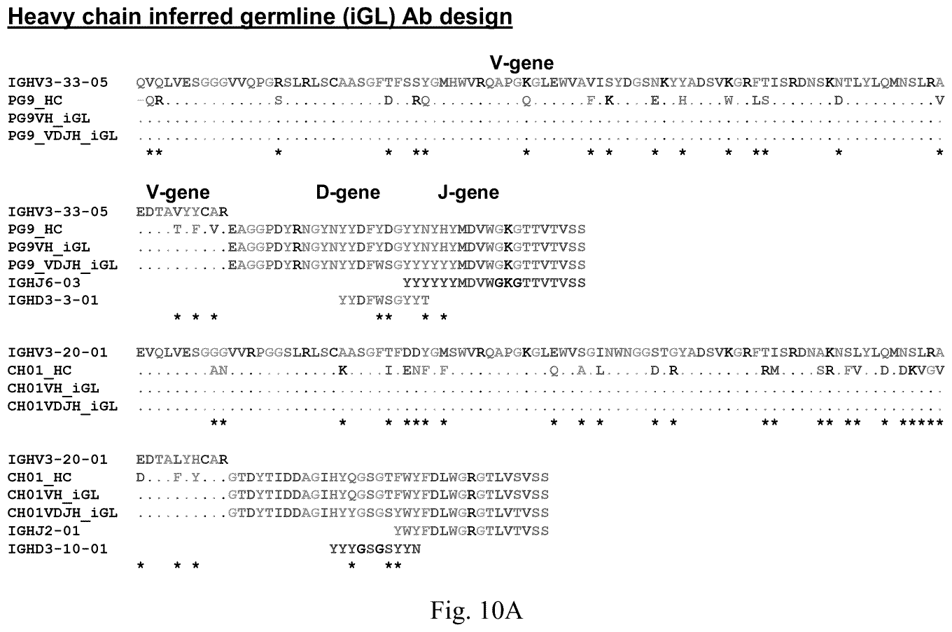

SOSIP.664 HIV-1 Env trimer design modifications were incorporated into envelope encoding sequences corresponding to four Chimpanzee (SIVcpzPtt) isolates (GAB1 [GenBank: P17281]; MB897 [GenBank: ABU53023]; EK505 [GenBank: ABD19499]; and MT145 [GenBank: ABD19508]) to express as soluble native trimers (FIG. 8A). These isolates are sensitive to the V2 apex bnAbs, PG9, PG16, and PGT145 (Barbian et al., 2015). Further characterization showed that one of these SIVcpzPtt Env sequences, MT145 SOSIP.664, could be expressed as a soluble Env trimer protein (FIG. 8B). PGT145 Ab affinity-purified MT145 trimer was efficiently cleaved into gp120 and gp41 subunits, and revealed well-ordered native-like trimer configurations that were highly thermostable, which are all properties displayed by natively folded HIV-1 soluble trimers (FIG. 9A-D) (Pugach et al., 2015; Sanders et al., 2013; Sharma et al., 2015).

Briefly, the following modifications were incorporated into these Envs for soluble trimer expression: a) the Env leader sequence was replaced by Tissue Plasminogen Activator (TPA) signal sequence for higher protein expression; b) a disulfide bond was introduced between gp120 and gp41 subunits by substituting residues A501-C and T605-C respectively in gp120 and gp41; c) the gp120 REKR (SEQ ID NO:358) cleavage site was replaced by Furin inducible R6 site (RRRRRR) (SEQ ID NO:359) for enhancing cleavage efficiency between gp120 and gp41; and d) an 1559P substitution in gp41 to stabilize the soluble trimer protein. In addition, a GS-linker and a His-tag were added to the gp41EcTo C-terminus at HXB2 residue 664 position. The codon-optimized SOSIP.664 gp140 gene constructs were synthesized (Geneart, Life Technologies) and cloned into the phCMV3 vector (Genlantis). Recombinant envelope proteins were expressed in HEK293F cells as described elsewhere (Sanders et al., 2013). Briefly, HIV-1 Env trimers WITO, C108, ZM197-ZM233V1V2 and the 4 chimpanzee SIV SOSIP.664 Env-encoding trimer plasmids were cotransfected with a plasmid encoding for Furin (3:1 ratio) into HEK293F cells using PEI-MAX 4000 transfection reagent (Polysciences, Inc.). The secreted soluble trimers proteins were purified from cell supernatants after 5 days using agarose-bound Gallanthus Nivalis Lectin (GNL) (Vector Labs) or CNBr-activated Sepharose 4B bead (GE Healthcare) bound PGT145 bnAb antibody affinity columns as described previously (Pugach et al., 2015). The affinity-purified proteins were size exclusion chromatography (SEC)-purified with a Superdex 200 10/300 GL column (GE Healthcare) in PBS/TBS. The purified trimers for the immunization experiments were quality control tested for antigenicity with a range of HIV-1 Env-specific neutralizing and non-neutralizing mAbs.

### Example 2: Binding to bnAB Precursors

MT145K Trimer Binds Prototype V2 Apex bnAb Precursors.

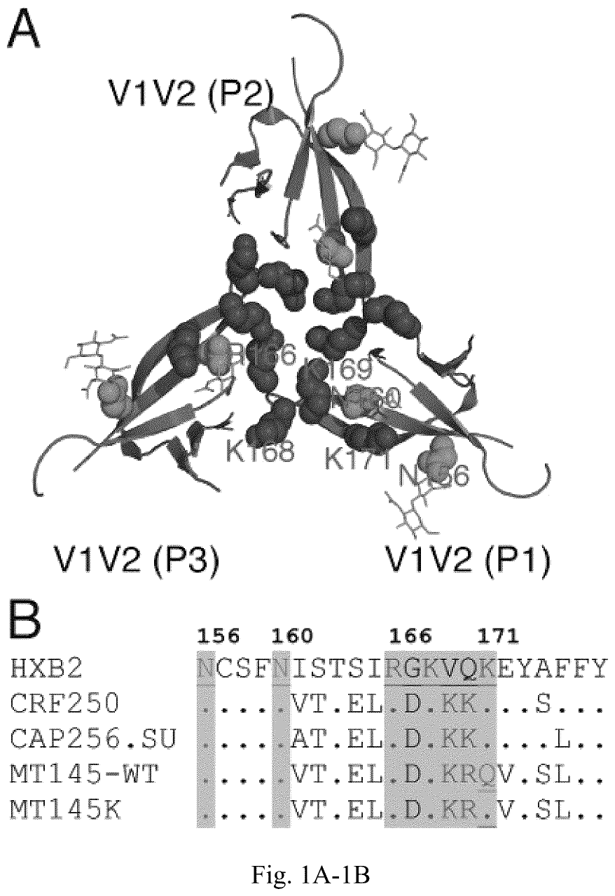

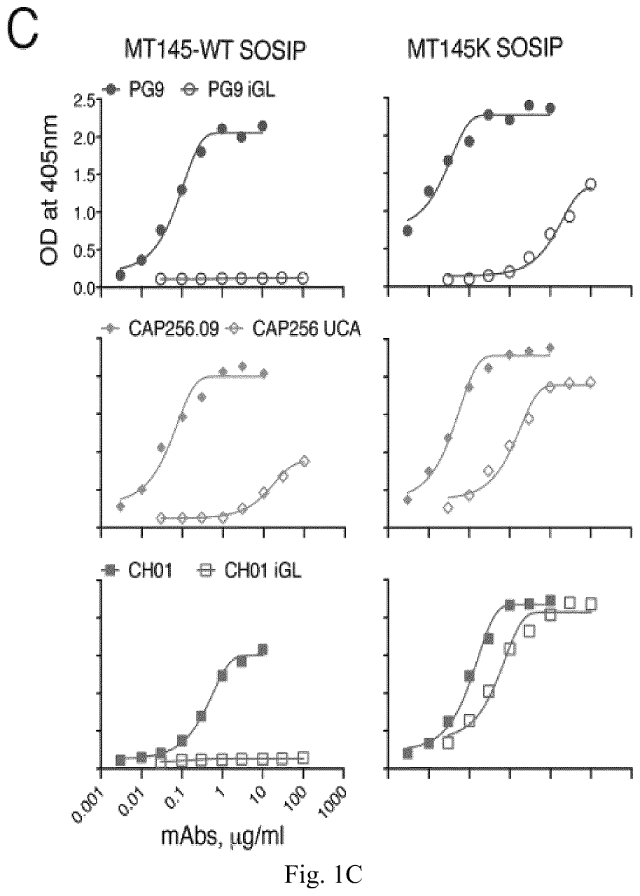

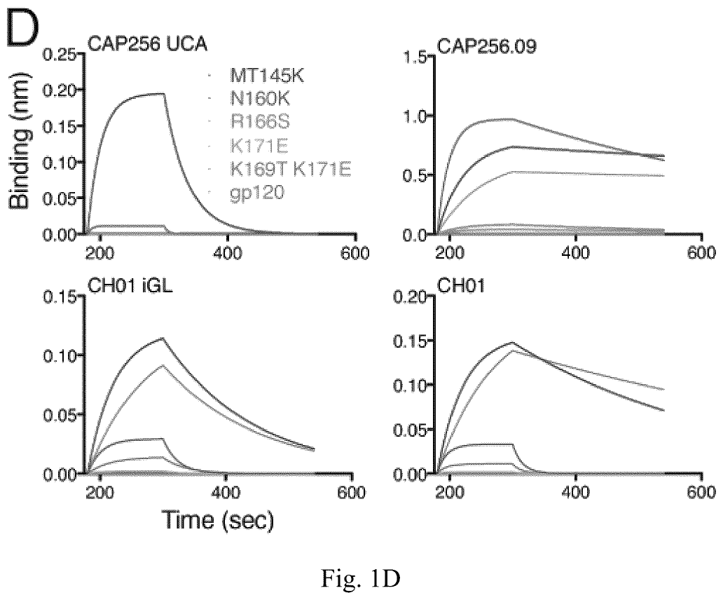

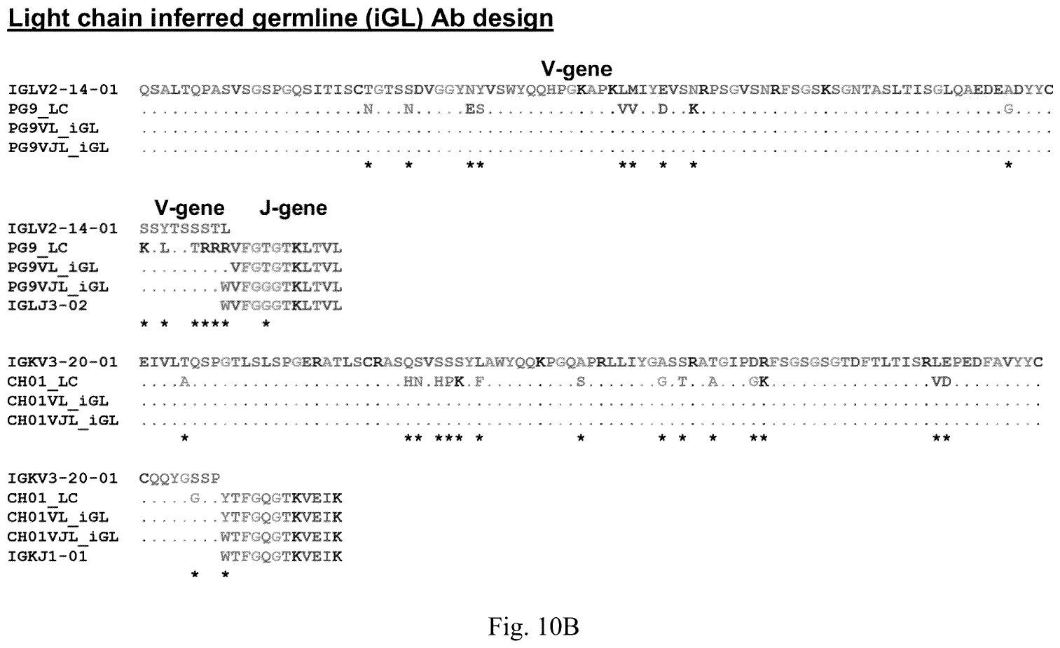

One property thought to be critical for vaccine immunogens to select rare bnAb precursors is the ability to effectively bind to UCA B cell receptors (Dosenovic et al., 2015; Escolano et al., 2016; Jardine et al., 2015; McGuire et al., 2016; Steichen et al., 2016a). Therefore, to gain or improve binding of the V2 apex bnAb inferred precursor Abs to MT145 Env trimer, glutamine (Q) was substituted with lysine (K) at residue (HXB2 position 171) in strand C of the V2 apex bnAb core epitope (FIG. 1A-B). This substitution is based on the presence of a positively charged motif (KKKK) (SEQ ID NO:5) in CRF250 and CP256.SU strand C V2 Env sequences, both of which bind V2 apex bnAb prototype precursors (Andrabi et al., 2015; Doria-Rose et al., 2014; Gorman et al., 2016). ELISA binding revealed strong binding of the mature V2 apex bnAb prototypes with the MT145-WT trimer and weak but detectable binding with one of the UCA Abs, CAP256 UCA (FIG. 1C). Strikingly, binding with a V2-engineered MT145 trimer (henceforth referred to as “MT145K”) not only improved binding to CAP256 UCA Ab but also conferred binding on both PG9 and CH01 iGL Abs (FIG. 1C). The PG9 and CH01 iGL Abs used here had diversity (D; heavy chain) and joining (J; both heavy and light chains) genes reverted to their corresponding germline gene families in the CDRH3s, in addition to the VH and VL regions reported previously (FIG. 10) (Andrabi et al., 2015; Gorman et al., 2016).

Previous mapping studies have examined the HIV core epitope recognized by mature V2 apex bnAbs (Andrabi et al., 2015; Gorman et al., 2016; Landais et al., 2017; Lee et al., 2017; Mclellan et al., 2011; Pancera et al., 2013; Walker et al., 2009). To evaluate the contributions of V2 apex core epitope glycan and protein residues to binding by V2 apex bnAb inferred germline-reverted (iGL) Ab versions, MT145K strand C peptide and glycan trimer variants known to eliminate interactions of V2 apex bnAbs with the Env trimer were generated (see Andrabi et al., 2015; Mclellan et al., 2011; Pancera et al., 2013). Bio-Layer Interferometry (BLI or octet) binding analyses of the iGL Abs with these trimer variants showed that glycan/peptide epitope requirements of precursor Abs were largely similar to the requirements of corresponding mature Abs (FIG. 1D), suggesting that most contacts with the MT145K V2 apex core epitope are already encoded in the germline configuration for this class of bnAbs. Notably, the mature Abs showed slightly more tolerance to changes within the core protein epitope, particularly for the CAP256.09 bnAb, suggesting that part of the affinity maturation in this class of Abs may be to accommodate variation within the bnAb V2 apex core epitope. Overall, the strand C V2-modification in the MT145 SOSIP.664 trimer conferred binding to multiple V2 apex bnAb germline prototypes.

### Example 3: Structure

### Architecture of the MT145K Trimer.

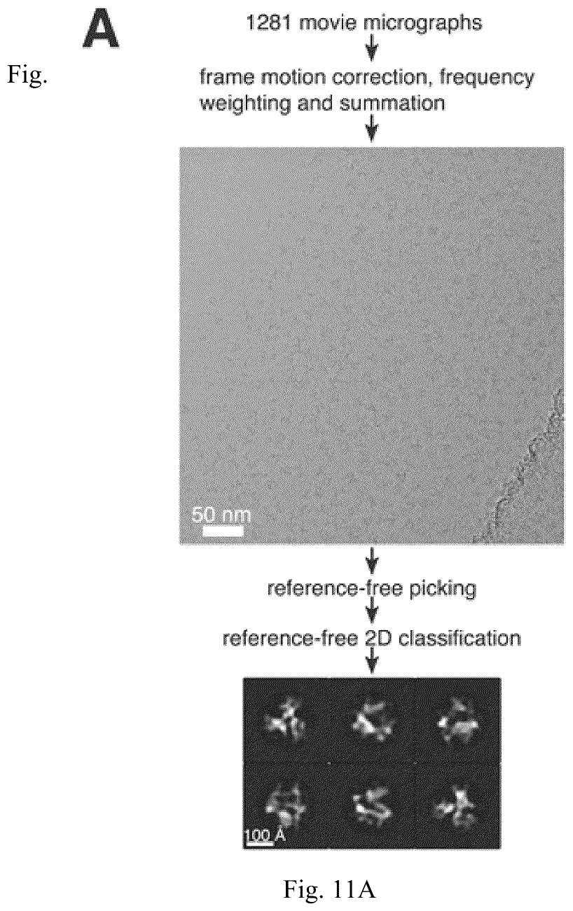

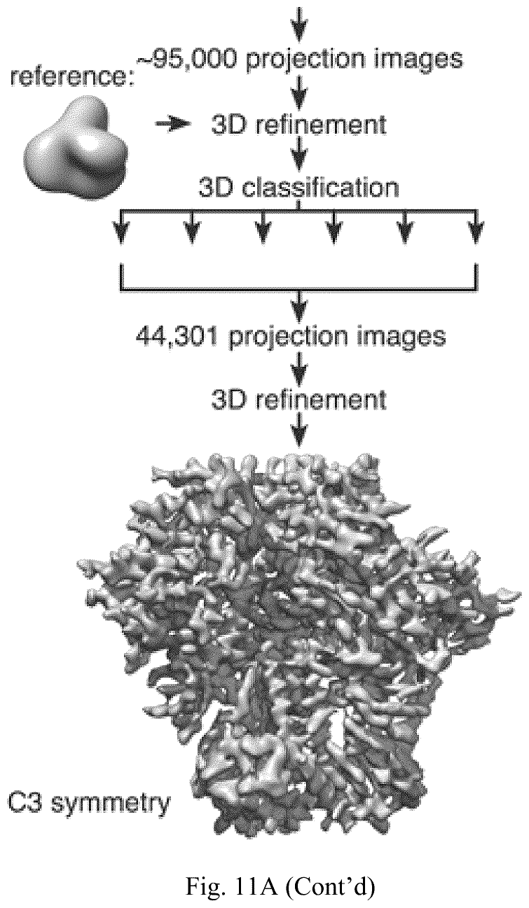

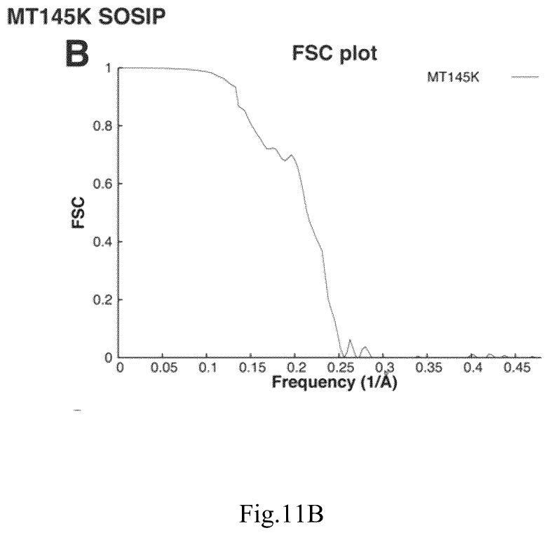

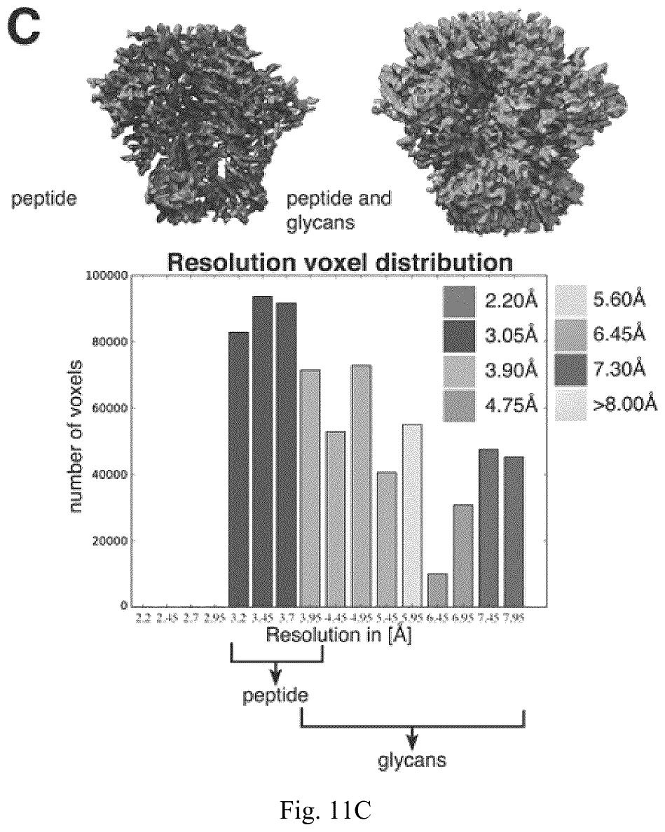

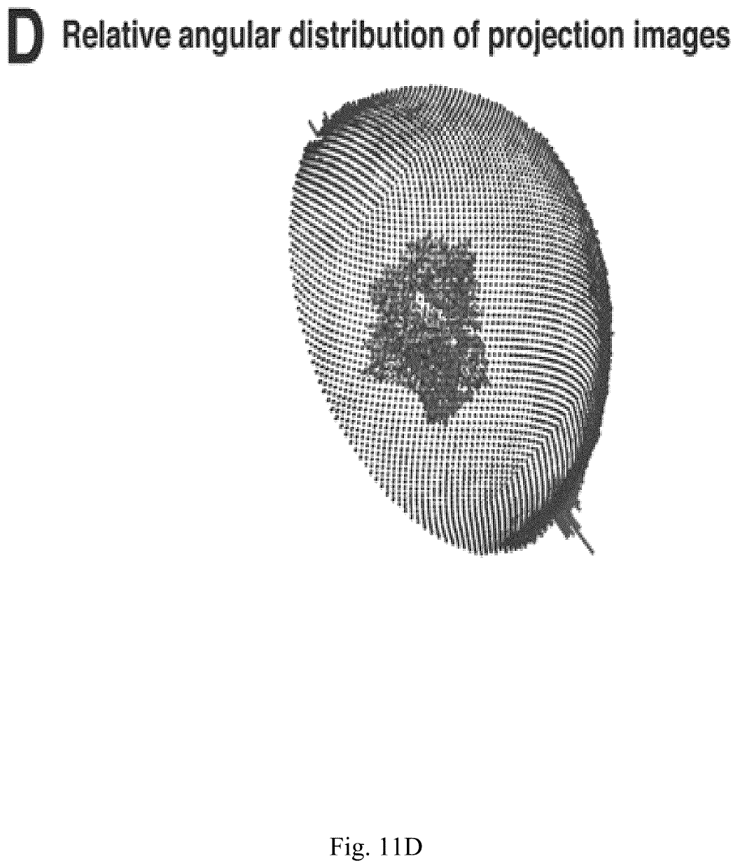

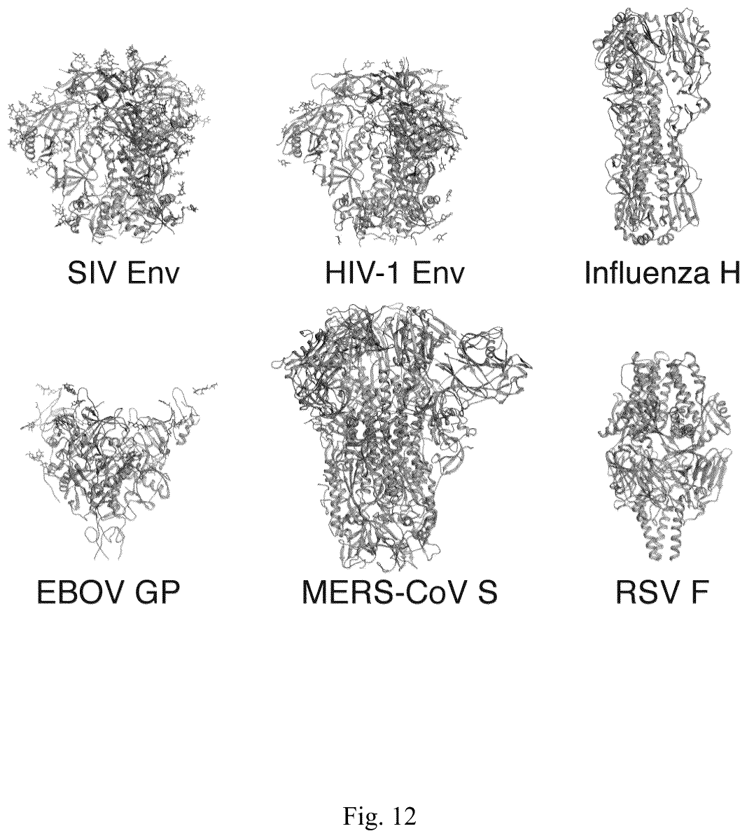

The structure of the MT145K trimer was solved by cryo-EM to a global resolution of ˜4.1 Å (FIG. 11). Data collection methods are summarized in the following table.

| Data collection | |
|---|---|
| Microscope | FEI Titan Krios |
| Voltage (kV) | 300 |
| Detector | Gatan K2 Summit |
| Recording mode | Counting |
| Magnification (incl. post-magnification) | 49,020 |
| Movie micrograph pixelsize (Å) | 1.02 |
| Dose rate (e⁻/[(camera pixel)*s]) | 10 |
| Number of frames per movie micrograph | 50 |
| Frame exposure time (ms) | 200 |
| Movie micrograph exposure time (s) | 10 |
| Total dose (e⁻/Å²) | 94 |
| Defocus range (μm) | 1.0-3.6 |
| EM data processing | |
| Number of movie micrographs | 1,281 |
| Number of molecular projection images in map | 44,301 |
| Symmetry | C3 |
| Map resolution (FSC 0.143; Å) | 4.1 |
| Mapsharpening B-factor (Å²) | −130 |
| Structure Building and Validation | |
| Number of atoms in depositedmodel | 15,192 |
| gp120 | 3,547 |
| gp41 | 964 |
| glycans | 553 |
| MolProbity score | 1.72 (88%) |
| Clashscore | 3.55 |
| EMRinger score | 2.26 |
| Deviations from ideal | |
| Bond length outliers | 0 (0%) |
| Bond angles outliers | 0 (0%) |
| Ramachandran plot | |
| Favored (%) | 88.9 |
| Allowed(%) | 10.5 |
| Outliers (%) | 0.5 |

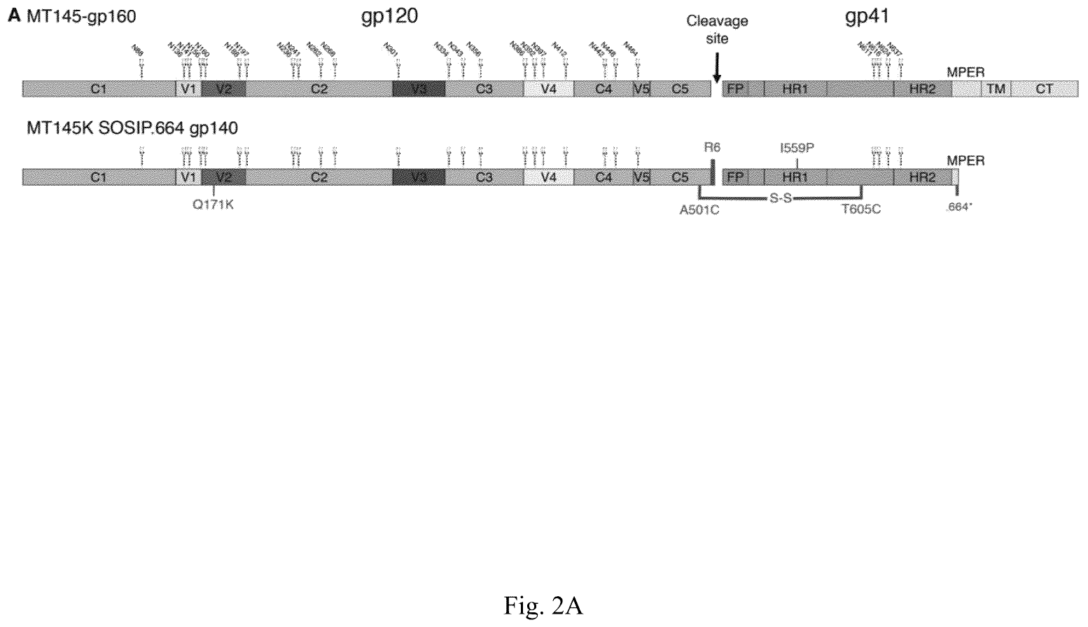

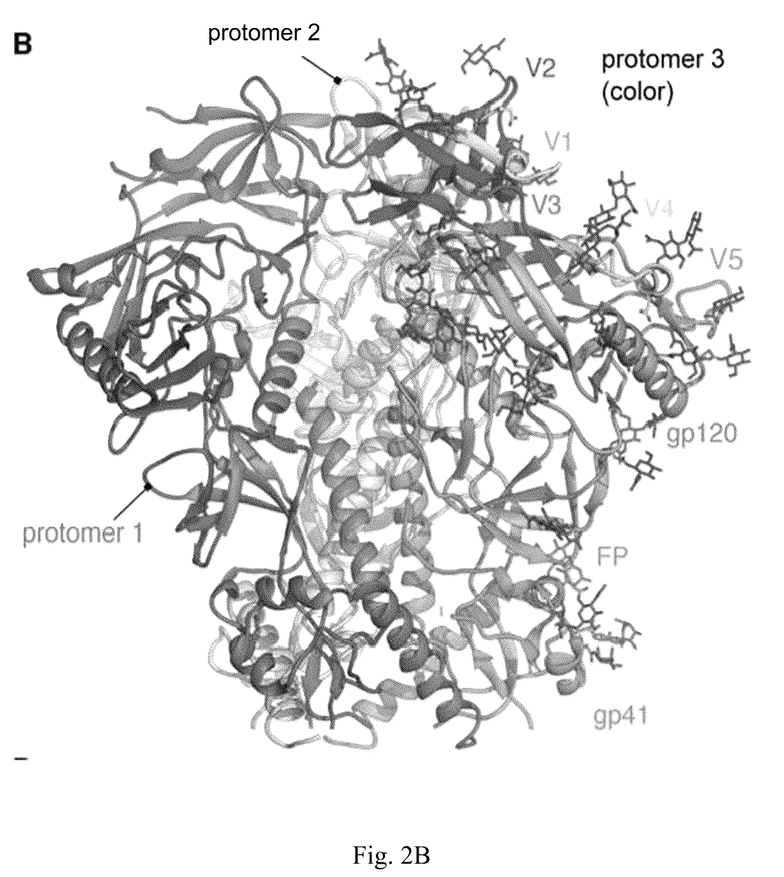

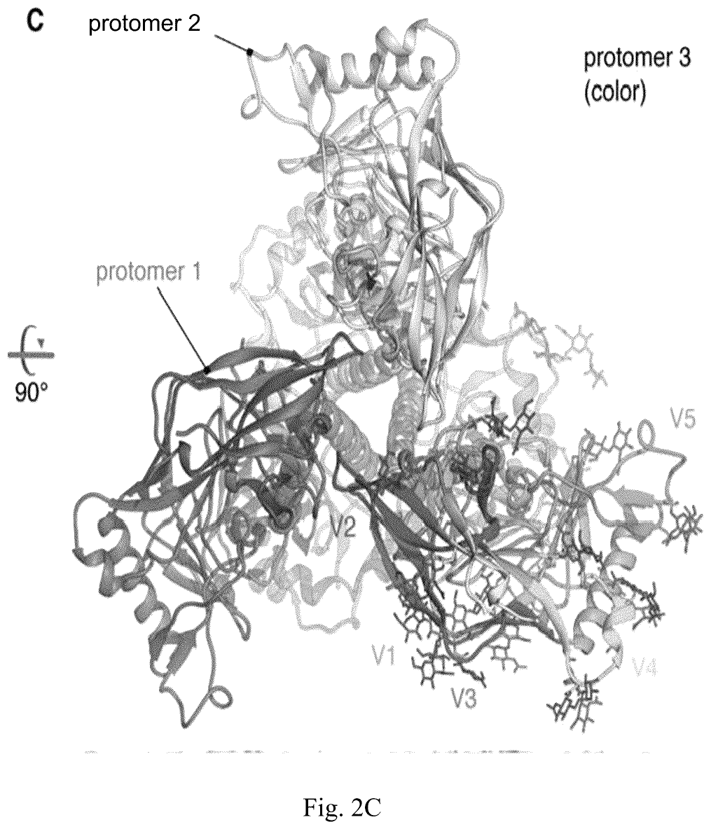

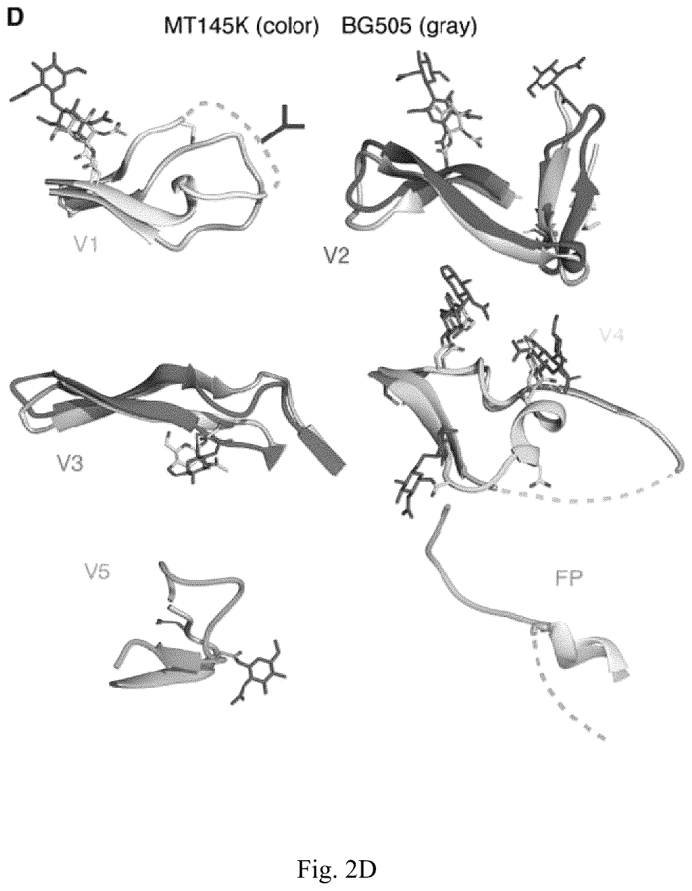

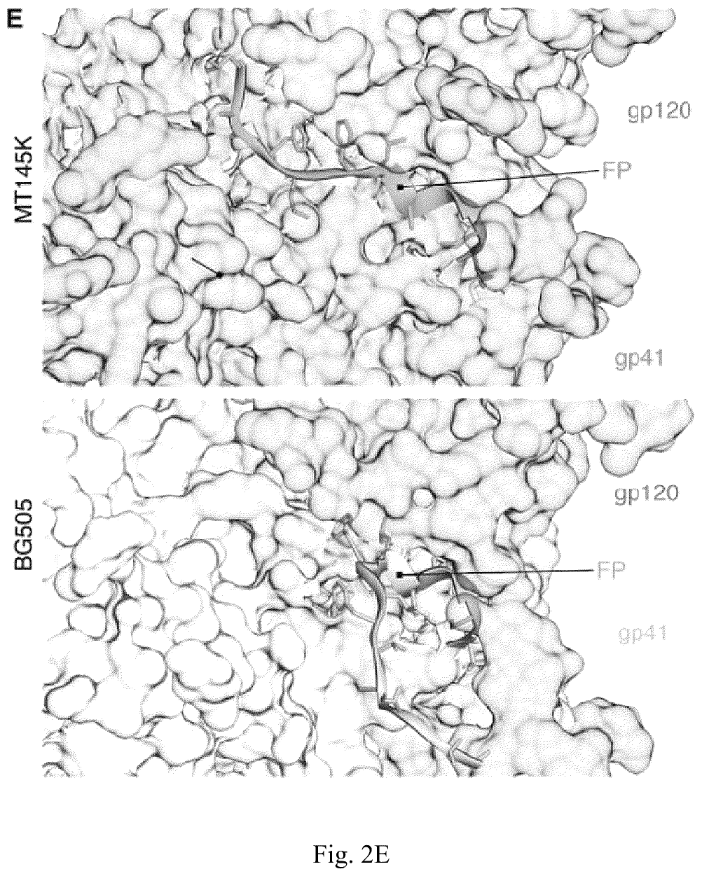

The structure represents the first atomic level structure of an SIV Env trimer. Like other class I fusion proteins, protomers (gp120 and gp41) of MT145K trimerize to form a metastable pre-fusion Env trimer (FIGS. 2A-B, 12). The trimer architecture exhibits a mushroom-like shape with subunits gp120 and gp41 constituting the envelope-distal and proximal entities, respectively (FIG. 2B). Overall, the MT145K trimer configuration closely resembles that of the trimeric HIV-1 Env spike, with an overall Ca root mean square deviation (rmsd) of 1.9 Å (Kwon et al., 2015). Arrangement of V-loops in the MT145K Env trimer is reminiscent of the V-loop arrangement in the HIV Env trimer and is suggestive of a similar role in immune evasion by steric occlusion of underlying conserved epitopes (Julien et al., 2013a; Pancera et al., 2014). Notably, the V1 and V2 loops are largely solvent-exposed and occlude access to the underlying V3 loop (FIG. 2C). Inaccessibility of the V3 loop is mediated by intra-protomer interactions of VIV2 to V3 and by extensive inter-protomer VIV2 trimer interactions at the apex of the spike. The SIV Env trimer exhibits well-ordered V2-V5 loops, while V1 is somewhat disordered (FIG. 2D).

Proximal to the viral membrane is the gp41 subunit that forms the base of the trimer spike and is arranged into heptad repeat-1 (HR1), HR2 and the fusion peptide (FP) (FIG. 2B). Similar to the HIV Env trimer, the three C-terminal helices of HRI are centrally positioned along the trimer axis perpendicular to the viral membrane (Julien et al., 2013a; Lyumkis et al., 2013; Pancera et al., 2014). Intriguingly, the FP region, which has been observed solvent-exposed on the outside of the HIV-1 Env, is positioned in a pocket inside the MT145K trimer and remains sequestered in all three protomers (FIG. 2E).

### Example 4: Glycan Analysis

### Conservation of the Glycan Shield on HIV and SIV Env Trimers.

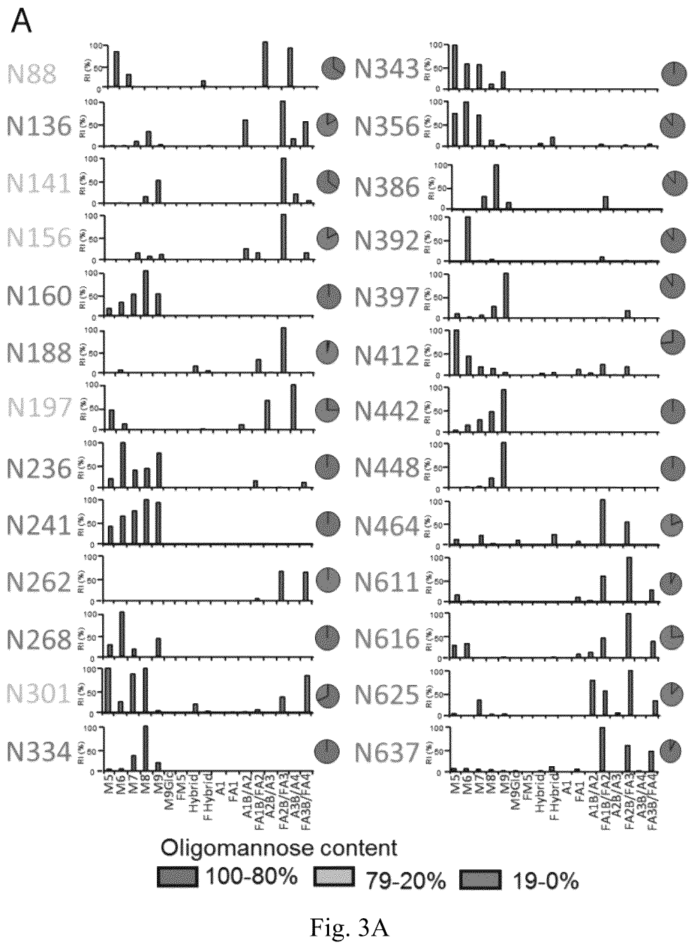

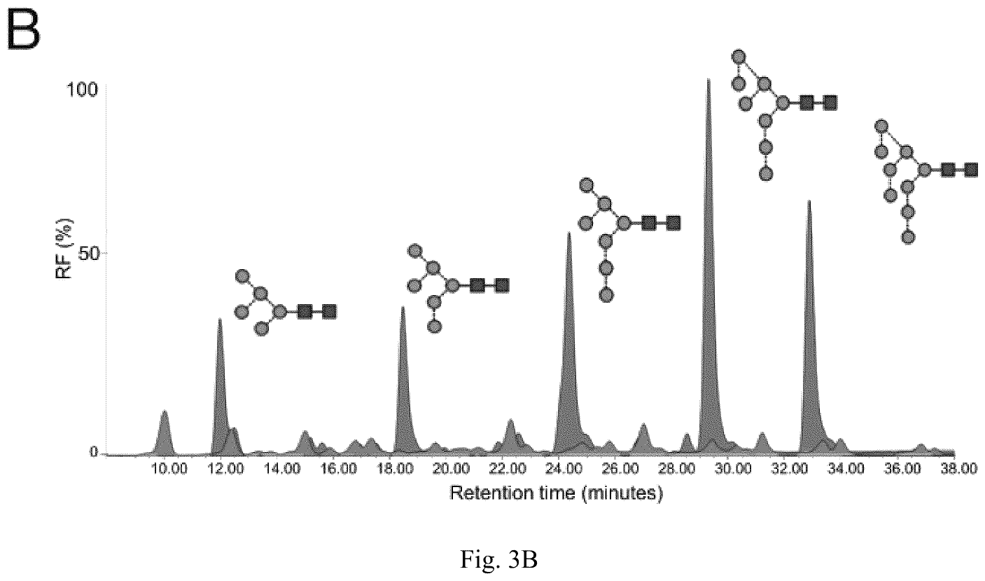

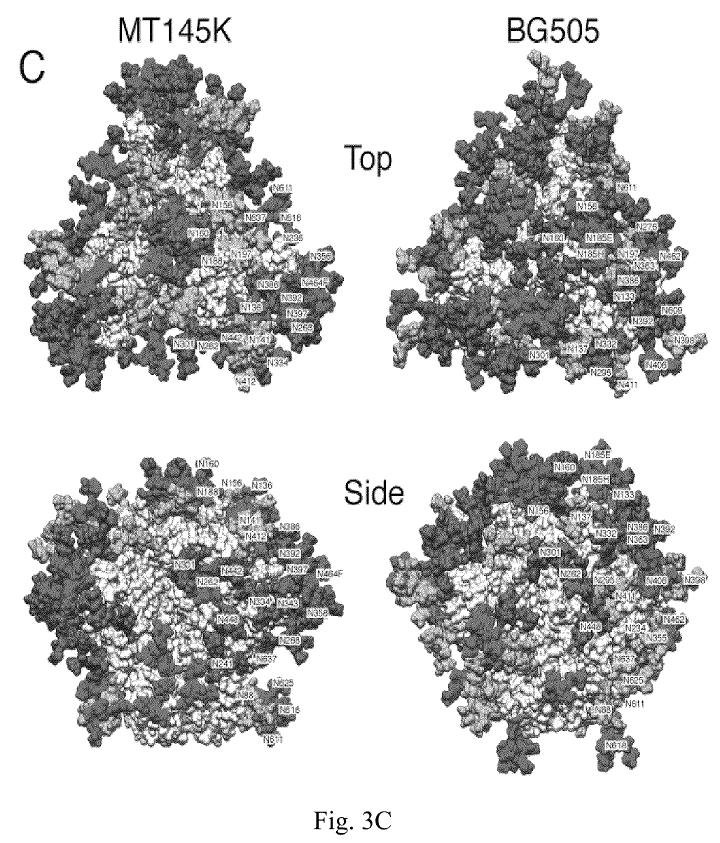

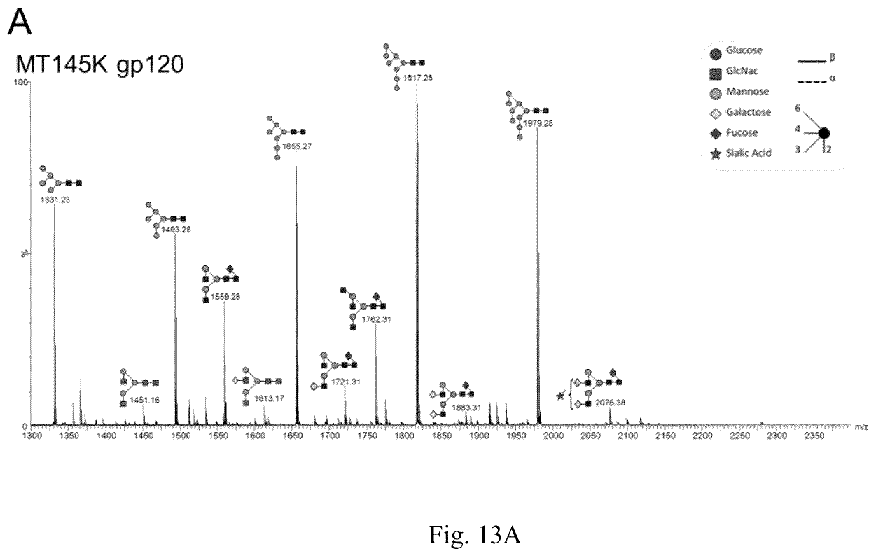

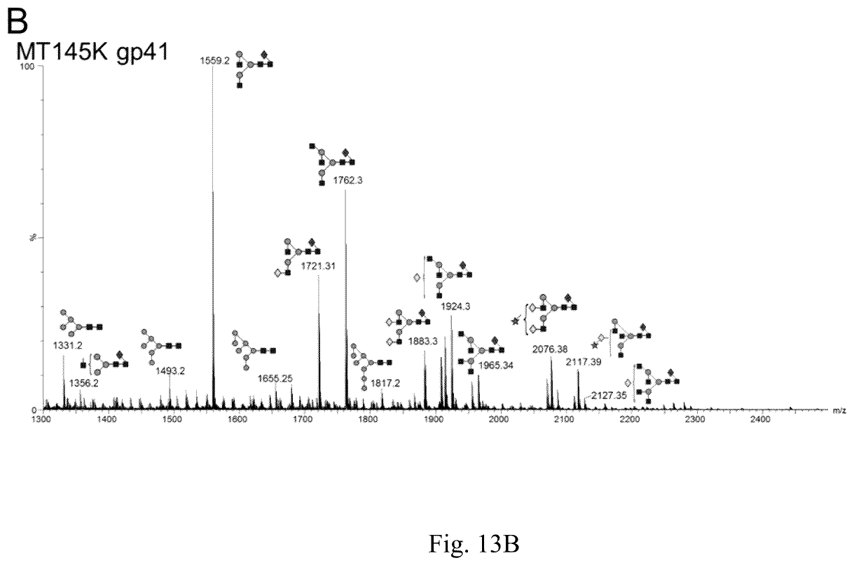

To compare the nature of the glycan shield on SIVcpzPtt Env and HIV Env, site-specific glycan analysis of the MT145K trimer was performed. The overall oligomannose content of the MT145K trimer is similar to HIV Env (FIG. 3A-B) (Panico et al., 2016; Pritchard et al., 2015). However, although the distributions differed from the HIV-1 clade A strain BG505, which is dominated by Man₉GlcNAc₂ oligomannose-type glycans, MT145K is predominantly Man₈GlcNAc₂ (Behrens et al., 2016). In addition, further processing was evident in the MT145K trimer which showed elevated Man₆₋₇GlcNAc₂ structures (FIGS. 3B, 13). The outer domain of gp120 presents a high density of oligomannose glycans that form the intrinsic mannose patch (Bonomelli et al., 2011), which was a highly conserved feature across the two viral species. The apex of the MT145K trimer possesses oligomannose-type glycans at N160 that correspond to the trimer associated mannose patch (TAMP) also observed on HIV-1 Env (Behrens et al., 2017). As for HIV-1, glycans at the base of the trimer at N88 and on gp41 of the MT145K trimer are extensively processed (FIGS. 3A, C, 13).

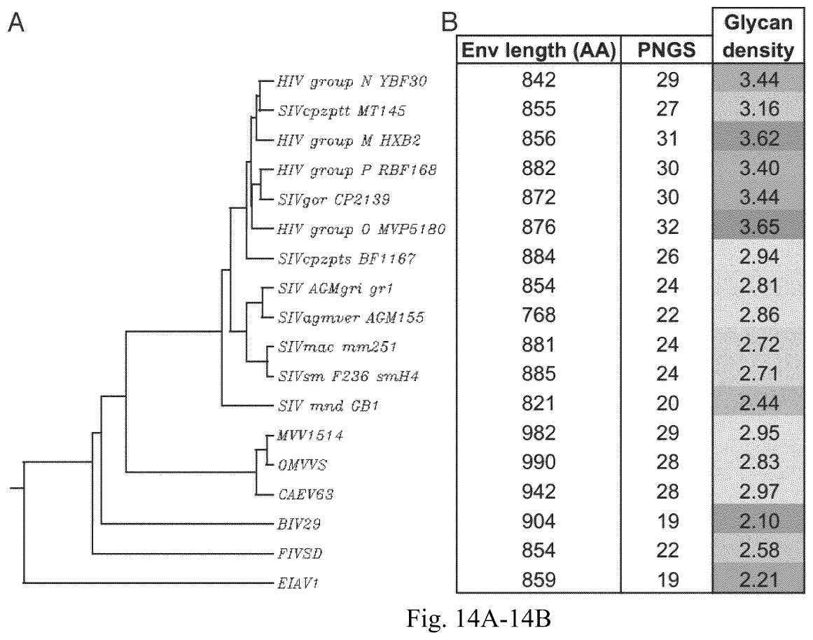

In the MT145K trimer, glycans at N156 and N262 are predominantly complex-type, whereas the corresponding glycans are oligomannose-type in HIV Env (Behrens et al., 2016). These differences may arise due to the proximity of neighbouring glycans. For instance, the HIV Env glycans at positions N295 and N332, adjacent to the N262 glycan, are absent on MT145K Env, which may lead to increased processing of N262 (FIGS. 3A, C). The remarkable conservation in the overall architecture of the SIV and HIV Env glycan shield, despite sharing only ˜62% of the amino-acid sequence identity, suggests that the glycan shield has an indispensable role in immune evasion and potentially maintaining functional integrity of the trimer spike. Indeed, the glycan shield is integral to all lentiviral envelopes and appears to have evolved somewhat specifically to mammalian host (FIG. 14). Over the course of lentiviral evolution, the Env glycan density shows an overall gradual progression, and likely peaked in retroviruses infecting non-human primates and plateaued in HIV Envs (FIG. 14) (Zhang et al., 2004). Therefore, the high-density Env glycan shield on HIV must have been established well before chimpanzee SIV crossed into humans. Nevertheless, several glycan positions on HIV-1 Env appear to have subtly shifted after the species cross-over that presumably resulted as an adaptation to the human immune system (FIG. 15).

### Example 5: Binding Specificity

MT145K Binds V2 Apex bnAbs Almost Exclusively.

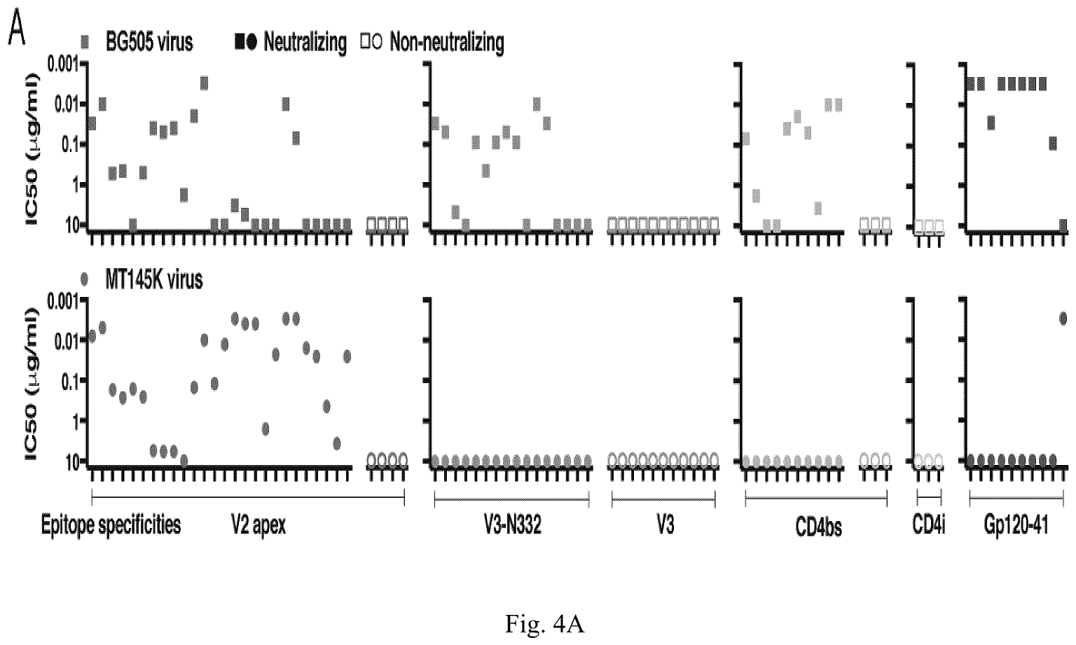

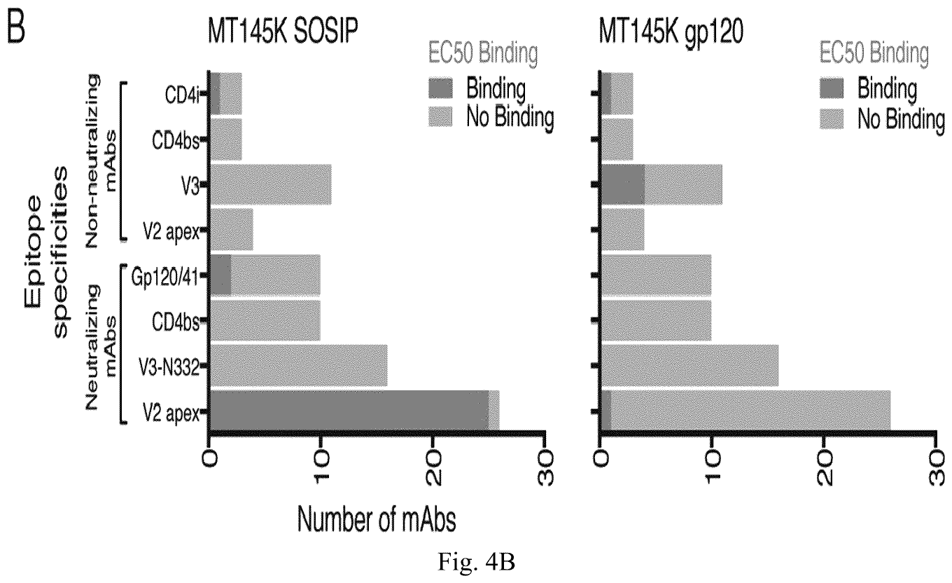

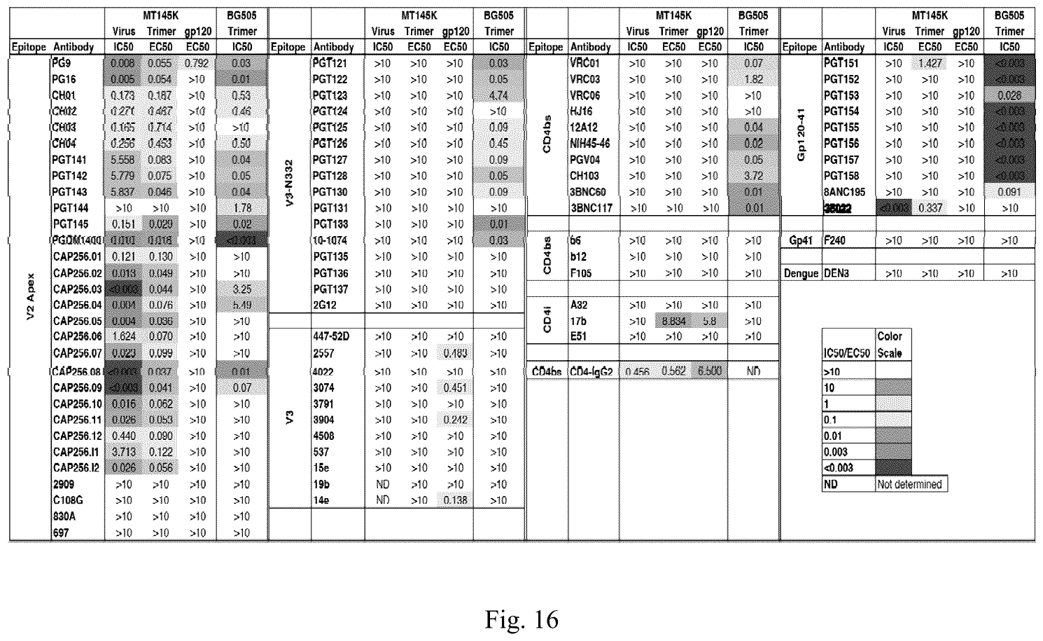

To define the overall antigenicity of the MT145K trimer, neutralization sensitivity of MT145K virus (MT145-Q171K) to a broad panel of HIV-1 Env-specific neutralizing and non-neutralizing (nnAbs) mAbs was assessed and the profiles compared to the clade A BG505 HIV virus (FIGS. 4A, 16) (Sanders et al., 2013; Voss et al., 2017). Remarkably, the V2 apex bnAbs, but essentially no other bnAbs or nnAbs (except 35022 gp120-41 interface mAb), exhibited potent neutralizing activities against MT145K virus (FIG. 4A, 16). As previously observed, the BG505 isolate was sensitive to neutralization by all of the bnAbs in the panel, but none of the nn Abs (FIG. 4A FIG. 16).

Next, binding of MT145K trimer and monomeric gp120 to a panel of mAbs was evaluated by ELISA. Consistent with the neutralization results above, bnAbs to the V2 apex site showed robust binding to the MT145K trimer (FIGS. 4B, 16), but other bnAbs and nnAbs did not bind, except for a few mAbs that displayed very weak binding (FIGS. 4B, 16). PG9, 17b and some of the linear V3-loop directed mAbs (2557, 3074, 3904 and 14e) (FIGS. 4B, 16) that bound to the MT145K gp120 monomer. The results suggest that the sequence-dependent epitopes for some of the non-neutralizing V3-loop mAbs are present on monomeric MT145K gp120, but are obscured on the MT145K trimer, as indicated by the MT145K structure. Virus neutralization and trimer binding by mAbs is strongly correlated (p=0.003), consistent with the notion that the MT145K soluble trimer adopts a native-like trimeric Env configuration and displays antigenic properties optimal for a vaccine immunogen.

### Example 6: Epitope Analysis

HIV bnAb Epitopes on SIV Env.

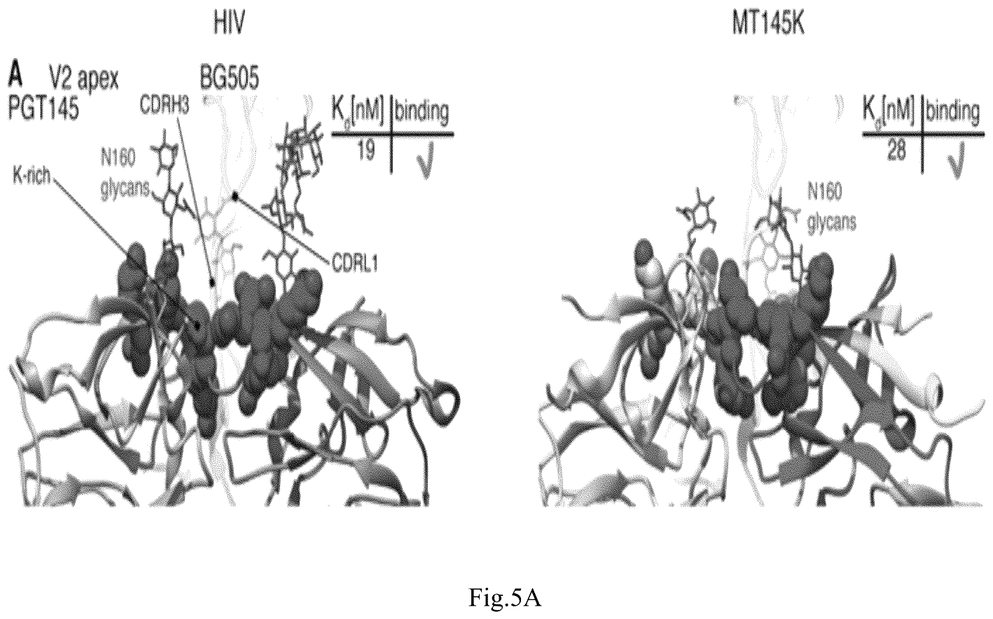

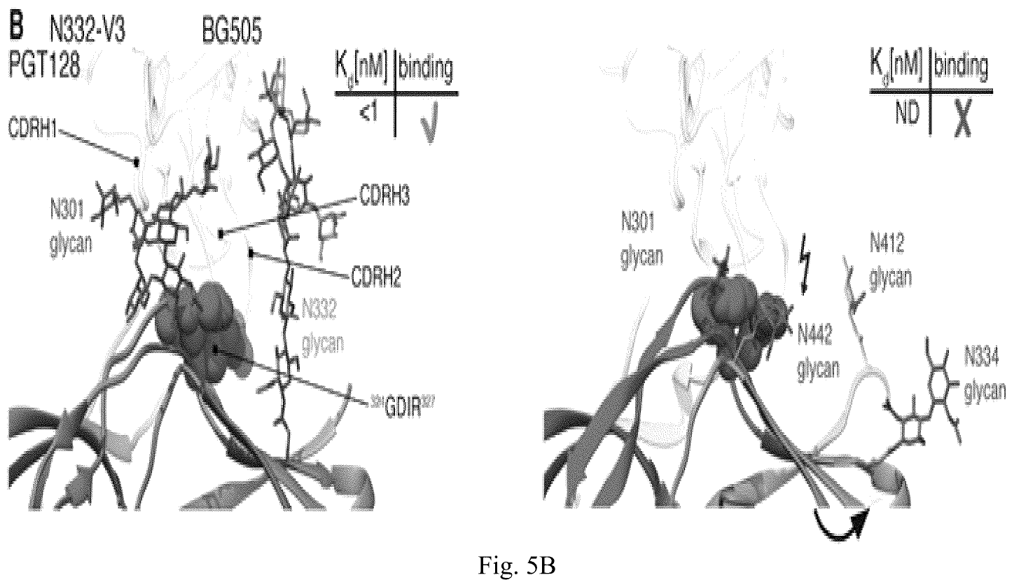

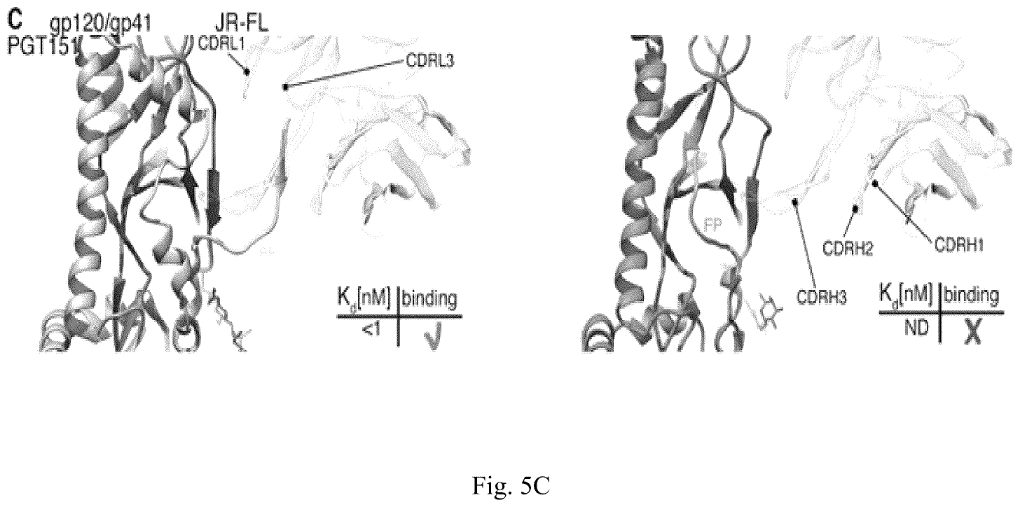

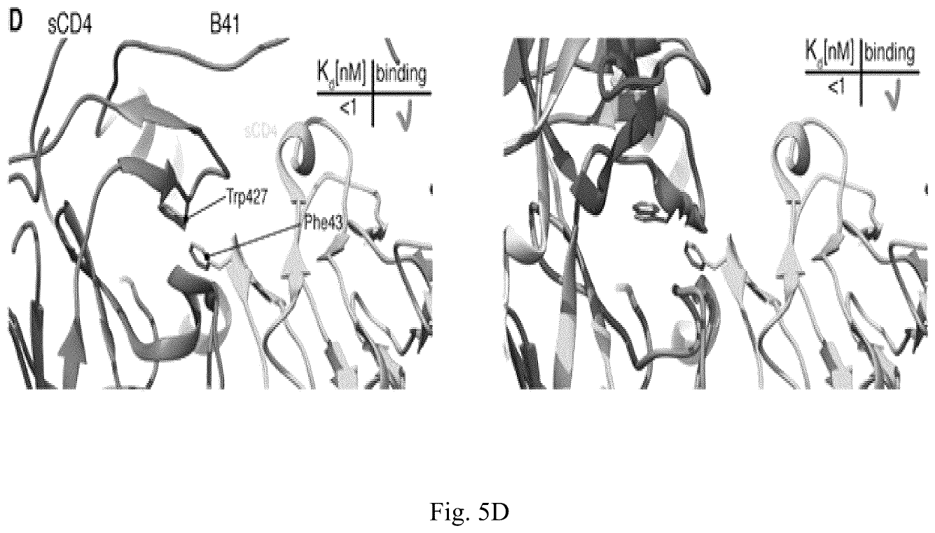

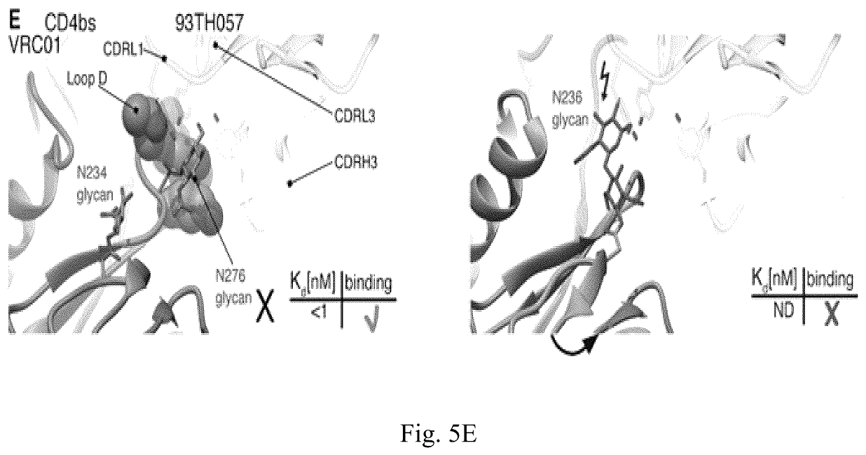

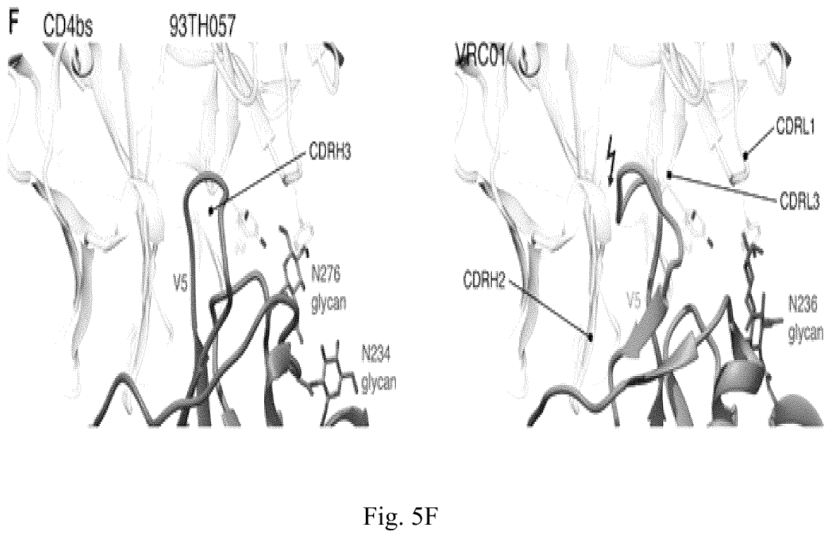

To gain insight into the differences in the HIV-1 Env bnAb epitopes on MT145K SIV Env that may potentially explain the reactivity of V2 apex bnAbs and non-reactivity of HIV bnAbs targeting other Env epitopes, we took advantage of the previously determined structures of human HIV bnAbs in complex with various HIV Env forms and compared the corresponding epitope regions with the MT145K Env (Garces et al., 2014; Lee et al., 2017; Lee et al., 2016; Ozorowski et al., 2017; Pejchal et al., 2011; Wu et al., 2010). A lysine-rich patch in strand C of the V2 loop (¹⁶⁶RDKKQK¹⁷¹ (SEQ ID NO:355) on BG505 Env) and two nearby glycans N160 and N156 form the core epitope for V2 apex bnAbs on HIV Envs (FIG. 5A, 17) (Gorman et al., 2016; Julien et al., 2013b; Lee et al., 2017; Mclellan et al., 2011; Pancera et al., 2013). Both of these features are conserved on the MT145K trimer, thus enabling the human V2 apex bnAbs to be highly effective against the SIV Envs (FIGS. 5A, 15, 17) (Barbian et al., 2015).

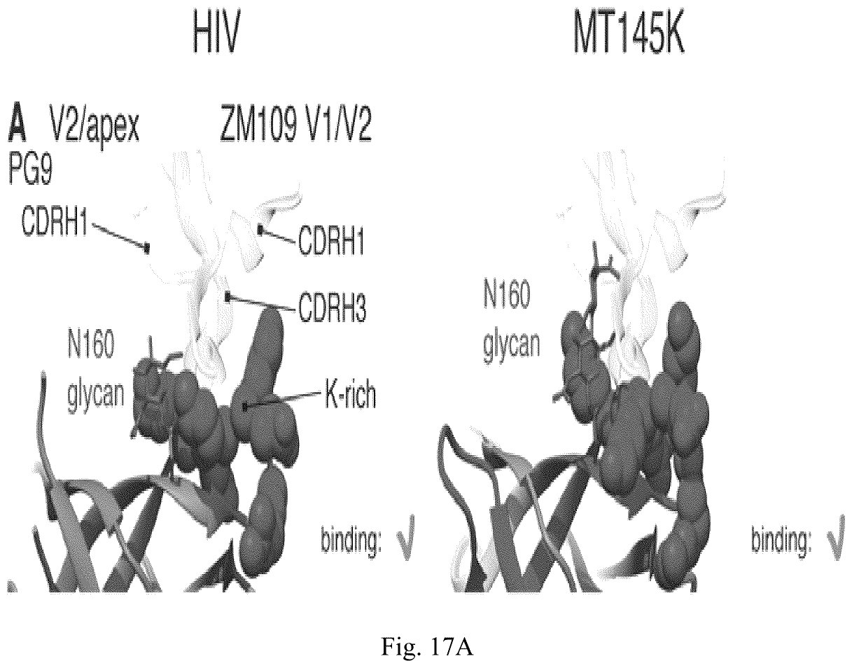

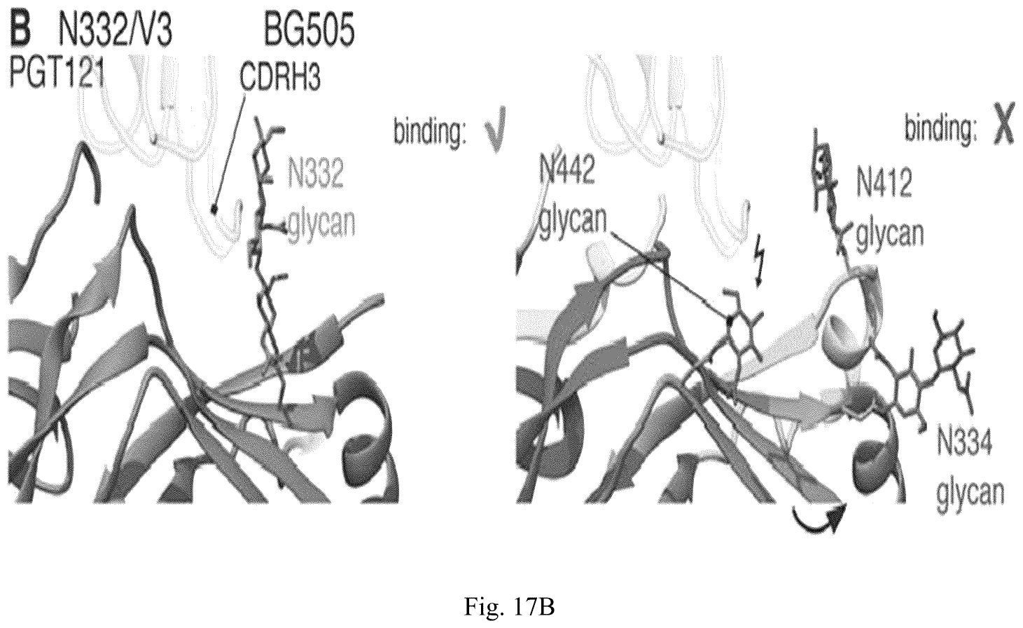

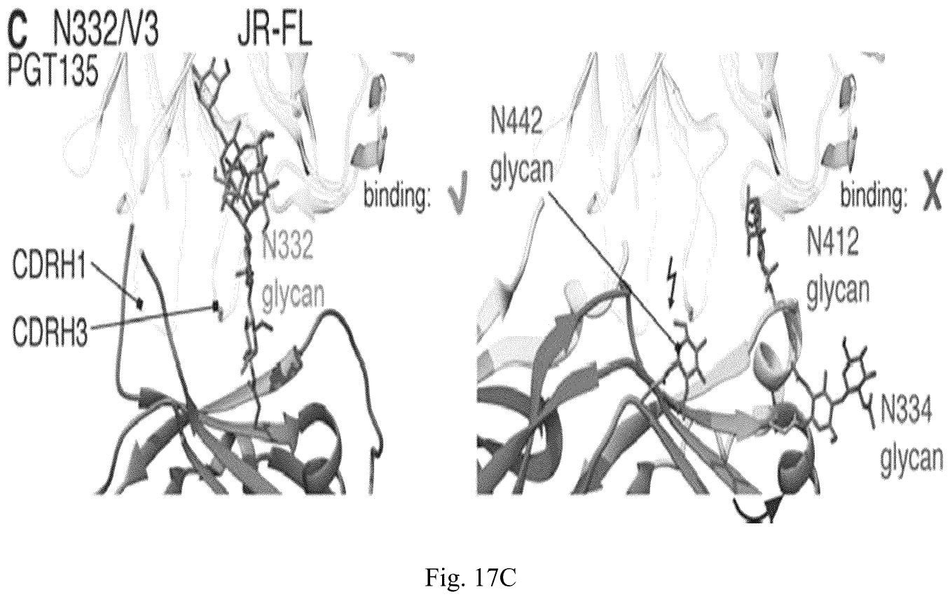

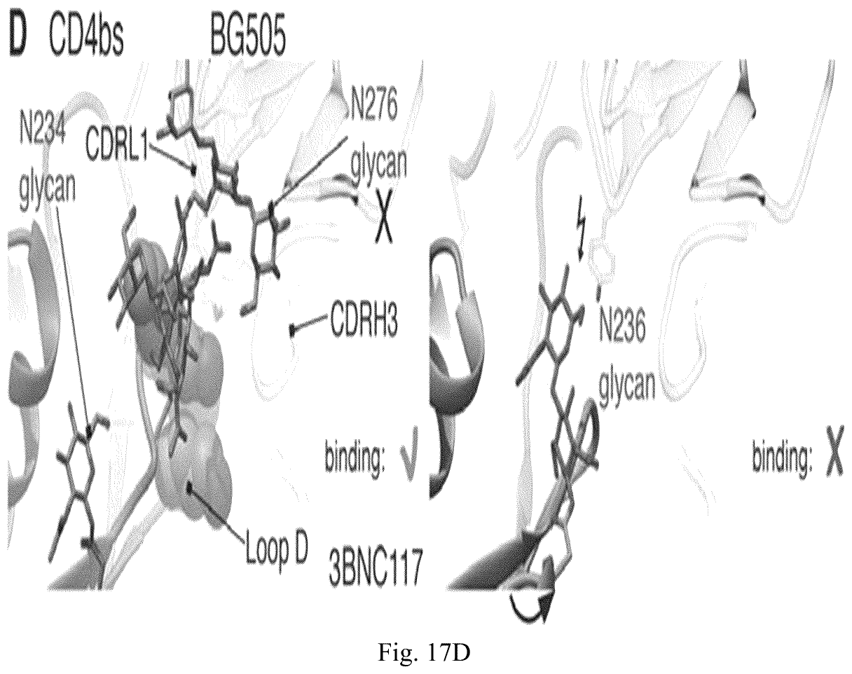

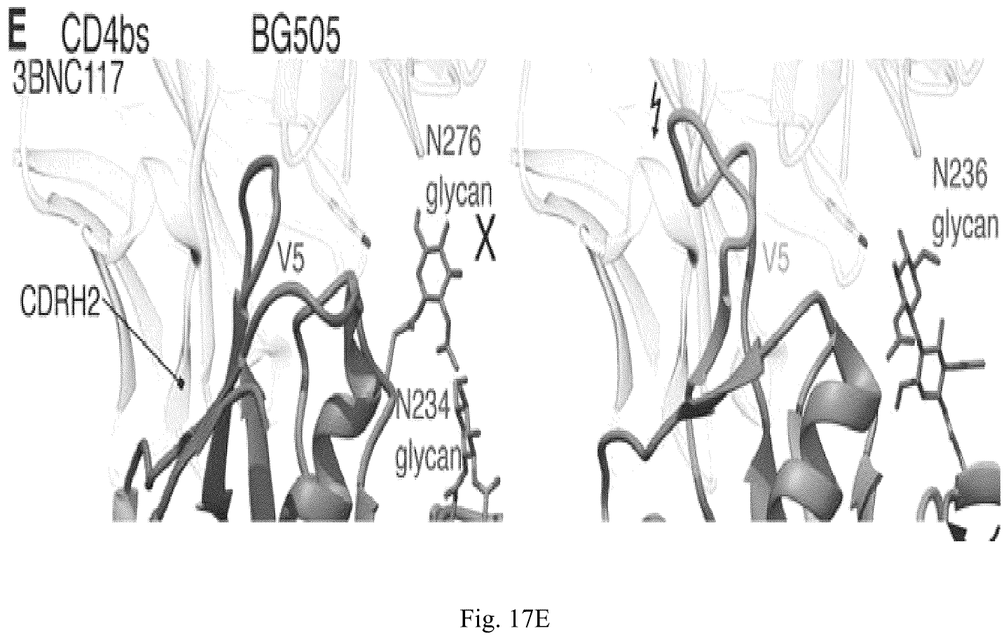

Binding of one of the N332-V3 epitope specific bnAbs, PGT128, predominantly relies on the N332 glycan and a neighboring peptide motif ³²⁴GDIR³²⁷ (SEQ ID NO:356) at the base of the V3 loop (FIGS. 5B, 17) (Garces et al., 2014; Pejchal et al., 2011; Sok et al., 2016b). The lack of binding to the MT145K trimer by PGT128 and other bnAbs in this class can be explained by the absence of the N332 glycan on this Env. In contrast, 3 of the 4 core protein epitope residues 324G-325D-327R are conserved on MT145K trimer and, in fact, on other chimpanzee SIV Envs (FIGS. 5B, 15, 17). For the PGT128 class bnAbs, the interaction with glycan N332 can be substituted by the N295 glycan observed in some HIV isolates, but not by glycan N334 that is present on the MT145K trimer (Sok et al., 2014a). In fact, the MT145K N334 glycan points in a different direction away from the N332-V3 epitope site making it impossible to facilitate bnAb binding to this epitope. Strikingly, the majority of known SIVcpz Env sequences possess an N334 glycan in place of the more common N332 glycan on the HIV Env, which appears to be a significant glycan shift upon species cross-over as the virus established itself in humans (FIG. 15). In addition, the glycan at N412 in the gp120-V4 region of MT145K Env may obstructively interfere with bnAb binding, and, particularly, the glycan at N442, unique to the MT145K Env trimer and several other SIVcpz Envs, would clash with CDRH2 of PGT128 and other bnAbs in this class and may prevent them from accessing the epitope (FIG. 5B, 15).

PGT151 represents another glycan-targeting bnAb class (Blattner et al., 2014; Falkowska et al., 2014; Lee et al., 2016) that recognizes several glycans on gp120 (N88, N448) and gp41 (N611 and N637) as well as the fusion peptide. All glycans and fusion peptide residues that contribute to the PGT151 epitope are conserved between HIV and SIVcpz Envs (FIG. 15). Therefore, the lack of PGT151 binding to MT145K is most likely attributable to inaccessibility of the FP on MT145K (FIG. 5C).

The CD4bs is conserved between HIV and SIV to the extent that there is cross-species reactivity with sCD4. Human CD4-IgG2 immunoadhesin binds well to the MT145K trimer, indicating a strong cross-species conservation of the Env CD4bs. Phe43 in domain-1 of human sCD4 would fit well inside the Trp427 Env cavity on the MT145K trimer reminiscent of its interaction with the HIV Env BG505 trimer (FIG. 5D) (Ozorowski et al., 2017). However, the MT145K trimer is nonreactive with CD4bs bnAbs. VRC01, one of the bnAbs in this class, binds to the HIV Env CD4bs bnAb epitope formed by discontinuous protein backbone elements including loop D of the gp120-C2 region and bordered by a glycan at N276 (FIGS. 5E-F, 17) (Wu et al., 2010). MT145K lacks the N276 glycan and the proximal N234 glycan, present in most HIV-1 Envs, but instead has a glycan at position 236. Differences in the loop D sequence (FIG. 15) and the glycan at N236, which would clash with VRC01 CDRL1 and CDRL3 loops (FIG. 5F) on the MT145K trimer likely impose the biggest impediment to VRC01 binding. Further, the MT145K gp120-V5 loop has a 6-amino acid insertion at HXB2 position 456 compared to HIV-1 Envs that would clash with the VRC01 LC (FIG. 5F, 15).

Overall, the non-reactivity of HIV Env bnAbs with the MT145K trimer can be largely ascribed to subtle glycan shifts that have occurred in HIV-1 from chimpanzee SIV Env as the virus established itself in humans.

### Example 7: Activation of B Cell Precursors

### The Engineered MT145K but not the MT145-WT Trimer Activates V2 Apex UCA-Expressing B Cell Precursors In Vivo.

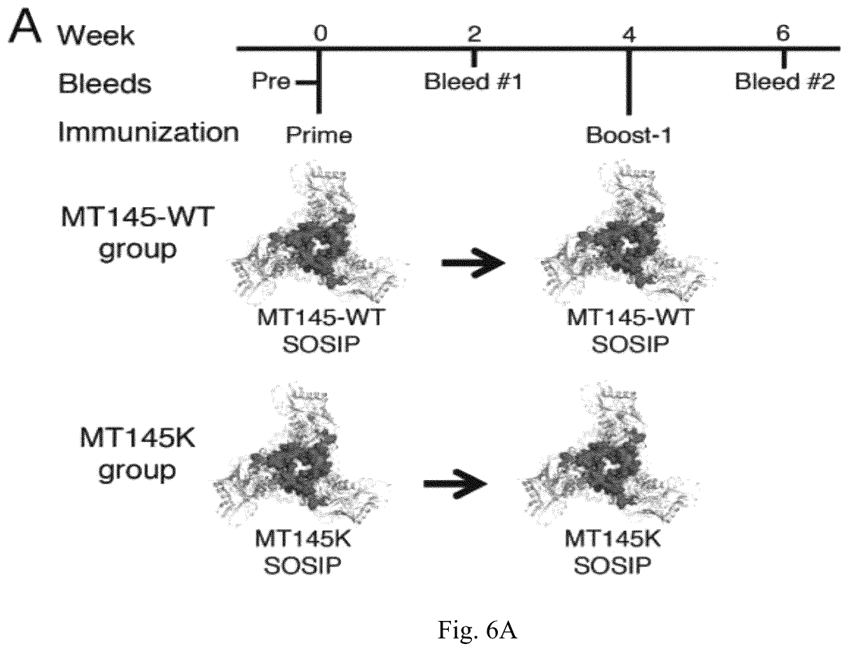

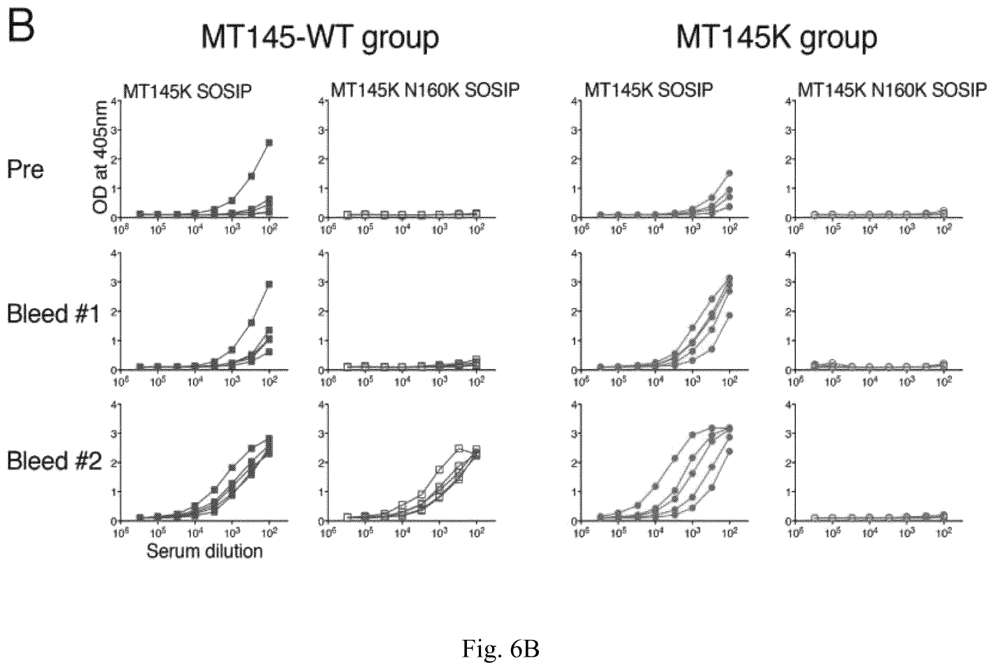

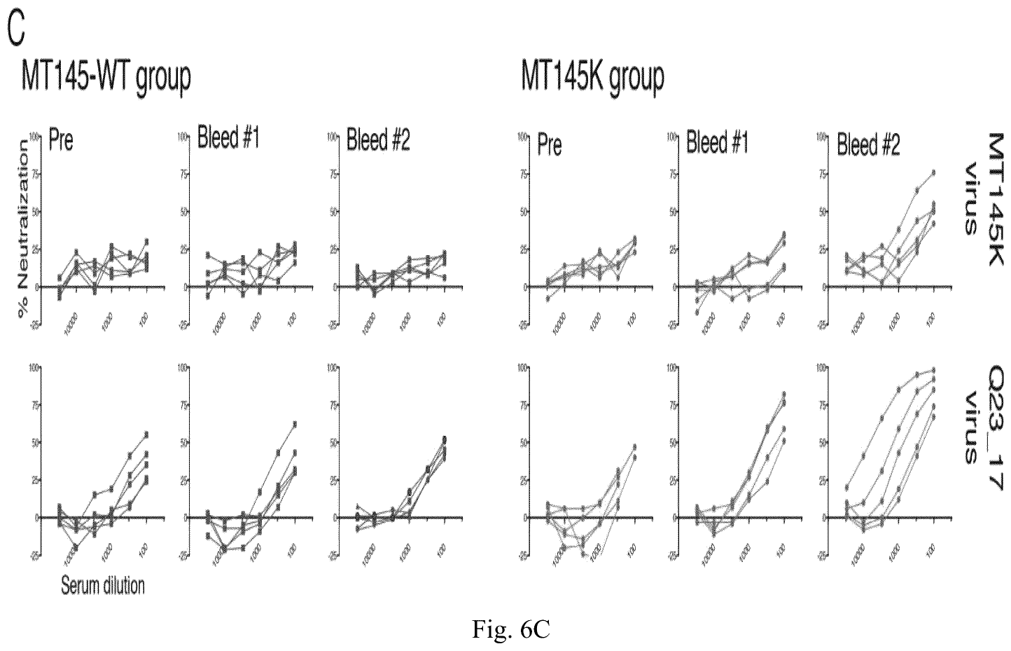

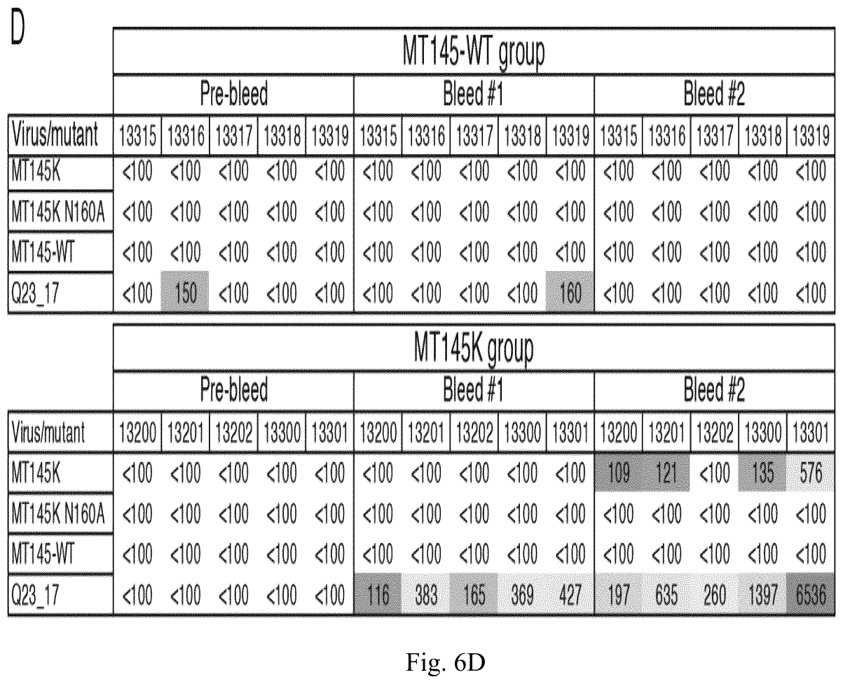

To determine whether the engineered chimpanzee MT145K trimer could efficiently activate HIV V2 apex Ab germline-encoding precursor B cells in vivo and how it compares with the MT145-WT trimer, immunization experiments were conducted in the CH01 unmutated common ancestor (UCA) “HC only” knock-in (KI) mouse model. This KI-mouse model expresses the pre-rearranged heavy chain (V_{H}DDJ_{H}) of the CH01 V2 apex bnAb UCA paired with WT mouse light chains. Two groups of 5 CH01 UCA “HC only” KI mice were immunized, each with two repeated doses (at week-0 and week-4) of MT145-WT or MT145K trimer (FIG. 6A). To track the development of Ab responses, ELISA assays were performed on the pre-bleed, 2-week (day 14) post prime (Bleed #1) and 2-week post boost-1 (day 42) (Bleed #2) serum samples with MT145K SOSIP trimer protein and its N160 glycan knock-out variant (MT145K N160K) (FIG. 6B). The pre-bleed serum samples in both immunization groups exhibited weak binding activity with the MT145K trimer that was dependent on the N160 glycan, consistent with the presence of CH01 UCA Abs that do show some binding to MT145K trimer as described above (FIG. 6B).

The immunogen-specific titers of the serum Ab responses post prime immunizations (Bleed #1 samples) marginally increased in the MT145K group but remained largely unchanged in the MT145-WT trimer immunized group. The serum Ab titers post boost-1 immunization (Bleed #2) increased in both the groups and were orders of magnitude higher as compared to the pre-bleed or the post prime Ab binding responses (FIG. 6B). At this immunization step, the serum Ab responses in the MT145K trimer immunized group were solely dependent on the N160 glycan while the MT145-WT trimer immunization group responses targeted the MT145 Env that were mostly independent of the N160 glycan, which forms part of the core V2 apex bnAb epitope (FIG. 6B). Therefore, we conclude that the engineered MT145K trimer but not the MT145-WT, efficiently triggers the epitope specific V2-apex bnAb UCA encoding B cell precursors in vivo. Remarkably, immunizations with Q171K substituted engineered MT145K trimer also appeared to eliminate the non-V2 apex bnAb site Env specific off-target B cell responses that were elicited in the MT145-WT trimer immunization group (FIG. 6B). The results demonstrate that the activation of the HIV Env bnAb-encoding unmutated B cell precursor by immunogens that display binding to their UCA Ab versions is critical for eliciting epitope-specific Ab responses and the findings are consistent with studies that specifically use germline-targeting immunogen molecules to kick-off the bnAb precursor encoding B cell responses in vivo (Dosenovic et al., 2015; Escolano et al., 2016; Jardine et al., 2015; McGuire et al., 2013; Sok et al., 2016a; Steichen et al., 2016b; Tian et al., 2016).

Immune sera were evaluated for neutralization of autologous and heterologous viruses. Reproducible MT145K autologous virus-specific neutralizing Ab responses were induced in the MT145K immunization group but not in the MT145-WT immunization group (FIG. 6C-D). As for the ELISA binding responses, the nAb titers in the MT145K trimer immunized group increased at 2 weeks post prime, as indicated by nAb titers against a CH01 sensitive HIV Env-encoding virus (Q23_17), and further significantly increased after the boost-1 immunization (FIG. 6C). At this point, all animals in the MT145K group developed autologous virus specific nAb responses (FIG. 6C). The nAb responses in MT145K trimer-immunized animals mapped to the glycan N160 and strand C K171 residue, both of which form part of the core epitope for V2-apex bnAbs, suggesting that the MT145K trimer successfully primed V2 apex UCA B cells in an epitope-specific manner in vivo.

Overall, we conclude that the engineered MT145K but not the MT145-WT trimer activated the V2-apex specific bnAb precursor B cells in a UCA-expressing mouse model and further drove maturation along favorable B cell pathways.

### Example 8: Immunization Strategies

Combining Chimpanzee SIV MT145K Trimer with HIV Env Trimer Immunizations In the CH01 UCA Model.

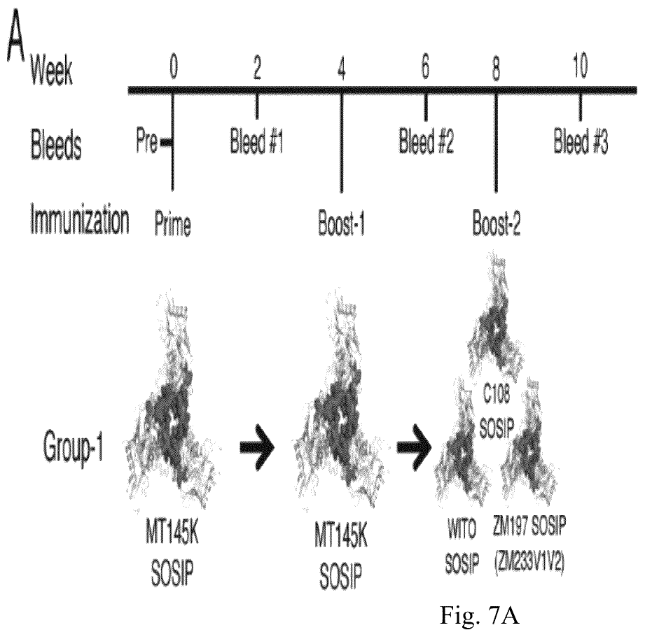

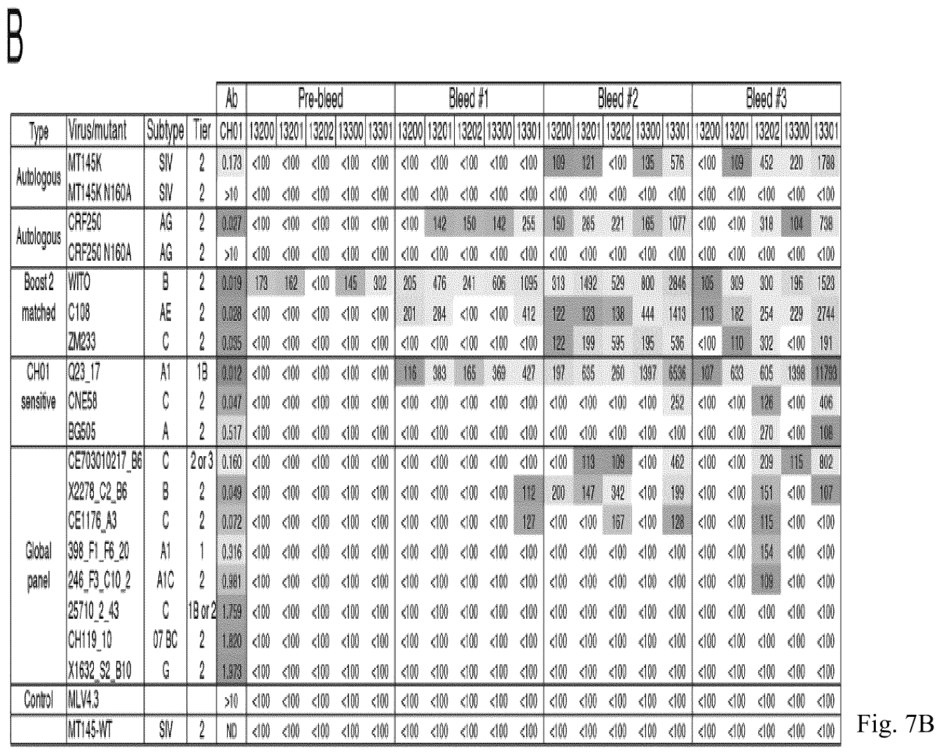

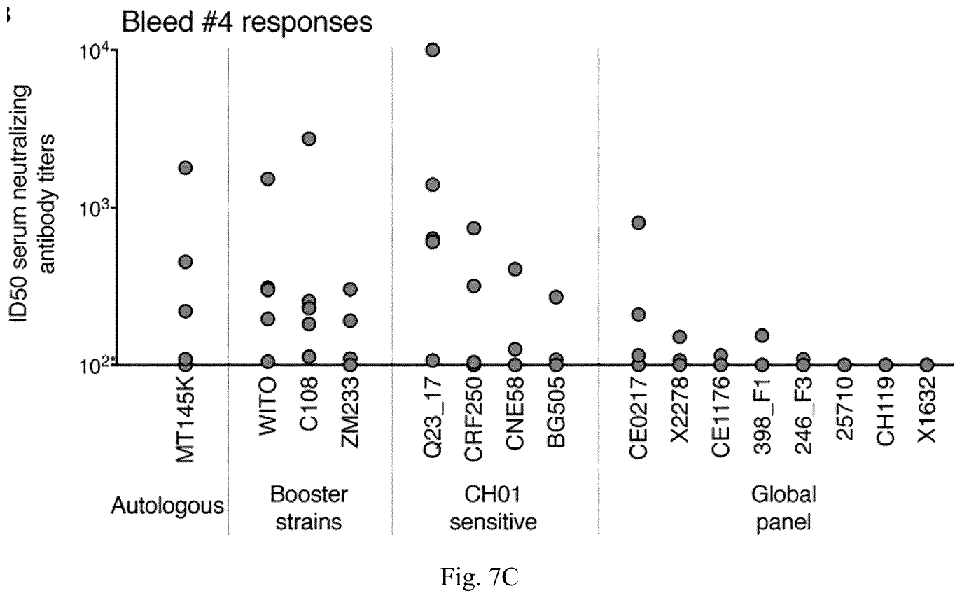

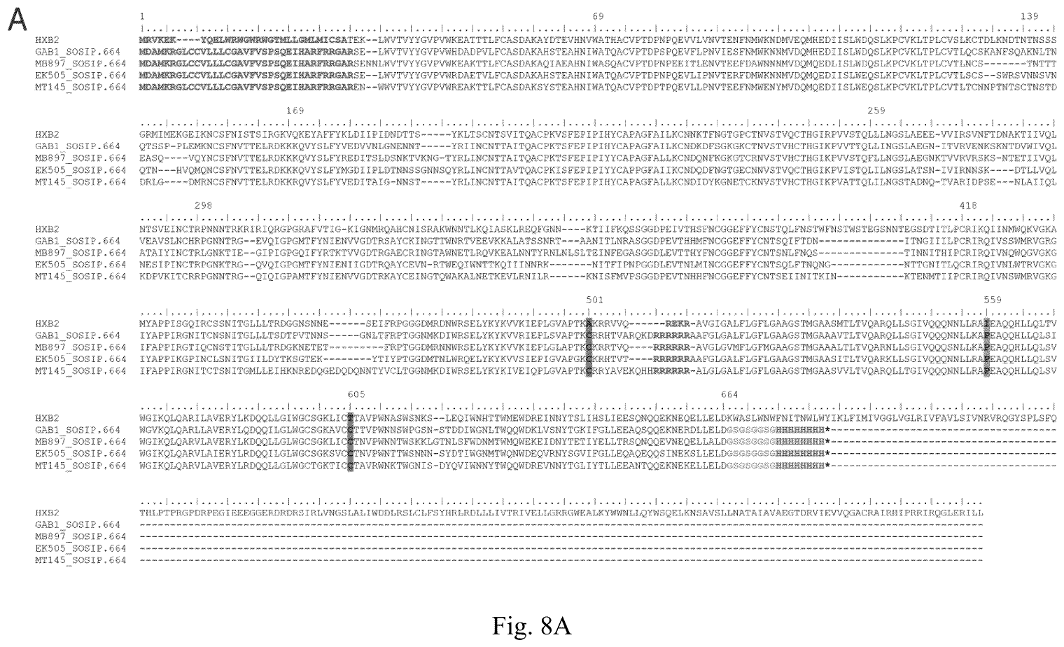

Evaluation of the utility of the MT145K trimer in a sequential HIV immunization regime will be best carried out in humans. Nevertheless, we were interested to investigate the effects of combining the chimpanzee SIV MT145K and HIV Env trimers in a prime-boost immunization in the CH01 UCA “HC only” KI mice. We immunized a group of CH01 UCA KI mice twice with chimpanzee SIV MT145K (as above) and boosted with an HIV Env derived 3-trimer cocktail (C108, WITO and ZM197-ZM233VIV2) (FIG. 7A). NAb titers developed against a few viruses from a global HIV panel that were very sensitive to the mature CH01 bnAb (FIG. 7B). However, viruses that were only somewhat sensitive to CH01 bnAb were not neutralized, suggesting that further affinity maturation would be required for a broad response. The cross-neutralizing responses mapped to the V2 apex bnAb epitope that is shared between HIV and SIV Envs, including to the N160 glycan and the stand C K171 residue.

Overall, the analysis of the immune responses revealed that, due to the extraordinary conservation of the V2 apex bnAb epitope region between HIV and chimpanzee SIV, the MT145K trimer successfully primed human V2 apex bnAb UCA-encoding mouse B cells and induced a V2-focused cross-neutralizing HIV Env specific response that could be further boosted by HIV Env derived trimers.

### Example 9: Materials and Methods

Antibodies, expression and purification. HIV-1 envelope specific mAbs to a broad range of epitopes were used, including those that target V2-apex, V3-N332, linear V3, CD4bs, CD4i and gp120-41 Env sites. A dengue antibody (DEN3) was used as control Ab for binding experiments. For PG9 and CH01 V2-apex bnAb inferred germline antibody designs, the heavy and the light chain V-gene of the mature Abs were reverted to their corresponding closest inferred germline gene sequence as determined using the ImMunoGeneTics (IMGT) website (imgt.cines.fr/) (Brochet et al., 2008). The reverted variable heavy and light chain nucleotide sequences were synthesized by Geneart (Life Technologies) and cloned into corresponding Igyl, Igk, and Ig) expression vectors as previously described (Tiller et al., 2008), using the Gibson cloning method (NEB, USA). The antibodies were expressed and purified using methods described previously (Sok et al., 2014b). Briefly, the heavy and light chain encoding plasmids were reconstituted (1:1 ratio) in Opti-MEM (Life Technologies), and cotransfected HEK293F cells (Invitrogen) using 293fectin (Invitrogen). The suspension cells were cultured for 4-5 days in a shaker incubator at 8% CO2, 37.0° C., and 125 rpm. The antibody containing supernatants were harvested, filtered through a 0.22 mm Steriflip units (EMD Millipore) and passed over a protein A or protein G affinity column (GE Healthcare). The bound antibody was eluted from the columns in 0.1 M citric acid, pH 3.0. Column fractions containing IgG were neutralized (2M Tris-base), pooled, and dialyzed against phosphate-buffered saline (PBS), pH 7.4. IgG purity was determined by sodium dodecyl sulfate-polyacrylamide gel electrophoresis, and the concentration was determined by measuring the relative absorbance at 280 nm.

Site-directed mutagenesis. The amino-acid point mutations in Env-encoding plasmids were incorporated by using a QuikChange site-directed mutagenesis kit (Agilent Technologies, USA), according to the manufacturer's instructions. All of the mutations were confirmed by DNA sequence analysis (Eton Bioscience, San Diego, CA).

Differential Scanning calorimetry. Thermal denaturation was analyzed with a differential scanning calorimetry (DSC) using a MicroCal VP-Capillary DSC instrument (Malvern), at a scanning rate of 1 K/min under 3.0 atmospheres of pressure. Samples were dialyzed in PBS pH 7.4 overnight and protein concentration was adjusted to 0.5 mg/mL prior to measurement. DSC data were analyzed after buffer correction, normalization, and baseline subtraction using MicroCal VP-Capillary DSC analysis software provided by the manufacturer.

Negative stain electron microscopy and data treatment. Purified M145K sample was deposited on thin-carbon-coated (Edwards Auto 306 carbon evaporator) a C-flat EM grid (Cu400 mesh, 2 μm hole diameter, 2 μm hole spacing) (Protochips, Morrisville, NC, USA) and embedded in 2% (w/V) uranyl formate. The carbon-coated grids were Ar/O₂-plasma-cleaned (Gatan Solarus Model 950 Advanced Plasma System; Gatan Inc., Pleasanton, CA, USA) prior to sample deposition. The uranyl-stained EM sample was then inserted into an FEI Tecnai 12 microscope (Thermo Fisher Scientific, Waltham, MA, USA) equipped with a US4000 CMOS detector (Gatan Inc., Pleasanton, CA, USA). The data was collected at 52,000× nominal magnification resulting in a pixel size of 2.05 Å at the object level. Data was binned by a factor of 2 prior to data treatment. Projection image identification in the micrographs was performed with a difference-of-Gaussians implementation (Voss et al., 2009). Projection images subsequently underwent 2D alignment and classification by iterative multi-reference alignment/multivariate statistical analysis (Ogura et al., 2003).

CryoEM sample preparation, data collection, processing and analysis. Purified MT145K sample was deposited on a C-flat EM grid (Cu400 mesh, 2 μm hole diameter, 2 μm hole spacing) (Protochips, Morrisville, NC, USA) that had been Ar/O2-plasma-cleaned (Gatan Solarus Model 950 Advanced Plasma System; Gatan Inc., Pleasanton, CA, USA) prior to sample deposition. Excess buffer was then blotted away from the grid followed by plunging into and vitrification in liquid ethane cooled by liquid nitrogen using a vitrobot (Thermo Fisher Scientific, Waltham, MA, USA). The resulting cryo-EM specimen was transferred into an FEI Titan Krios microscope (Thermo Fisher Scientific, Waltham, MA, USA) equipped with a Gatan K2 Summit direct electron detector (Gatan Inc., Pleasanton, CA, USA). Dose-fractionated data was collected in electron counting mode at a nominal magnification of 29,000× resulting in a pixel size of 1.02 Å at the object level. Micrograph movie frame exposure time was 200 ms and each movie micrograph was recorded over 10s (50 movie frames) corresponding to a total dose of 94e⁻/Å2. Movie micrograph frames were subsequently aligned (MotionCor2; (Zheng et al., 2017)), dose-weighted and signal-integrated resulting in 1,281 micrographs for further data processing. CTF models were determined using GCTF (Zhang, 2016). Candidate projection images of MT145K were identified using a difference-of-Gaussians implementation (Voss et al., 2009). The resulting set of candidate projection images subsequently underwent 2D alignment and classification by use of Relion 2.1b1 (Scheres, 2012). ˜95,000 projection images corresponding to well-formed class averages of MT145K were selected for further data processing. This data class was iteratively angularly refined and reconstructed using a B41 unliganded Env trimer map rendered at 60 Å resolution as an initial reference (Ozorowski et al., 2017). The data class then underwent 3D classification into six classes with the initial reconstruction rendered at 60 Å resolution as reference. From 3D classification, a subset of 44,301 projection images was selected for final data processing comprising CTF model adjustment at the projection-image level (Zhang, 2016) and angular refinement and reconstruction (Scheres, 2012).

Model building and refinement. A homology model (Modeller; (Webb and Sali, 2016)) was generated from sequence alignment of MT145K and BG505 and the structure of the latter (PDB: 4TVP). Significant manual rebuilding followed in Coot (Emsley and Cowtan, 2004). A fragment library was then created from the MT145K sequence containing 200 homologous, non-redundant sequences at each MT145K 7-mer position. Library fragment-based, density-guided, real-space rebuilding was then performed (DiMaio et al., 2015) with 319 decoys. The resulting models were evaluated geometrically (MolProbity; (Chen et al., 2010)) and by fit-to-map (EMRinger; (Barad et al., 2015). The overall best model was selected for further iterations of manual rebuilding and multi-decoy, density-guided, real-space, all-atom Rosetta FastRelax refinement. Finally, glycans were manually built in Coot and restricted, density-guided real-space refinement performed in Phenix 1.12 (Adams et al., 2002) followed by model evaluation by MolProbity, EMRinger and Privateer (Agirre et al., 2015).

Global N-linked glycan analysis. The quantifications and structural characterization of the total glycan pool was achieved by cleaving the N-inked glycans from the surface of the glycoprotein using an in-gel digestion with peptide N-glycosidase F (PNGaseF). The resultant glycans were separated into two aliquots. The first was derivatized with 2-aminobenzoic acid (2-AA) and subjected to HILIC-UPLC analysis using an Acquity UPLC (Waters). To quantify the oligomannose content of the released glycans, the labelled samples were treated with endoglycosidase H (endoH), which selectively cleaves oligomannose glycans. Data analysis and interpretation were performed using Empower software (Waters). The second aliquot of released glycans was subjected to negative ion electrospray ion mobility mass spectrometry using a Synapt G2Si mass spectrometer (Waters). Glycan compositions were determined using collision induced dissociation (CID) fragmentation. Data analysis was performed using Waters Driftscope (version 2.8) software and MassLynx™ (version 4.1). Spectra were interpreted as described previously (Harvey et al., 2009). The glycan compositions were used to generate a sample-specific glycan library that was used to search the glycopeptide data to minimize the number of false-positive assignments in site-specific analysis.

LC-MS glycopeptide analysis. Site-specific N-glycosylation analysis was performed using proteolytic digestion followed by tandem LC-MS. Prior to digestion, trimers were denatured, reduced and alkylated by incubation for 1 h at room temperature (RT) in a 50 mM Tris/HCl, pH 8.0 buffer containing 6 M urea and 5 mM dithiothreitol (DTT), followed by the addition of 20 mM iodacetamide (IAA) for a further 1 h at RT in the dark, and then additional DTT (20 mM) for another 1 h, to eliminate any residual IAA. The alkylated trimers were buffer-exchanged into 50 mM Tris/HCl, pH 8.0 using Vivaspin columns (GE healthcare) and digested separately with trypsin, elastase and chymotrypsin (Mass Spectrometry Grade, Promega) at a ratio of 1:30 (w/w). Glycopeptides were selected from the protease-digested samples using the ProteoExtract Glycopeptide Enrichment Kit (Merck Millipore) following the manufacturer's protocol. Enriched glycopeptides were analyzed by LC-ESI MS on an Orbitrap fusion mass spectrometer (Thermo Fisher Scientific), as previously described (Behrens et al., 2016), using higher energy collisional dissociation (HCD) fragmentation. Data analysis and glycopeptide identification were performed using Byonic™ (Version 2.7) and Byologic™ software (Version 2.3; Protein Metrics Inc.), as previously described (Behrens et al., 2016).

Glycan modeling. Man₉GlcNAc₂ oligomannose-type glycans were docked and rigid-body fitted at each of the corresponding Env glycan positions using the MT145K structure presented here or an unliganded BG505 SOSIP.664 structure (PDB: 4ZMJ).

Pseudovirus production. To produce pseudoviruses, Env-encoding plasmids were cotransfected with an Env-deficient backbone plasmid (pSG3ΔEnv) (1:2 ratio) using X-tremeGENE™ 9 (Sigma-Aldrich) DNA transfection reagent. Briefly, 1×10⁶ cells in 10 ml of Dulbecco's Modified Eagle Medium (DMEM) were seeded in a 100 mm×20 mm cell culture dish (Corning) one day prior to transfection. For transfection, 40 μl of X-tremeGENE™ 9 was added to 700 μl of Opti-MEM I reduced serum medium (Thermo Fisher) in tube 1. The Env-encoding plasmid (5 μg) and pSG3ΔEnv (10 μg) were added to tube 2 in 700 μl of Opti-MEM. The tube 1 and tube 2 solutions were mixed together and incubated for 25 min at room temperature. Next, the transfection mixture was added to the media with 293T cells seeded previously and then distributed uniformly. All pseudoviruses were harvested 48-72 h posttransfection, filtered through 0.22 mm Steriflip units (EMD Millipore) and aliquoted for use in neutralization assays.

Neutralization assay. Neutralization was measured by using single-round replication-defective HIV Env-pseudoviruses and TZM-b1 target cells (Montefiori, 2005; Seaman et al., 2010). 25 μl of 3-fold serially diluted mAbs or serum samples were pre-incubated at 37° C. for 1 h with 25 μl of tissue culture infective dose-50 (TCID50) Env-pseudotyped virus in a half-area 96-well tissue culture plate. TZM-b1 target cells (5,000 cells/well) in 50 μl of DMEM were added and the plates were allowed to grow in humidified incubator at 37° C. and 5% Co₂. The luciferase activity of the lysed cells was read on instrument (Biotek) after 2-3 days, by adding lysis buffer followed by Brightglow (Promega). The 50% inhibitory concentration (IC₅₀) or 50% inhibitory doses (ID₅₀) was reported as the antibody concentration or serum dilution required to reduce infection by half.

ELISA binding assay. ELISA binding experiments were performed as described previously with minor modification (Sanders et al., 2013). ELISA binding with SOSIP.664 trimer proteins with mAbs was carried out by either capturing the trimer proteins onto the anti-His capture antibodies or on the streptavidin coated plates through biotinylated trimers. For trimer biotinylation, the SOSIP.664 proteins were randomly biotinylated using a 2:1 molar ratio of biotin reagent to trimer using the EZ-link-NHS-PEG4-Biotin kit (Thermo Fisher Scientific, 21324). MaxiSorp plates (Thermo Fisher Scientific) were coated overnight at 4C with 2 μg/mL of anti-His Ab (Thermo Fisher Scientific) or 2 μg/mL streptavidin (Thermo Fisher Scientific). Plates were blocked for 1 hr with 3% BSA and washed three times with 0.05% Tween 20-PBS (PBS-T) (pH 7.4). Anti-His or Streptavidin-coated plates were incubated with biotinylated trimers in 1% BSA plus PBST for 1.5 hr and washed three times with PBST. 3-fold serially diluted mAbs or sera were added starting at a maximum concentration of 10 μg/mL (100 μg/ml for iGL Abs) (sera at 1:100 dilution) in 1% BSA plus PBST, and incubated at room temperature (RT) for 1.5 hr. Plates were washed three times with PBST. Alkaline-phosphatase-conjugated goat anti-human IgG Fc secondary antibody (Jackson ImmunoResearch Laboratories) was diluted 1:1000 in 1% BSA PBST and added to plates for 1 hr at RT. Plates were washed three times with PBST and incubated with phosphatase substrate (Sigma) for 15 mins and the absorbance at 405 nm recorded. The 50% binding (EC50) was recorded as the half of the maximum binding activity and was calculated by linear regression method using Prism 6 Software.

Bio Layer Interferometry (BLI) binding assay. The binding experiments of Abs to the affinity purified trimers were performed with an Octet K2 system (ForteBio, Pall Life Sciences). Briefly, the mAbs or IgGs (10 μg/mL in PBST) were immobilizing onto hydrated anti-human IgG-Fc biosensors (AHC: ForteBio) for 60 seconds to achieve a binding response of at least 1.0. After Ab capture, the sensor was placed in a PBST wash buffer to remove the unbound Ab to establish a baseline signal. Next, the IgG immobilized sensor was dipped into a solution containing SOSIP.664 trimer protein as analyte and incubated for 120 seconds at 1000 rpm. Following this, the trimer bound to IgG immobilized sensor was removed from the analyte solution and placed into the PBST buffer for 240 seconds at 1000 rpm. The 2 and 4 minute binding intervals respectively denote the association and dissociation binding curves reported in this study. The sensograms were corrected with the blank reference and fit (1:1 binding kinetics model) with the ForteBio Data Analysis version.9 software using the global fitting function. The data are represented as maximum binding response or the association and dissociation curve fits.

Trimer protein immunizations in CH01 UCA HC-only KI-mice. For the immunization experiments, groups of 5 CH01 UCA HC-only knock-in B cell expressing mice were immunized with 25 μg of the individual trimer protein or 25 μg total protein of the 3-trimer cocktail (Prime, week-0; Boost-1, week-4 and Boost-2, week-8) along with Glucopyranosyl Lipid Adjuvant in stable emulsion (GLA-SE) as adjuvant. Immunizations were administered intramuscular in the leg of each animal with 25 μg of total trimer immunogens. Blood samples were collected at pre-bleed, 2-weeks each, post-prime (Bleed #1), post boost-1 (Bleed #2) and post boost-2 (Bleed #3) immunization time-point for the isolation of sera that were tested for presence of neutralizing antibodies in TZM-b1 cell based assay. Serum samples were heat inactivated for potential complement activity at 56° C. for 0.5 h. Mice used in this study were approved by Duke University Institutional Animal Care and Use Committee-approved animal protocols.

Data availability. Cryo-EM reconstructions have been deposited in the Electron Microscopy Data Bank.

### Example 10: Immunization Strategies and Responses

### Sequential MT145K Trimer Boost Following HIV Trimer Prime Recalls “on Target” V2-Apex Specific Memory B Cell Responses and Disfavors Off-Target Responses.

In addition to priming the V2-apex specific B cell responses, the chimpanzee MT145K trimer can be used as a boost to recall HIV trimer induced V2-apex directed memory B cell responses.

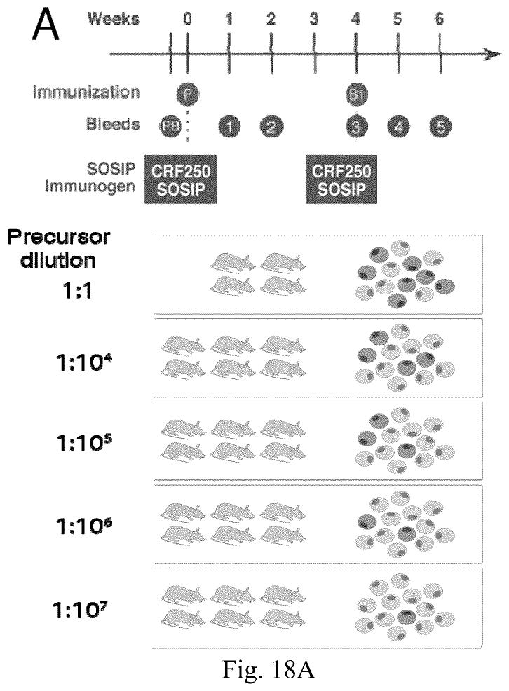

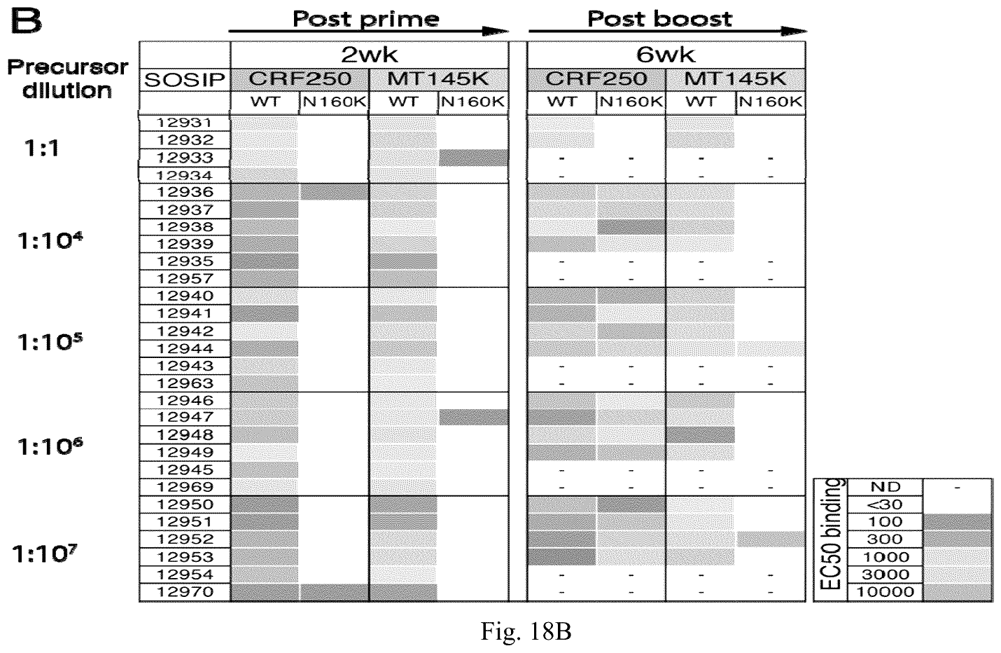

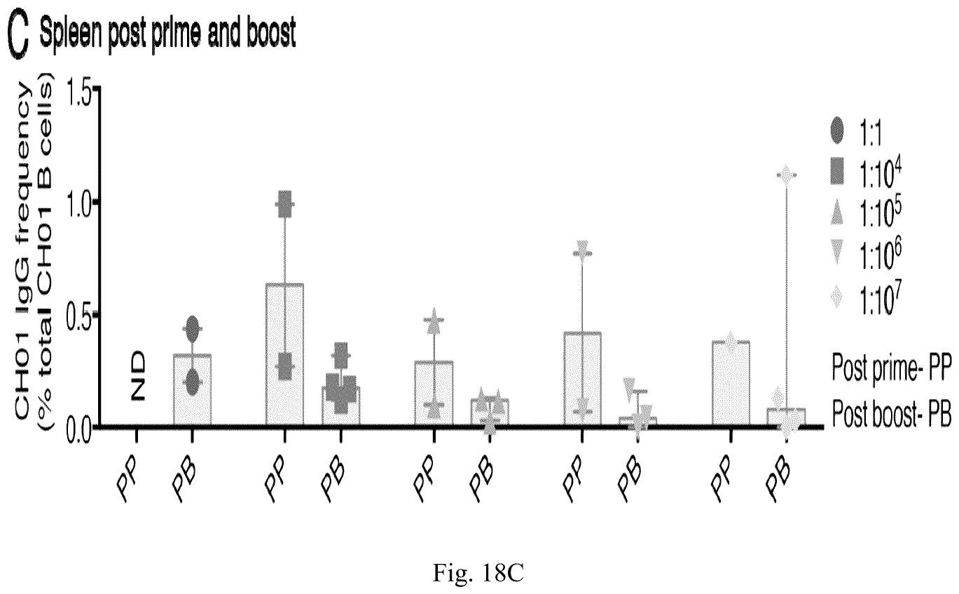

CH01 iGL B cell activation by HIV GT-trimer, CRF250, under rare B cell conditions to mimic their natural circulating frequencies (CH01 iGL B cell diluted up to 1 in 10⁷ cells) was investigated by adoptively transferring CH01 iGL B cells (CD45.2) into WT mice (CD45.1) (FIG. 18A). CRF250 GT-trimer binds CH01 UCA BCR with 100 nM KD affinity and efficiently activated the on-target V2-apex CH01 B cells even at 1 in 107 precursor frequency as determined by ELISA binding of the week-2 (2 wk) post prime Ab responses with CRF250 SOSIP trimer and its N160 glycan-eliminated V2-epitope specific knock-out variant (FIG. 18B). Further boosting with CRF250 trimer recalled the V2-specific Ab responses (6 wk), but also resulted in the development of a strong CRF250 Env-backbone specific off-target B cell responses targeting epitopes outside of the V2-apex bnAb site, as probed by N160 glycan-dependent binding (FIG. 18B). The development of CRF250-specific off-target B cell responses coincided with reduction of CH01-specific class-switched memory B cell responses, as determined by next generation sequencing of B cells from immunized mice (FIG. 18C). Both the CRF250 trimer-elicited post-prime and the post-boost Ab responses displayed strong binding to the chimpanzee SIV MT145K trimer in a V2-apex bnAb epitope-specific manner but largely no binding to the HIV Env backbone-specific off-target Ab responses (FIG. 18B). The results suggest that sequential prime boosting with HIV and SIV trimers that share V2-apex bnAb site can be advantageous, especially in that off-target responses should be disfavored.

### MT145KdV5 SOSIP Trimer Version Induces Superior Cross-Neutralizing Ab Responses Compared to the Parent MT145K SOSIP Trimer in CH01 iGL HC-Only KI Mouse Model.

Infection of non-human primates (NHPs) with Simian-Human Immunodeficiency Virus (SHIV)-encoding MT145K envelope induces a rapid and strong autologous neutralizing antibody response largely directed to the immunodominant hypervariable V5-loop of gp120. In response to this early wave of autologous nAb responses, the circulating MT145K SHIV viruses develop multiple escape solutions, including a 9 amino acid deletion in the V5-loop (George Shaw et al, unpublished). Multiple animals developed this common viral escape solution of 9AA V5-loop deletion. Structure of MT145K SOSIP trimer revealed a peripheral positioning of the flexible V5 loop, consistent with its in vivo B cell immunodominance effects.

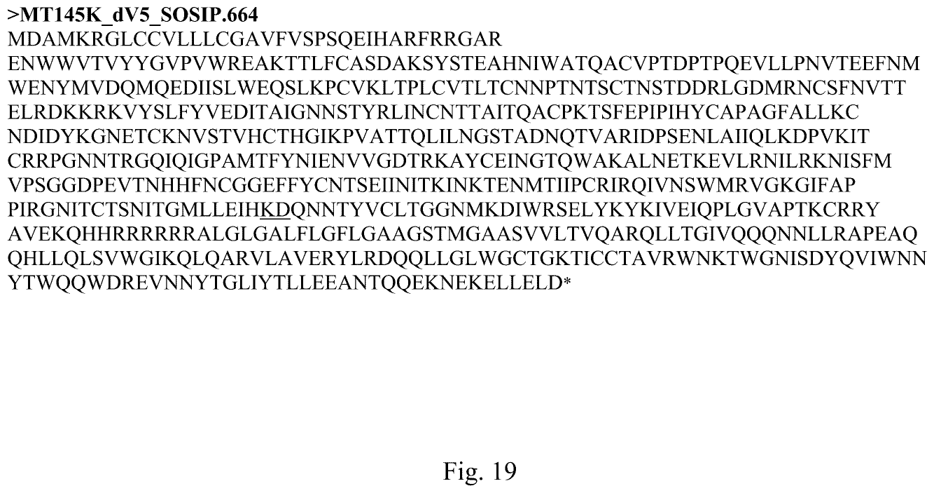

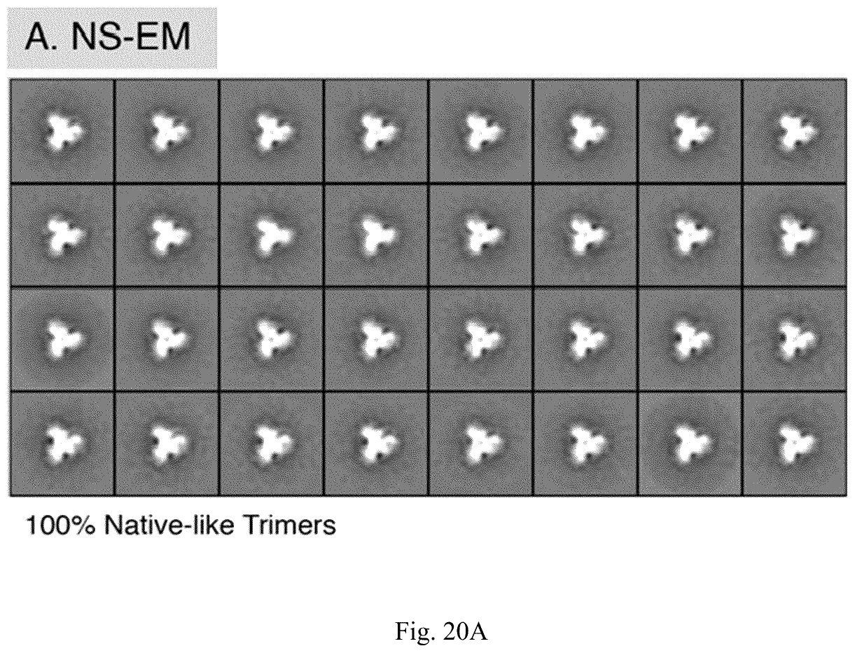

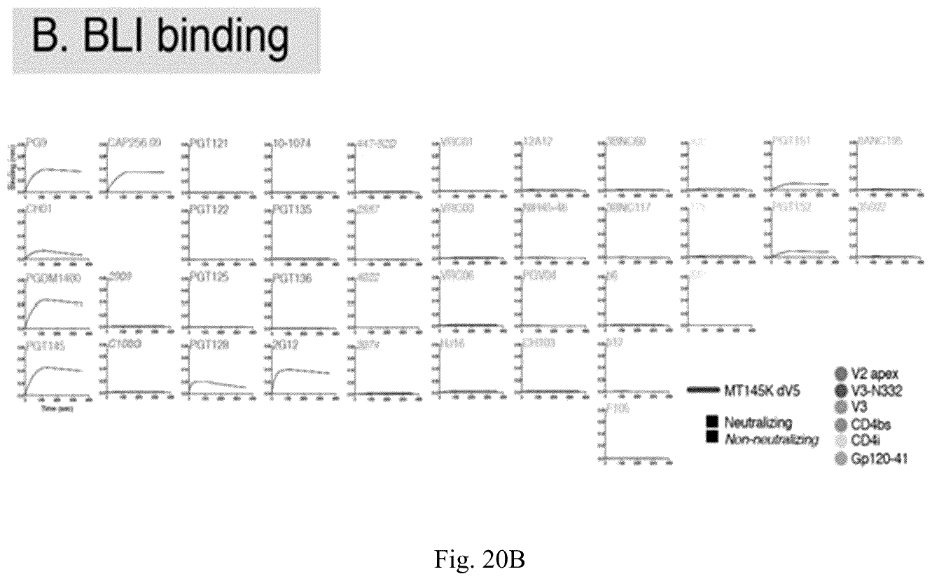

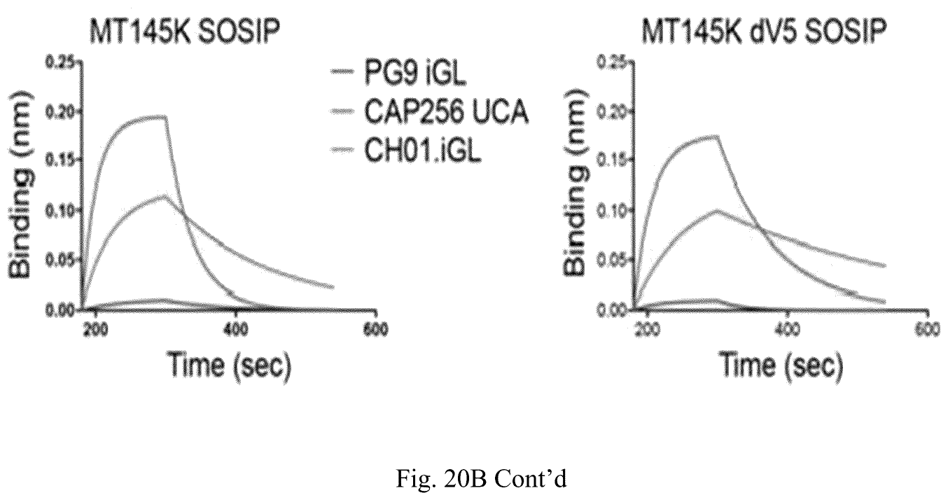

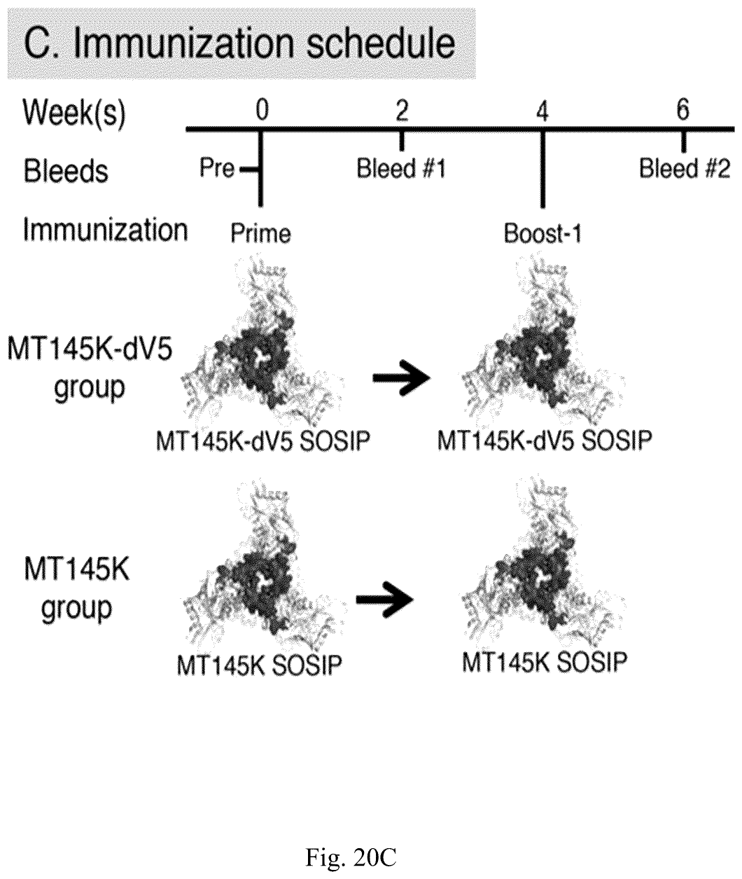

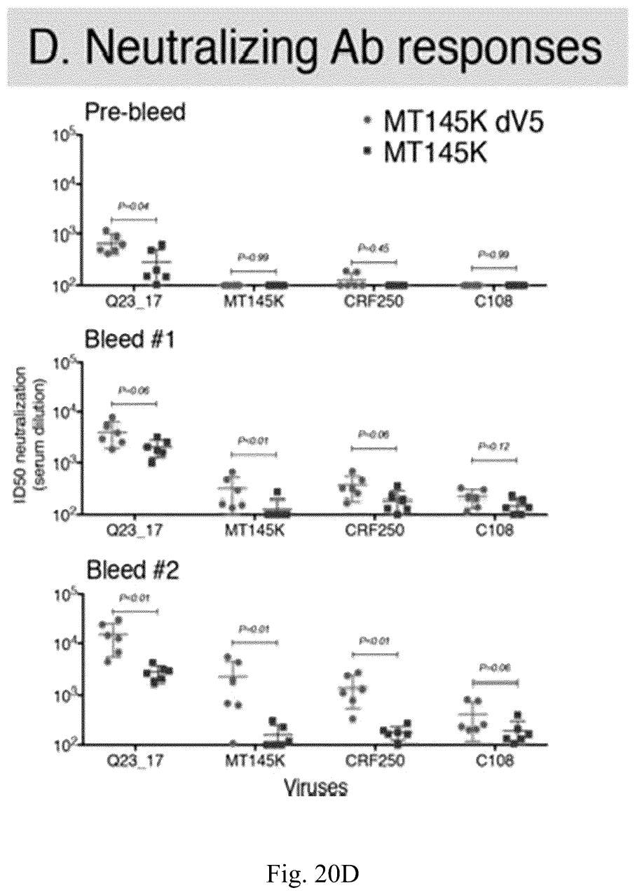

The MT145K SOSIP trimer was engineered to eliminate this 9AA immunodominant V5-loop (FIG. 19) and the resulting SOSIP trimer, MT145KdV5 with a 9AA V5-deletion showed native-like trimer antigenic and biophysical properties similar to the parent MT145K SOSIP trimer (FIG. 20). A head-to-head immunogencity comparison of MT145dV5 trimer version with MT145K in CH01 iGL HC-only KI mice showed induction of a more reproducible and superior cross-neutralizing serum antibody response by MT145KdV5 trimer. Deletion of the immunodominant V5-loop in the MT145KdV5 can reduce the development of non-desired off-target B cell responses and therefore, the modified version of the trimer can be more optimal for human vaccinations to induce V2-apex bnAbs.

### Chimpanzee SIV MT145KdV5 SOSIP Trimer Induces Autologous Neutralizing Antibodies in NHPs

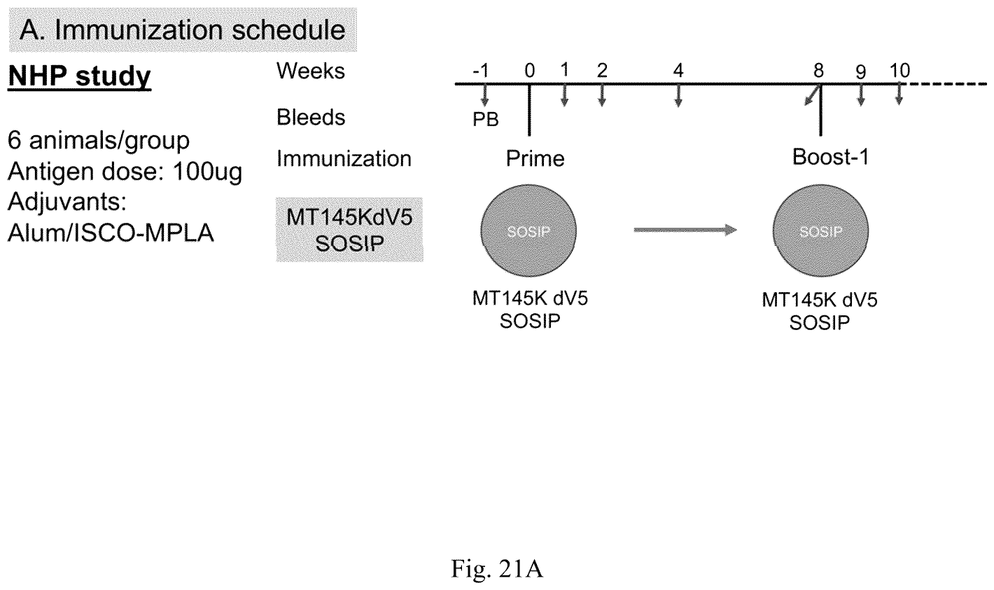

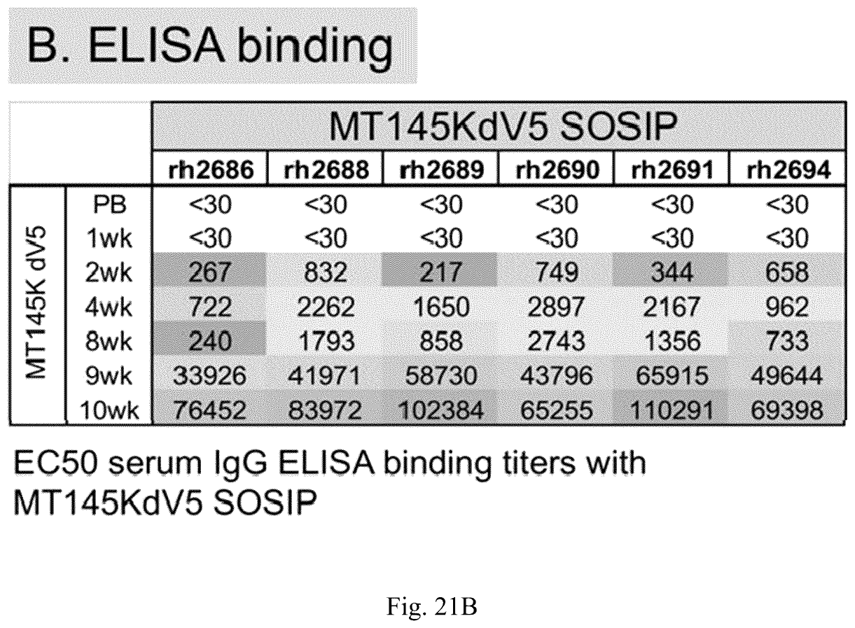

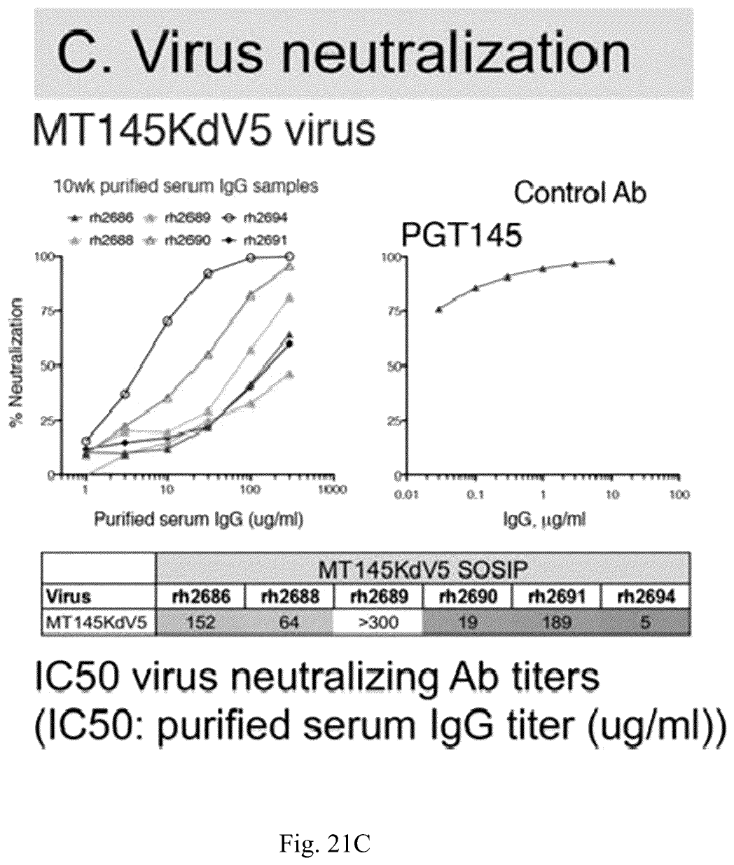

The immunogenicity of MT145KdV5 trimer was investigated in NHPs, whose B cell repertoires closely resemble to that of humans. A group of 6 NHPs was immunized twice (prime, week 0; boost-1, week 8) with MT145KdV5 SOSIP trimer and monitored the development of antibody responses by ELISA and neutralization assay (FIG. 21). ELISA binding of the post-prime serum antibodies revealed a strong development of autologous trimer immunogen specific binding responses (FIG. 21B). The titers of the Ab binding responses substantially increased post boost immunization indicating a B cell recall response. In addition to stronger Ab binding titers, the post boost (10 wk) serum antibody responses exhibited neutralizing activities against the autologous MT145KdV5 virus (FIG. 21C).

### REFERENCES

Adams, P. D., Grosse-Kunstleve, R. W., Hung, L. W., loerger, T. R., McCoy, A. J., Moriarty, N. W., Read, R. J., Sacchettini, J. C., Sauter, N. K., and Terwilliger, T. C. (2002). PHENIX: building new software for automated crystallographic structure determination. Acta Crystallogr D Biol Crystallogr 58, 1948-1954.
Agirre, J., Iglesias-Fernandez, J., Rovira, C., Davies, G. J., Wilson, K. S., and Cowtan, K. D. (2015). Privateer: software for the conformational validation of carbohydrate structures. Nature structural & molecular biology 22, 833-834.
Andrabi, R., Pallesen, J., Allen, J. D., Song, G., Zhang, J., de Val, N., Gegg, G., Porter, K., Su, C. Y., Pauthner, M., Newman, A., Bouton-Verville, H., Garces, F., Wilson, I. A., Crispin, M., Hahn,
B. H., Haynes, B. F., Verkoczy, L., Ward, A. B., Burton, D. R. (2019) The Chimpanzee SIV Envelope Trimer: Structure and Deployment as an HIV Vaccine Template. Cell Rep. 21, 2426-2441.
Andrabi, R., Su, C. Y., Liang, C. H., Shivatare, S. S., Briney, B., Voss, J. E., Nawazi, S. K., Wu, C. Y., Wong, C. H., and Burton, D. R. (2017). Glycans Function as Anchors for Antibodies and Help Drive HIV Broadly Neutralizing Antibody Development. Immunity 47, 524-537 e523.
Andrabi, R., Voss, J. E., Liang, C. H., Briney, B., McCoy, L. E., Wu, C. Y., Wong, C. H., Poignard, P., and Burton, D. R. (2015). Identification of Common Features in Prototype Broadly Neutralizing Antibodies to HIV Envelope V2 Apex to Facilitate Vaccine Design. Immunity 43, 959-973.
Barad, B. A., Echols, N., Wang, R. Y., Cheng, Y., DiMaio, F., Adams, P. D., and Fraser, J. S. (2015). EMRinger: side chain-directed model and map validation for 3D cryo-electron microscopy. Nature methods 12, 943-946.
Barbian, H. J., Decker, J. M., Bibollet-Ruche, F., Galimidi, R. P., West, A. P., Jr., Learn, G. H., Parrish, N. F., Iyer, S. S., Li, Y., Pace, C. S., et al. (2015). Neutralization properties of simian immunodeficiency viruses infecting chimpanzees and gorillas. mBio 6.
Behrens, A. J., Harvey, D. J., Milne, E., Cupo, A., Kumar, A., Zitzmann, N., Struwe, W. B., Moore, J. P., and Crispin, M. (2017). Molecular Architecture of the Cleavage-Dependent Mannose Patch on a Soluble HIV-1 Envelope Glycoprotein Trimer. Journal of virology 91.
Behrens, A. J., Vasiljevic, S., Pritchard, L. K., Harvey, D. J., Andev, R. S., Krumm, S. A., Struwe, W. B., Cupo, A., Kumar, A., Zitzmann, N., et al. (2016). Composition and Antigenic Effects of Individual Glycan Sites of a Trimeric HIV-1 Envelope Glycoprotein. Cell reports 14, 2695-2706.
Benichou S, Venet A, Beyer C, Tiollais P, and Madaule P. (1993). Characterization of B-cell epitopes in the envelope glycoproteins of simian immunodeficiency virus. Virology 194, 870-874.
Bhiman, J. N., Anthony, C., Doria-Rose, N. A., Karimanzira, O., Schramm, C. A., Khoza, T., Kitchin, D., Botha, G., Gorman, J., Garrett, N. J., et al. (2015). Viral variants that initiate and drive maturation of V1V2-directed HIV-1 broadly neutralizing antibodies. Nature medicine 21, 1332-1336.
Blattner, C., Lee, J. H., Sliepen, K., Derking, R., Falkowska, E., de la Pena, A. T., Cupo, A., Julien, J. P., van Gils, M., Lee, P. S., et al. (2014). Structural delineation of a quaternary, cleavage-dependent epitope at the gp41-gp120 interface on intact HIV-1 Env trimers. Immunity 40, 669-680.
Bonomelli, C., Doores, K. J., Dunlop, D. C., Thaney, V., Dwek, R. A., Burton, D. R., Crispin, M., and Scanlan, C. N. (2011). The glycan shield of HIV is predominantly oligomannose independently of production system or viral clade. PloS one 6, e23521.
Bonsignori, M., Hwang, K. K., Chen, X., Tsao, C. Y., Morris, L., Gray, E., Marshall, D. J., Crump, J. A., Kapiga, S. H., Sam, N. E., et al. (2011). Analysis of a clonal lineage of HIV-1 envelope V2/V3 conformational epitope-specific broadly neutralizing antibodies and their inferred unmutated common ancestors. Journal of virology 85, 9998-10009.
Brabant, M., Gong, E. Y., Plantier, J. C., Moreau, T., Alessandri, E., Simon, F., and Barin, F. (2013). Cross-group neutralization of HIV-1 and evidence for conservation of the PG9/PG16 epitopes within divergent groups. Aids 27, 1239-1244.
Briney, B., Sok, D., Jardine, J. G., Kulp, D. W., Skog, P., Menis, S., Jacak, R., Kalyuzhniy, O., de Val, N., Sesterhenn, F., et al. (2016). Tailored Immunogens Direct Affinity Maturation toward HIV Neutralizing Antibodies. Cell 166, 1459-1470 e1411.
Briney, B. S., Willis, J. R., and Crowe, J. E., Jr. (2012). Human peripheral blood antibodies with long HCDR3s are established primarily at original recombination using a limited subset of germline genes. PloS one 7, e36750.
Brochet, X., Lefranc, M. P., and Giudicelli, V. (2008). IMGT/V-QUEST: the highly customized and integrated system for IG and TR standardized V-J and V-D-J sequence analysis. Nucleic acids research 36, W503-508.
Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr D Biol Crystallogr 66, 12-21.
DiMaio, F., Song, Y., Li, X., Brunner, M. J., Xu, C., Conticello, V., Egelman, E., Marlovits, T., Cheng, Y., and Baker, D. (2015). Atomic-accuracy models from 4.5-A cryo-electron microscopy data with density-guided iterative local refinement. Nature methods 12, 361-365.
Doria-Rose, N. A., Schramm, C. A., Gorman, J., Moore, P. L., Bhiman, J. N., DeKosky, B. J., Ernandes, M. J., Georgiev, I. S., Kim, H. J., Pancera, M., et al. (2014). Developmental pathway for potent V1V2-directed HIV-neutralizing antibodies. Nature 509, 55-62.
Dosenovic, P., von Boehmer, L., Escolano, A., Jardine, J., Freund, N. T., Gitlin, A. D., McGuire, A. T., Kulp, D. W., Oliveira, T., Scharf, L., et al. (2015). Immunization for HIV-1 Broadly Neutralizing Antibodies in Human Ig Knockin Mice. Cell 161, 1505-1515.
Emsley, P., and Cowtan, K. (2004). Coot: model-building tools for molecular graphics. Acta Crystallogr D Biol Crystallogr 60, 2126-2132.
Escolano, A., Dosenovic, P., and Nussenzweig, M. C. (2017). Progress toward active or passive HIV-1 vaccination. The Journal of experimental medicine 214, 3-16.
Escolano, A., Steichen, J. M., Dosenovic, P., Kulp, D. W., Golijanin, J., Sok, D., Freund, N. T., Gitlin, A. D., Oliveira, T., Araki, T., et al. (2016). Sequential Immunization Elicits Broadly Neutralizing Anti-HIV-1 Antibodies in Ig Knockin Mice. Cell 166, 1445-1458 e1412.
Falkowska, E., Le, K. M., Ramos, A., Doores, K. J., Lee, J. H., Blattner, C., Ramirez, A., Derking, R., van Gils, M. J., Liang, C. H., et al. (2014). Broadly neutralizing HIV antibodies define a glycan-dependent epitope on the prefusion conformation of gp41 on cleaved envelope trimers. Immunity 40, 657-668.
Gao, F., Bailes, E., Robertson, D. L., Chen, Y., Rodenburg, C. M., Michael, S. F., Cummins, L. B., Arthur, L. O., Peeters, M., Shaw, G. M., et al. (1999). Origin of HIV-1 in the chimpanzee Pan troglodytes troglodytes. Nature 397, 436-441.
Garces, F., Lee, J. H., de Val, N., de la Pena, A. T., Kong, L., Puchades, C., Hua, Y., Stanfield, R. L., Burton, D. R., Moore, J. P., et al. (2015). Affinity Maturation of a Potent Family of HIV Antibodies Is Primarily Focused on Accommodating or Avoiding Glycans. Immunity 43, 1053-1063.
Garces, F., Sok, D., Kong, L., McBride, R., Kim, H. J., Saye-Francisco, K. F., Julien, J. P., Hua, Y., Cupo, A., Moore, J. P., et al. (2014). Structural evolution of glycan recognition by a family of potent HIV antibodies. Cell 159, 69-79.
Georgiev, I. S., Doria-Rose, N. A., Zhou, T., Kwon, Y. D., Staupe, R. P., Moquin, S., Chuang, G. Y., Louder, M. K., Schmidt, S. D., Altae-Tran, H. R., et al. (2013). Delineating antibody recognition in polyclonal sera from patterns of HIV-1 isolate neutralization. Science 340, 751-756.
Gorman, J., Soto, C., Yang, M. M., Davenport, T. M., Guttman, M., Bailer, R. T., Chambers, M., Chuang, G. Y., DeKosky, B. J., Doria-Rose, N. A., et al. (2016). Structures of HIV-1 Env V1V2 with broadly neutralizing antibodies reveal commonalities that enable vaccine design. Nat Struct Mol Biol 23, 81-90.
Harvey, D. J., Baruah, K., and Scanlan, C. N. (2009). Application of negative ion MS/MS to the identification of N-glycans released from carcinoembryonic antigen cell adhesion molecule 1 (CEACAMI). J Mass Spectrom 44, 50-60.
Havenar-Daughton, C., Lee, J. H., and Crotty, S. (2017). Tfh cells and HIV bnAbs, an immunodominance model of the HIV neutralizing antibody generation problem. Immunological reviews 275, 49-61.
Haynes, B. F., Kelsoe, G., Harrison, S. C., and Kepler, T. B. (2012). B-cell-lineage immunogen design in vaccine development with HIV-1 as a case study. Nature biotechnology 30, 423-433.
Haynes, B. F., and Mascola, J. R. (2017). The quest for an antibody-based HIV vaccine. Immunological reviews 275, 5-10.
Jardine, J., Julien, J. P., Menis, S., Ota, T., Kalyuzhniy, O., McGuire, A., Sok, D., Huang, P. S., MacPherson, S., Jones, M., et al. (2013). Rational HIV immunogen design to target specific germline B cell receptors. Science 340, 711-716.
Jardine, J. G., Ota, T., Sok, D., Pauthner, M., Kulp, D. W., Kalyuzhniy, O., Skog, P. D., Thinnes, T. C., Bhullar, D., Briney, B., et al. (2015). HIV-1 VACCINES. Priming a broadly neutralizing antibody response to HIV-1 using a germline-targeting immunogen. Science 349, 156-161.
Julien, J. P., Cupo, A., Sok, D., Stanfield, R. L., Lyumkis, D., Deller, M. C., Klasse, P. J., Burton, D. R., Sanders, R. W., Moore, J. P., et al. (2013a). Crystal structure of a soluble cleaved HIV-1 envelope trimer. Science 342, 1477-1483.
Julien, J. P., Lee, J. H., Cupo, A., Murin, C. D., Derking, R., Hoffenberg, S., Caulfield, M. J., King, C. R., Marozsan, A. J., Klasse, P. J., et al. (2013b). Asymmetric recognition of the HIV-1 trimer by broadly neutralizing antibody PG9. Proceedings of the National Academy of Sciences of the United States of America 110, 4351-4356.
Kepler, T. B., Liao, H. X., Alam, S. M., Bhaskarabhatla, R., Zhang, R., Yandava, C., Stewart, S., Anasti, K., Kelsoe, G., Parks, R., et al. (2014). Immunoglobulin gene insertions and deletions in the affinity maturation of HIV-1 broadly reactive neutralizing antibodies. Cell Host Microbe 16, 304-313.
Klein, F., Diskin, R., Scheid, J. F., Gaebler, C., Mouquet, H., Georgiev, I. S., Pancera, M., Zhou, T., Incesu, R. B., Fu, B. Z., et al. (2013). Somatic mutations of the immunoglobulin framework are generally required for broad and potent HIV-1 neutralization. Cell 153, 126-138.
Korber, B., Muldoon, M., Theiler, J., Gao, F., Gupta, R., Lapedes, A., Hahn, B. H., Wolinsky, S., and Bhattacharya, T. (2000). Timing the ancestor of the HIV-1 pandemic strains. Science 288, 1789-1796.
Kwon, Y. D., Pancera, M., Acharya, P., Georgiev, I. S., Crooks, E. T., Gorman, J., Joyce, M. G., Guttman, M., Ma, X., Narpala, S., et al. (2015). Crystal structure, conformational fixation and entry-related interactions of mature ligand-free HIV-1 Env. Nature structural & molecular biology 22, 522-531.
Landais, E., Huang, X., Havenar-Daughton, C., Murrell, B., Price, M. A., Wickramasinghe, L., Ramos, A., Bian, C. B., Simek, M., Allen, S., et al. (2016). Broadly Neutralizing Antibody Responses in a Large Longitudinal Sub-Saharan HIV Primary Infection Cohort. PLOS pathogens 12, e1005369.
Landais, E., Murrell, B., Briney, B., Murrell, S., Rantalainen, K., Berndsen, Z. T., Ramos, A., Wickramasinghe, L., Smith, M. L., Eren, K., et al. (2017). HIV Envelope Glycoform Heterogeneity and Localized Diversity Govern the Initiation and Maturation of a V2 Apex Broadly Neutralizing Antibody Lineage. Immunity 47, 990-1003 e1009.
Lee, J. H., Andrabi, R., Su, C. Y., Yasmeen, A., Julien, J. P., Kong, L., Wu, N.C., McBride, R., Sok, D., Pauthner, M., et al. (2017). A Broadly Neutralizing Antibody Targets the Dynamic HIV Envelope Trimer Apex via a Long, Rigidified, and Anionic beta-Hairpin Structure. Immunity 46, 690-702.
Lee, J. H., de Val, N., Lyumkis, D., and Ward, A. B. (2015). Model Building and Refinement of a Natively Glycosylated HIV-1 Env Protein by High-Resolution Cryoelectron Microscopy. Structure 23, 1943-1951.
Lee, J. H., Ozorowski, G., and Ward, A. B. (2016). Cryo-EM structure of a native, fully glycosylated, cleaved HIV-1 envelope trimer. Science 351, 1043-1048.
Lyumkis, D., Julien, J. P., de Val, N., Cupo, A., Potter, C. S., Klasse, P. J., Burton, D. R., Sanders, R. W., Moore, J. P., Carragher, B., et al. (2013). Cryo-EM structure of a fully glycosylated soluble cleaved HIV-1 envelope trimer. Science 342, 1484-1490.
McCoy, L. E., and Burton, D. R. (2017). Identification and specificity of broadly neutralizing antibodies against HIV. Immunological reviews 275, 11-20.
McGuire, A. T., Gray, M. D., Dosenovic, P., Gitlin, A. D., Freund, N. T., Petersen, J., Correnti, C., Johnsen, W., Kegel, R., Stuart, A. B., et al. (2016). Specifically modified Env immunogens activate B-cell precursors of broadly neutralizing HIV-1 antibodies in transgenic mice. Nature communications 7, 10618.
McGuire, A. T., Hoot, S., Dreyer, A. M., Lippy, A., Stuart, A., Cohen, K. W., Jardine, J., Menis, S., Scheid, J. F., West, A. P., et al. (2013). Engineering HIV envelope protein to activate germline B cell receptors of broadly neutralizing anti-CD4 binding site antibodies. The Journal of experimental medicine 210, 655-663.
Mclellan, J. S., Pancera, M., Carrico, C., Gorman, J., Julien, J. P., Khayat, R., Louder, R., Pejchal, R., Sastry, M., Dai, K., et al. (2011). Structure of HIV-1 gp120 V1/V2 domain with broadly neutralizing antibody PG9. Nature 480, 336-343.
Montefiori, D. C. (2005). Evaluating neutralizing antibodies against HIV, SIV, and SHIV in luciferase reporter gene assays. Current protocols in immunology/edited by John E Coligan [et al] Chapter 12, Unit 12.11.
Moore, P. L., Gray, E. S., Sheward, D., Madiga, M., Ranchobe, N., Lai, Z., Honnen, W. J., Nonyane, M., Tumba, N., Hermanus, T., et al. (2011). Potent and broad neutralization of HIV-1 subtype C by plasma antibodies targeting a quaternary epitope including residues in the V2 loop. Journal of virology 85, 3128-3141.
Morgand, M., Bouvin-Pley, M., Plantier, J. C., Moreau, A., Alessandri, E., Simon, F., Pace, C. S., Pancera, M., Ho, D. D., Poignard, P., et al. (2016). V1/V2 Neutralizing Epitope is Conserved in Divergent Non-M Groups of HIV-1. J Acquir Immune Defic Syndr 71, 237-245.
Ogura, T., Iwasaki, K., and Sato, C. (2003). Topology representing network enables highly accurate classification of protein images taken by cryo electron-microscope without masking. J Struct Biol 143, 185-200.
Ozorowski, G., Pallesen, J., de Val, N., Lyumkis, D., Cottrell, C. A., Torres, J. L., Copps, J., Stanfield, R. L., Cupo, A., Pugach, P., et al. (2017). Open and closed structures reveal allostery and pliability in the HIV-1 envelope spike. Nature 547, 360-363.
Pancera, M., Shahzad-Ul-Hussan, S., Doria-Rose, N. A., Mclellan, J. S., Bailer, R. T., Dai, K., Loesgen, S., Louder, M. K., Staupe, R. P., Yang, Y., et al. (2013). Structural basis for diverse N-glycan recognition by HIV-1-neutralizing V1-V2-directed antibody PG16. Nature structural & molecular biology 20, 804-813.
Pancera, M., Zhou, T., Druz, A., Georgiev, I. S., Soto, C., Gorman, J., Huang, J., Acharya, P., Chuang, G. Y., Ofek, G., et al. (2014). Structure and immune recognition of trimeric pre-fusion HIV-1 Env. Nature 514, 455-461.
Panico, M., Bouche, L., Binet, D., O'Connor, M. J., Rahman, D., Pang, P. C., Canis, K., North, S. J., Desrosiers, R. C., Chertova, E., et al. (2016). Mapping the complete glycoproteome of virion-derived HIV-1 gp120 provides insights into broadly neutralizing antibody binding. Sci Rep 6, 32956.
Pejchal, R., Doores, K. J., Walker, L. M., Khayat, R., Huang, P. S., Wang, S. K., Stanfield, R. L., Julien, J. P., Ramos, A., Crispin, M., et al. (2011). A potent and broad neutralizing antibody recognizes and penetrates the HIV glycan shield. Science 334, 1097-1103.
Pritchard, L. K., Harvey, D. J., Bonomelli, C., Crispin, M., and Doores, K. J. (2015). Cell- and Protein-Directed Glycosylation of Native Cleaved HIV-1 Envelope. Journal of virology 89, 8932-8944.
Pugach, P., Ozorowski, G., Cupo, A., Ringe, R., Yasmeen, A., de Val, N., Derking, R., Kim, H. J., Korzun, J., Golabek, M., et al. (2015). A native-like SOSIP.664 trimer based on a HIV-1 subtype B env gene. Journal of virology.
Riedel, S. (2005). Edward Jenner and the history of smallpox and vaccination. Proc (Bayl Univ Med Cent) 18, 21-25.
Sanders, R. W., Derking, R., Cupo, A., Julien, J. P., Yasmeen, A., de Val, N., Kim, H. J., Blattner, C., de la Pena, A. T., Korzun, J., et al. (2013). A next-generation cleaved, soluble HIV-1 Env trimer, BG505 SOSIP.664 gp140, expresses multiple epitopes for broadly neutralizing but not non-neutralizing antibodies. PLOS pathogens 9, e1003618.
Sanders, R. W., van Gils, M. J., Derking, R., Sok, D., Ketas, T. J., Burger, J. A., Ozorowski, G., Cupo, A., Simonich, C., Goo, L., et al. (2015). HIV-1 VACCINES. HIV-1 neutralizing antibodies induced by native-like envelope trimers. Science 349, aac4223.
Saunders, K. O., Verkoczy, L. K., Jiang, C., Zhang, J., Parks, R., Chen, H., Housman, M., Bouton-Verville, H., Shen, X., Trama, A. M., et al. (2017). Vaccine Induction of Heterologous Tier 2 HIV-1 Neutralizing Antibodies in Animal Models. Cell reports 21, 3681-3690.
Scheres, S. H. (2012). RELION: implementation of a Bayesian approach to cryo-EM structure determination. J Struct Biol 180, 519-530.
Seaman, M. S., Janes, H., Hawkins, N., Grandpre, L. E., Devoy, C., Giri, A., Coffey, R. T., Harris, L., Wood, B., Daniels, M. G., et al. (2010). Tiered categorization of a diverse panel of HIV-1 Env pseudoviruses for assessment of neutralizing antibodies. Journal of virology 84, 1439-1452.
Sharma, S. K., de Val, N., Bale, S., Guenaga, J., Tran, K., Feng, Y., Dubrovskaya, V., Ward, A. B., and Wyatt, R. T. (2015). Cleavage-independent HIV-1 Env trimers engineered as soluble native spike mimetics for vaccine design. Cell reports 11, 539-550.
Sharp, P. M., and Hahn, B. H. (2011). Origins of HIV and the AIDS pandemic. Cold Spring Harbor perspectives in medicine 1, a006841.
Sok, D., Briney, B., Jardine, J. G., Kulp, D. W., Menis, S., Pauthner, M., Wood, A., Lee, E. C., Le, K. M., Jones, M., et al. (2016a). Priming HIV-1 broadly neutralizing antibody precursors in human Ig loci transgenic mice. Science.
Sok, D., Doores, K. J., Briney, B., Le, K. M., Saye-Francisco, K. L., Ramos, A., Kulp, D. W., Julien, J. P., Menis, S., Wickramasinghe, L., et al. (2014a). Promiscuous glycan site recognition by antibodies to the high-mannose patch of gp120 broadens neutralization of HIV. Science translational medicine 6, 236ra263.
Sok, D., Pauthner, M., Briney, B., Lee, J. H., Saye-Francisco, K. L., Hsueh, J., Ramos, A., Le, K. M., Jones, M., Jardine, J. G., et al. (2016b). A Prominent Site of Antibody Vulnerability on HIV Envelope Incorporates a Motif Associated with CCR5 Binding and Its Camouflaging Glycans. Immunity 45, 31-45.
Sok, D., van Gils, M. J., Pauthner, M., Julien, J. P., Saye-Francisco, K. L., Hsueh, J., Briney, B., Lee, J. H., Le, K. M., Lee, P. S., et al. (2014b). Recombinant HIV envelope trimer selects for quaternary-dependent antibodies targeting the trimer apex. Proceedings of the National Academy of Sciences of the United States of America 111, 17624-17629.
Steichen, J. M., Kulp, D. W., Tokatlian, T., Escolano, A., Dosenovic, P., Stanfield, R. L., McCoy, L. E., Ozorowski, G., Hu, X., Kalyuzhniy, O., et al. (2016a). HIV Vaccine Design to Target Germline Precursors of Glycan-Dependent Broadly Neutralizing Antibodies. Immunity 45, 483-496.
Steichen, J. M., Kulp, D. W., Tokatlian, T., Escolano, A., Dosenovic, P., Stanfield, R. L., McCoy, L. E., Ozorowski, G., Hu, X., Kalyuzhniy, O., et al. (2016b). HIV Vaccine Design to Target Germline Precursors of Glycan-Dependent Broadly Neutralizing Antibodies. Immunity 45, 483-496.
Tas, J. M., Mesin, L., Pasqual, G., Targ, S., Jacobsen, J. T., Mano, Y. M., Chen, C. S., Weill, J. C., Reynaud, C. A., Browne, E. P., et al. (2016). Visualizing antibody affinity maturation in germinal centers. Science 351, 1048-1054.
Tian, M., Cheng, C., Chen, X., Duan, H., Cheng, H. L., Dao, M., Sheng, Z., Kimble, M., Wang, L., Lin, S., et al. (2016). Induction of HIV Neutralizing Antibody Lineages in Mice with Diverse Precursor Repertoires. Cell 166, 1471-1484 e1418.
Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. Journal of immunological methods 329, 112-124.
Verkoczy, L., Diaz, M., Holl, T. M., Ouyang, Y. B., Bouton-Verville, H., Alam, S. M., Liao, H. X., Kelsoe, G., and Haynes, B. F. (2010). Autoreactivity in an HIV-1 broadly reactive neutralizing antibody variable region heavy chain induces immunologic tolerance. Proceedings of the National Academy of Sciences of the United States of America 107, 181-186.
Voss, J. E., Andrabi, R., McCoy, L. E., de Val, N., Fuller, R. P., Messmer, T., Su, C. Y., Sok, D., Khan, S. N., Garces, F., et al. (2017). Elicitation of Neutralizing Antibodies Targeting the V2 Apex of the HIV Envelope Trimer in a Wild-Type Animal Model. Cell reports 21, 222-235.
Voss, N. R., Yoshioka, C. K., Radermacher, M., Potter, C. S., and Carragher, B. (2009). DoG Picker and TiltPicker: software tools to facilitate particle selection in single particle electron microscopy. J Struct Biol 166, 205-213.
Walker, L. M., Huber, M., Doores, K. J., Falkowska, E., Pejchal, R., Julien, J. P., Wang, S. K., Ramos, A., Chan-Hui, P. Y., Moyle, M., et al. (2011). Broad neutralization coverage of HIV by multiple highly potent antibodies. Nature 477, 466-470.
Walker, L. M., Phogat, S. K., Chan-Hui, P. Y., Wagner, D., Phung, P., Goss, J. L., Wrin, T., Simek, M. D., Fling, S., Mitcham, J. L., et al. (2009). Broad and potent neutralizing antibodies from an African donor reveal a new HIV-1 vaccine target. Science 326, 285-289.
Wang, S., Mata-Fink, J., Kriegsman, B., Hanson, M., Irvine, D. J., Eisen, H. N., Burton, D. R., Wittrup, K. D., Kardar, M., and Chakraborty, A. K. (2015). Manipulating the Selection Forces during Affinity Maturation to Generate Cross-Reactive HIV Antibodies. Cell 160, 785-797.
Ward, A. B., and Wilson, I. A. (2017). The HIV-1 envelope glycoprotein structure: nailing down a moving target. Immunological reviews 275, 21-32.
Webb, B., and Sali, A. (2016). Comparative Protein Structure Modeling Using MODELLER. Curr Protoc Bioinformatics 54, 5 6 1-5 6 37.
Wibmer, C. K., Bhiman, J. N., Gray, E. S., Tumba, N., Abdool Karim, S. S., Williamson, C., Morris, L., and Moore, P. L. (2013). Viral escape from HIV-1 neutralizing antibodies drives increased plasma neutralization breadth through sequential recognition of multiple epitopes and immunotypes. PLOS pathogens 9, e1003738.
Williams, W. B., Zhang, J., Jiang, C., Nicely, N. I., Fera, D., Luo, K., Moody, M. A., Liao, H. X., Alam, S. M., Kepler, T. B., et al. (2017). Initiation of HIV neutralizing B cell lineages with sequential envelope immunizations. Nature communications 8, 1732.
Worobey, M., Gemmel, M., Teuwen, D. E., Haselkorn, T., Kunstman, K., Bunce, M., Muyembe, J. J., Kabongo, J. M., Kalengayi, R. M., Van Marck, E., et al. (2008). Direct evidence of extensive diversity of HIV-1 in Kinshasa by 1960. Nature 455, 661-664.
Wu, X., Yang, Z. Y., Li, Y., Hogerkorp, C. M., Schief, W. R., Seaman, M. S., Zhou, T., Schmidt, S. D., Wu, L., Xu, L., et al. (2010). Rational design of envelope identifies broadly neutralizing human monoclonal antibodies to HIV-1. Science 329, 856-861.
Xiao, X., Chen, W., Feng, Y., Zhu, Z., Prabakaran, P., Wang, Y., Zhang, M. Y., Longo, N. S., and Dimitrov, D. S. (2009). Germline-like predecessors of broadly neutralizing antibodies lack measurable binding to HIV-1 envelope glycoproteins: implications for evasion of immune responses and design of vaccine immunogens. Biochemical and biophysical research communications 390, 404-409.
Zhang, K. (2016). Gctf: Real-time CTF determination and correction. J Struct Biol 193, 1-12.
Zhang, M., Gaschen, B., Blay, W., Foley, B., Haigwood, N., Kuiken, C., and Korber, B. (2004). Tracking global patterns of N-linked glycosylation site variation in highly variable viral glycoproteins: HIV, SIV, and HCV envelopes and influenza hemagglutinin. Glycobiology 14, 1229-1246.
Zheng, S. Q., Palovcak, E., Armache, J. P., Verba, K. A., Cheng, Y., and Agard, D. A. (2017). MotionCor2: anisotropic correction of beam-induced motion for improved cryo-electron microscopy. Nature methods 14, 331-332.
Zhou, T., Georgiev, I., Wu, X., Yang, Z. Y., Dai, K., Finzi, A., Kwon, Y. D., Scheid, J. F., Shi, W., Xu, L., et al. (2010). Structural basis for broad and potent neutralization of HIV-1 by antibody VRC01. Science 329, 811-817.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An engineered Simian Immunodeficiency Virus (SIV) envelope (Env) glycoprotein trimer that binds to a germline or reverted Human Immunodeficiency Virus (HIV) broadly neutralizing antibody (bnAb) directed to a V2 apex region of an HIV envelope glycoprotein.

2. The engineered SIV Env glycoprotein trimer of claim 1, wherein the trimer comprises a substitution of an amino acid at position 171 with Lys (K).

3. The engineered SIV Env glycoprotein trimer of claim 1, wherein the trimer comprises a deletion of the amino acids at positions 460-468 within the V5 loop, wherein the positions are numbered according to SEQ ID NO: 28.

4. The engineered SIV Env glycoprotein of claim 1, wherein the HIV bnAb comprises:
one or more complementarity determining regions (CDRs) of a heavy chain variable domain selected from SEQ ID Nos: 33-35 or SEQ ID NOs: 50-52, and/or one or more CDRs of a light chain variable domain selected from SEQ ID Nos: 42-44 or SEQ ID NOs: 59-61; or
an amino acid comprising a heavy chain having at least 95% sequence identity to the amino acid sequence of SEQ ID NO: 30 and a light chain having at least 95% sequence identity to the amino acid sequence of SEQ ID NO: 39, or an antibody comprising a heavy chain having at least 95% sequence identity to the amino acid sequence of SEQ ID NO: 47 and a light chain having at least 95% sequence identity to the amino acid sequence of SEQ ID NO: 56.

5. (canceled)

6. The engineered SIV Env glycoprotein of claim 1, wherein the trimer comprises a complex of gp120 and gp41, or variants thereof.

7. The engineered SIV Env glycoprotein trimer of any claim 1, wherein the trimer is a SOSIP, NFL, or UFO stabilized trimer.

8-9. (canceled)

10. The engineered SIV Env glycoprotein trimer of claim 1, wherein the SIV is SIVcpzPtt, SIVcpzPts, or SIVgor.

11. The engineered SIV Env glycoprotein trimer of claim 10, wherein the Env glycoprotein sequence is derived from the SIVcpzPtt isolate MT145.

12. The engineered SIV Env glycoprotein trimer of claim 1, wherein the trimer comprises:
(a) a substitution of an amino acid at position 171 with lysine; and
(b) a deletion of a plurality of amino acids within a V5 loop.

13-16. (canceled)

17. The engineered SIV Env glycoprotein trimer of claim 1, wherein the V2 apex region of the Env glycoprotein sequence comprises N-C-X₁-F-N-X₂-X₃-X₄-X₅-X₆-X₇-X₈-X₉-X₁₀-X₁₁-X₁₂-X₁₃-X₁₄-X₁₅-X₁₆, wherein;
X₁ comprises S, T, N, or F;
X₂ comprises I, V, T, Q, of M;
X₃ comprises S or T;
X₄ comprises S or T;
X₅ comprises S, E, or G;
X₆ comprises I, L, or V;
X₇ comprises K or R;
X₈ comprises G or D;
X₉ comprises K, R, E, or Q;
X₁₀ comprises K, R, E, or Q;
X₁₁ comprises K, R, E, or Q;
X₁₂ comprises K, E, or Q;
X₁₃ comprises E, T, V, I, or M;
X₁₄ comprises Y or K;
X₁₅ comprises A or S; and
X₁₆ comprises F, I, or L.

18-20. (canceled)

21. The engineered SIV Env glycoprotein trimer of claim 1, wherein the V2 apex region of the Env sequence glycoprotein comprises:
Asn-Cys-Ser-Phe-Asn-Val-Thr-Thr-Glu-Leu-Arg-Asp-
Lys-Lys-Arg-Gln-Val-Tyr-Ser-Leu-Phe-Tyr;
Asn-Cys-Ser-Phe-Asn-Val-Thr-Thr-Glu-Leu-Arg-Asp-
Lys-Lys-Arg-Lys-Val-Tyr-Ser-Leu-Phe-Tyr;
Asn-Cys-Ser-Phe-Asn-Val-Thr-Thr-Glu-Leu-Arg-Asp-
Lys-Lys-Lys-Lys-Glu-Tyr-Ser--Phe-Phe-Tyr;
or
Asn-Cys-Ser-Phe-Asn-Ala-Thr-Thr-Glu-Leu-Arg-Asp-
Lys-Lys-Lys-Lys-Glu-Tyr-Ala-Leu-Phe-Tyr.

22. The engineered SIV Env glycoprotein trimer of claim 1, wherein the Env glycoprotein sequence comprises the amino acid sequence Asn-Xxxₐ-Asn-Xxx_{b}-Xxxc-Xxx_{d}, wherein:
Xxxₐ comprises two or three amino acids,
Xxx_{b} comprises five to seven amino acids,
Xxx_{c} comprises four amino acids, each of the four amino acids selected from Lys or Arg, and
Xxx_{d} comprises any amino acid.

23-26. (canceled)

27. A nucleic acid encoding the engineered SIV Env glycoprotein trimer claim 1.

28. A vector comprising a regulatory element operable in a eukaryotic cell operably linked to the nucleic acid of claim 27.

29-30. (canceled)

31. A method of eliciting an immune response in a subject comprising administering an immunologically effective amount of the engineered SIV Env glycoprotein trimer of claim 1.

32-33. (canceled)

34. The method of claim 31, wherein the subject is a human.

35-41. (canceled)

42. The method of claim 31, further comprising administering one or more HIV Env glycoprotein trimers in a sequential prime-boost immunization regimen.

43. (canceled)

44. The method of claim 31, wherein the molecule is administered with an adjuvant.

45-46. (canceled)

47. The method of claim 44, wherein the adjuvant is ISCOMATRIX, Adjuplex, or alum.

48. (canceled)

49. The method of claim 31, wherein the trimer is administered in a liposome or in a nanoparticle.

50-61. (canceled)


SEQUENCE LISTING (359 sequences)

SEQ ID NO: 1-4 - skipped (4 entries)

SEQ ID NO: 5 - protein, 4 aa, synthetic construct
  REGION 1..4: note = Synthetic
KKKK

SEQ ID NO: 6 - skipped

SEQ ID NO: 7 - protein, 20 aa, synthetic construct
  REGION 1..20: note = Synthetic
  VARIANT 3: note = Xaa comprises S, T, N, or F.
  VARIANT 6: note = Xaa comprises I, V, T, Q, or M.
  VARIANT 7: note = Xaa comprises S or T.
  VARIANT 8: note = Xaa comprises S or T.
  VARIANT 9: note = Xaa comprises S, E, or G.
  VARIANT 10: note = Xaa comprises I, L, V, or F.
  VARIANT 11: note = Xaa comprises K or R.
  VARIANT 12: note = Xaa comprises G or D.
  VARIANT 13: note = Xaa comprises K, R, E, or Q.
  VARIANT 14: note = Xaa comprises K, R, E, or Q.
  VARIANT 15: note = Xaa comprises K, R, E, or Q.
  VARIANT 16: note = Xaa comprises K, E, or Q.
  VARIANT 17: note = Xaa comprises E, T, V, I, or M.
  VARIANT 18: note = Xaa comprises Y or K.
  VARIANT 19: note = Xaa comprises A or S.
  VARIANT 20: note = Xaa comprises F, I, or L.
NCXFNXXXXXXXXXXXXXXX

SEQ ID NO: 8 - protein, 20 aa, synthetic construct
  REGION 1..20: note = Synthetic
  VARIANT 3: note = Xaa comprises S, T, N, or F.
  VARIANT 6: note = Xaa comprises I, V, T, Q, or M.
  VARIANT 7: note = Xaa comprises S or T.
  VARIANT 8: note = Xaa comprises S or T.
  VARIANT 9: note = Xaa comprises S, E, or G.
  VARIANT 10: note = Xaa comprises I, L, V, or F.
  VARIANT 11: note = Xaa comprises K or R.
  VARIANT 12: note = Xaa comprises G or D.
  VARIANT 13: note = Xaa comprises K, R, E, or Q.
  REGION 13..16: note = At least three of four Xaa comprise basic amino acids.
  VARIANT 14: note = Xaa comprises K, R, E, or Q.
  VARIANT 15: note = Xaa comprises K, R, E, or Q.
  VARIANT 16: note = Xaa comprises K, E, or Q.
  VARIANT 17: note = Xaa comprises E, T, V, I, or M.
  VARIANT 18: note = Xaa comprises Y or K.
  VARIANT 19: note = Xaa comprises A or S.
  VARIANT 20: note = Xaa comprises F, I, or L.
NCXFNXXXXXXXXXXXXXXX

SEQ ID NO: 9 - protein, 20 aa, synthetic construct
  REGION 1..20: note = Synthetic
  VARIANT 3: note = Xaa comprises S, T, N, or F.
  VARIANT 6: note = Xaa comprises I, V, T, Q, or M.
  VARIANT 7: note = Xaa comprises S or T.
  VARIANT 8: note = Xaa comprises S or T.
  VARIANT 9: note = Xaa comprises S, E, or G.
  VARIANT 10: note = Xaa comprises I, L, V, or F.
  VARIANT 11: note = Xaa comprises K or R.
  VARIANT 12: note = Xaa comprises G or D.
  VARIANT 13: note = Xaa comprises K or R.
  VARIANT 14: note = Xaa comprises K or R.
  VARIANT 15: note = Xaa comprises K or R.
  VARIANT 16: note = Xaa comprises K or R.
  VARIANT 17: note = Xaa comprises E, T, V, I, or M.
  VARIANT 18: note = Xaa comprises Y or K.
  VARIANT 19: note = Xaa comprises A or S.
  VARIANT 20: note = Xaa comprises F, I, or L.
NCXFNXXXXXXXXXXXXXXX

SEQ ID NO: 10 - protein, 20 aa, synthetic construct
  REGION 1..20: note = Synthetic
  VARIANT 3: note = Xaa comprises S, T, N, or F.
  VARIANT 6: note = Xaa comprises I, V, T, Q, or M.
  VARIANT 7: note = Xaa comprises S or T.
  VARIANT 8: note = Xaa comprises S or T.
  VARIANT 9: note = Xaa comprises S, E, or G.
  VARIANT 10: note = Xaa comprises I, L, V, or F.
  VARIANT 11: note = Xaa comprises K or R.
  VARIANT 12: note = Xaa comprises G or D.
  VARIANT 17: note = Xaa comprises E, T, V, I, or M.
  VARIANT 18: note = Xaa comprises Y or K.
  VARIANT 19: note = Xaa comprises A or S.
  VARIANT 20: note = Xaa comprises F, I, or L.
NCXFNXXXXXXXKKKKXXXX

SEQ ID NO: 11 - protein, 20 aa, synthetic construct
  REGION 1..20: note = Synthetic
NCSFNVTTELRDKKRQVYSL

SEQ ID NO: 12 - protein, 20 aa, synthetic construct
  REGION 1..20: note = Synthetic
NCSFNVTTELRDKKRKVYSL

SEQ ID NO: 13 - protein, 20 aa, synthetic construct
  REGION 1..20: note = Synthetic
NCSFNVTTELRDKKKKEYSF

SEQ ID NO: 14 - protein, 20 aa, synthetic construct
  REGION 1..20: note = Synthetic
NCSFNATTELRDKKKKEYAL

SEQ ID NO: 15 - protein, 23 aa, synthetic construct
  REGION 1..23: note = Synthetic
NCSFNVTTELRDKKRQVYSLFFY

SEQ ID NO: 16 - protein, 22 aa, synthetic construct
  REGION 1..22: note = Synthetic
NCSFNVTTELRDKKRQVYSLFY

SEQ ID NO: 17 - protein, 23 aa, synthetic construct
  REGION 1..23: note = Synthetic
NCSFNVTTELRDKKRKVYSLFFY

SEQ ID NO: 18 - protein, 22 aa, synthetic construct
  REGION 1..22: note = Synthetic
NCSFNVTTELRDKKRKVYSLFY

SEQ ID NO: 19 - protein, 23 aa, synthetic construct
  REGION 1..23: note = Synthetic
NCSFNVTTELRDKKKKEYSFFFY

SEQ ID NO: 20 - protein, 22 aa, synthetic construct
  REGION 1..22: note = Synthetic
NCSFNVTTELRDKKKKEYSFFY

SEQ ID NO: 21 - protein, 23 aa, synthetic construct
  REGION 1..23: note = Synthetic
NCSFNATTELRDKKKKEYALFFY

SEQ ID NO: 22 - protein, 22 aa, synthetic construct
  REGION 1..22: note = Synthetic
NCSFNATTELRDKKKKEYALFY

SEQ ID NO: 23 - protein, 22 aa, Simian immunodeficiency virus
NCSFNISTSIRGKVQKEYAFFY

SEQ ID NO: 24 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 25 - protein, 678 aa, Simian immunodeficiency virus
MDAMKRGLCCVLLLCGAVFVSPSQEIHARFRRGARSELWVTVYYGVPVWHDADPVLFCASDAKAHSTEAHNIWATQACVPTDPSPQEVFLPNVIESFNMWKNNMVDQMHEDIISLWDQSLKPCVKLTPLCVTLQCSKANFSQAKNLTNQTSSPPLEMKNCSFNVTTELRDKKKQVYSLFYVEDVVNLGNENNTYRIINCNTTAITQACPKTSFEPIPIHYCAPAGFAILKCNDKDFSGKGKCTNVSTVHCTHGIKPVVTTQLLINGSLAEGNITVRVENKSKNTDVWIVQLVEAVSLNCHRPGNNTGREVQIGPGMTFYNIENVVGDTRSAYCKINGTTWNRTVEEVKKALATSSNRTAANITLNRASGGDPEVTHHMFNCGGEFFYCNTSQIFTDNITNGIIILPCRIRQIVSSWMRVGRGIYAPPIRGNITCNSNITGLLLTSDTPVTNNSGNLTFRPTGGNMKDIWRSELYKYKVVRIEPLSVAPTKCRRHTVARQKDRRRRRRAAFGLGALFLGFLGAAGSTMGAAAVTLTVQARQLLSGIVQQQNNLLKAPEAQQHLLQLSIWGVKQLQARLLAVERYLQDQQILGLWGCSGKAVCCTTVPWNNSWPGSNSTDDIWGNLTWQQWDKLVSNYTGKIFGLLEEAQSQQEKNERDLLELDGSGSGGSGHHHHHHHH

SEQ ID NO: 26 - protein, 669 aa, Simian immunodeficiency virus
MDAMKRGLCCVLLLCGAVFVSPSQEIHARFRRGARSENNLWVTVYYGVPVWKEAKTTLFCASDAKAQIAEAHNIWASQACVPTDPNPEEITLENVTEEFDAWNNNMVDQMQEDLISLWDQSLKPCVKLTPLCVTLNCSTNTTTEASQVQYNCSFNVTTELRDKKKQVYSLFYREDITSLDSNKTVKNGTYRLINCNTTAITQACPKTSFEPIPIYYCAPAGFALLKCNDQNFKGKGTCRNVSTVHCTHGIKPVVSTQFLLNGSLAEGNKTVVRVRSKSNTETIIVQLATAIYINCTRLGNKTIEGIPIGPGQIFYRTKTVVGDTRGAECRINGTAWNETLRQVKEALNNTYRNLNLSLTEINFEGASGGDLEVTTHYFNCGGEFFYCNTSNLFNQSTINNITHIPCRIRQIVNQWQGVGKGIFAPPIRGTIQCNSTITGLLLTRDGKNETETFRPTGGDMRNNWRSELYKYKVVKIEPLGLAPTKCKRRTVQRRRRRRAVGLGVMFLGFMGAAGSTMGAAALTLTVQARNLLSGIVQQQSNLLKAPEAQQHLLQLSVWGIKQLQARVLAVERYLKDQQLLGLWGCSGKLICCTNVPWNNTWSKKLGTNLSFWDNMTWMQWEKEIDNYTETIYELLTRSQNQQEVNEQELLALDGSGSGGSGHHHHHHHH

SEQ ID NO: 27 - protein, 665 aa, Simian immunodeficiency virus
MDAMKRGLCCVLLLCGAVFVSPSQEIHARFRRGARSELWVTVYYGVPVWHDAETVLFCASDAKAHSTEAHNIWATQACVPTDPNPQEVLIPNVTERFDMWKNNMVDQMQEDIISLWEQSLKPCVKLTPLCVTLSCSSWRSVNNSVNQTNHVQMQNCSFNVTTELRDKKKQVYSLFYMGDIIPLDTNNSSGNNSQYRLINCNTTAVTQACPKISFEPIPIYYCAPPGFAIIKCNDQDFNGTGECNNVSTVQCTHGIKPVISTQLILNGSLATSNIVIRNNSKDTLLVQLNESIPINCTRPGNKTRGQVQIGPGMTFYNIENIIGDTRQAYCEVNRTWEQIWNTTKQIIINNRKNITFIPNPGGDLEVTNLMINCGGEFFYCNTSQLFTKQNGNTTGNITLQCRIRQIVNLWTRVGKGIYAPPIKGPINCLSNITGIILDYTKSGTEKYTIYPTGGDMTNLWRQELYKYKVVSIEPIGVAPGKCKRHTVTRRRRRRAAFGLGALFLGFLGAAGSTMGAASITLTVQARKLLSGIVQQQNNLLRAPEAQQHLLQLSVWGIKQLQARVLAIERYLRDQQILGLWGCSGKSVCCTNVPWNTTWSNNNSYDTIWGNMTWQNWDEQVRNYSGVIFGLLEQAQEQQSINEKSLLELDGSGSGGSGHHHHHHHH

SEQ ID NO: 28 - protein, 675 aa, Simian immunodeficiency virus
MDAMKRGLCCVLLLCGAVFVSPSQEIHARFRRGARSEWWVTVYYGVPVWREAKTTLFCASDAKSYSTEAHNIWATQACVPTDPTPQEVLLPNVTEEFNMWENYMVDQMQEDIISLWEQSLKPCVKLTPLCVTLTCNNPTNTSCTNSTDDRLGDRMNCSFNVTTELRDKKKQVYSLFYVEDITAIGNNSTYRLINCNTTAITQACPKTSFEPIPIHYCAPAGFALLKCNDIDYKGNETCKNVSTVHCTHGIKPVATTQLILNGSTADNQTVARIDPSENLAIIQLKDPVKITCRRPGNNTRGQIQIGPAMTFYNIENVVGDTRKAYCEINGTQWAKALNETKEVLRNILRKNISFMVPSGGDPEVTNHHFNCGGEFFYCNTSDIINITKINKTENMTIIPCRIRQIVNSWMRVGKGIFAPPIRGNITCTSNITGMLLEIHKNREDQGEDQDQNNTYVCLTGGNMKDIWRSELYKYKIVEIQPLGVAPTKCRRYAVEKQHHRRRRRRALGLGALFLGFLGAAGSTMGAASVVLTVQARQLLTGIVQQQNNLLRAPEAQQHLLQLSVWGIKQLQARVLAVERYLRDQQLLGLWGCTGKTICCTAVRWNKTWGNISDYQVIWNNYTWQQWDREVNNYTGLIYTLLEEANTQQEKNEKELLELDGSGSGGSGHHHHHHHH

SEQ ID NO: 29 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAR

SEQ ID NO: 30 - protein, 136 aa, Homo sapiens
QRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSS

SEQ ID NO: 31 - protein, 137 aa, unidentified
  REGION 1..137: note = synthetic sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFYDGYYNYHYMDVWGKGTTVTVSS

SEQ ID NO: 32 - protein, 137 aa, unidentified
  REGION 1..137: note = synthetic sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFWSGYYYYYYMDVWGKGTTVTVSS

SEQ ID NO: 33 - protein, 5 aa, Homo sapiens
SYGMH

SEQ ID NO: 34 - protein, 17 aa, Homo sapiens
VISYDGSNKYYADSVKG

SEQ ID NO: 35 - protein, 28 aa, Homo sapiens
EAGGPDYRNGYNYYDFWSGYYYYYYMDV

SEQ ID NO: 36 - protein, 20 aa, unidentified
  REGION 1..20: note = synthetic sequence
YYYYYYMDVWGKGTTVTVSS

SEQ ID NO: 37 - protein, 10 aa, unidentified
  REGION 1..10: note = synthetic sequence
YYDFWSGYYT

SEQ ID NO: 38 - protein, 99 aa, Human immunodeficiency virus 1
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 39 - protein, 110 aa, unidentified
  REGION 1..110: note = synthetic sequence
QSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVL

SEQ ID NO: 40 - protein, 110 aa, unidentified
  REGION 1..110: note = synthetic sequence
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLVFGTGTKLTVL

SEQ ID NO: 41 - protein, 110 aa, unidentified
  REGION 1..110: note = synthetic sequence
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTWVFGGGTKLTVL

SEQ ID NO: 42 - protein, 14 aa, Homo sapiens
TGTSSDVGGYNYVS

SEQ ID NO: 43 - protein, 7 aa, Homo sapiens
EVSNRPS

SEQ ID NO: 44 - protein, 10 aa, Homo sapiens
SSYTSSSTWV

SEQ ID NO: 45 - protein, 12 aa, unidentified
  REGION 1..12: note = synthetic sequence
WVFGGGTKLTVL

SEQ ID NO: 46 - protein, 98 aa, Human immunodeficiency virus 1
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 47 - protein, 133 aa, Human immunodeficiency virus 1
EVQLVESGANVVRPGGSLRLSCKASGFIFENFGFSWVRQAPGKGLQWVAGLNWNGGDTRYADSVKGRFRMSRDNSRNFVYLDMDKVGVDDTAFYYCARGTDYTIDDAGIHYQGSGTFWYFDLWGRGTLVSVSS

SEQ ID NO: 48 - protein, 133 aa, unidentified
  REGION 1..133: note = synthetic sequence
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARGTDYTIDDAGIHYQGSGTFWYFDLWGRGTLVSVSS

SEQ ID NO: 49 - protein, 133 aa, unidentified
  REGION 1..133: note = synthetic sequence
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARGTDYTIDDAGIHYYGSGSYWYFDLWGRGTLVTVSS

SEQ ID NO: 50 - protein, 5 aa, Homo sapiens
DYGMS

SEQ ID NO: 51 - protein, 17 aa, Homo sapiens
GINWNGGSTGYADSVKG

SEQ ID NO: 52 - protein, 24 aa, Homo sapiens
GTDYTIDDAGIHYYGSGSYWYFDL

SEQ ID NO: 53 - protein, 17 aa, unidentified
  REGION 1..17: note = synthetic sequence
YWYFDLWGRGTLVTVSS

SEQ ID NO: 54 - protein, 10 aa, unidentified
  REGION 1..10: note = synthetic sequence
YYYGSGSYYN

SEQ ID NO: 55 - protein, 97 aa, Human immunodeficiency virus 1
EIVLTQSPGTLSLSPGERATLSCRASQSVSSSYLAWYQQKPGQAPRLLIYGASSRATGIPDRFSGSGSGTDFTLTISRLEPEDFAVYYCCQQYGSSP

SEQ ID NO: 56 - protein, 109 aa, Human immunodeficiency virus 1
EIVLAQSPGTLSLSPGERATLSCRASHNVHPKYFAWYQQKPGQSPRLLIYGGSTRAAGIPGKFSGSGSGTDFTLTISRVDPEDFAVYYCCQQYGGSPYTFGQGTKVEIK

SEQ ID NO: 57 - protein, 109 aa, unidentified
  REGION 1..109: note = synthetic sequence
EIVLTQSPGTLSLSPGERATLSCRASQSVSSSYLAWYQQKPGQAPRLLIYGASSRATGIPDRFSGSGSGTDFTLTISRLEPEDFAVYYCCQQYGSSPYTFGQGTKVEIK

SEQ ID NO: 58 - protein, 109 aa, unidentified
  REGION 1..109: note = synthetic sequence
EIVLTQSPGTLSLSPGERATLSCRASQSVSSSYLAWYQQKPGQAPRLLIYGASSRATGIPDRFSGSGSGTDFTLTISRLEPEDFAVYYCCQQYGSSPWTFGQGTKVEIK

SEQ ID NO: 59 - protein, 12 aa, Homo sapiens
RASQSVSSSYLA

SEQ ID NO: 60 - protein, 7 aa, Homo sapiens
GASSRAT

SEQ ID NO: 61 - protein, 9 aa, Homo sapiens
QQYGSSPWT

SEQ ID NO: 62 - protein, 12 aa, unidentified
  REGION 1..12: note = synthetic sequence
WTFGQGTKVEIK

SEQ ID NO: 63 - protein, 20 aa, Human immunodeficiency virus 1
NCSFNISTSIRGKVQKEYAF

SEQ ID NO: 64 - protein, 33 aa, Human immunodeficiency virus 1
GKIGNMRQAHCNISRAKWNNTLKQIASKLREQF

SEQ ID NO: 65 - protein, 39 aa, Human immunodeficiency virus 1
EGSDTITLPCRIKQIINMWQKVGKAMYAPPISGQIRCSS

SEQ ID NO: 66 - protein, 36 aa, Human immunodeficiency virus 1
STQLLLNGSLAEEEVVIRSVNFTDNAKTIIVQLNTS

SEQ ID NO: 67 - protein, 18 aa, Human immunodeficiency virus 1
CNNKTFNGTGPCTNVSTV

SEQ ID NO: 68 - protein, 12 aa, Human immunodeficiency virus 1
NITGLLLTRDGG

SEQ ID NO: 69 - protein, 10 aa, Human immunodeficiency virus 1
VVLVNVTENF

SEQ ID NO: 70 - protein, 8 aa, Human immunodeficiency virus 1
NITGLLLT

SEQ ID NO: 71 - protein, 28 aa, Human immunodeficiency virus 1
QREKRAVGIGALFLGFLGAAGSTMGAAS

SEQ ID NO: 72 - protein, 10 aa, Human immunodeficiency virus 1
TTAVPWNASW

SEQ ID NO: 73 - protein, 26 aa, Human immunodeficiency virus 1
QIWNHTTWMEWDREINNYTSLIHSLI

SEQ ID NO: 74 - protein, 20 aa, Simian immunodeficiency virus
NCSFNVTTELRDKKRQVYSL

SEQ ID NO: 75 - protein, 34 aa, Simian immunodeficiency virus
ENVVGDTRKAYCEINGTQWAKALNETKEVLRNIL

SEQ ID NO: 76 - protein, 39 aa, Simian immunodeficiency virus
KTENMTIIPCRIRQIVNSWMRVGKGIFAPPIRGNITCTS

SEQ ID NO: 77 - protein, 33 aa, Simian immunodeficiency virus
TTQLILNGSTADNQTVARIDPSENLAIIQLKDP

SEQ ID NO: 78 - protein, 18 aa, Simian immunodeficiency virus
CNDIDYKGNETCKNVSTV

SEQ ID NO: 79 - protein, 17 aa, Simian immunodeficiency virus
NITGMLLEIHKNREDQG

SEQ ID NO: 80 - protein, 10 aa, Simian immunodeficiency virus
VLLPNVTEEF

SEQ ID NO: 81 - protein, 8 aa, Simian immunodeficiency virus
NITGMLLE

SEQ ID NO: 82 - protein, 28 aa, Simian immunodeficiency virus
HREKRALGLGALFLGFLGAAGSTMGAAS

SEQ ID NO: 83 - protein, 10 aa, Simian immunodeficiency virus
PTAVRWNKTW

SEQ ID NO: 84 - protein, 26 aa, Simian immunodeficiency virus
VIWNNYTWQQWDREVNNYTGLIYTLL

SEQ ID NO: 85 - protein, 20 aa, Simian immunodeficiency virus
NCSFNVTTELRDKKKQVYSL

SEQ ID NO: 86 - protein, 34 aa, Simian immunodeficiency virus
KTVVGDTRGAECRINGTAWNETLRQVKEALQHIS

SEQ ID NO: 87 - protein, 39 aa, Simian immunodeficiency virus
TINNITHIPCRIRQIVNQWQGVGKGIFAPPIRGTIQCNS

SEQ ID NO: 88 - protein, 36 aa, Simian immunodeficiency virus
STQFLLNGSLAEGNKTVVRVRSKSNTETIIVQLATA

SEQ ID NO: 89 - protein, 18 aa, Simian immunodeficiency virus
CNDQNFKGKGTCRNVSTV

SEQ ID NO: 90 - protein, 11 aa, Simian immunodeficiency virus
TITGLLLTRDG

SEQ ID NO: 91 - protein, 10 aa, Simian immunodeficiency virus
ITLENVTEEF

SEQ ID NO: 92 - protein, 8 aa, Simian immunodeficiency virus
TITGLLLT

SEQ ID NO: 93 - protein, 28 aa, Simian immunodeficiency virus
QREKRAVGLGVMFLGFMGAAGSTMGAAA

SEQ ID NO: 94 - protein, 10 aa, Simian immunodeficiency virus
TTNVPWNNTW

SEQ ID NO: 95 - protein, 25 aa, Simian immunodeficiency virus
FWDNMTWMQWEKEIDNYTETIYELL

SEQ ID NO: 96 - protein, 20 aa, Simian immunodeficiency virus
NCSFNTTTELRDKKKQTYSL

SEQ ID NO: 97 - protein, 34 aa, Simian immunodeficiency virus
ETVVGDTREAHCEINQTHWYKILNQVKRELTTVF

SEQ ID NO: 98 - protein, 39 aa, Simian immunodeficiency virus
TLHNNLTLPCRIRQIVNLWLRVGKGIFAPPIRGNIRCNS

SEQ ID NO: 99 - protein, 31 aa, Simian immunodeficiency virus
STQLILNGSLAEEKIMIRYKADKVLVQLNTS

SEQ ID NO: 100 - protein, 18 aa, Simian immunodeficiency virus
CNNKTYNGSGPCNNVSTV

SEQ ID NO: 101 - protein, 8 aa, Simian immunodeficiency virus
TITGLILE

SEQ ID NO: 102 - protein, 10 aa, Simian immunodeficiency virus
IELSNVTENF

SEQ ID NO: 103 - protein, 8 aa, Simian immunodeficiency virus
TITGLILE

SEQ ID NO: 104 - protein, 28 aa, Simian immunodeficiency virus
DREKRAAGLGVFFLGFLGAAGSTMGAAS

SEQ ID NO: 105 - protein, 10 aa, Simian immunodeficiency virus
TTSVPWNRTW

SEQ ID NO: 106 - protein, 26 aa, Simian immunodeficiency virus
DIWNNLTWQQWDKLITNYTGTIFGLL

SEQ ID NO: 107 - protein, 20 aa, Simian immunodeficiency virus
NCTFNTTTELRDKKKQVYSL

SEQ ID NO: 108 - protein, 34 aa, Simian immunodeficiency virus
DHIFGDTRKAFCELNGTTWNETLQKVRESLIKEI

SEQ ID NO: 109 - protein, 39 aa, Simian immunodeficiency virus
PFHENMTIPCRIRQIVNSWMRVGRGIYAPPIPGHITCNS

SEQ ID NO: 110 - protein, 36 aa, Simian immunodeficiency virus
TTQLIINGSLATENITVRVNNASKNTHDWIVQLSTA

SEQ ID NO: 111 - protein, 18 aa, Simian immunodeficiency virus
CNDVNFSGKGKCRNVSTV

SEQ ID NO: 112 - protein, 12 aa, Simian immunodeficiency virus
LITGLILTRDHV

SEQ ID NO: 113 - protein, 10 aa, Simian immunodeficiency virus
VVLHNVTEDF

SEQ ID NO: 114 - protein, 8 aa, Simian immunodeficiency virus
LITGLILT

SEQ ID NO: 115 - protein, 29 aa, Simian immunodeficiency virus
RRQKRAAFGLGALFLGFLGAAGSTMGAAA

SEQ ID NO: 116 - protein, 10 aa, Simian immunodeficiency virus
YTTVPWNKTW

SEQ ID NO: 117 - protein, 26 aa, Simian immunodeficiency virus
DIWNNLTWQQWDKLITNYTGTIFGLL

SEQ ID NO: 118 - protein, 20 aa, Simian immunodeficiency virus
NCSFNVTTELRDKKKQVYSL

SEQ ID NO: 119 - protein, 33 aa, Simian immunodeficiency virus
ENVVGDTRSAYCKINGTTWNRTVEEVKGFSNLI

SEQ ID NO: 120 - protein, 39 aa, Simian immunodeficiency virus
ITNGIIILPCRIRQIVSSWMRVGRGIYAPPIRGNITCNS

SEQ ID NO: 121 - protein, 36 aa, Simian immunodeficiency virus
TTQLLINGSLAEGNITVRVENKSKNTDVWIVQLVEA

SEQ ID NO: 122 - protein, 18 aa, Simian immunodeficiency virus
CNDKDFSGKGKCTNVSTV

SEQ ID NO: 123 - protein, 12 aa, Simian immunodeficiency virus
NITGLLLTSDTP

SEQ ID NO: 124 - protein, 10 aa, Simian immunodeficiency virus
VFLPNVIESF

SEQ ID NO: 125 - protein, 8 aa, Simian immunodeficiency virus
NITGLLLT

SEQ ID NO: 126 - protein, 29 aa, Simian immunodeficiency virus
DRQKRAAFGLGALFLGFLGAAGSTMGAAA

SEQ ID NO: 127 - protein, 10 aa, Simian immunodeficiency virus
YTTVPWNNSW

SEQ ID NO: 128 - protein, 26 aa, Simian immunodeficiency virus
DIWGNLTWQQWDKLVSNYTGKIFGLL

SEQ ID NO: 129 - protein, 20 aa, Simian immunodeficiency virus
NCSFNATTELRDRKKQVYSL

SEQ ID NO: 130 - protein, 34 aa, Simian immunodeficiency virus
ETVIGDTRQAFCQLNKTVWTNTFKKVRHALNETY

SEQ ID NO: 131 - protein, 39 aa, Simian immunodeficiency virus
TKDDNITIPCRIRQIVRLWQRVGRGIFLPPIRGTINCIS

SEQ ID NO: 132 - protein, 35 aa, Simian immunodeficiency virus
STQFLLNGTLEEKVTVLDRNVSNDMDTIIVKLNET

SEQ ID NO: 133 - protein, 18 aa, Simian immunodeficiency virus
CNDKNFTGIGQCKNVSTV

SEQ ID NO: 134 - protein, 10 aa, Simian immunodeficiency virus
NITGILFAQQ

SEQ ID NO: 135 - protein, 10 aa, Simian immunodeficiency virus
IELTNVTEYF

SEQ ID NO: 136 - protein, 8 aa, Simian immunodeficiency virus
NITGILFA

SEQ ID NO: 137 - protein, 29 aa, Simian immunodeficiency virus
HREKRAAVGLGALFLGFLGAAGSTMGAAS

SEQ ID NO: 138 - protein, 10 aa, Simian immunodeficiency virus
TTSVPWNTTW

SEQ ID NO: 139 - protein, 26 aa, Simian immunodeficiency virus
DIWYNMTWMQWDKEVSNYTDVIYNLL

SEQ ID NO: 140 - protein, 12 aa, Simian immunodeficiency virus
NCTFNVTTELRG

SEQ ID NO: 141 - protein, 34 aa, Simian immunodeficiency virus
ENIVGNTRKAFCKVNGSQWWNMKQNIIQRFKAEH

SEQ ID NO: 142 - protein, 39 aa, Simian immunodeficiency virus
KTNITIILPCKIRQIVNSWMRVGKGIYAPPIRGNLSCNS

SEQ ID NO: 143 - protein, 35 aa, Simian immunodeficiency virus
TTQLILNGSITQQIMIRSKNISSNSFNIIVQFNET

SEQ ID NO: 144 - protein, 18 aa, Simian immunodeficiency virus
CNEENSQEMGYCENVSTV

SEQ ID NO: 145 - protein, 12 aa, Simian immunodeficiency virus
NITGLLLTRDGP

SEQ ID NO: 146 - protein, 10 aa, Simian immunodeficiency virus
VFLKNVKENF

SEQ ID NO: 147 - protein, 8 aa, Simian immunodeficiency virus
NITGLLLT

SEQ ID NO: 148 - protein, 18 aa, Simian immunodeficiency virus
HRAKRAAFAAGSTMGAAA

SEQ ID NO: 149 - protein, 10 aa, Simian immunodeficiency virus
YTNVPWNRNW

SEQ ID NO: 150 - protein, 26 aa, Simian immunodeficiency virus
EIWNNLTWNEWDKQVSNYTTEIFKAL

SEQ ID NO: 151 - protein, 20 aa, Simian immunodeficiency virus
NCSFNVTTELRDKKKQVYSL

SEQ ID NO: 152 - protein, 34 aa, Simian immunodeficiency virus
ENVVGDTRKAYCTLNGSNWEEAMSKVKKELEKIT

SEQ ID NO: 153 - protein, 39 aa, Simian immunodeficiency virus
NKSEEIILPCRIKQIVNLWTRVGKGIYAPPIRGNISCKS

SEQ ID NO: 154 - protein, 36 aa, Simian immunodeficiency virus
TTQLILNGSLAEGNVTIRVKNASSNSGNYIVQLARS

SEQ ID NO: 155 - protein, 18 aa, Simian immunodeficiency virus
CNDTNYLGTGKCHNVSTV

SEQ ID NO: 156 - protein, 12 aa, Simian immunodeficiency virus
  VARIANT 10: note = Xaa can be any naturally occurring amino acid
NITGILLARXEG

SEQ ID NO: 157 - protein, 10 aa, Simian immunodeficiency virus
VFLPNVTENF

SEQ ID NO: 158 - protein, 8 aa, Simian immunodeficiency virus
NITGILLA

SEQ ID NO: 159 - protein, 29 aa, Simian immunodeficiency virus
KRQKRAAFGLGALFLGFLGAAGSTMGAAA

SEQ ID NO: 160 - protein, 10 aa, Simian immunodeficiency virus
YTTVPWNKTW

SEQ ID NO: 161 - protein, 26 aa, Simian immunodeficiency virus
YIWNNLTWEQWDVHVSNYTGVIFGLL

SEQ ID NO: 162 - protein, 20 aa, Simian immunodeficiency virus
NCTFNMTTEVRDKEKKVYSL

SEQ ID NO: 163 - protein, 34 aa, Simian immunodeficiency virus
ENIVGDTRRAYCQINRTVWDERLNETGQALRELF

SEQ ID NO: 164 - protein, 39 aa, Simian immunodeficiency virus
KGDNTTFFPCRIRQIVNSWMRVGKGIYAPPIRGVISCTS

SEQ ID NO: 165 - protein, 31 aa, Simian immunodeficiency virus
  VARIANT 23: note = Xaa can be any naturally occurring amino acid
  VARIANT 26: note = Xaa can be any naturally occurring amino acid
  VARIANT 30..31: note = Xaa can be any naturally occurring amino acid
TTQLILNGSVAENKTIARRNGYXFLXQFQXX

SEQ ID NO: 166 - protein, 20 aa, Simian immunodeficiency virus
CNMPNFNGTGTGRCNNISTV

SEQ ID NO: 167 - protein, 12 aa, Simian immunodeficiency virus
NVTGIILETGHG

SEQ ID NO: 168 - protein, 10 aa, Simian immunodeficiency virus
VQIPNVTENF

SEQ ID NO: 169 - protein, 8 aa, Simian immunodeficiency virus
NVTGIILE

SEQ ID NO: 170 - protein, 29 aa, Simian immunodeficiency virus
SREKRAAFGLGALFLGFLGAAGSTMGAAS

SEQ ID NO: 171 - protein, 10 aa, Simian immunodeficiency virus
YTTVPWNNTW

SEQ ID NO: 172 - protein, 26 aa, Simian immunodeficiency virus
EIWNNLTWQDWDKRVKNYSGVIFSLI

SEQ ID NO: 173 - protein, 20 aa, Simian immunodeficiency virus
NCSFNQTTEFRDKKKQIYSL

SEQ ID NO: 174 - protein, 34 aa, Simian immunodeficiency virus
ENVVGDTRKAYCHVNATLWDQSLSRAMEAINKTL

SEQ ID NO: 175 - protein, 39 aa, Simian immunodeficiency virus
HRNGTLVAPCRLRQIVNHWGIVSKGIYLPPRRGTLKCNS

SEQ ID NO: 176 - protein, 35 aa, Simian immunodeficiency virus
ATALHLNGTLEENETKAYFADVNVNPPLLVKFNES

SEQ ID NO: 177 - protein, 18 aa, Simian immunodeficiency virus
CNDQGYTGNGTCSNVTVV

SEQ ID NO: 178 - protein, 12 aa, Simian immunodeficiency virus
NITGLIMTAEKG

SEQ ID NO: 179 - protein, 10 aa, Simian immunodeficiency virus
VPLANVSIDF

SEQ ID NO: 180 - protein, 8 aa, Simian immunodeficiency virus
NITGLIMT

SEQ ID NO: 181 - protein, 28 aa, Simian immunodeficiency virus
TRSRRDVGIGLLFLGFLSAAGSTMGAAS

SEQ ID NO: 182 - protein, 10 aa, Simian immunodeficiency virus
HSSVPWNLSW

SEQ ID NO: 183 - protein, 26 aa, Simian immunodeficiency virus
CIWNNLTWQEWDRLVQNASETIYSLL

SEQ ID NO: 184 - protein, 20 aa, Simian immunodeficiency virus
NCSFQQTTEFRDKKKQIYSL

SEQ ID NO: 185 - protein, 33 aa, Simian immunodeficiency virus
ENVVGDTRKAYCWVNATLWYKSLDNAWRLPKHR

SEQ ID NO: 186 - protein, 40 aa, Simian immunodeficiency virus
ALNKTHLLAQCRLRQIVNHWGIVSKGIYLPPRKGHIKCVS

SEQ ID NO: 187 - protein, 35 aa, Simian immunodeficiency virus
ATALHLNGTLEEKETKAYFVNATNNPPLLVKFNES

SEQ ID NO: 188 - protein, 18 aa, Simian immunodeficiency virus
CKDQNYTGNGICYNVTVV

SEQ ID NO: 189 - protein, 12 aa, Simian immunodeficiency virus
NITGLIMTAEGG

SEQ ID NO: 190 - protein, 10 aa, Simian immunodeficiency virus
VPIANVSIKF

SEQ ID NO: 191 - protein, 8 aa, Simian immunodeficiency virus
NITGLIMT

SEQ ID NO: 192 - protein, 28 aa, Simian immunodeficiency virus
TRSRRDVGIGLLFLGFLSAAGSTMGAAS

SEQ ID NO: 193 - protein, 10 aa, Simian immunodeficiency virus
HSSVPWNLSW

SEQ ID NO: 194 - protein, 26 aa, Simian immunodeficiency virus
CIWNNLTWQEWDKLVQNSSETIYSLL

SEQ ID NO: 195 - protein, 20 aa, Simian immunodeficiency virus
NCSFNQTTEFRDKKKQIYSL

SEQ ID NO: 196 - protein, 34 aa, Simian immunodeficiency virus
ENVVGDTRKAYCSVNATTWYRNLDWAMAAINTTM

SEQ ID NO: 197 - protein, 39 aa, Simian immunodeficiency virus
NTHGTLVAPCRLRQIVNHWGIVSKGVYLPPRRGTVKCHS

SEQ ID NO: 198 - protein, 35 aa, Simian immunodeficiency virus
ATALHLNGSLEEEETKAYFVNTSVNTPLLVKFNVS

SEQ ID NO: 199 - protein, 18 aa, Simian immunodeficiency virus
CRDQNFTGKGQCSNVSVV

SEQ ID NO: 200 - protein, 12 aa, Simian immunodeficiency virus
NITGLIMTAEKD

SEQ ID NO: 201 - protein, 10 aa, Simian immunodeficiency virus
VTLANVSIRF

SEQ ID NO: 202 - protein, 8 aa, Simian immunodeficiency virus
NITGLIMT

SEQ ID NO: 203 - protein, 28 aa, Simian immunodeficiency virus
TRSRRDVGIGLLFLGFLSAAGSTMGAAS

SEQ ID NO: 204 - protein, 10 aa, Simian immunodeficiency virus
HSSVPWNLTW

SEQ ID NO: 205 - protein, 26 aa, Simian immunodeficiency virus
CIWNNLTWQEWDRLVENSTGTIYSLL

SEQ ID NO: 206 - protein, 20 aa, Simian immunodeficiency virus
KCSFNQTTEFRDKKKQIYSL

SEQ ID NO: 207 - protein, 34 aa, Simian immunodeficiency virus
ENVVGDTRKAFCIVNATEWSNALNESKIAINETL

SEQ ID NO: 208 - protein, 39 aa, Simian immunodeficiency virus
GKNGSIVAQCRLRQIVNHWGIVSKGIYLPPRRGTIKCTS

SEQ ID NO: 209 - protein, 35 aa, Simian immunodeficiency virus
ATALHLNGTLEENKTTAYFADSTHNQPLLVKFHKA

SEQ ID NO: 210 - protein, 18 aa, Simian immunodeficiency virus
CKDQNFTGKGPCSNVSVV

SEQ ID NO: 211 - protein, 12 aa, Simian immunodeficiency virus
NITGLILTAEGG

SEQ ID NO: 212 - protein, 10 aa, Simian immunodeficiency virus
VPLANMTVDF

SEQ ID NO: 213 - protein, 8 aa, Simian immunodeficiency virus
NITGLILT

SEQ ID NO: 214 - protein, 28 aa, Simian immunodeficiency virus
TRSKRDVGIGLLFLGFLSAAGSTMGAAS

SEQ ID NO: 215 - protein, 10 aa, Simian immunodeficiency virus
HSSVPWNITW

SEQ ID NO: 216 - protein, 26 aa, Simian immunodeficiency virus
CIWNNLTWQEWDKLVKNSSETIYSLL

SEQ ID NO: 217 - protein, 20 aa, Simian immunodeficiency virus
NCSFNQTTEFRDKKKQIYSL

SEQ ID NO: 218 - protein, 34 aa, Simian immunodeficiency virus
ENIVGNTRKAYCTVNYQEWSHAIGEAKKVAEEAL

SEQ ID NO: 219 - protein, 39 aa, Simian immunodeficiency virus
NSSRPLVAPCKLRQIVNHWGIVSKGIYLPPRRGNLTCRS

SEQ ID NO: 220 - protein, 35 aa, Simian immunodeficiency virus
ATALHLNGTLEKHNTTAYFATPTHNKPLLIKFNKS

SEQ ID NO: 221 - protein, 18 aa, Simian immunodeficiency virus
CNSANFTGVGTCNNVSVV

SEQ ID NO: 222 - protein, 10 aa, Simian immunodeficiency virus
NITGFITTWD

SEQ ID NO: 223 - protein, 10 aa, Simian immunodeficiency virus
VPLSNVSVDF

SEQ ID NO: 224 - protein, 8 aa, Simian immunodeficiency virus
NITGFITT

SEQ ID NO: 225 - protein, 28 aa, Simian immunodeficiency virus
TRSKRDVGIGLLFLGFLSAAGSTMGAAS

SEQ ID NO: 226 - protein, 10 aa, Simian immunodeficiency virus
HSNVVWNMTW

SEQ ID NO: 227 - protein, 26 aa, Simian immunodeficiency virus
CIWNNLTWQQWEKLVANSTEEIYTLL

SEQ ID NO: 228 - protein, 20 aa, Simian immunodeficiency virus
NCSFNQTTEFRDKKKNIYSL

SEQ ID NO: 229 - protein, 34 aa, Simian immunodeficiency virus
ENIFGDTRKAFCYVNATEWRRTLEMAKTALREAT

SEQ ID NO: 230 - protein, 39 aa, Simian immunodeficiency virus
TDQTNLVAPCRLRQIVNHWGIVAKAIYLPPRRGTVKCVS

SEQ ID NO: 231 - protein, 35 aa, Simian immunodeficiency virus
ASWLHLNGTYEKEKTKVYHTNVTNNPPLLVKFNET

SEQ ID NO: 232 - protein, 18 aa, Simian immunodeficiency virus
CNDANFTGAGECTNVSVV

SEQ ID NO: 233 - protein, 8 aa, Simian immunodeficiency virus
NITGFLMT

SEQ ID NO: 234 - protein, 10 aa, Simian immunodeficiency virus
VPLPNVSIEF

SEQ ID NO: 235 - protein, 8 aa, Simian immunodeficiency virus
NITGFLMT

SEQ ID NO: 236 - protein, 28 aa, Simian immunodeficiency virus
TRSKRDVGIGLLFLGFLSAAGSTMGAAS

SEQ ID NO: 237 - protein, 10 aa, Simian immunodeficiency virus
HSTVVWNDTW

SEQ ID NO: 238 - protein, 26 aa, Simian immunodeficiency virus
CIWQNLTWQQWSELVDNSTDTIYTLL

SEQ ID NO: 239 - protein, 20 aa, Simian immunodeficiency virus
ECFFNQTTEFRDKKKQIYSL

SEQ ID NO: 240 - protein, 35 aa, Simian immunodeficiency virus
RNVIGDTRKAFCYVNKTLWENALRQQKQTKLLKTP

SEQ ID NO: 241 - protein, 39 aa, Simian immunodeficiency virus
EIDGMLIAPCRLRQIVNHWGIVSRGIYLPPREGQVKCVS

SEQ ID NO: 242 - protein, 35 aa, Simian immunodeficiency virus
ATALHLNGSVEEGETKAYYVEAKHNAPLLIKFNKA

SEQ ID NO: 243 - protein, 18 aa, Simian immunodeficiency virus
CKDENFRGKGNCTTVRIV

SEQ ID NO: 244 - protein, 8 aa, Simian immunodeficiency virus
SITGFIMT

SEQ ID NO: 245 - protein, 9 aa, Simian immunodeficiency virus
IPINITTEF

SEQ ID NO: 246 - protein, 8 aa, Simian immunodeficiency virus
SITGFIMT

SEQ ID NO: 247 - protein, 28 aa, Simian immunodeficiency virus
SRSKREIGMGMIFMGFLSAAGSTMGAAS

SEQ ID NO: 248 - protein, 10 aa, Simian immunodeficiency virus
HSSLEWNSSW

SEQ ID NO: 249 - protein, 26 aa, Simian immunodeficiency virus
CIWSQYTWQQWDEEIRNISDVIYLSL

SEQ ID NO: 250 - protein, 20 aa, Simian immunodeficiency virus
ECNFNQSTGFKDKKQKMKAI

SEQ ID NO: 251 - protein, 34 aa, Simian immunodeficiency virus
EIATGDTRKAFCTVNKTLWEQARNKTEHVLAEHW

SEQ ID NO: 252 - protein, 39 aa, Simian immunodeficiency virus
ITNGALIAHCRIKQIVNHWGIVSKGIYLAPRRGNVSCTS

SEQ ID NO: 253 - protein, 32 aa, Simian immunodeficiency virus
ATWLLLNGTYQTNTSVVMNGAKMNLCLDLEKN

SEQ ID NO: 254 - protein, 18 aa, Simian immunodeficiency virus
CEDEDFTGVGMCKNVSVV

SEQ ID NO: 255 - protein, 9 aa, Simian immunodeficiency virus
SITGIMLEG

SEQ ID NO: 256 - protein, 9 aa, Simian immunodeficiency virus
VRLNTSVWF

SEQ ID NO: 257 - protein, 8 aa, Simian immunodeficiency virus
SITGIMLE

SEQ ID NO: 258 - protein, 28 aa, Simian immunodeficiency virus
SRQKRGIGIGLFFLGLLSAAGSTMGAAS

SEQ ID NO: 259 - protein, 10 aa, Simian immunodeficiency virus
HTTVPWNNSW

SEQ ID NO: 260 - protein, 26 aa, Simian immunodeficiency virus
CIWENMTWQEWDRLVQNSTGQIYNIL

SEQ ID NO: 261 - protein, 20 aa, Simian immunodeficiency virus
NCTFNTTTELRDKKQQIYSL

SEQ ID NO: 262 - protein, 34 aa, Simian immunodeficiency virus
EALIGNPRKASCHLNGTLWNNILNRIKQKIKNST

SEQ ID NO: 263 - protein, 39 aa, Simian immunodeficiency virus
SDTSEYILPCKIRQVVNSWMRVGKGIFAPPRRGTITCNS

SEQ ID NO: 264 - protein, 39 aa, Simian immunodeficiency virus
TTQLILNGSLAEEEEVVFRTKNMTAPGLSDTVIVQLKRA

SEQ ID NO: 265 - protein, 18 aa, Simian immunodeficiency virus
CNDKDYPGKGKCKNVSTV

SEQ ID NO: 266 - protein, 12 aa, Simian immunodeficiency virus
TITGLLLEVQNG

SEQ ID NO: 267 - protein, 10 aa, Simian immunodeficiency virus
VILINVTEEF

SEQ ID NO: 268 - protein, 8 aa, Simian immunodeficiency virus
TITGLLLE

SEQ ID NO: 269 - protein, 29 aa, Simian immunodeficiency virus
DRSKRAAFGLGALFLGFLGAAGSTMGAAS

SEQ ID NO: 270 - protein, 10 aa, Simian immunodeficiency virus
YSTVPWNTSW

SEQ ID NO: 271 - protein, 26 aa, Simian immunodeficiency virus
DIWSNLTWQQWDKLVENHTGTIFQLL

SEQ ID NO: 272 - protein, 20 aa, Simian immunodeficiency virus
NCSFNVTTELRDKKKQVYSL

SEQ ID NO: 273 - protein, 31 aa, Simian immunodeficiency virus
ENIIGDTRQAYCEVNRTWEQIWNTTKQIIIN

SEQ ID NO: 274 - protein, 39 aa, Simian immunodeficiency virus
NTTGNITLQCRIRQIVNLWTRVGKGIYAPPIKGPINCLS

SEQ ID NO: 275 - protein, 32 aa, Simian immunodeficiency virus
STQLILNGSLATSNIVIRNNSKDTLLVQLNES

SEQ ID NO: 276 - protein, 18 aa, Simian immunodeficiency virus
CNDQDFNGTGECNNVSTV

SEQ ID NO: 277 - protein, 10 aa, Simian immunodeficiency virus
NITGIILDYT

SEQ ID NO: 278 - protein, 10 aa, Simian immunodeficiency virus
VLIPNVTERF

SEQ ID NO: 279 - protein, 8 aa, Simian immunodeficiency virus
NITGIILD

SEQ ID NO: 280 - protein, 29 aa, Simian immunodeficiency virus
TRQKRAAFGLGALFLGFLGAAGSTMGAAS

SEQ ID NO: 281 - protein, 10 aa, Simian immunodeficiency virus
YTNVPWNTTW

SEQ ID NO: 282 - protein, 26 aa, Simian immunodeficiency virus
TIWGNMTWQNWDEQVRNYSGVIFGLL

SEQ ID NO: 283 - protein, 20 aa, Simian immunodeficiency virus
NCTFNMTTEVRDKKKEVYSL

SEQ ID NO: 284 - protein, 34 aa, Simian immunodeficiency virus
ENIVGDPRQAHCNVSKAQWMKKLNETALAIAKYD

SEQ ID NO: 285 - protein, 39 aa, Simian immunodeficiency virus
SDNSTLTISCRIRQIVNSWMRVGKGIYAPPIRGNITCIS

SEQ ID NO: 286 - protein, 35 aa, Simian immunodeficiency virus
TTQVILNGSLAEEGPAVARSEDLRHKIIIIQFKEG

SEQ ID NO: 287 - protein, 18 aa, Simian immunodeficiency virus
CNDQKFNGTGPCTNISTV

SEQ ID NO: 288 - protein, 11 aa, Simian immunodeficiency virus
NITGLLLEAGG

SEQ ID NO: 289 - protein, 10 aa, Simian immunodeficiency virus
VYMPNVSESF

SEQ ID NO: 290 - protein, 8 aa, Simian immunodeficiency virus
NITGLLLE

SEQ ID NO: 291 - protein, 29 aa, Simian immunodeficiency virus
AREKRAAFGLGALFLGFLGAAGSTMGAAS

SEQ ID NO: 292 - protein, 10 aa, Simian immunodeficiency virus
YTTVPWNATW

SEQ ID NO: 293 - protein, 26 aa, Simian immunodeficiency virus
EIWNNLTWQDWDKKVKNYSGVIFSLI

SEQ ID NO: 294 - protein, 20 aa, Simian immunodeficiency virus
NCSFNITTELRDKKKQVYSL

SEQ ID NO: 295 - protein, 34 aa, Simian immunodeficiency virus
PKIVGDVREAHCNISKLTWEKQRKYTLEIIKKEA

SEQ ID NO: 296 - protein, 39 aa, Simian immunodeficiency virus
TDNTTITLKCRIRQIVNQWMRVGKGIFAPPIKGVLSCNS

SEQ ID NO: 297 - protein, 33 aa, Simian immunodeficiency virus
TTQLIINGSLATKNVTVRSKNFADIILVQFSEG

SEQ ID NO: 298 - protein, 18 aa, Simian immunodeficiency virus
CNEKDFKGKGECKNVSTV

SEQ ID NO: 299 - protein, 11 aa, Simian immunodeficiency virus
NITGMFLTAAQ

SEQ ID NO: 300 - protein, 10 aa, Simian immunodeficiency virus
VHLPNVTEKF

SEQ ID NO: 301 - protein, 8 aa, Simian immunodeficiency virus
NITGMFLT

SEQ ID NO: 302 - protein, 29 aa, Simian immunodeficiency virus
KREKRAAFGLGALFLGFLGAAGSTMGAAS

SEQ ID NO: 303 - protein, 10 aa, Simian immunodeficiency virus
YTTVPWNDTW

SEQ ID NO: 304 - protein, 26 aa, Simian immunodeficiency virus
AIWGNLTWQEWDRKVRNYSGTIFSLI

SEQ ID NO: 305 - protein, 20 aa, Simian immunodeficiency virus
KCFFNMTTELRDKQKQVYSL

SEQ ID NO: 306 - protein, 34 aa, Simian immunodeficiency virus
  VARIANT 14: note = Xaa can be any naturally occurring amino acid
  VARIANT 23..24: note = Xaa can be any naturally occurring amino acid
  VARIANT 27: note = Xaa can be any naturally occurring amino acid
ENIIGDTRQAYCNXSAAEWEQRXXDTXRAIKSLK

SEQ ID NO: 307 - protein, 39 aa, Simian immunodeficiency virus
INSSNIIINCRIRQIVNSWMRVGKGIYAPPIRGTISCTS

SEQ ID NO: 308 - protein, 36 aa, Simian immunodeficiency virus
  VARIANT 16: note = Xaa can be any naturally occurring amino acid
  VARIANT 17: note = Xaa can be any naturally occurring amino acid
  VARIANT 21: note = Xaa can be any naturally occurring amino acid
  VARIANT 23..24: note = Xaa can be any naturally occurring amino acid
  VARIANT 27: note = Xaa can be any naturally occurring amino acid
TTQLILNGSLAENSTXXRTEXMXXMDXTIIVQFHKR

SEQ ID NO: 309 - protein, 18 aa, Simian immunodeficiency virus
CNEPDFNGTGKCRNISTV

SEQ ID NO: 310 - protein, 15 aa, Simian immunodeficiency virus
NITGLLLELDRGLPR

SEQ ID NO: 311 - protein, 10 aa, Simian immunodeficiency virus
VHVPNITETF

SEQ ID NO: 312 - protein, 8 aa, Simian immunodeficiency virus
NITGLLLE

SEQ ID NO: 313 - protein, 29 aa, Simian immunodeficiency virus
TREKRAAFGLGALFLGFLGAAGSTMGAAS

SEQ ID NO: 314 - protein, 10 aa, Simian immunodeficiency virus
YTTVPWNSTW

SEQ ID NO: 315 - protein, 26 aa, Simian immunodeficiency virus
EIWNNLTWQDWDKRVKNYSGVIFDLI

SEQ ID NO: 316 - protein, 650 aa, Simian immunodeficiency virus
MDAMKRGLCCVLLLCGAVFVSPSQEIHARFRRGARENWWVTVYYGVPVWREAKTTLFCASDAKSYSTEAHNIWATQACVPTDPTPQEVLLPNVTEEFNMWENYMVDQMQEDIISLWEQSLKPCVKLTPLCVTLTCNNPTNTSCTNSTDDRLGDMRNCSFNVTTELRDKKRKVYSLFYVEDITAIGNNSTYRLINCNTTAITQACPKTSFEPIPIHYCAPAGFALLKCNDIDYKGNETCKNVSTVHCTHGIKPVATTQLILNGSTADNQTVARIDPSENLAIIQLKDPVKITCRRPGNNTRGQIQIGPAMTFYNIENVVGDTRKAYCEINGTQWAKALNETKEVLRNILRKNISFMVPSGGDPEVTNHHFNCGGEFFYCNTSEIINITKINKTENMTIIPCRIRQIVNSWMRVGKGIFAPPIRGNITCTSNITGMLLEIHKDQNNTYVCLTGGNMKDIWRSELYKYKIVEIQPLGVAPTKCRRYAVEKQHHRRRRRRALGLGALFLGFLGAAGSTMGAASVVLTVQARQLLTGIVQQQNNLLRAPEAQQHLLQLSVWGIKQLQARVLAVERYLRDQQLLGLWGCTGKTICCTAVRWNKTWGNISDYQVIWNNYTWQQWDREVNNYTGLIYTLLEEANTQQEKNEKELLELD

SEQ ID NO: 317 - protein, 836 aa, Simian immunodeficiency virus
MKVMEKKKRLWLSYCLLSSLIIPGLSSLWATVYYGVPVWRDVETTLFCASDAKAYKQEAHNIWATQACVPTDPNPQEVHLPNVTEKFDMWENNMAEQMQEDIISLWDQSLKPCIKLTPLCVTMTCLNPDSNSSAVNTTDIMRNCSFNITTELRDKKKQVYSLFYVDDLAHINNNTYRLINCNTTAITQACPKTSFEPIPIHYCAPPGFAILKCNEKDFKGKGECKNVSTVQCTHGIKPVVTTQLIINGSLATKNVTVRSKNFADIILVQFSEGVNMTCIRPGNNTVGNVQLGPGMTFYNIPKIVGDVREAHCNISKLTWEKQRKYTLEIIKKEANLTKVELIPNAGGDPEVVNMMLNCGGEFFYCNTIPLFNMTYNNTDNTTITLKCRIRQIVNQWMRVGKGIFAPPIKGVLSCNSNITGMILDISISAVNNDSRNITVMPTGGDMTALWKNELHKYKVVSIEPIGVAPGKAKRHTVKREKRAAFGLGALFLGFLGAAGSTMGAASVVLTVQARQLLSGIVQQQNNLLRAIEAQQHLLQLSVWGIKQLQARVLAVERYLKDQQILGLWGCSGKTICYTTVPWNDTWSNNLSYDAIWGNLTWQEWDRKVRNYSGTIFSLIEQAQEQQNTNEKSLLELDQWSSLWNWFDITNWLWYIKIFLIVVASLVGIRIVGVIFSLVAKVRQGYSPLSLQTLFPTTREPDRPEGTEEGAGKTDNVRSTRLVSGFLALVWEDLRNLLIFLYHRLEDLLLILRRTVQILGQNINKGLQLLNELRARCWGVIAYWARELKVSATSLLDTTAIAVAEGTDRIIELTRRLFLGIIHIPRRIRQGLERSLL

SEQ ID NO: 318 - protein, 859 aa, Simian immunodeficiency virus
  VARIANT 154: note = Xaa can be any naturally occurring amino acid
  VARIANT 284: note = Xaa can be any naturally occurring amino acid
  VARIANT 287: note = Xaa can be any naturally occurring amino acid
  VARIANT 291..292: note = Xaa can be any naturally occurring amino acid
  VARIANT 303: note = Xaa can be any naturally occurring amino acid
MKVMEKIKGSLIEKYMFMGLIIPCLTGSDQLWATVYYGVPVWKEADTTLFCASEASALNKEAHNIWASQACVPTDPNPQEVQIPNVTENFNMWNNTMAEQMNTDIISLWDQSLKPCVKLTPICVTMECRKVTFNSTSNRNKTSTMTTNSPNEKXDSTVKNCTFNMTTEVRDKEKKVYSLFYVDDLVLIDNDTDTYRLINCNTTAITQACPKTSFEPIPIHYCATPGYAIMKCNMPNFNGTGTGRCNNISTVQCTHGIRPVVTTQLILNGSVAENKTIARRNGYXFLXQFQXXVSINCTRPGNXSRGQIQIGPGMTFYNIENIVGDTRRAYCQINRTVWDERLNETGQALRELFTNLTQVNFTVSPGGDPEVTNMMFNCGGEFFYCNTTTLFNYTWKNNNITKGDNTTFFPCRIRQIVNSWMRVGKGIYAPPIRGVISCTSNVTGIILETGHGINNSITNITLYPTGGNMVDLWRLELHKYKVVSIEPIGVAPSKAKRHTVSREKRAAFGLGALFLGFLGAAGSTMGAASVVLTVQARQLLSGIVQQQNNLLRAIEAQQHLLQLSVWGIKQLQARVLAVERYLRDQQILGLWGCSGKAICYTTVPWNNTWSANTSFDEIWNNLTWQDWDKRVKNYSGVIFSLIEQAQEQQNTNEKSLLELDQWSSLWNWFDITRWLWYIKLFIMIVAGLVGIRIVGAIISFVAKVRQGYSPLSLQTLIPTTREPDRPGGTEEDVGEPGKGRSIRLVSGFLALIWEDLRNLLLFCYHRLRDLLLILGRTLENLGQSLNKGLQQLRNFSRYLWGVITYWGRELQTSAISLLDATAIAVAEGTDRILEVAQIIGRGILHIPRRIRQGLERSLL

SEQ ID NO: 319 - protein, 856 aa, Simian immunodeficiency virus
MKVMGKTRRRWQPYCIIMALIIPCLKTESDQWWVTVYYGVPVWKEATTPLFCAANATALKEEPHNIWATQACVPTDPSPEEVILINVTEEFNVWDNAMVEQMQEDITSLWDQSLRPCVKLNPLCVQLTCTSINATGNETNATGNGIEKDGLAQDMRNCTFNTTTELRDKKQQIYSLFWKNDLVGTNNTFRLINCNTTAITQACPKTSFEPIPIHYCAPAGFALLKCNDKDYPGKGKCKNVSTVHCTHGIKPTVTTQLILNGSLAEEEEVVFRTKNMTAPGLSDTVIVQLKRAIPINCSRPGNNTGRAINLSPGTTFFNTEALIGNPRKASCHLNGTLWNNILNRIKQKIKNSTTWHRGDITFTKHPGGDPEVVNFMFNCGGEFFYCNTSRLITCNSSDTSEYILPCKIRQVVNSWMRVGKGIFAPPRRGTITCNSTITGLLLEVQNGTGNNTEVYLSGGDMRDIWRSELYKYKIVKIEPLGVAPTKAKRYTVAKALDRSKRAAFGLGALFLGFLGAAGSTMGAASVMLTVQARNLLSGIVQQQNNLLRAIEAQQHLLQLSVWGIKQLQARVLAVERYLKDQQVLALWGCSGKTVCYSTVPWNTSWNSNKSYEDIWSNLTWQQWDKLVENHTGTIFQLLQRAHEQQNSNEKELLELDQWSSLWNWFDITNWLWYIKMFIILVGGVIGLRIVIGVVNIIRRSRQGYSPLSFQTLIPATGGPEVPEGIGGGGGEQDRGRSVRLVSGFLALCWDDIRNLTIFLYRLLRDCLSVLWSLLEQLGQQVLRGLRLLRELLSQLKGIGQYWLQELRTSAISLLDTTAIIVAERTDTIIEVATRIGRGILHIPRRIRQGLERALLE

SEQ ID NO: 320 - protein, 871 aa, Simian immunodeficiency virus
MKNLIGITLILIITILGIGFSTYYTTVFYGVPVWKEAQPTLFCASDADITSRDKHNIWATHNCVPLDPNPYEVTLANVSIRFNMEENYMVQEMKEDILSLFQQSFKPCVKLTPFCIKMTCTMTNTTNKTLNSATTTLTPTVNLSSIPNYEVYNCSFNQTTEFRDKKKQIYSLFYREDIVKEDGNNNSYYLHNCNTSVITQECDKSTFEPIPIRYCAPAGFALLKCRDQNFTGKGQCSNVSVVHCTHGIYPMIATALHLNGSLEEEETKAYFVNTSVNTPLLVKFNVSINLTCERTGNNTRGQVQIGPGMTFYNIENVVGDTRKAYCSVNATTWYRNLDWAMAAINTTMRARNETVQQTFQWQRDGDPEVTSFWFNCQGEFFYCNLTNWTNTWTANRTNNTHGTLVAPCRLRQIVNHWGIVSKGVYLPPRRGTVKCHSNITGLIMTAEKDNNNSYTPQFSAVVEDYWKVELARYKVVEIQPLSVAPRPGKRPEIKANHTRSRRDVGIGLLFLGFLSAAGSTMGAASIALTAQARGLLSGIVQQQQNLLQAIEAQQHLLQLSVWGIKQLQARMLAVEKYIRDQQLLSLWGCANKLVCHSSVPWNLTWAEDSTKCNHSDAKYYDCIWNNLTWQEWDRLVENSTGTIYSLLEKAQTQQEKNKQELLELDKWSSLWDWFDITQWLWYIKIAIIIVAGLVGLRILMFIVNVVKQVRQGYTPLFSQIPTQAEQDPEQPGGIAGGGGGRDNIRWTPSPAGFFSIVWEDLRNLLIWIYQTFQNFIWILWISLQALKQGIISLAHSLVIVHRTIIVGVRQIIEWSSNTYASLRVLLIQAIDRLANFTGWWTDLIIEGVVYIARGIRNIPRRIRQGLELALN

SEQ ID NO: 321 - protein, 865 aa, Simian immunodeficiency virus
MKAMERKKLNWIICYMLMGLITPCLTGHEWWATVYYGVPVWKEAETTLFCASEASALNKEAHNIWASQACVPVDPNPQEVYMPNVSESFNMWKNNMAEQMQEDISSLWDQSLKPCVKLTPICVTMQCTTLKNISTTNTNSTELKNIKEIMKNCTFNMTTEVRDKKKEVYSLFYVDDIVPIGGNKSNDTYRLINCNTTTVTQACPKTSFEPIPIHYCAPPGFAIMKCNDQKFNGTGPCTNISTVQCTHGIRPVVTTQVILNGSLAEEGPAVARSEDLRHKIIIIQFKEGINIICMRPGNNSRGQLQIGPGVSFYNIENIVGDPRQAHCNVSKAQWMKKLNETALAIAKYDKSIAKVTFTPNPGGDPEITNMMFNCGGEFFYCNTTPLFNKFNYTCDNQNNCTRNNTNDTNSDNSTLTISCRIRQIVNSWMRVGKGIYAPPIRGNITCISNITGLLLEAGGKWENNTITVYPSGGEMRDLWRLELNRYKVVSIEPIGVAPTKAKRHTVAREKRAAFGLGALFLGFLGAAGSTMGAASVVLTVQARHLLSGIVQQQNNLLRAIEAQQHLLQLSVWGIKQLQARVLAVERYLKDQQILSLWGCSGKAICYTTVPWNATWSANTSYDEIWNNLTWQDWDKKVKNYSGVIFSLIEQAQEQQNTNEKDLLELDQWSSLWSWFNITQWLWYIKIFLIVVAGLIGFRLIGIVMSVIAKVRQGYSPLSLQTLIPTTREPDRRGGTEEDGGEPGRGRSTRLVSGFLALVWEDLRNLLIFFYHRLIDLLSILWRTVQILGQSINKGLQQLKDFSTYLKGVLTYWGREIQISATSLLDTTAIAVANGTDRIIEFVQIVGRGILHIPRRIRQGLERSLL

SEQ ID NO: 322 - protein, 864 aa, Simian immunodeficiency virus
MKVMEERKRSFNGFYMTMTICMALLIPCLTSDEWVTVYYGVPVWREVNTVLFCASDAKAHSTEAHNIWATHACVPTDPNPQEVVLHNVTEDFNMWNNQMVEQMQEDISSLWDQSLKPCVKLTPLCVTMKCSNVTRQNSTSNSNNNPTLKNNTSGKNETGVLQMKNCTFNTTTELRDKKKQVYSLFYVDDLQSLGSGTGDTYTMINCNTTAITQACPKVSFEPIPIHYCAPAGFAILKCNDVNFSGKGKCRNVSTVHCTHGIKPVVTTQLIINGSLATENITVRVNNASKNTHDWIVQLSTAVNLTCKRVGNNTRGKVQIGPGMTFYNMDHIFGDTRKAFCELNGTTWNETLQKVRESLIKEIKANANGTYNITFEPSSGGDPEIANHMFNCGGEFFYCDTRKMFNESEPFHENMTIPCRIRQIVNSWMRVGRGIYAPPIPGHITCNSLITGLILTRDHVNNTNNTFRPIGGDMKNIWRSELYKYKVVRIEPLSVAPTKAKRHTVGERRQKRAAFGLGALFLGFLGAAGSTMGAAAVTLTVQARQLLSGIVQQQNNLLRAIEAQQHLLQLTVWGVKQLQARLLAVERYLQDQQILGLWGCSGKSICYTTVPWNKTWSGKSMSDIWNNLTWQQWDKLITNYTGTIFGLLEEAQSQQEKNEKDLLELDQWASLWNWFDITNWLWYIKIFLMAVGGIIGLRIIMSVVSVIRRVRQGYSPLSLQTLIPAQREPGTLGETGGEGGEPGSGRSVRLVTGCWPLIWDDLRNLVIWSYRSLTSLACIVWRQLEKLGQLLINILRLIQEKLTLLRGIIQYWGRELRTSATSLLDATAIAVGEGTDRIIRAVQIVFRIIGNIPRRIRQGLERTLL

SEQ ID NO: 323 - protein, 880 aa, Simian immunodeficiency virus
MKAMETQRNCRTLSLKEIILCTLVLGIIGIIKCEDNMWVTVYYGVPVWREADTTLFCASDAKAQNPEVHNVWASQACVSTNPNPEEIELTNVTEYFNAWENNMVEQMHEDIVNLWDQSVKPCVKLIPLCVTLNCSLFKCIKENGNTTNCTVQISTGNDSTANNITVGTIDMYNCSFNATTELRDRKKQVYSLFYRQDLEPLEGNKPPEGDKNALYRLYNCNTTAMTQACSKVSLEPIPIHYCAPAGFALLKCNDKNFTGIGQCKNVSTVHCTHGIRPVVSTQFLLNGTLEEKVTVLDRNVSNDMDTIIVKLNETVRLNCTRTGNNTIKGIPIGPSQIFYGIETVIGDTRQAFCQLNKTVWTNTFKKVRHALNETYKGYLGNETITFGPSTGGDLEVTNLHLICGGEFFYCNTSILFNTSIIFNETKDDNITIPCRIRQIVRLWQRVGRGIFLPPIRGTINCISNITGILFAQQKTDRMNKSAMFTPVGGEMRNNWRSELYKYKVVRIEPLGVAPTKAKRRTVHREKRAAVGLGALFLGFLGAAGSTMGAASLTLTVQARQLLSGIVQQQSNLLRAIEAQQHLLQLSVWGIKQLQARVLAVERYLKDQQLLGLWGCSGKLICTTSVPWNTTWTNKSYDDIWYNMTWMQWDKEVSNYTDVIYNLLEKAQTQQENNEKELLELDKWASLWNWFDITSWLWYIKIFIIIVGGLIGLRIVFALLSIVNRVRQGYSPLSFQTLIPARRDRDRPEEIEEGGGEPDNVRSIRLVSGFLALAWNDLRDLCLFLYHRLRDLLLIVLRTLELVGQTLLKGLRRGREALIHLRGILQYWGQELKTSAISLLDTTAIAVAEGTDRIIEIAQRFGRGILNIPRRIRQGLERALL

SEQ ID NO: 324 - protein, 845 aa, Simian immunodeficiency virus
MRVKGTWTSCMPYYVVGILSLCSLIISSTKNENLWVTVYYGVPVWKEAEATLFCASDAKAQKAEAHNIWASQACVPTDPDPKAIELSNVTENFNMWTNAMVNQMQQDVISLWDQSLKPCIKLTPLCVTLHCSIPKFDNSSINSSNETMKNCSFNTTTELRDKKKQTYSLFYVDDLTQINKTTESYKLINCNTTAITQACPKISFEPIPIHYCAPAGFALLRCNNKTYNGSGPCNNVSTVHCTHGIKPVISTQLILNGSLAEEKIMIRYKADKVLVQLNTSIHINCTRVGNKTIKGIPLGPGQLFYGTETVVGDTREAHCEINQTHWYKILNQVKRELTTVFNESNKTVHFANSSGGDPEVANLHFNCGGEFFYCNTSALFNDTLLRNKTLLNETLHNNLTLPCRIRQIVNLWLRVGKGIFAPPIRGNIRCNSTITGLILEKHTNTDNITFRPIGGDMTDIWRSELYNYKIVKIEPLGVAPTRARRRTVDREKRAAGLGVFFLGFLGAAGSTMGAASLTLTVQARQLLTGIVQQQSNLLRAIEAQQHLLQLSVWGIKQLQARVLAIERYLKDQQLLGIWGCSGKLICTTSVPWNRTWSNKTYNEIWDNMTWMEWDREVRNYTEIIYGLIEQAQDQQENNEKKLLELDHWTSLWNWFDISHWLWYIKIFIMIIGGLIVCRIIFAVLAIVNRVRQGYSPLSFQTLSPIPRGLAQPGGTEEEGGEQGTDRSVRLLDGFLALAWNDLRDLCLFLYHRLTDLVLIARRTLEIVGQYTLKGLRLVWEALLYLQGILQYWGKELKTSATSLLDTTAIAVAEGTDRIIEIVQRIGRGILHIPRRIRQGFERALL

SEQ ID NO: 325 - protein, 835 aa, Simian immunodeficiency virus
MKVTEMQKNWLICCLLIGLIKIIGSELWVTVYYGVPVWRDAETVLFCASDAKAHSTEAHNIWATQACVPTDPNPQEVLIPNVTERFDMWKNNMVDQMQEDIISLWEQSLKPCVKLTPLCVTLSCSSWRSVNNSVNQTNHVQMQNCSFNVTTELRDKKKQVYSLFYMGDIIPLDTNNSSGNNSQYRLINCNTTAVTQACPKISFEPIPIYYCAPPGFAIIKCNDQDFNGTGECNNVSTVQCTHGIKPVISTQLILNGSLATSNIVIRNNSKDTLLVQLNESIPINCTRPGNKTRGQVQIGPGMTFYNIENIIGDTRQAYCEVNRTWEQIWNTTKQIIINNRKNITFIPNPGGDLEVTNLMINCGGEFFYCNTSQLFTNQNGNTTGNITLQCRIRQIVNLWTRVGKGIYAPPIKGPINCLSNITGIILDYTKSGTEKYTIYPTGGDMTNLWRQELYKYKVVSIEPIGVAPGKAKRHTVTRQKRAAFGLGALFLGFLGAAGSTMGAASITLTVQARKLLSGIVQQQNNLLRAIEAQQHLLQLSVWGIKQLQARVLAIERYLRDQQILGLWGCSGKSVCYTNVPWNTTWSNNNSYDTIWGNMTWQNWDEQVRNYSGVIFGLLEQAQEQQSINEKSLLELDQWSSLWNWFDITKWLWYIKIFIMVVAGIVGIRIISIIMSMVARVRQGYSPLSLQTLIPTTRGPDRPERTEEDAGELDNGRSVRLVSGFLALAWEDFRNLLLFLYHRLTDCLSILRRTLELLRQNIHKGLQLLNELRIYLWGIIAYWGRELKISAINLLDTTAVAVAEGTDRIIELVQRIGRGILHIPRRIRQGLERALL

SEQ ID NO: 326 - protein, 855 aa, Simian immunodeficiency virus
MKVMGMKYYWWRNSCRSISIKLILIGWIASCFGEENWWVTVYYGVPVWREAKTTLFCASDAKSYSTEAHNIWATQACVPTDPTPQEVLLPNVTEEFNMWENYMVDQMQEDIISLWEQSLKPCVKLTPLCVTLTCNNPTNTSCTNSTDDRLGDMRNCSFNVTTELRDKKRQVYSLFYVEDITAIGNNSTYRLINCNTTAITQACPKTSFEPIPIHYCAPAGFALLKCNDIDYKGNETCKNVSTVHCTHGIKPVATTQLILNGSTADNQTVARIDPSENLAIIQLKDPVKITCRRPGNNTRGQIQIGPAMTFYNIENVVGDTRKAYCEINGTQWAKALNETKEVLRNILRKNISFMVPSGGDPEVTNHHFNCGGEFFYCNTSEIINITKINKTENMTIIPCRIRQIVNSWMRVGKGIFAPPIRGNITCTSNITGMLLEIHKNREDQGEDQDQNNTYVCLTGGNMKDIWRSELYKYKIVEIQPLGVAPTKSRRYAVEKQHHREKRALGLGALFLGFLGAAGSTMGAASVVLTVQARQLLTGIVQQQNNLLRAIEAQQHLLQLSVWGIKQLQARVLAVERYLRDQQLLGLWGCTGKTICPTAVRWNKTWGNISDYQVIWNNYTWQQWDREVNNYTGLIYTLLEEANTQQEKNEKELLELDSWANLWSWFDITNWLWYIKMFLIVVGGIIGLRICFAIGSLINRVRKGYSPLSLQTLIPANQGPDGLGETEKGGGENVRGRSIRLVSGFLPLVWEDLRNLLSFLYHQLRDCASLIWRLLEILGQYSLRGVQQIGTLLHQLRGTLQYWTTEIKNSAISLLDTTAIAVGEGTDRILEAITRLGRGILHIPRRIRQGLERALL

SEQ ID NO: 327 - protein, 876 aa, Simian immunodeficiency virus
MRNLIGTTLTLIIIILGIGSKEYYTTVFYGVPIWKEATPSLFCASNADIASRDKHNIWATHNCVPLDPNPYEVPLANVSIDFDMEKNYMVEEMKEDLLSLFQQSFKPCVKLTPFCVTMKCTVVNTTVVNATAPTPKVSATVATTPTINLTNIKEYEIFNCSFNQTTEFRDKKKQIYSLFYREDIMKVGSESTKNTDYYLHNCNTSAITQECDKSTFEPIPIRYCAPAGFAMLKCNDQGYTGNGTCSNVTVVHCTHGIFPMIATALHLNGTLEENETKAYFADVNVNPPLLVKFNESVSLTCERTGNNTRGQVQIGPGMTFYNIENVVGDTRKAYCHVNATLWDQSLSRAMEAINKTLALYNKAVNEKFEWSKGDPEVSSFWFNCQGEFFYCNLTGWTNPWNNTNHRNGTLVAPCRLRQIVNHWGIVSKGIYLPPRRGTLKCNSNITGLIMTAEKGGNNTVPTFSGKVEDYWKVELARYKVVEIQPLSVAPRPGKRPEIKANHTRSRRDVGIGLLFLGFLSAAGSTMGAASIALTAQARGLLSDIVQQQQNLLQAIEAQQHLLQLSVWGIKQLQARMLAVEKYIRDQQYLSIWGCANKLVCHSSVPWNLSWAEDSQKCNSTNTKYYECIWNNLTWQEWDRLVQNASETIYSLLEIAQTQQEKNKQELLELDKWSSLWDWFDITQWLWYIRLAIIIVAGLVGLRILMFIVNVVKKVRQGYMPLFSQTPTQAQQDPEQPGGIAEGGGGRGNIRWTPSPTGFFSIVWEDLRNLLIWLYQTCRNFIWVLWTILQALKQGTISLANNLVIVHRYIVVKIRQIIEWCHNTYASLRASLIHAIDRLADFTGWWTDLIIEGITYIGRGIRNIPRRIRQGLEIALN

SEQ ID NO: 328 - protein, 896 aa, Simian immunodeficiency virus
MKMRNLIGITLILIILTLGIGSRDYYTTVFYGVPIWKDATPSLFCASDADVTSREKHNIWATHNCVPLDPNPYEVPIANVSIKFNMEENYMVKEMKEDLLSLFQQSFKPCVKLTPFCVVMNCTKSQNVTTPASTSTAAPVNATTIPVNLTDIPNYELFNCSFQQTTEFRDKKKQIYSLFYREDIMKDNRSMQDNRSNSSKKSNNSNNSGYYLHNCNTSAITQECDKSTFEPIPIRYCAPAGYAMLKCKDQNYTGNGICYNVTVVHCTHGIFPMIATALHLNGTLEEKETKAYFVNATNNPPLLVKFNESVPLTCGRTGNNVRGQVQIGPGMTFYNTENVVGDTRKAYCWVNATLWYKSLDNAMEAITKTLKKSGLNVTRRFDWHKGGDLEVQNFWFNCQGEFFYCNLTTWTGNWTRNRTHQTSALNKTHLLAQCRLRQIVNHWGIVSKGIYLPPRKGHIKCVSNITGLIMTAEGGNSNRTPTFSSKVEDYWKVELARYKVVEIQPLSVAPRPGKRPEIKANHTRSRRDVGIGLLFLGFLSAAGSTMGAASLALTAQARGLLSGIVQQQQNLLQAIEAQQHLLQLSVWGIKQLQARMLAVEKYIRDQQYLSIWGCANKLVCHSSVPWNLSWAGTSSKCNYTGAKYYECIWNNLTWQEWDKLVQNSSETIYSLLETAQTQQERNKQELLELDKWSSLWDWFDITQWLWYIKIAIIIVAGLVGLRILMFIVNVVKKVRQGYMPLFSQTPTQAEQDPEQPGGTAGGGGGGGNFRWTPSPTGFFSIVWEDLRNLLIWIYQTCQNFIWVLWTILQALKQGTISLAHNLVIVHRYIIVRVRQTIEWCGNTYASLRASLIHAIDRLADFTGWWTDLLIEGVVYIARGIRNIPRRIRQGLEIALN

SEQ ID NO: 329 - protein, 871 aa, Simian immunodeficiency virus; identical to SEQ ID NO: 320

SEQ ID NO: 330 - protein, 876 aa, Simian immunodeficiency virus
  VARIANT 81: note = Xaa can be any naturally occurring amino acid
  VARIANT 91: note = Xaa can be any naturally occurring amino acid
  VARIANT 179: note = Xaa can be any naturally occurring amino acid
  VARIANT 428: note = Xaa can be any naturally occurring amino acid
MRNLIGTTLTLIIIILGIGSKEYYTTVFYGVPIWKEATPSLFCASNADIASRDKHNIWATHNCVPLDPNPYEVPLANVSIXFDMEKNYMVXEMKEDLLSLFQQSFKPCVKLTPFCVTMKCTVVNTTVVNATAPTPKVSATVATTPTINLTNIKEYEIFNCSFNQTTEFRDKKKQIYSLXYREDIMKVGSESTKNTDYYLHNCNTSAITQECDKSTFEPIPIRYCAPAGFAMLKCNDQGYTGNGTCSNVTVVHCTHGIFPMIATALHLNGTLEENETKAYFADVNVNPPLLVKFNESVSLTCERTGNNTRGQVQIGPGMTFYNIENVVGDTRKAYCHVNATLWDQSLSRAMEAINKTLALYNKAVNEKFEWSKGDPEVSSFWFNCQGEFFYCNLTGWTNPWNNTNHRNGTLVAPCRLRQIVNHWGIVSXGIYLPPRRGTLKCNSNITGLIMTAEKGGNNTVPTFSGKVEDYWKVELARYKVVEIQPLSVAPRPGKRPEIKANHTRSRRDVGIGLLFLGFLSAAGSTMGAASIALTAQARGLLSDIVQQQQNLLQAIEAQQHLLQLSVWGIKQLQARMLAVEKYIRDQQYLSIWGCANKLVCHSSVPWNLSWAEDSQKCNSTNTKYYECIWNNLTWQEWDRLVQNASETIYSLLEIAQTQQEKNKQELLELDKWSSLWDWFDITQWLWYIRLAIIIVAGLVGLRILMFIVNVVKKVRQGYMPLFSQTPTQAQQDPEQPGGIAEGGGGRGNIRWTPSPTGFFSIVWEDLRNLLIWLYQTCRNFIWVLWTILQALKQGTISLANNLVIVHRYIVVKIRQIIEWCHNTYASLRASLIHAIDRLADFTGWWTDLIIEGITYIGRGIRNIPRRIRQGLEIALN

SEQ ID NO: 331 - protein, 896 aa, Simian immunodeficiency virus
  VARIANT 259: note = Xaa can be any naturally occurring amino acid
  VARIANT 287: note = Xaa can be any naturally occurring amino acid
  VARIANT 322: note = Xaa can be any naturally occurring amino acid
  VARIANT 336: note = Xaa can be any naturally occurring amino acid
  VARIANT 338: note = Xaa can be any naturally occurring amino acid
  VARIANT 340: note = Xaa can be any naturally occurring amino acid
  VARIANT 361: note = Xaa can be any naturally occurring amino acid
  VARIANT 371: note = Xaa can be any naturally occurring amino acid
  VARIANT 379..380: note = Xaa can be any naturally occurring amino acid
  VARIANT 382: note = Xaa can be any naturally occurring amino acid
MKMRNLIGITLILIILTLGIGSRDYYTTVFYGVPIWKDATPSLFCASDADVTSREKHNIWATHNCVPLDPNPYEVPIANVSIKFNMEENYMVKEMKEDLLSLFQQSFKPCVKLTPFCVVMNCTKSQNVTTPASTSTAAPVNATTIPVNLTDIPNYELFNCSFQQTTEFRDKKKQIYSLFYREDIMKDNRSMQDNRSNSSKKSNNSNNSGYYLHNCNTSAITQECDKSTFEPIPIRYCAPAGYAMLKCKDQNYTGNGICXNVTVVHCTHGIFPMIATALHLNGTLEEXETKAYFVNATNNPPLLVKFNESVPLTCGRTGBNVXGQVQIGPGMTFYNXEXVXGDTRKAYCWVNATLWYKSLDXAMEAITKTLXKSGLBVTXXFXWHKGGDLEVQNFWFNCQGEFFYCNLTTWTGNWTRNRTHQTSALNKTHLLAQCRLRQIVNHWGIVSKGIYLPPRKGHIKCVSNITGLIMTAEGGNSNRTPTFSSKVEDYWKVELARYKVVEIQPLSVAPRPGKRPEIKANHTRSRRDVGIGLLFLGFLSAAGSTMGAASLALTAQARGLLSGIVQQQQNLLQAIEAQQHLLQLSVWGIKQLQARMLAVEKYIRDQQYLSIWGCANKLVCHSSVPWNLSWAGTSSKCNYTGAKYYECIWNNLTWQEWDKLVQNSSETIYSLLETAQTQQERNKQELLELDKWSSLWDWFDITQWLWYIKIAIIIVAGLVGLRILMFIVNVVKKVRQGYMPLFSQTPTQAEQDPEQPGGTAGGGGGGGNFRWTPSPTGFFSIVWEDLRNLLIWIYQTCQNFIWVLWTILQALKQGTISLAHNLVIVHRYIIVRVRQTIEWCGNTYASLRASLIHAIDRLADFTGWWTDLLIEGVVYIARGIRNIPRRIRQGLEIALN

SEQ ID NO: 332 - protein, 852 aa, Simian immunodeficiency virus
  VARIANT 174: note = Xaa can be any naturally occurring amino acid
  VARIANT 178..179: note = Xaa can be any naturally occurring amino acid
  VARIANT 179: note = Xaa can be any naturally occurring amino acid
  VARIANT 261..262: note = Xaa can be any naturally occurring amino acid
  VARIANT 266: note = Xaa can be any naturally occurring amino acid
  VARIANT 268..269: note = Xaa can be any naturally occurring amino acid
  VARIANT 272: note = Xaa can be any naturally occurring amino acid
  VARIANT 322: note = Xaa can be any naturally occurring amino acid
  VARIANT 331..332: note = Xaa can be any naturally occurring amino acid
  VARIANT 335: note = Xaa can be any naturally occurring amino acid
MRVMERIRGSLWLMGLIIPCLTGSDQLWATVYYGVPVWKDAETTLFCAADASALNKEAHNIWASQACVPTDPNPQEVHVPNITETFDMWENNMVEQMQTDIISLWDQSLKPCVKLTPLCVTMHCIPYNISSTGNDTRNSSTSQMVKKCFFNMTTELRDKQKQVYSLFYVDDIVXINGXXSYRLINCNTTAITQACPKTSFEPIPIHYCAPPGFAIMKCNEPDFNGTGKCRNISTVQCTHGIKPVVTTQLILNGSLAENSTXXRTEXMXXMDXTIIVQFHKRVQMTCIRPGNNSRGQIQIGPAMSFYNLENIIGDTRQAYCNXSAAEWEQRXXDTXRAIKSLKPGSNITFTDQQGGDPEIVNMMFNCGGEFFYCNTTPLFNNSWTNTTSSDINSSNIIINCRIRQIVNSWMRVGKGIYAPPIRGTISCTSNITGLLLELDRGLPRNGTNTNNKLTLYPTGGEMRDLWRLELHKYKVVSIEPIGVAPSKAKRHTVTREKRAAFGLGALFLGFLGAAGSTMGAASLTLTVQARQLLSGIVQQQNNLLRAIEAQQHLLQLSVWGIKQLQARVLAVERYLKDQQILGLWGCSGKSICYTTVPWNSTWGANTSYDEIWNNLTWQDWDKRVKNYSGVIFDLIEQAQEQQNTNERSLLELDQWSSLWNWFDISNWLWYIKIFLMAVAGLIGIRIVGVIMSLIAKVRQGYSPLSLQTLIPTTRGPDRPGGTEEEGGEPGRGTSTRLVSGFLALAWDDLRNLLLFLYHRLIDLLSILRRTLEILGQNINKGLQWLRDISRHLWGVIAYWGRELQISATSLLDTTAIAVAEGTDRIIEVVQRIGRGILHIPRRIRQGLERSLL

SEQ ID NO: 333 - protein, 844 aa, Simian immunodeficiency virus
MKVMGTQRSWKQFLSLRIVMCLWLLGVIRSENNLWVTVYYGVPVWKEAKTTLFCASDAKAQIAEAHNIWASQACVPTDPNPEEITLENVTEEFDAWNNNMVDQMQEDLISLWDQSLKPCVKLTPLCVTLNCSTNTTTEASQVQYNCSFNVTTELRDKKKQVYSLFYREDITSLDSNKTVKNGTYRLINCNTTAITQACPKTSFEPIPIYYCAPAGFALLKCNDQNFKGKGTCRNVSTVHCTHGIKPVVSTQFLLNGSLAEGNKTVVRVRSKSNTETIIVQLATAIYINCTRLGNKTIEGIPIGPGQIFYRTKTVVGDTRGAECRINGTAWNETLRQVKEALNNTYRNLNLSLTEINFEGASGGDLEVTTHYFNCGGEFFYCNTSNLFNQSTINNITHIPCRIRQIVNQWQGVGKGIFAPPIRGTIQCNSTITGLLLTRDGKNETETFRPTGGDMRNNWRSELYKYKVVKIEPLGLAPTKAKRRTVQREKRAVGLGVMFLGFMGAAGSTMGAAALTLTVQARNLLSGIVQQQSNLLKAIEAQQHLLQLSVWGIKQLQARVLAVERYLKDQQLLGLWGCSGKLICTTNVPWNNTWSKKLGTNLSFWDNMTWMQWEKEIDNYTETIYELLTRSQNQQEVNEQELLALDKWSSLWNWFDITQWLWYIKLFVMIVGGLIGIRIVFAMLSIVNRVRQGYSPLSFQTPFPVRRDPDRPEGIEEGGGENDNVRSVRLVSGFLALAWDDLRNLCLFSYHRLRDLLWIVRRTLEIVGQKFLAGLRLVWETLTYLKSILQYWGQELKTSAISLLDATAIAVAEGTDRIIEIAQRVGRGILHIPRRIRQGLERALL

SEQ ID NO: 334 - protein, 867 aa, Simian immunodeficiency virus
  VARIANT 86: note = Xaa can be any naturally occurring amino acid
  VARIANT 141: note = Xaa can be any naturally occurring amino acid
  VARIANT 554: note = Xaa can be any naturally occurring amino acid
MRDMRAMRKKQLNLWKGVMSLGLILTCIEATHYATVYYGVPVWQEANVTLFCAADANYVSQEQHNIWATQACVPTDPSPIEFPLEXVTETFDIWNNNMVDQMQDDIISLWDQSLKPCVKLTVMCVTLNCTKTTETTTQTNXTNTTIPPYIPQLDIYNCSFNVTTVLKDKKTKQQALFYKQDIIKTDETNETKEYSEYRLINCNTSTISQACPKVSFEPLPIQYCAPAGYALMKCNDKQFNGTGECKNVSIVHCTHGIRPTVSTQLILNGTLAKEHATIISKNSTDSGKDIIVKLARSVEITCERTSNNTRGQIQVGPMTIYNSENIVGNTRKAFCKYNKTNWQNALKDTVRALKANQIRFQNSSGGDPEVTFLHFNCHGEFFYCDTTKMFNYNCTKESCDCITGNCTNYIPCHLKQVVMSWMRVGSGLFAPPIRGTLRCKSNITGIILQRDVPLNKTNENSTDYNTLRPIGGDMTNIWRSELYPYKVVKVKALSVAPTKARRPVISHNREKRAAGLGMLFLGFMSAAGSTMGAAAVTLTVQARQVLHGIVQQQXNMLRAIAAQQELLRLSVWGIRQLRARLLAIETYLRDQQLLGLWGCTGRLICYTNVPWNKTWTGKNETELDDIWGNMTWQQWDKLVDNYTDTIFLEIQKAQEQQEVNEKALLELDKWADLWSWLDITQWLWYIKIFIMIIAGLVGLRILMAIINMCHRVRQGYSPLSFQILARNQVPAGIVPEIGEEGGRADSDRSIRLLAGFLPLIWDDLRTLIVWIYRTLVILLSGIKEITLSLIEHLTRLLRRVNNLTRDCFAFIAYWGQELKQSAISLLDCVAVWTADWTDQVIAIAQRIGRGILNIPRRIRQGLERSLL

SEQ ID NO: 335 - protein, 872 aa, Simian immunodeficiency virus
MRDMRAMRKKQLNLWKWVMSLGLILTCIEATHYATVYYGVPVWREANVTLFCAADANYVSKEQHNIWATQACVPTDPRPTEFPLENVTETFNIWKNYMVDQMQDDIVSLWDQSLKPCVKLTVMCVTLNCTQLNTTNTTQTEMTNTTIPPHTTIPPHIPQLDIYNCSFNVTTVLKDKTTKQQALFYRQDIIETGETGTNGIKGYRLINCNTSTISQACPKVSFEPLPIQYCAPAGYALMKCNDIQFNGTGECRNVSIVHCTHGIRPTVSTQLILNGTLAEGNATIISKNSSDSGKDIIVKLARPVEITCERTGNNTRGQIQIGPMTIYNSENIVGNTRKAFCKYNETNWQGALKDTVQALNESLIHFRNSSGGDSEVTFLHFNCHGEFFYCNTTKMFSYNCTTEGCNTTETSNTTETTKYIPCHLKQVVRSWMRVGSGLFAPPIRGTLRCISNITGIILQRDAPLKTDENSTLRPIGGDMTNIWRSELYPYKVVRVKALTVAPTKAKRPVIGHNREKRAAGLGMLFLGFMSAAGSTMGAAAVTLTVQARQVLHGIVQQQNNMLRAIAAQQELLRLSVWGIRQLRARLLAIETYLRDQQLLGLWGCTGRLICYTNVPWNKTWTGKNDTELDDIWGNMTWQQWDKLVDNYTDTIFLEIQRAQEQQEANGKALLELDKWADLWSWLDITQWLWYIKIFIIIIAGLVGLRILMAIINMCHRVRQGYSPLSFQILGRNQVPAGIVPEIGEEGGRADNDRSIRLLAGFLPLIWDDIRTLVVWIYRTLVILLSGIKEITLSLIEYLTRLLRRVNNLTRDCFAFIAYWGQELKQSAISLLDCVAVWTANWTDQVIAIAQRIGRGILNIPRRIRQGLERSLL

SEQ ID NO: 336 - protein, 872 aa, Simian immunodeficiency virus
  VARIANT 630: note = Xaa can be any naturally occurring amino acid
  VARIANT 633..634: note = Xaa can be any naturally occurring amino acid
  VARIANT 666: note = Xaa can be any naturally occurring amino acid
  VARIANT 696..697: note = Xaa can be any naturally occurring amino acid
  VARIANT 728: note = Xaa can be any naturally occurring amino acid
  VARIANT 808: note = Xaa can be any naturally occurring amino acid
MRDMRAMRKKQLNLWKWVMSLGLILTCIEATHYATVYYGVPVWREANVTLFCAADANYVSKEQHNIWVTQACVPTDPRPTEFPLENVTETFNIWKNYMVDQMQDDIVSLWDQSLKPCVKLTVMCVTLNCTQLNTTNTTQTEMTNTTIPPHTTIPPHIPQLDIYNCSFNVTTVLKDKTTKQQALFYRQDIIETGETGTNGIKGYRLINCNTSTISQACPKVSFEPLPIQYCAPAGYALMKCNDIQFNGTGECRNVSIVHCTHGIRPTVSTQLILNGTLAEGNATIISKNSSDSGKDIIVKLARPVEITCERTGNNTRGQIQIGPMTIYNSENIVGNTRKAFCKYNETNWQGALKDTVQALNESLIHFRNSSGGDPEVTFLHFNCHGEFFYCNTTKMFSYNCTTEGCNTTETSNTTETTKYIPCHLKQVVRSWMRVGSGLFAPPIRGTLRCISNITGIILQRDAPLKTDENSTLRPIGGDMTNIWRSELYPYKVVRVKALTVAPTKAKRPVIGHNREKRAAGLGMLFLGFMSAAGSTMGAAAVTLTVQARQVLHGIVQQQNNMLRAIAAQQELLRLSVWGIRQLRARLLAIETYLRDQQLLGLWGCTGRLICYTNVPWNKTWTGKNDTELDXIWXXMTWQQWDKLVDNYTDTIFLEIQRAQEQQEANXKALLELDKWADLWSWLDITQWLWYIKIFIXXIAGLVGLRILMAIINMCHRVRQGYSPLSFQXLGRNQVPAGIVPEIGEEGGRADNDRSIRLLAGFLPLIWDDIRTLVVWIYRTLVILLSGIKEITLSLIZYLTRLLRRVNNXTRDCFAFIAYWGQELKQSAISLLDCVAVWTANWTDQVIAIAQRIGRGILNIPRRIRQGLERSLL

SEQ ID NO: 337 - protein, 872 aa, Simian immunodeficiency virus
MRDMRAMRKKQLNLWKWVMSLGLILTCIEATHYATVYYGVPVWREANVTLFCAADANYVSKEQHNIWATQACVPTDPRPTEFPLENVTETFNIWKNYMVDQMQDDIVSLWDQSLKPCVKLTVMCVTLNCTQLNTTNTTQTEMTNTTIPPHTTIPPHIPQLDIYNCSFNVTTVLKDKTTKQQALFYRQDIIETGETGTNGIKGYRLINCNTSTISQACPKVSFEPLPIQYCAPAGYALMKCNDIQFNGTGECRNVSIVHCTHGIRPTVSTQLILNGTLAEGNATIISKNSSDSGKDIIVKLARPVEITCERTGNNTRGQIQIGPMTIYNSENIVGNTRKAFCKYNETNWQGALKDTVQALNESLIHFRNSSGGDPEVTFLHFNCHGEFFYCNTTKMFSYNCTTEGCNTTETSNTTETTKYIPCHLKQVVRSWMRVGSGLFAPPIRGTLRCISNITGIILQRDAPLKTDENSTLRPIGGDMTNIWRSELYPYKVVRVKALTVAPTKAKRPVIGHNREKRAAGLGMLFLGFMSAAGSTMGAAAVTLTVQARQVLHGIVQQQNNMLRAIAAQQELLRLSVWGIRQLRARLLAIETYLRDQQLLGLWGCTGRLICYTNVPWNKTWTGKNDTELDDIWGNMTWQQWDKLVDNYTDTIFLEIQRAQEQQEANEKALLELDKWADLWSWLDITQWLWYIKIFIIIIAGLVGLRILMAIINMCHRVRQGYSPLSFQILGRNQVPAGIVPEIGEEGGRADNDRSIRLLAGFLPLIWDDIRTLVVWIYRTLVILLSGIKEITLSLIEYLTRLLRRVNNLTRDCFAFIAYWGQELKQSAISLLDCVAVWTANWTDQVIAIAQRIGRGILNIPRRIRQGLERSLL

SEQ ID NO: 338 - protein, 876 aa, Simian immunodeficiency virus
  VARIANT 345: note = Xaa can be any naturally occurring amino acid
  VARIANT 446: note = Xaa can be any naturally occurring amino acid
  VARIANT 450: note = Xaa can be any naturally occurring amino acid
  VARIANT 454: note = Xaa can be any naturally occurring amino acid
  VARIANT 455: note = Xaa can be any naturally occurring amino acid
  VARIANT 460: note = Xaa can be any naturally occurring amino acid
  VARIANT 467: note = Xaa can be any naturally occurring amino acid
  VARIANT 801: note = Xaa can be any naturally occurring amino acid
  VARIANT 807: note = Xaa can be any naturally occurring amino acid
MKDMRAMRKKQLNLWKGVMSLGLLLTCIEAKTEHYATVYYGVPVWREAKVTLFCAADASFVSKEQHNIWATQACVPTDPSPTEFLLENVTETFDIWKNYMVEQMQEDIISLWDQSLKPCVKLTVMCVTMTCSNDTKGSNKSTATTTSANTPTSTETTIPTNIPELDIFNCTFNVTTVLKDKKTKQQALFYKQDITETDKNEYRLINCNTSTISQACPKVSFEPLPIQYCAPAGYPLLKCNDEQFNGTGECNNVSIVHCTHGIRPTVSTQLILNGTLAKGNNATIISSNSSDSATDIIVKLANPVEITCERTGNNTRGQIQVGPLTIYNSENIIGNTRKAFCKYNXTAWQKALQATVRALKTNLTHYFNKSSGGDPEVTSLHFNCHGEFFYCNTTKMFNYKCTGEHCNCTDKELCNKTAEMEYIPCRLKQVVNSWMRVGSGLFAPPXRGTXRCIXXITGIXLQRDTPXAENNNNTTLRPLGGEMRNIWRSELYPYKVVKVKALSVAPTKAKRPVVSHNREKRAAGLGMLFLGFMSAAGSTMGAAAITLTVQARQVLHGIVQQQNNMLRAIAAQQELLRLSVWGIRQLRARLLAIETYLRDQQLLGLWGCTGKLICYTNVPWNTTWTGKSDSELDGIWGNLTWQQWDKLVDNYTDTIFLEIQKAQEQQEANEKALLELDKWADLWSWLDITQWIWYIRIFIIVIAGLVGLKILMAIINICHRVRQGYSPLSFQILGRNQVPAGIVPEIGEEGGRADSDRSIRLLAGFLPLIWDDLRTLVVWIYRTLVILLSGIKEITLSLIEXLTRLLXRVNNLTRDCFAFIAYWGQELKQSAISLLDCVAVWTADWTDQVIAIAQRIGRGILNIPRRIRQGLERSLL

SEQ ID NO: 339 - protein, 870 aa, Simian immunodeficiency virus
  VARIANT 459: note = Xaa can be any naturally occurring amino acid
  VARIANT 621: note = Xaa can be any naturally occurring amino acid
  VARIANT 682: note = Xaa can be any naturally occurring amino acid
MRAMERTKRSWNILSRSMVLLTLCLTFNVATDYWVTVYYGVPVWKDAETTLFCASEAKPHSSEAHNIWATQACVPIDPNPEEVFLPNVTENFSMWDNPMVSQMQEDIISLWEQSLKPCVKLTPLCVTLNCSTAIFRANRTSTNTTTPSTIATSPTTDTIYEMKNCSFNVTTELRDKKKQVYSLFYVDDVVKLNDGDSTNSSTYRLINCNTTAITQACPKISFEPIPIHYCAPAGFAILKCNDTNYLGTGKCHNVSTVHCTHGIRPAVTTQLILNGSLAEGNVTIRVKNASSNSGNYIVQLARSVEILCRREGNNTRGQVQIGPAMTFYNIENVVGDTRKAYCTLNGSNWEEAMSKVKKELEKITNVTNITFADQRQGSDPEVENHMFNCGGEFFYCNTSKLFNNKTGRGGNKSEEIILPCRIKQIVNLWTRVGKGIYAPPIRGNISCKSNITGILLARXEGKDNQNNLTFRPIGGNMEDLWRSELYKYKVVRIEPLAVAPSKAKRRTVETEKRQKRAAFGLGALFLGFLGAAGSTMGAAAVTLTVQARQLLSGIVQQQNNLLRAIEAQQHLLQLSIWGVKQLQGRLLAVERYLQDQQILGLWGCSGKAICYTTVPWNKTWXGNYSYEYIWNNLTWEQWDVHVSNYTGVIFGLLEEAQGQQEKNERELLELDKWSSLWSWFBXTHWLWYIKIFIMVVAGIVGLRICMIVISVIRRVRQGYSPLSLQTLLPVQRGPGVLDGTDEEGGEQGRGRSVRLVTGCLPLIWDDLRSLLIWFYQTLSNSACVVQRHLERLIQLILKGLRLLWEKIRLCWGVLQYWKRELKTSAISLLDATAIRVAEGTDQIIFAAQRIGRGFLNIPRRIRQGLERALL

SEQ ID NO: 340 - protein, 868 aa, Simian immunodeficiency virus
  VARIANT 461: note = Xaa can be any naturally occurring amino acid
  VARIANT 463: note = Xaa can be any naturally occurring amino acid
  VARIANT 767: note = Xaa can be any naturally occurring amino acid
MRAMERTKRSWNILSRSMVLLTLCLTFNVATEYWVAVYYGVPVWKDAETTLFCASEAKPHSSEAHNIWATQACVPIDPNPEEVFLTNVTENFSMWDNPMVSQMQEDIISLWEQSLKPCVKLTLYVLTPNCSTAEFKNNTNTNTTTTGPANVTNYEMKNCSFNVTTELRDRKKRVYSLFYVDDVEKLNDGNSTNSTYRLINCNTTAITQACPKISFEPIPIHYCAPAGFAILKCNDTNYSGTGKCHNVSTVHCTHGIMPAVTTQLILNGSLAEGNVTIRVKNASSNSGNFIVQLAKPVEILCRREGNNTRGQVQIGPGMTYYNIENVIGNTRKAYCKLNRSNWEDAMSKVKKELEKITNVTKVIFANQSHGSDPEVENHMFNCGGEFFYCNTSKLFNNKTGINDTTGKGTNESEIILPCRLRQIVNLWTRVGKGIYAPPIRGNISCKSNITGILLAREEGBXNXTNLTFKPIGGNMEDLWRSELYKYKVVRIEPLAVAPSKAKRRTVETEKRQKRAAFGLGALFLGFLGAAGSTMGAAAVTLTVQARQLLSGIVQQQNNLLRAIEAQQHLLQLSIWGVKQLQGRLLAVERYLQDQQILGLWGCSGKAICYTTVPWNKTWFGNYSYEYIWNNLTWEQWDVHVSNYTGVIFGLLEEAQGQQEKNERELLELDKWSSLWSWFDITHWLWYIKIFIMVVAGIVGLRICMIVISVIRRVRQGYSPLSLQTLLPVQRGPGVLDGTDEEGGEQGRGRSVRLVTGCLPLIWDDLRXLLIWFYQTLSNSACVVQRHLERLIQLILKGLRLLWEKIRLCWGVLQYWTRELKTSAISLLDAIAIRVAEGTDQIIFAAQRIGRGFLNIPGRIRQGLLRALL

SEQ ID NO: 341 - protein, 798 aa, Simian immunodeficiency virus
MRVREMKKLWSFWVLGLGFLALSLTSDSNWWVTVYLGVPVWKDAETTLFCASDAKAYSTEAHNIWATQACVPIDPNPQEVFLKNVKENFNMWDNPMVDQMQEDIISLWDQSLKPCVKLTPLCVTLNCTNVNATSPTTKPPTTTPTTVSTVSTTIPLNDSIFEDMCNCTFNVTTELRGSSNNSYNSYRLINCNTTAITQACPKTSFEPIPIHYCAPAGFAILRCNEENSQEMGYCENVSTVHCTHGIKPVVTTQLILNGSITQQIMIRSKNISSNSFNIIVQFNETIPIKCIRPGNNTRGQIQIGPAMTFYNIENIVGNTRKAFCKVNGSQWWNMKQNIIQRFKAEHKLNVTFNSSAGGDPEITNFMVNCHGEFFYCNTTPLFTGNKTNITIILPCKIRQIVNSWMRVGKGIYAPPIRGNLSCNSNITGLLLTRDGPDKNINETETLRPGGGDMKDIWRSELYKYKVVKIEPLAVAPTKARRYTINMEKHRAKRAAFAAGSTMGAAAVTLTVQARNLLSGIVQQQNNLLRAIEAQQHLLQLSVWGVKQLQARLLAVERYLKDQQILGLWGCSGKAICYTNVPWNRNWTNSSQDYNEIWNNLTWNEWDKQVSNYTTEIFKALEEAQGQQEKNEKELLELDKWSSLWSWLDITQWLWYIKIFLMVVGAIIGLKIVMTVVSLIKRVRQGYSPLSLQTLIPAAGERGPPEEKEEGVGDNGRTRSIRLANGFFALFWEDLRSLFLFSYHRLRDLTSILSRILQLLYRQAVTGLQTVRKALSLLKAHIAYWGSELKSSAISLLDS

SEQ ID NO: 342 - protein, 864 aa, Simian immunodeficiency virus
MRRLIGTILTLSITILGIGYSVHYATVFYGVPVWKDAKPQLFCASDADITSRDPHNIWATHNCVPLDPHPYEVPLSNVSVDFDMENNYMVEEMKKDLIALFQQSFKPCVKLTPFCVTMNCVKLKETTTTIATITNPLVNLTGIEDYKVYNCSFNQTTEFRDKKKQIYSLFYREDIMKTDNNDSYYLHNCNTSAITQECEKSSFEPIPIRYCAPAGYAMLKCNSANFTGVGTCNNVSVVHCTHGIYPMIATALHLNGTLEKHNTTAYFATPTHNKPLLIKFNKSVDMNCTRTGNNSRGQVQIGPGMTFYNIENIVGNTRKAYCTVNYQEWSHAIGEAKKVAEEALKKNITFRWNQGGDLEVTNFWFNCQGEFFYCNLTNWTNITWLETWLKTNNSSRPLVAPCKLRQIVNHWGIVSKGIYLPPRRGNLTCRSNITGFITTWDNDTENVLVTFSAKVEDYWKVEMSRYKVVEIQPLAIAPTTGKRPEIRANHTRSKRDVGIGLLFLGFLSAAGSTMGAASIALTAQARGLLSGIVQQQHNLLQAIEAQQHLLQLSVWGIKQLQARMLAVEKYIRDQQLLSVWGCANKLVCHSNVVWNMTWAENGTTCPQGSEDYYNCIWNNLTWQQWEKLVANSTEEIYTLLEKAQVQQETNKKELFELDKWSTLWDWFDITQWLWYIKIAILIVAGLVGLRIVMFIVNVIRQVRQGYMPLFSQIPTQAHQDPEQPEETAGGGGGTGNVRWTPLHRGFFIVVWEDLRTLLLWIYQTCQNFGWLLWTILKTLRQGIISLTQRLIIVQRDIILKSRQLFDWLSNTYSILRTSLIQAIDRLANFTGWWTDLVIAGVAFVAQGIRNIPRRIRQGLEIALN

SEQ ID NO: 343 - protein, 875 aa, Simian immunodeficiency virus
MRNLIGITLTLIITTLGIGFSQYYATVFYGVPIWKDAKPSLFCASDADVTSRDEHNIWATHNCVPLDPNPYEVPLANMTVDFNMEENYMVQEMKEDLISLFQQSFKPCVKLTPFCVSMSCNLTVPSTTVSPATKVSSTTKEPEIKRLGKNITNYEVYKCSFNQTTEFRDKKKQIYSLFYKEDIMKNNNTNNNNSYYLHNCNTSAITQECAKSTFEPIPIRYCAPAGFAMLKCKDQNFTGKGPCSNVSVVHCTHGIYPMIATALHLNGTLEENKTTAYFADSTHNQPLLVKFHKAVNLTCVRTGNNTRGQVQIGPGMTFYNIENVVGDTRKAFCIVNATEWSNALNESKIAINETLRKYNKTSYGNFTWNTGDPEVSNFWFNCQGEFFYCNLTNWTSPDYIGKNGSIVAQCRLRQIVNHWGIVSKGIYLPPRRGTIKCTSNITGLILTAEGGNETITPTFSSKVEDYWKVELARYKVVEIQPLSVAPRPGKRPEIKANHTRSKRDVGIGLLFLGFLSAAGSTMGAASIALTAQARGLLSGIVQQQQNLLQAIAAQQHLLQLSVWGIKQLQARMLAIEKYIRDQQLLSIWGCANKLVCHSSVPWNITWAGNSTKCNHSDTMDAKYYECIWNNLTWQEWDKLVKNSSETIYSLLEIAQTQQEKNKQDLLELDKWSSLWDWFDITQWLWYIKIAIIIVAGLIGLRILMFIINVVRQVRQGYMPLFSQTPTQAEQDPEQPGGIAEGGGGGGKLRWTPSPAGFFTIVWVDLRNLLLWIYQTCRNFIWILWSILQALKQGIISLVDNLVIIHRSIVVGVRQALEWSGNTYASIRASLIQAIDRLAEFTGWWTDIVIEAVVYIARGIRNIPRRIRQGLEIALN

SEQ ID NO: 344 - protein, 835 aa, Simian immunodeficiency virus; identical to SEQ ID NO: 325

SEQ ID NO: 345 - protein, 844 aa, Simian immunodeficiency virus; identical to SEQ ID NO: 333

SEQ ID NO: 346 - protein, 855 aa, Simian immunodeficiency virus; identical to SEQ ID NO: 326

SEQ ID NO: 347 - protein, 870 aa, Simian immunodeficiency virus
MKNSSGLILTLITTILGIGFSTYYATVFYGVPVWKDATPPLFCASDADVASTQAHNIWATHNCVPLDPHPYEVPLPNVSIEFDMSTNYMVEEMKTDLISLFQQSFKPCVKLTPFCIRMTCANVTEATTTTPPTTSTSTTEPPTSKTTVNTSEIEDYKVLNCSFNQTTEFRDKKKNIYSLFYREDVMKESQNKTNNSEYYYLHNCNTTAITQSCSKSSFEPIPIRYCAPAGYAMLKCNDANFTGAGECTNVSVVHCTHGIMPMIASWLHLNGTYEKEKTKVYHTNVTNNPPLLVKFNETIHITCERTGNNTRGQVQIGPGMTFYNRENIFGDTRKAFCYVNATEWRRTLEMAKTALREATKKPTLNITRPKGDPEVENFWFNCQGEFFYCNLTKWINETWLNETDQTNLVAPCRLRQIVNHWGIVAKAIYLPPRRGTVKCVSNITGFLMTNEETGATFSGKVEDYWRVELAKYKIVEIQPLSVAPTTGKRPEIKANHTRSKRDVGIGLLFLGFLSAAGSTMGAASIALVAQARGVLSGIVQQQHNLLQAIEAQQHLLQLTVWGVKQLQARLLAMEKYIRDQQLLSVWGCANKLVCHSTVVWNDTWAARKPCSNTSGPTDYDCIWQNLTWQQWSELVDNSTDTIYTLLEVAQIQQEKNKKELLELDKWSTLWDWFDITQWLWYIKLAIIIIAGLIGLRILMFIVNVLRQVRQGYMPLFSQIPTQAEQDPEQPGGIAEGGGDRGRIRWTPSPRGFFSIVWEDLRNLLLWIYQTCLNFVWLLGTILQALKQGTISFIQTIIIVHRNLVLKWRLFRDWLANAYVIVKTSFIRNLDRLANFTAWWTDILIEGAVNIFRGIRNIPTRIRQGLEIALN

SEQ ID NO: 348 - protein, 884 aa, Simian immunodeficiency virus
MKMALLIGWILTIIFCCKLGIGTYYTTVYYGVPVWKEATPTLFCASSAKVASTQPHNIWATHNCVPLDPQPYEIPINITTEFNMETNYMVEEMKKDLISLFQQSLKPCVKLTPFCVKMNCSEFTRNSTTGNNATKNNTSLEDKIKSNLTSLEMYECFFNQTTEFRDKKKQIYSLFYKEDIMKDKNDSTNTSYYLINCNTTAITQECEKSSFEPVPIQYCAPPGYAMLKCKDENFRGKGNCTTVRIVHCTHSIFPMIATALHLNGSVEEGETKAYYVEAKHNAPLLIKFNKATVFNITCVRPGNNTRGQVQIGPGMTFYNIRNVIGDTRKAFCYVNKTLWENATEAAKVAINETLKNTTFKNTTYTFRTDGDLKWISGGDQETQTHWFNCQGEFFYCDLKNWTNNGIEIDGMLIAPCRLRQIVNHWGIVSRGIYLPPREGQVKCVSSITGFIMTAETNDTDGTITPTFSAKVEDYWKVEMSRYKVVEIQPLSVAPTKGKRPVVGDPEAKSRSKREIGMGMIFMGFLSAAGSTMGAASIALTVQARGLLSGIVQQQANLLQAIEAQQHLLQLSVWGIKQLQARLLAVEKYLRDQQLLGLWGCANKLICHSSLEWNSSWLNKTLCGNKTNNYENNYECIWSQYTWQQWDEEIRNISDVIYLSLETAQVQQERNHKQLLELDKWSSLWNWFDITNWLWYIKIAIIIVASLVGLRIVMFIINLVGKLRQGYSLLSPQIPIQTARGPGQPEGTEEGGGESGRDRWRGYPVGFLQLIWEDCRQLLLWIYQTSVNLLWHLWTLIKLIHQWSLLLWETVRQLLRRLIHLLTQFLHLLETWGILIRQHCIVCIDTLAEFTGWWTDGVIEALRVAVDIIRHIPTRIRQGLEIALN

SEQ ID NO: 349 - protein, 912 aa, Simian immunodeficiency virus
  VARIANT 61: note = Xaa can be any naturally occurring amino acid
  VARIANT 202: note = Xaa can be any naturally occurring amino acid
  VARIANT 267: note = Xaa can be any naturally occurring amino acid
  VARIANT 304..305: note = Xaa can be any naturally occurring amino acid
  VARIANT 307: note = Xaa can be any naturally occurring amino acid
  VARIANT 310: note = Xaa can be any naturally occurring amino acid
  VARIANT 317: note = Xaa can be any naturally occurring amino acid
MRVMGTKKSCVLYYCLVILSLFSLTIDSKEELWVTVYYGVPVWRDAETTLFCASDAKAQEXEAHNIWASQACVPTDPNPEEIHLPNVTEYFDMWKNNMVEQMQEDIISLWDQSLKPCVKLTPLCVTLNCTNCIWGNGTFREWNDTREDLRNCSFNAGDIRDKKKRVHSLFYAEDIILIDRTRTTKQGNSTQAPATTPIASTXVPDSNDTCSPNNTLEYRLISCNTSVITQACPKTSFEPIPIHYCAPAGFALLKCNDTNFSGQGICXNVSTVHCTHGIRPVVSTHLLLNGSLAKGDIMIRVKBXXQXTDXIIVQLNXSINITCIRPGNNTIKGIPIGPGQHFYGTETVVGDTRKAYCKINGTQWFDTLKRVAWELKETFNLTRVTFDQASPGDPEIQKHYFNCGGEFFYCETSSLFNATIDITPNTTLNETTWDYPAENNINQEMIIHCRIKQIVSMWQRVGKGIFSPPIRGTIQCNSTITGLLLTRDGNNKADSENKTRIETGQNKAEEINRTDIFRPAGGEMRDNWRSELYRYKVVKIEPLGLAPTKAKRRTVNREKRAPGLGLMFLGFLGAAGSTMGAASLTLTVQARQLLTGIVQQQSNLLRAIEAQQHLLQLSVWGIKQLQARVLAIERYLKDQQLLGIWGCSGKLICTTDVKWNETWSNKSYNEIWDNMTWMQWDKEVRNYTETIYDLIEQAQNQQEYNEKSLLELNQWASLWNWFDISNWLWYIKIFIMIVGGLIGCRIVFALLSIVNRVRQGYSPLSFQTLFPIPRGPEQPGETGEEGGEQDNVRSIRLLNGFLPLVWNDLRDLCRFLYHRLRDFLLIARRTLELVGQHLLKGLRLVWETLLYLKGVLQYWGRELKVSAISLLDTTAIAVAEGTDRIIEVAQRFGRGILNIPTRIRQGLERALL

SEQ ID NO: 350 - protein, 889 aa, Simian immunodeficiency virus
  VARIANT 37: note = Xaa can be any naturally occurring amino acid
MKDMTVMKKKKSSLGNWGICLALIIYFNAISYVNCTXYVTVYYGVPVWQDAKTTLFCAADADLASKEQHNIWATQACVPLDPTPIELKLNITESFNIWENYMVEQMQEDIVSLWDQSLKPCVKLTFLCVTMNCSEYKGYNCSTEGNTTAAPCNTTENTTKENSTGMLTCNFNVTTVLKDKKEQKQALFYREDLASLESNNSYRLINCNSSTITQACPKVSFEPLPIQYCAPAGYALMKCNRTDFNGTGTCNETSIVHCTHGIRPTVSTQLVLNGTLAKGKPLVITKNVSETGAPIIVKLSASTAITCIRPGNNTRGEVQLGPMTWYNMRHYIGDIRKAHCTVSRGNWTKVLQNVSAALWEAYPAEWKNKTQDKNHTILFRASSGGDPEVASLHFNCHGEFFYCNTSALFNHSCTKNMTKNEWICTPNNATGTLRLPCRLKQVVNSWMRVGSGLFAPPIPGSLTCKSNITGILLERDLPLGNMTNTTLRPIGGDMKNIWRSELYPYKVVQVKALGVAPTKISRPTIMGPHREKRGAGLGMLFLGFLSAAGSTMGAAAVTLTVQAKQLLHGIVQQQNNLLRAIQAQQELLRLSVWGIRQLRARLLAIETYLKDQQLLGLWGCSGKLVCYTNVPWQKNWTTYQSDSQLDAIWDNLTWQEWDKQVNNYTDLIFLEIQIAQEQQEQNQKKLLELDQWAQLWSWLDITQWLWYIKIFIMIVGGLIGLRIFIAVVNVVKRVRQGYSPLSLQTLGQNRGPAGIATGTDEEGGGAGSVRSIRLLDGFLPLIWDDLKNLVVWIYQILVSCILGIKDLLAILWIHLRHLLNKGLNCVRDCLAVCGYWVQELQRSATNLLDTTAVAVAEWTDQVILIGQRIGRGILNIPRRIRQGLERSLL

SEQ ID NO: 351 - protein, 892 aa, Simian immunodeficiency virus
  VARIANT 248: note = Xaa can be any naturally occurring amino acid
  VARIANT 250: note = Xaa can be any naturally occurring amino acid
  VARIANT 254: note = Xaa can be any naturally occurring amino acid
MKDMRVMRKKKRSLGNWGLSLALLIICFNTGSYVMGTRYVTVYYGVPVWREAKTTLFCAADANLASKEQHNIWATQACVPTDPTPIKVKLNISEPYNIWENYIVKQMQKNIISLLNQSLKPCVKLTVICVTINCSKFKPATTNTTSAPINASLQSSTTAQNNLNFYNCSFNVTTVLKDKKEKKQALFYKENLVPLNQTSKEKLYRLINCNTTTITQACPKVSFKPLPIQYCAPAKYALIKCKQKGFNXTXLCNXTVITHCTHGIRPTVSTQLIFNGSLAEEEALVMSKDVTATGKNIIIKLSTGVNITCIRTGNNTRGQTQIGPMTWYNSVNYIGNIRRAYCQVEETDWKGILKNVSKALIKRYNGKVNETHTFEFKNASGGDPEVTHLHFNCHGEFFYCNTSKLFNYTYVCNQTDQGNANYSCHSTSTLENGTLVIPCKLRQVVNSWMRVGSGLFAPPVPGSLTCHSNITGLILQRDWPLNNNTTTILRPEGGDMKDIWRSELYPYKVVQVKALAVAPTKISRPTIMAQTHQREKRGAGLGMLFLGFMSAAGSTMGAAAVTLTVQAKQLLHGIVQQQNNLLRAIEAQQELLRLSVWGIRQLRARLLAIETYLRDQQLLGLWGCTGQIVCYTNVPWNQTWTGRNDTELDSIWNKLTWQEWDKLVDNYTDTIFVEIQKANEQQKENEKKLLELDQWTQLWSWFDITKWLWYIKIFIMIVGGLIGLKFLLAIINIVKRVRQGYSPLSLQILGLNRGLAGIAPGTDEEGGEIDNGRSIRLLDGLLPIVWDDLKNLVVWIYRILATCILGTKDLLNILWIHLRHLLNKGINCLRDCLAVCGYWAQELQQSATSLLDTVAVRVADWTDQVILVGQRIGRGILNIPRRLRQGLERSLL

SEQ ID NO: 352 - protein, 884 aa, Simian immunodeficiency virus
  VARIANT 15: note = Xaa can be any naturally occurring amino acid
  VARIANT 128: note = Xaa can be any naturally occurring amino acid
  VARIANT 130: note = Xaa can be any naturally occurring amino acid
  VARIANT 150: note = Xaa can be any naturally occurring amino acid
  VARIANT 202: note = Xaa can be any naturally occurring amino acid
  VARIANT 473: note = Xaa can be any naturally occurring amino acid
  VARIANT 621: note = Xaa can be any naturally occurring amino acid
  VARIANT 661: note = Xaa can be any naturally occurring amino acid
  VARIANT 711: note = Xaa can be any naturally occurring amino acid
  VARIANT 839: note = Xaa can be any naturally occurring amino acid
MKVMKKKKEKSWILXIVMAFIIPCSSSRPLYATVYYGVPVWRDAQTTLFCAADAKMASSEMHNVWATQACVPTDPQPIELKLTNVTETFNIWKSGMVEQMQEDIISLWDQSLKPCVKLTVMCVTMNCXRXTPNTTTHSLNMTGPAPRTSXPTTSSHNMTSTPNIPELEVYNCTFNVTTVLKDKKSQQQALFYREDLSQIGDXNSTYRLINCNTSTISQACPKVSFEPLPIQYCAPAGYALMKCNQSKFNGTGTCNETLITHCTHGIRPTVSTQFIFNGTLEKELLMLSKNISDSGKTIIVKLKKAVTFKCERTGNNTRGQIQIGPMTIYNSENIVGKTRKAYCAYNRTEWEQALKTISEAFAKLENVTKVQWKNSSGGDLEVAMLHFNCHGEFFYCNTTTMFNYTYNCSHHSCKAQSKNISEGSGDSYIPCKLKQVVNSWMRVGSGLFAPPIRGTLKCMSNITGLLLERDVPXNITATBRNITLRPTGGEMKDIWRSELYPYKVVQVKALSVAPTKIKRPIIGLNREKRGAGLGMLFLGFMSAAGSTMGAASLTLTVQAKQLLHGIVQQQNNMLRAIAAQQELLRLSVWGIRQLRARLLAIETYLRDQQLLGLWGCAGKLXCYTNVPWNNTWTNKSBTELEHIWENLTWQEWDKLVDNYTXDIFLKIQZANTQQEVNEKKLLELDKWADLWSWFDITKWLWYIKIAIMIVXALIGLRVVMVVLSLVKNIRKGYQPLSLQTPSPHPTEPGTPEKTGGEGGEGDKHKWTPLPPGFLHLLYLDLRTIILWSYHLLSSLVSRIQRILSLLGLGLRILGQKTFEACKTLKATAQYWLQELQRSXTNLLDTVAVAVANWTDSIILGVQRFGRGILNIPRRIRQGLELSLL

SEQ ID NO: 353 - protein, 858 aa, Simian immunodeficiency virus
  VARIANT 99: note = Xaa can be any naturally occurring amino acid
  VARIANT 482: note = Xaa can be any naturally occurring amino acid
  VARIANT 538: note = Xaa can be any naturally occurring amino acid
MRKPIHIIWGLALLIQFIEKGTNEDYVTVFYGVPVWRNATPTLFCATNASMTSTEVHNVWATTSCVPIDPDPIVVRLNTSVWFNAYKNYMVESMTEDMXQLFQQSHKPCVKLTPMCIKMNCTGYNGTPTTPSTTTSTVTPKTTTPIVDGMKLQECNFNQSTGFKDKKQKMKAIFYKGDLMKCQDNNETNCYYLWHCNTTTITQSCEKSTFEPIPIHYCAPAGYAILRCEDEDFTGVGMCKNVSVVHCTHGISPMVATWLLLNGTYQTNTSVVMNGRKNESVLVRFGKEFENLTITCIRPGNRTVRNLQIGPGMTFYNVEIATGDTRKAFCTVNKTLWEQARNKTEHVLAEHWKKVDNKTNAKTIWTFQDGDPEVKVHWFNCQGEFFYCDITPWFNATYTGNLITNGALIAHCRIKQIVNHWGIVSKGIYLAPRRGNVSCTSSITGIMLEGQIYNETVKVSPAARVADQWRAELSRYQVVEIXPLSVAPTTGKRPEIKQHSRQKRGIGIGLFFLGLLSAAGSTMGAASIALTAQTRNLXHGIVQQQANLLQAIETQQHLLQLSVWGVKQLQARMLAVEKYLRDQQLLSLWGCADKVTCHTTVPWNNSWVNFTQTCAKNSSDIQCIWENMTWQEWDRLVQNSTGQIYNILQIAHEQQERNKKELYELDKWSSLWNWFDITQWLWYIKIFIMIVGAIVGLRILLVLVSCLRKVRQGYHPLSFQIPTQNQQDPEQPEEIREEGGRKDRIRWRALQHGFFALLWVDLTSIIQWIYQICRTCLLNLWAVLQHLCRITFRLCNHLENNLSTLWTIIRTEIIKNIDRLAIWVGEKTDSILLALQTIVRIIREVPRRIRQGLEIALN

SEQ ID NO: 354 - protein, 854 aa, Simian immunodeficiency virus
MKVMEKKKRDWNSLSIITIITIILLTPCLTSELWVTVYYGVPVWHDADPVLFCASDAKAHSTEAHNIWATQACVPTDPSPQEVFLPNVIESFNMWKNNMVDQMHEDIISLWDQSLKPCVKLTPLCVTLQCSKANFSQAKNLTNQTSSPPLEMKNCSFNVTTELRDKKKQVYSLFYVEDVVNLGNENNTYRIINCNTTAITQACPKTSFEPIPIHYCAPAGFAILKCNDKDFSGKGKCTNVSTVHCTHGIKPVVTTQLLINGSLAEGNITVRVENKSKNTDVWIVQLVEAVSLNCHRPGNNTRGEVQIGPGMTFYNIENVVGDTRSAYCKINGTTWNRTVEEVKKALATSSNRTAANITLNRASGGDPEVTHHMFNCGGEFFYCNTSQIFTDNITNGIIILPCRIRQIVSSWMRVGRGIYAPPIRGNITCNSNITGLLLTSDTPVTNNSGNLTFRPTGGNMKDIWRSELYKYKVVRIEPLSVAPTKARRHTVARQKDRQKRAAFGLGALFLGFLGAAGSTMGAAAVTLTVQARQLLSGIVQQQNNLLKAIEAQQHLLQLSIWGVKQLQARLLAVERYLQDQQILGLWGCSGKAVCYTTVPWNNSWPGSNSTDDIWGNLTWQQWDKLVSNYTGKIFGLLEEAQSQQEKNERDLLELDQWASLWNWFDITKWLWYIKIFLMAVGGIIGLRIIMTVFSVVRRVRQGYSPLSLQTLIPVQREQGRLGEIDEGGGEQDRSRSVRLVEGCLPLIWDDLRNLGIWSYQSLTSLACNVWRQLKTLGHLILHSLRLLRERLCLLGGIIQYWGKELKISAISLLDATAIAVAEGTDRIIEAFQVTLRIIRNIPRRIRQGLERALL

SEQ ID NO: 355 - protein, 6 aa, synthetic construct
  REGION 1..6: note = C K-rich region
RDKKQK

SEQ ID NO: 356 - protein, 4 aa, synthetic construct
  REGION 1..4: note = V3 loop protein backbone residues
GDIR

SEQ ID NO: 357 - protein, 5 aa, synthetic construct
  REGION 1..5: note = Linker
GGGGS

SEQ ID NO: 358 - protein, 4 aa, synthetic construct
  REGION 1..4: note = Cleavage site
REKR

SEQ ID NO: 359 - protein, 6 aa, synthetic construct
  REGION 1..6: note = Furin inducible R6 site
RRRRRR